(19) United States
(12) Patent Application Publication
(10) Pub. No.: US 2023/0181722 A1
(43) Pub. Date: Jun. 15, 2023

(54) CORONAVIRUS ANTIGEN COMPOSITIONS AND THEIR USES

(71) Applicant: Flagship Pioneering Innovations VI, LLC, Cambridge, MA (US)

(72) Inventors: Avak KAHVEJIAN, Lexington, MA (US); Alexandra Sophie DE BOER, Somerville, MA (US); Yann Paul Guy Régis ECHELARD, Jamaica Plain, MA (US); Nicholas McCartney PLUGIS, Duxbury, MA (US); Roger Joseph HAJJAR, Lexington, MA (US); Michael Donato MELFI, Boston, MA (US); Jennifer A. NELSON, Brookline, MA (US)

(21) Appl. No.: 17/925,920

(22) PCT Filed: May 20, 2021
(86) PCT No.: PCT/US2021/033490

§ 371 Date: Nov. 17, 2022

Related U.S. Application Data
(60) Provisional application No. 63/125,778, filed on Dec. 15, 2020.
(60) Provisional application No. 63/074,442, filed on Sep. 3, 2020.
(60) Provisional application No. 63/068,936, filed on Aug. 21, 2020.
(60) Provisional application No. 63/027,932, filed on May 20, 2020.

(51) Int. Cl. A61K 39/215; A61K 39/09; A61P 31/14

(52) U.S. Cl. CPC A61K 39/215; A61K 39/092; A61P 31/14

(57) ABSTRACT

The disclosure provides compositions and methods comprising circular polyribonucleotides comprising a sequence encoding a coronavirus antigen, and compositions and methods comprising linear polyribonucleotides comprising a sequence encoding a coronavirus antigen. Compositions and methods are provided that are related to generating polyclonal antibodies, for example, using the disclosed circular polyribonucleotides or the disclosed linear polyribonucleotides.

### SEQUENCE LISTING

The instant application contains a Sequence Listing which has been submitted electronically in ASCII format and is hereby incorporated by reference in its entirety. Said ASCII copy, created on May 20, 2021 is named 51509-020WO6_Sequence_Listing_5.20.21_ST25 and is 207,385 bytes in size.

### BACKGROUND

There is an urgent need for vaccines and therapeutics that are active against coronaviruses.

### SUMMARY

The disclosure generally relates to circular polyribonucleotides comprising a sequence encoding a coronavirus antigen and to immunogenic compositions comprising the circular polyribonucleotide. This disclosure further relates to methods of using the circular polyribonucleotides comprising a sequence encoding a coronavirus antigen and the immunogenic composition. In some embodiments, the circular polyribonucleotides and immunogenic compositions of this disclosure are used in methods of generating polyclonal antibodies. The produced polyclonal antibodies can be used in methods of prophylaxis in subjects (e.g., human subjects) or methods of treatment for subjects (e.g., human subjects) having a coronavirus infection. The produced polyclonal antibodies can be administered to subjects at high risk for exposure to coronavirus infection.

The disclosure also relates to linear polyribonucleotides comprising a sequence encoding a sequence of SEQ ID NO selected from TABLE 3 and to immunogenic compositions comprising the linear polyribonucleotide. This disclosure further relates to methods of using the linear polyribonucleotide comprising a sequence encoding a coronavirus antigen and the immunogenic composition comprising the linear polyribonucleotide. In some embodiments, the linear polyribonucleotides and immunogenic compositions of this disclosure are used in methods of generating polyclonal antibodies. The produced polyclonal antibodies can be used in methods of prophylaxis in subjects (e.g., human subjects for treatment) or methods of treatment for subjects (e.g., human subjects for treatment) having a coronavirus infection. The produced polyclonal antibodies can be administered to subjects for treatment at high risk for exposure to coronavirus infection.

In one aspect, the invention features a composition (e.g., an immunogenic composition) comprising (a) a circular polyribonucleotide comprising a sequence encoding a coronavirus antigen, e.g., a sequence selected from a SEQ ID NO. in TABLE 1 or TABLE 2, or (b) a linear polyribonucleotide comprising a sequence selected from a SEQ ID NO. in TABLE 3.

In one embodiment, the composition further comprises plasma from a non-human animal (e.g., a non-human animal comprising a humanized immune system) or a human subject (e.g., after immunization of a subject for immunization).

In one embodiment, the composition further comprises plasma from a non-human animal (e.g., a non-human animal comprising a humanized immune system) and the coronavirus antigen (e.g., after immunization of a non-human animal subject for immunization). In one embodiment, the composition further comprises plasma from a human subject (e.g., after immunization of a human subject for immunization) and the coronavirus antigen.

In some embodiments, the composition further comprises a non-human B cell comprising a humanized immunoglobulin gene locus and a humanized B cell receptor, wherein the humanized B cell receptor binds to the coronavirus antigen. In some embodiments, the composition or immunogenic composition further comprises a plurality of non-human B cells, wherein a non-human B cell of the plurality comprises a humanized immunoglobulin gene locus, wherein the plurality of non-human B cells comprises a first B cell that binds to a first epitope of the coronavirus antigen and a second B cell that binds to a second epitope of the coronavirus antigen.

In some embodiments, the coronavirus antigen is from a betacoronavirus or a fragment thereof or a sarbecovirus or a fragment thereof. In some embodiments, the coronavirus antigen is from severe acute respiratory syndrome (SARS)-related coronavirus or a fragment thereof. In some embodiments, the coronavirus antigen is from severe acute respiratory syndrome coronavirus 2 (SARS-CoV-2) or a fragment thereof, severe acute respiratory syndrome coronavirus 1 (SARS-CoV-1) or a fragment thereof, or Middle East respiratory syndrome coronavirus (MERS-CoV) or a fragment thereof.

In some embodiments, the coronavirus antigen is a membrane protein or a variant or fragment thereof, an envelope protein of a virus or a variant or fragment thereof, a spike protein of a virus or a variant or fragment thereof, a nucleocapsid protein of a virus or a variant or fragment thereof, an accessory protein of a virus or a variant or fragment thereof. In some embodiments, the coronavirus antigen is a receptor binding domain of spike protein or a variant or fragment thereof. In some embodiments, the spike protein lacks a cleavage site. In some embodiments, an accessory protein of a coronavirus is selected from a group consisting of ORF3a, ORF7a, ORF7b, ORF8, ORF10, or any variant or fragment thereof. In some embodiments, the coronavirus antigen comprises a sequence having at least about 80%, 85%, 90%, 95%, 97%, 98%, 99%, or 100% sequence identity to a sequence selected from TABLE 1 or a sequence of a SEQ ID NO. selected from TABLE 2. In some embodiments, the circular polyribonucleotide comprises a sequence having at least about 80%, 85%, 90%, 95%, 97%, 98%, 99%, or 100% sequence identity to a sequence of a SEQ ID NO. selected from TABLE 2.

In some embodiments, the polyribonucleotide comprises a plurality of sequences, each encoding an antigen, and at least one sequence of the plurality encodes a coronavirus antigen. In some embodiments, the circular polyribonucleotide comprises two or more ORFs. In some embodiments, the circular polyribonucleotide comprises at least five sequences, each encoding an antigen and at least one of the antigens is a coronavirus antigen. In some embodiments, the circular polyribonucleotide comprises at least two ORFs, e.g., at least 2, 3, 4 or 5 ORFs. In some embodiments, the circular polyribonucleotide comprises between 5 and 20 sequences, each encoding an antigen and at least one of the antigens is a coronavirus antigen. In some embodiments, the circular polyribonucleotide comprises between 5 and 10 sequences, each encoding an antigen and at least one of the antigens is a coronavirus antigen. In some embodiments, the circular polyribonucleotide comprises sequences encoding antigens from at least two different microorganisms, and at least one microorganism is a coronavirus. In some embodiments, the linear polyribonucleotide comprises sequences encoding two or more antigens and at least one antigen is a coronavirus antigen encoded by a sequence of a SEQ ID NO. in TABLE 3. In some embodiments, the linear polyribonucleotide comprises sequences encoding at least 2, 3, 4 or 5 antigens and at least one antigen is a coronavirus antigen encoded by a sequence of a SEQ ID NO. in TABLE 3. In some embodiments, the coronavirus antigen comprises an epitope. In some embodiments, the coronavirus antigen comprises an epitope recognized by a B cell. In some embodiments, the coronavirus antigen comprises at least two epitopes.

In some embodiments, the composition or immunogenic composition comprising a circular polyribonucleotide further comprises a second circular polyribonucleotide comprising a sequence encoding a second antigen. In some embodiments, the composition or immunogenic composition further comprises a second circular polyribonucleotide comprising a second ORF. In some embodiments, the composition or immunogenic composition further comprises a third, fourth, or fifth circular polyribonucleotide comprising a sequence encoding a third, fourth, or fifth antigen. In some embodiments, the composition or immunogenic composition further comprises a second linear polyribonucleotide comprising a sequence encoding a second antigen. In some embodiments, the composition or immunogenic composition comprising the linear polyribonucleotide further comprises a second linear polyribonucleotide comprising a second ORF. In some embodiments, the composition or immunogenic composition further comprises a third, fourth, or fifth linear polyribonucleotide comprising a sequence encoding a third, fourth, or fifth antigen. In some embodiments, the first antigen, second antigen, third antigen, fourth antigen, and fifth antigen are different antigens.

In some embodiments, the composition or immunogenic composition further comprises a pharmaceutically acceptable carrier or excipient. In some embodiments, the polyribonucleotide is administered without a carrier (“naked”). In other embodiments, the polyribonucleotide is formulated with a carrier, e.g., an LNP, VLP, liposome, or the like.

In some embodiments, the composition further comprises an adjuvant. In some embodiments, the composition or immunogenic composition further comprises a diluent. In some embodiments, the composition or immunogenic composition further comprises protamine.

In another aspect, the invention features methods including (a) administering a composition described herein (e.g., a composition comprising (i) a circular polyribonucleotide comprising a sequence encoding a coronavirus antigen, e.g., a sequence selected from a SEQ ID NO. in TABLE 1, 2 or 3, or (ii) a linear polyribonucleotide comprising a sequence selected from a SEQ ID NO. in TABLE 3) to a non-human animal or to a human subject (e.g., to induce an immune response against the antigen or to produce polyclonal antibodies against the antigen in the non-human animal or human subject for immunization) and (b) optionally, collecting antibodies against the antigen from the non-human animal or the human subject (e.g., the non-human animal or human subject for immunization).

In some embodiments, the method further comprises administering an adjuvant (e.g., Addavax™ adjuvant, MF59, AS03, complete Freund's adjuvant) to the non-human animal or to the human subject (e.g., the non-human animal or human subject for immunization). The adjuvant may be co-formulated and co-administered with the polyribonucleotide, or it may be formulated and administered separately.

In some embodiments, the method further comprises pre-administering (priming) the non-human animal or human subject (e.g., the non-human animal or human subject for immunization) with an agent, e.g., antigen, to improve immunogenic response. For example, the method includes administering the protein antigen to the non-human animal or human subject (e.g., the non-human animal or human subject for immunization) prior (e.g., from 1-7 days, e.g., 1, 2, 3, 4, 5, 6, 7 days prior) to administration of the polyribonucleotide comprising a sequence encoding the antigen. The protein antigen may be administered as a protein preparation, or encoded in a plasmid (pDNA), or presented in a virus-like-particle (VLP), formulated in a lipid nanoparticle (LNP), or the like.

In some embodiments, the method further comprises administering or immunizing the subject (e.g., the subject for immunization) with protamine.

In some embodiments, the polyribonucleotide is administered without a carrier (“naked”). In other embodiments, the polyribonucleotide is formulated with a carrier, e.g., an LNP, VLP, liposome, or the like.

In some embodiments, the method further comprises administering or immunizing the subject (e.g., the subject for immunization) with the polyribonucleotide (e.g., the circular or linear polyribonucleotide) at least two times, e.g., 2, 3, 4, 5 times.

In some embodiments, the method further comprises collecting plasma from the subject (e.g., after immunization of the subject for immunization). In some embodiments, the method further comprises purifying polyclonal antibodies from the subject (e.g., after immunization of the subject for immunization). In some embodiments, the method further comprises administering or immunizing the subject (e.g., the subject for immunization) with a vaccine. In some embodiments, the vaccine is pneumococcal polysaccharide vaccine (e.g., PCV13 or PPSV23). In some embodiments, the vaccine is for a bacterial infection. In some embodiments, the subject (e.g., the subject for immunization) is immunized with the circular RNA by injection. In some embodiments, the subject (e.g., the subject for immunization) is immunized with the linear RNA by injection.

In embodiments, the subject is a human subject (e.g., the human subject for immunization). In some embodiments, the human subject (e.g., the human subject for immunization) is a subject at risk for a coronavirus-related disease, e.g., a human over 50 years old; an immune-compromised human; a human with a chronic health condition such as obesity, diabetes, cancer; a health care worker.

In embodiments, the subject is a non-human animal (e.g., the non-human animal for immunization). In some embodiments, the non-human animal (e.g., the non-human animal for immunization) is an agricultural animal, e.g., a cow, pig, sheep, horse, goat; a pet, e.g., a cat or dog; or a zoo animal, e.g., a feline.

In some embodiments, the non-human animal (e.g., the non-human animal for immunization) is a mammal, e.g., a rodent (e.g., a rabbit, rat or mouse), or an ungulate, e.g., a pig, cow, goat, or sheep. In some embodiments, the non-human animal (e.g., the non-human animal for immunization) is a transchromosomal non-human animal comprising a humanized immunoglobulin gene locus. In some embodiments, the non-human animal is a transchromosomal cow comprising a human artificial chromosome (HAC) vector that comprises the humanized immunoglobulin gene locus. In some embodiments, the humanized immunoglobulin gene locus encodes an immunoglobulin heavy chain. In some embodiments, the humanized immunoglobulin heavy chain comprises an IgG isotype heavy chain. In some embodiments, the humanized immunoglobulin heavy chain comprises an IgG1, IgG2, IgG3, or IgG4 isotype heavy chain.

In some embodiments, the non-human animal (e.g., the non-human animal for immunization) comprises a B cell having a B cell receptor, the B cell receptor binds to the coronavirus antigen. In some embodiments, the non-human animal comprises a plurality of B cells comprising a first B cell that binds to a first epitope of the coronavirus antigen and a second B cell that binds to a second epitope of the coronavirus antigen.

In some embodiments, the non-human animal (e.g., the non-human animal for immunization) comprises a T cell, wherein the T cell comprises a T Cell Receptor that binds to the coronavirus antigen. In some embodiments, upon activation, the T cell enhances production of an antibody that that binds to the antigen. In some embodiments, upon activation, the T cell enhances antibody production by a B cell that binds to the coronavirus antigen. In some embodiments, upon activation, the T cell enhances survival, proliferation, plasma cell differentiation, somatic hypermutation, immunoglobulin class switching, or a combination thereof of a B cell that that binds to the coronavirus antigen.

In some embodiments, the non-human animal or human subject (e.g., the non-human animal or human subject for immunization) produces an antibody that specifically binds to the coronavirus antigen. In some embodiments, the antibody is a humanized antibody or a fully human antibody. In some embodiments, the antibody is antibody an IgG, IgA, or IgM isotype antibody. In some embodiments, the antibody is an IgG1, IgG2, IgG3, or IgG4 isotype antibody. In some embodiments, the non-human animal (e.g., the non-human animal for immunization) comprises plurality of polyclonal antibodies that specifically bind at least two epitopes that are encoded by the circular polyribonucleotide. In some embodiments, the non-human animal or human subject (e.g., the non-human animal or human subject for immunization) comprises plurality of polyclonal antibodies that specifically bind at least two epitopes that are encoded by the linear RNA. In some embodiments, the plurality of antibodies comprises humanized antibodies. In some embodiments, the plurality of polyclonal antibodies comprises fully human antibodies. In some embodiments, the plurality of polyclonal antibodies comprise IgG antibodies, IgG1 antibodies, IgG2 antibodies, IgG3 antibodies, IgG4 antibodies, IgM antibodies, IgA antibodies, or a combination thereof. In some embodiments, the immunoglobulin heavy chain comprises an IgM or IgA isotype heavy chain. In some embodiments, the humanized immunoglobulin gene locus encodes an immunoglobulin light chain. In some embodiments, the immunoglobulin light chain comprises a kappa light chain or a lambda light chain.

In some embodiments, the method further includes collecting blood from the non-human animal or human subject (e.g., after immunizing the non-human animal or human subject for immunization), and purifying antibodies against the antigen from the blood.

In another aspect, the invention features an anti-coronavirus antibody preparation (e.g., a polyclonal antibody preparation) produced by (a) administering a composition comprising a polyribonucleotide described herein to a non-human animal described herein (e.g., a cow having a humanized immune system as described herein) or to a human subject (e.g., the non-human animal or human subject for immunization), and (b) collecting antibodies against the antigen from the non-human animal or human subject (e.g., after immunizing the non-human animal or human subject for immunization).

In embodiments, the polyribonucleotide is (a) a circular polyribonucleotide comprising a sequence encoding a coronavirus antigen, e.g., a sequence selected from a SEQ ID NO. in TABLE 1, 2 or 3, or (b) a linear polyribonucleotide comprising a sequence selected from a SEQ ID NO. in TABLE 3.

In embodiments, the antibody preparation is formulated as a pharmaceutical composition.

In another aspect, the invention features a method of delivering antibodies against a coronavirus to a subject (e.g., the subject for treatment) having a coronavirus infection, at risk of exposure to a coronavirus infection, or in need thereof, e.g., a method of preventing or treating the subject (e.g., the subject for treatment) for a coronavirus infection. The method includes administering to the subject (e.g., the subject for treatment) having a coronavirus infection, at risk of exposure to a coronavirus infection, or in need thereof polyclonal antibodies produced from an animal (e.g., a mammal) having a human or humanized immune system, that has been immunized with a polyribonucleotide described herein, e.g., (a) a circular polyribonucleotide comprising a sequence encoding a coronavirus antigen, e.g., a sequence selected from a SEQ ID NO. in TABLE 1, 2 or 3, or (b) a linear polyribonucleotide comprising a sequence selected from a SEQ ID NO. in TABLE 3.

In certain embodiments, the method further includes one or more of: immunizing a non-human animal (e.g., a non-human animal for immunization) that has been genetically modified to produce human antibodies with a polyribonucleotide disclosed herein, collecting blood from the non-human animal, purifying antibodies from the non-human animal, formulating the antibodies for pharmaceutical use, and administering the formulated antibodies to the human subject (e.g., the human subject for treatment).

In some embodiments, the mammal having a human or humanized immune system is a human (e.g., a human subject for immunization).

In some embodiments, the mammal having a human or humanized immune system is a non-human animal that has been genetically modified to produce human antibodies, e.g. a non-human animal comprising a humanized immunoglobulin gene locus, e.g., a transchromosomal cow comprising a human artificial chromosome (HAC) vector that comprises a human immunoglobulin gene locus.

In embodiments, the subject (e.g., the subject for treatment) having a coronavirus infection or in need thereof is a human subject diagnosed with a coronavirus-related disease, e.g., Covid-19, SARS, MERS. In some embodiments, the subject (e.g., the subject for treatment) at risk of exposure to a coronavirus infection or in need thereof is a subject at risk for a coronavirus-related disease, e.g., a human over 50 years old; an immune-compromised human; a human with a chronic health condition such as obesity, diabetes, cancer; a health care worker.

In some embodiments, the administration or immunization is before, after, or simultaneously with risk of exposure to the coronavirus.

In some embodiments, the method further comprises monitoring the human subject (e.g., the subject for treatment) for the presence of antibodies to coronavirus, e.g., before and/or after administration.

Exemplary embodiments of the invention are described in the enumerated paragraphs below.

E1. An immunogenic composition comprising:

a) a circular polyribonucleotide comprising a sequence encoding a coronavirus antigen; or b) a linear polyribonucleotide comprising a sequence selected from any one of SEQ ID NOs: 13, 15, and 12.

E2. A immunogenic composition comprising a circular polyribonucleotide comprising a sequence encoding a coronavirus antigen, wherein the coronavirus antigen comprises a sequence having at least about 80%, 85%, 90%, 95%, 97%, 98%, 99%, or 100% sequence identity to a coronavirus antigen selected from any one of SEQ ID NOs: 1-10, 13, 15, 17 19, 21, 23, 25-30, 48, and 49, or the circular polyribonucleotide comprises a sequence having at least about 80%, 85%, 90%, 95%, 97%, 98%, 99%, or 100% sequence identity to a circular polyribonucleotide selected from SEQ ID NOs: 12, 14, 16, 18, 20, 22, and 24.

E3. The immunogenic composition of any one of the preceding embodiments, further comprising plasma from a non-human animal (e.g., a non-human animal comprising a humanized immune system; e.g., a non-human animal for immunization) or a human subject (e.g., a human subject for immunization).

E4. The immunogenic composition of any one of the preceding embodiments, further comprising the coronavirus antigen.

E5. The immunogenic composition of any one of the preceding embodiments, wherein the composition further comprises a non-human B cell comprising a humanized immunoglobulin gene locus and a humanized B cell receptor, wherein the humanized B cell receptor binds to the coronavirus antigen.

E6. The immunogenic composition of any one of the preceding embodiments, wherein the composition further comprises a plurality of non-human B cells, wherein a non-human B cell of the plurality comprises a humanized immunoglobulin gene locus, wherein the plurality of B cells comprises a first B cell that binds to a first epitope of the coronavirus antigen and a second B cell that binds to a second epitope of the coronavirus antigen.

E7. The immunogenic composition of any one of the preceding embodiments, wherein the coronavirus antigen is from a betacoronavirus or a fragment thereof or a sarbecovirus or a fragment thereof.

E8. The immunogenic composition of any one of the preceding embodiments, wherein the coronavirus antigen is from severe acute respiratory syndrome-related coronavirus or a fragment thereof.

E9. The immunogenic composition of any one of the preceding embodiments, wherein the coronavirus antigen is from severe acute respiratory syndrome (SARS)-related coronavirus or a fragment thereof.

E10. The immunogenic composition of any one of the preceding embodiments, wherein the coronavirus antigen is from severe acute respiratory syndrome coronavirus 2 (SARS-CoV-2) or a fragment thereof, severe acute respiratory syndrome coronavirus 1 (SARS-CoV-1) or a fragment thereof, or Middle East respiratory syndrome coronavirus (MERS-CoV) or a fragment thereof.

E11. The immunogenic composition of any one of the preceding embodiments, wherein the coronavirus antigen is a membrane protein or a variant or fragment thereof, an envelope protein of a virus or a variant or fragment thereof, a spike protein of a virus or a variant or fragment thereof, a nucleocapsid protein of a virus or a variant or fragment thereof, an accessory protein of a virus or a variant or fragment thereof.

E12. The immunogenic composition of any one of the preceding embodiments wherein the coronavirus antigen is a receptor binding domain of spike protein or a variant or fragment thereof.

E13. The immunogenic composition of embodiment 8, wherein the spike protein lacks a cleavage site.

E14. The immunogenic composition of any one of the preceding embodiments, wherein an accessory protein of a coronavirus is selected from a group consisting of ORF3a, ORF7a, ORF7b, ORF8, ORF10, or any variant or fragment thereof.

E15. The immunogenic composition of any one of the preceding embodiments, wherein the circular polyribonucleotide comprises a plurality of sequences, each encoding an antigen, and at least one sequence encodes a coronavirus antigen.

E16. The immunogenic composition of any one of the preceding embodiments, wherein the circular polyribonucleotide comprises two or more ORFs.

E17. The immunogenic composition of any one of the preceding embodiments, wherein the circular polyribonucleotide comprises at least five sequences, each encoding an antigen, and at least one antigen is a coronavirus antigen.

E18. The immunogenic composition of any one of the preceding embodiments, wherein the circular polyribonucleotide comprises at least two ORFs (e.g., at least 2, 3, 4, or 5).

E19. The immunogenic composition of any one of the preceding embodiments, wherein the circular polyribonucleotide comprises sequences encoding antigens from at least two different microorganisms, and at least one microorganism is a coronavirus.

E20. The immunogenic composition of any one of the preceding embodiments, wherein the linear polyribonucleotide comprises sequences encoding two or more antigens and at least one antigen is the coronavirus antigen.

E21. The immunogenic composition of any one of the preceding embodiments, wherein the linear polyribonucleotide comprises sequences encoding at least 2, 3, 4, or 5 antigens and at least one antigen is a coronavirus antigen encoded by a sequence of SEQ ID NO. in TABLE 3.

E22. The immunogenic composition of any one of the preceding embodiments, wherein the coronavirus antigen comprises an epitope.

E23. The immunogenic composition of any one of the preceding embodiments, wherein the coronavirus antigen comprises an epitope recognized by a B cell.

E24. The immunogenic composition of any one of the preceding embodiments, wherein the coronavirus antigen comprises at least two epitopes.

E25. The immunogenic composition of any one of the preceding embodiments, further comprising a second circular polyribonucleotide comprising a sequence encoding a second antigen.

E26. The immunogenic composition of any one of the preceding embodiments, further comprising a second circular polyribonucleotide comprising a second ORF.

E27. The immunogenic composition of any one of the preceding embodiments, further comprising a third, fourth, or fifth circular polyribonucleotide comprising a sequence encoding a third, fourth, or fifth antigen.

E28. The immunogenic composition of any one of the preceding embodiments, further comprising a second linear polyribonucleotide comprising a sequence encoding a second antigen.

E29. The immunogenic composition of any one of the preceding embodiments, further comprising a second linear polyribonucleotide comprising a second ORF.

E30. The immunogenic composition of any one of the preceding embodiments, further comprising a third, fourth, or fifth linear polyribonucleotide comprising a sequence encoding a third, fourth, or fifth antigen.

E31. The immunogenic composition of any one of the preceding embodiments, wherein the first antigen, second antigen, third antigen, fourth antigen, and fifth antigen are different antigens.

E32. The immunogenic composition of any one of the preceding embodiments, wherein the immunogenic composition further comprises a pharmaceutically acceptable carrier or excipient.

E33. The immunogenic composition of any one of the preceding embodiments, wherein the immunogenic composition further comprises a pharmaceutically acceptable excipient and is free of any carrier.

E34. The immunogenic composition of any one of the preceding embodiments, wherein the circular polyribonucleotide, linear polyribonucleotide, or immunogenic composition is formulated with a carrier (e.g., a lipid nanoparticle, virus-like particle, or a liposome).

E35. The immunogenic composition of any one of the preceding embodiments, wherein the immunogenic composition further comprises an adjuvant.

E36. The immunogenic composition of embodiment 35, wherein the adjuvant is a saponin or an oil emulsion.

E37. The immunogenic composition of embodiment 36, wherein the oil emulsion is a squalene-water emulsion (e.g., Addavax™ adjuvant, MF59 or AS03).

E38. The immunogenic composition of any one of the preceding embodiments, wherein the immunogenic composition further comprises a diluent.

E40. A lipid nanoparticle (LNP) comprising the immunogenic composition of any one of the preceding embodiments.

E41. The LNP of embodiment 40, comprising an ionizable lipid.

E42. The LNP of embodiment 40, comprising a cationic lipid.

E43. The LNP of embodiment 42, wherein the cationic lipid has a structure according to:

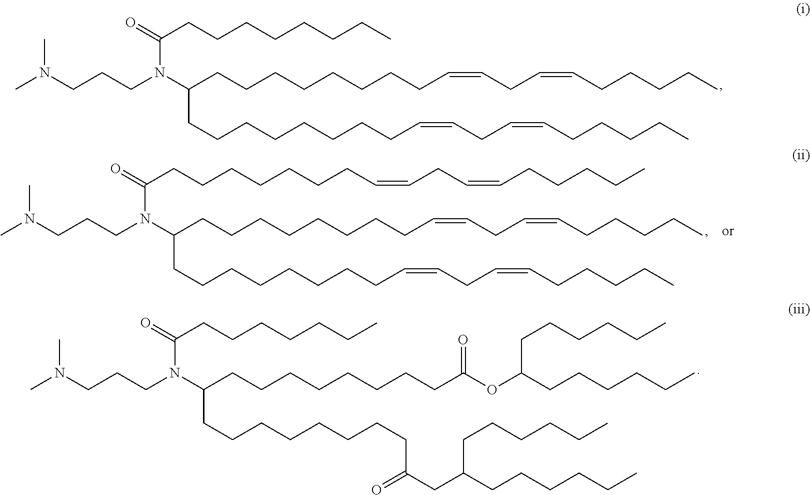

E44. The LNP of any one of embodiments 40 to 43, further comprising one or more neutral lipids, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

E45. A method of delivering an immunogenic composition to a non-human animal or human subject (e.g., a non-human animal or human subject for immunization) comprising: a) administering the immunogenic composition of any one of the preceding embodiments to the non-human animal or human subject, and b) optionally, collecting antibodies against the coronavirus antigen from the non-human animal or human subject.

E46. A method of inducing an immune response against a coronavirus antigen in a non-human animal or human subject (e.g., a non-human animal or human subject for immunization) comprising: a) administering the immunogenic composition of any one of the preceding embodiments to the non-human animal or human subject, and b) optionally, collecting antibodies against the coronavirus antigen from the non-human animal or human subject.

E47. The method of any one of the preceding embodiments, further comprising administering an adjuvant to the non-human animal or human subject (e.g., a non-human animal or human subject for immunization).

E48. The method of embodiment 47, wherein the adjuvant is co-formulated and co-administered with the immunogenic composition, or is formulated and administered separately from the immunogenic composition.

E49. The method of any one of the preceding embodiments, further comprising administering (e.g., pre-administering or priming) the non-human animal or human subject (e.g., a non-human animal or human subject for immunization) with the coronavirus antigen prior to administration of the immunogenic composition.

E50. The method of any one of the preceding embodiments, further comprising administering the coronavirus antigen to the non-human animal or human subject (e.g., a non-human animal or human subject for immunization) from 1 to 7 days prior (e.g., 1, 2, 3, 4, 5, 6, or 7 days prior) to administering the immunogenic composition.

E51. The method of any one of the preceding embodiments, wherein the coronavirus antigen is administered as a protein preparation, encoded in a plasmid (pDNA), presented in a virus-like particle (VLP), or formulated in a lipid nanoparticle (LNP).

E52. The method of any one of the preceding embodiments, further comprising administering the circular polyribonucleotide or linear polyribonucleotide without a carrier.

E53. The method of any one of the preceding embodiments, further comprising formulating the immunogenic composition with a carrier (e.g., lipid nanoparticle, virus-like particle, or liposome).

E54. The method of any one of the preceding embodiments, further comprising administering or immunizing the circular polyribonucleotide or linear polyribonucleotide at least two times, (e.g., 2, 3, 4, or 5 times) to the non-human animal or human subject (e.g., a non-human animal or human subject for immunization).

E55. The method of any one of the preceding embodiments, further comprising collecting plasma from the non-human animal or human subject (e.g., a non-human animal or human subject for immunization).

E56. The method of any one of the preceding embodiments, further comprising purifying polyclonal antibodies from the plasma of a non-human animal or human subject (e.g., a non-human animal or human subject for immunization).

E57. The method of any one of the preceding embodiments, further comprising administering or immunizing the non-human animal or human subject (e.g., a non-human animal or human subject for immunization) with a vaccine.

E58. The method of embodiment 51, wherein the vaccine is pneumococcal polysaccharide vaccine (e.g., PCV13 or PPSV23).

E59. The method of embodiment 57, wherein the vaccine is for a bacterial infection.

E60. The method of any one of the preceding embodiments, wherein the non-human animal or human subject (e.g., a non-human animal or human subject for immunization) is immunized with the circular polyribonucleotide or linear polyribonucleotide by injection.

E61. The method of any one of the preceding embodiments, wherein the human subject (e.g., the human subject for immunization) at risk for a coronavirus-related disease.

E62. The method of any one of the preceding embodiments, wherein the human subject (e.g., the human subject for immunization) is a human over 50 years old, an immune-compromised human, a human with a chronic health condition (e.g., obesity, diabetes, cancer), or a health care worker.

E63. The method of any one of the preceding embodiments, wherein the non-human animal (e.g., the non-human animal subject for immunization) is an agricultural animal (e.g., a pig, cow, goat, chicken, sheep).

E64. The method of any one of the preceding embodiments, wherein the non-human animal (e.g., the non-human animal subject for immunization) is a pet (e.g., a dog or cat), a zoo animal (e.g., a feline), a mammal (e.g., an ungulate (e.g., a pig, a cow, a goat, a sheep), a rodent (e.g., a rabbit, a rat, a mouse).

E65. The method of any one of the preceding embodiments, wherein the non-human animal is a transchromosomal non-human animal comprising a humanized immunoglobulin gene locus.

E66. The method of embodiment 65, wherein the non-human animal is a transchromosomal cow comprising a human artificial chromosome (HAC) vector that comprises the humanized immunoglobulin gene locus.

E67. The method of any one of embodiments 65 or 66, wherein the humanized immunoglobulin gene locus encodes an immunoglobulin heavy chain.

E68. The method of embodiment 67, wherein the humanized immunoglobulin heavy chain comprises an IgG isotype heavy chain.

E69. The method of any one of embodiments 67 or 68, wherein the humanized immunoglobulin heavy chain comprises an IgG1, IgG2, IgG3, or IgG4 isotype heavy chain.

E70. The method of any one of embodiments 65-69, wherein the humanized immunoglobulin gene locus encodes an immunoglobulin light chain.

E71. The method of embodiment 70, wherein the immunoglobulin light chain comprises a kappa light chain or a lambda light chain.

E72. The method of any one of the preceding embodiments, wherein the non-human animal comprises a B cell having a B cell receptor, and wherein the B cell receptor binds to the antigen.

E73. The method of any one of the preceding embodiments, wherein the non-human animal comprises a plurality of B cells comprising a first B cell that binds to a first epitope of the coronavirus antigen and a second B cell that binds to a second epitope of the coronavirus antigen.

E74. The method of any one of the preceding embodiments, wherein the non-human animal comprises a T cell, and wherein the T cell comprises a T cell receptor that binds to the coronavirus antigen.

E75. The method of any one of the preceding embodiments, wherein upon activation, the T cell enhances production of an antibody that that binds to the coronavirus antigen.

E76. The method of any one of the preceding embodiments, wherein upon activation, the T cell enhances antibody production by a B cell that binds to the coronavirus antigen.

E77. The method of any one of the preceding embodiments, wherein upon activation, the T cell enhances survival, proliferation, plasma cell differentiation, somatic hypermutation, immunoglobulin class switching, or a combination thereof of a B cell that that binds to the coronavirus antigen.

E78. The method of any one of the preceding embodiments, further comprising purifying polyclonal antibodies against the coronavirus antigen from the plasma of the non-human animal or human subject (e.g., the non-human animal or human subject for immunization).

E79. The method of any one of the preceding embodiments, wherein an antibody of the polyclonal antibodies specifically binds to the coronavirus antigen.

E80. The method of any one of the preceding embodiments, wherein an antibody of the polyclonal antibodies is a humanized antibody or a fully human antibody.

E81. The method of any one of the preceding embodiments, wherein an antibody of the polyclonal antibodies is an IgG an IgG, IgA, or IgM isotype antibody.

E82. The method of any one of the preceding embodiments, wherein an antibody of the polyclonal antibodies is an IgG1, IgG2, IgG3, or IgG4 isotype antibody.

E83. The method of any one of the preceding embodiments, wherein the non-human animal comprises a plurality of polyclonal antibodies that specifically bind at least two epitopes that are encoded by the circular polyribonucleotide.

E84. The method of any one of the preceding embodiments, wherein the non-human animal comprises a plurality of polyclonal antibodies that specifically bind at least two epitopes that are encoded by the linear polyribonucleotide.

E85. The method of any one of the embodiments 81 or 82, wherein the plurality of polyclonal antibodies comprises humanized antibodies.

E86. The method of any one of the embodiments 83 or 84, wherein the plurality of polyclonal antibodies comprises fully human antibodies.

E87. The method of any one of the embodiments 83-86, wherein the plurality of polyclonal antibodies comprise IgG antibodies, IgG1 antibodies, IgG2 antibodies, IgG3 antibodies, IgG4 antibodies, IgM antibodies, IgA antibodies, or a combination thereof.

E88. The method of any one of embodiments 83-86, wherein the plurality of polyclonal antibodies comprise humanized immunoglobulin gene loci comprising an IgM or IgA isotype heavy chains.

E89. The method of any one of embodiments 83-88, wherein the plurality of polyclonal antibodies comprise humanized immunoglobulin gene loci encoding immunoglobulin light chains.

E90. The method of embodiment 89, wherein the immunoglobulin light chains comprises kappa light chains or lambda light chains.

E91. The method of any one of the preceding embodiments, further comprising collecting blood from the non-human animal or human subject (e.g., the non-human animal or human subject for immunization) and purifying antibodies against the coronavirus antigen from the blood.

E92. A method of producing a polyclonal antibody preparation against a coronavirus antigen (e.g., an anti-coronavirus antibody preparation), comprising:

a) administering to the immunogenic composition of any one of the preceding embodiments to a non-human animal or human subject (e.g., the non-human animal or human subject for immunization); and
b) collecting blood or plasma from the non-human animal or human subject.

E93. The method of embodiment 92, wherein the polyclonal antibody preparation is formulated as a pharmaceutical composition or veterinary composition.

E94. A method of delivering a polyclonal antibody preparation against a coronavirus to a subject (e.g. a subject for treatment) having a coronavirus infection, comprising administering the polyclonal antibody preparation of any one of the preceding embodiments to the subject having a coronavirus infection.

E95. A method of delivering a polyclonal antibody preparation to a subject (e.g. a subject for treatment) at risk for exposure to a coronavirus infection, comprising administering the polyclonal antibodies preparation of any one of the preceding embodiments to the subject at risk for exposure to a coronavirus infection.

E96. A method of preventing or treating a subject (e.g. a subject for treatment) in need thereof for coronavirus infection comprising administering the polyclonal antibodies preparation of any one of the preceding embodiments to the subject in need thereof.

E97. The method of any one for the preceding embodiments, further comprising:

a) immunizing a non-human animal that has been genetically modified to produce human antibodies with the circular polyribonucleotide of any one of the preceding embodiments or the linear polyribonucleotide of any one of the preceding embodiments;
b) collecting blood from the non-human animal;
c) purifying antibodies form the non-human animal;
d) formulating the antibodies for pharmaceutical use; and
e) administering the formulated antibodies to a human subject.

E98. The method of embodiment 97, wherein the non-human animal has a humanized immune system.

E99. The method of embodiment 97, wherein the non-human animal has a humanized immunoglobulin gene locus.

E100. The method of embodiment 97, wherein the non-human animal is a transchromosomal cow comprising a human artificial chromosome (HAC) vector that comprises a human immunoglobulin gene locus

E101. The method of any one of the preceding embodiments, wherein the administration or immunization is before, after, or simultaneously with the subject in need thereof's risk of exposure to the coronavirus.

E102. The method of any one of the preceding embodiments, wherein the subject (e.g. a subject for treatment) having a coronavirus infection, the subject at risk for exposure to a coronavirus infection, or the subject in need thereof is a human subject.

E103. The method of embodiment 102, wherein the human subject (e.g. a human subject for treatment) is a human over 50 years old, an immune-compromised human, a human with a chronic health condition (e.g., obesity, diabetes, or cancer), or a health care worker.

E104. The method of any one of the preceding embodiments, wherein the subject (e.g. a subject for treatment) at risk for exposure to a coronavirus infection, or the subject in need thereof is a human subject at risk for a coronavirus related disease.

E105. The method of any one of the preceding embodiments, wherein the subject (e.g. a subject for treatment) having a coronavirus infection, the subject at risk for exposure to a coronavirus infection, or the subject in need thereof is a human subject diagnosed with a coronavirus-related disease (e.g., Covid-19, SARS, MERS).

E106. The method of any one of the preceding embodiments, wherein the subject (e.g. a subject for treatment) having a coronavirus infection, the subject (e.g. a subject for treatment) at risk for exposure to a coronavirus infection, or the subject (e.g. a subject for treatment) in need thereof is a non-human animal subject.

E107. The method of any one of the preceding embodiments, wherein the subject (e.g. a subject for treatment) having a coronavirus infection, the subject (e.g. a subject for treatment) at risk for exposure to a coronavirus infection, or the subject (e.g. a subject for treatment) in need thereof is an agricultural animal (e.g., cow, pig, sheep, horse, goat), pet (e.g., a cat or dog), or zoo animal (e.g., a feline).

E108. The method of any one of the preceding embodiments, further comprising monitoring the subject (e.g. a subject for treatment) having a coronavirus infection, the subject (e.g. a subject for treatment) at risk for exposure to a coronavirus infection, or the subject in need thereof for the presence of the polyclonal antibodies.

E109. The method of any one of the preceding embodiments, wherein the monitoring is prior to administration of the polyclonal antibodies and/or after the administration of the polyclonal antibodies.

### Definitions

The present invention will be described with respect to particular embodiments and with reference to certain figures but the invention is not limited thereto but only by the claims. Terms as set forth hereinafter are generally to be understood in their common sense unless indicated otherwise.

As used herein, the terms “circRNA,” “circular polyribonucleotide,” and “circular RNA” are used interchangeably and mean a polyribonucleotide molecule that has a structure having no free ends (i.e., no free 3′ and/or 5′ ends), for example a polyribonucleotide that forms a circular or endless structure through covalent or non-covalent bonds.

As used herein, the terms “circRNA preparation,” “circular polyribonucleotide preparation,” and “circular RNA preparation” are used interchangeably and mean a composition comprising circRNA molecules and a diluent, carrier, first adjuvant, or a combination thereof. An “immunogenic” circRNA preparation is that which when introduced into an animal causes the animal's immune system to become reactive against the antigen(s) expressed by the circRNA

As used herein, the terms “linear RNA,” “linear polyribonucleotide,” and “linear polyribonucleotide molecule” are used interchangeably and mean a monoribonucleotide molecule or polyribonucleotide molecule having a 5′ and 3′ end. One or both of the 5′ and 3′ ends may be free ends or joined to another moiety. In some embodiments, the linear RNA has a 5′ end or 3′ end that is modified or protected from degradation (e.g., by a 5′ end protectant or a 3′ end protectant). In some embodiments, the linear RNA has non-covalently linked 5′ or 3′ ends. A linear RNA can be used as a starting material for circularization through, for example, splint ligation, or chemical, enzymatic, ribozyme- or splicing-catalyzed circularization methods.

As used herein, the terms “linear RNA preparation” and “linear polyribonucleotide preparation” are used interchangeably and mean a composition comprising linear RNA molecules and a diluent, carrier, first adjuvant, or a combination thereof. An “immunogenic” linear RNA preparation is that which when introduced into an animal causes the animal's immune system to become reactive against the antigen(s) expressed by the circRNA.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules.

As used herein, the term “fragment” means any portion of a nucleotide molecule that is at least one nucleotide shorter than the nucleotide molecule. For example, a nucleotide molecule can be a linear polyribonucleotide molecule and a fragment thereof can be a monoribonucleotide or any number of contiguous polyribonucleotides that are a portion of the linear polyribonucleotide molecule. As another example, a nucleotide molecule can be a circular polyribonucleotide molecule and a fragment thereof can be a polyribonucleotide or any number of contiguous polyribonucleotides that are a portion of the circular polyribonucleotide molecule. A fragment of a nucleotide molecule includes at least 10 nucleic acid residues, e.g., at least 20 nucleic acid residues, at least 50 nucleic acid residues, and at least 100 nucleic acid residues. A fragment also means any portion of a polypeptide molecule that is at least one peptide shorter than the polypeptide molecule. For example, a fragment of a polypeptide can be a polypeptide or any number of contiguous amino acids that are a portion of the full-length polypeptide molecule. A fragment of a polypeptide includes at least 5 amino acid residues, e.g., at least 10 amino acids residues, at least 20 amino acids residues, at least 50 amino acid residues, at least 100 amino acid residues.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the term “modified ribonucleotide” is a nucleotide with at least one modification to the sugar, the nucleobase, or the internucleoside linkage.

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPG P, where x=any amino acid. In some embodiments, the stagger element may include a chemical moiety, such as glycerol, a non-nucleic acid linking moiety, a chemical modification, a modified nucleic acid, or any combination thereof.

As used herein, the term “substantially resistant” is one that has at least 50%, 55%, 60%, 65%, 70%, 75%, 80%, 85%, 90%, 95%, 96%, 97%, 98% or 99% resistance to an effector as compared to a reference.

As used herein, the term “stoichiometric translation” is a substantially equivalent production of expression products translated from the circular polyribonucleotide. For example, for a circular polyribonucleotide having two expression sequences, stoichiometric translation of the circular polyribonucleotide means that the expression products of the two expression sequences have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%, or any percentage therebetween.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its non-circular starting material.

As used herein, the term “adaptive immune response” means either a humoral or cell-mediated immune response. The humoral immune response (also called antibody immune response) is mediated by B lymphocytes, which release antibodies that specifically bind to an antigen. The cell-mediated immune response (also called cellular immune response) involves the binding of cytotoxic T lymphocytes (CTL) to foreign or infected cells, followed by the lysis of these cells.

As used herein, the term “adjuvant” refers to a compound that, when used in combination with a circular RNA molecule, augments or otherwise alters or modifies the resultant immune response.

Modification of the immune response includes intensification or broadening the specificity of either or both antibody and cellular immune responses. Modification of the immune response can also mean decreasing or suppressing certain antigen-specific immune responses.

As used herein, the terms “human antibody,” “human immunoglobulin,” and “human polyclonal antibody” are used interchangeably and mean an antibody or antibodies produced in a non-human animal that is otherwise indistinguishable from antibody produced in a human vaccinated by the same circular RNA preparation. This is in contrast to “humanized antibodies” which are modified to have human characteristics, such as through generation of chimeras, but that maintain attributes of the host animal in which they are produced. Because human antibody made according to the method disclosed herein is comprised of IgG that are fully human, no enzymatic treatment is needed to eliminate the risk of anaphylaxis and serum sickness associated with heterologous species IgG.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide, such as a lipid nanoparticle or LNP), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked,” “naked delivery,” and its cognates means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier. A naked delivery formulation may comprise non-carrier pharmaceutical excipients or diluents.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate, lactose, sucrose, cellulose, microcrystalline cellulose, kaolin, mannitol, sorbitol, inositol, sodium chloride, dry starch, cornstarch, or powdered sugar.

As used herein, a “subject for immunization” is a subject that is administered an immunogenic composition (e.g., a composition comprising a circular polyribonucleotide that comprises a sequence encoding a coronavirus antigen or a composition comprising a linear polyribonucleotide that comprises a sequence selected from the SEQ ID NO. in TABLE 3). A subject for immunization is a non-human animal (“non-human animal subject for immunizations”) (e.g., an agricultural animal, pet, zoo animal, etc.) or human subject (“human subject for immunization”).

As used herein, a “subject for treatment” is a subject that is administered polyclonal antibodies against a coronavirus (e.g., a polyclonal antibody preparation against a coronavirus) as a prophylactic treatment or to treat a coronavirus infection. A prophylactic treatment includes administration of the polyclonal antibodies against a coronavirus to a subject at risk for exposure to a coronavirus (e.g., a healthcare worker) or at risk for coronavirus related disease (e.g., a human over 50 years old; an immune-compromised human; a human with a chronic health condition such as obesity, diabetes, cancer). A subject for treatment is a non-human animal (“non-human animal subject for treatment”) (e.g., an agricultural animal, pet, zoo animal, etc.) or human subject (“human subject for treatment”).

As used herein, a “variant” refers to a polypeptide which includes at least one alteration, e.g., a substitution, insertion, deletion, and/or fusion, at one or more residue positions, as compared to the parent or wild-type polypeptide. A variant may include between 1 and 10, 10 and 20, 20 and 50, 50 and 100, or more alterations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows exemplary circular polyribonucleotides comprising a sequence encoding a coronavirus antigen (e.g., a spike protein, a receptor binding domain (RBD) protein of a spike protein).

FIG. 2 shows a schematic for generating human polyclonal antibodies that bind to a coronavirus antigen to be administered to human subjects.

FIG. 3 shows an RBD antigen encoded by a circular RNA was detected in BJ Fibroblasts and HeLa cells, and was not detected in BJ Fibroblasts and HeLa cells with the vehicle control.

FIG. 4 shows that a sustainable anti-RBD antibody response was attained following administration of a circular RNA encoding a SARS-CoV-2 RBD antigen, formulated with a cationic polymer (e.g., protamine), in a mouse model.

FIG. 5 shows that an anti-Spike response was attained following administration of a circular RNA encoding a SARS-CoV-2 RBD antigen, formulated with a cationic polymer (e.g., protamine), in a mouse model.

FIG. 6 shows anti-RBD IgG2a and IgG1 isotype levels that were obtained after administration of a circular RNA encoding a SARS-CoV-2 RBD antigen, formulated with a cationic polymer (e.g., protamine), in a mouse model.

FIG. 7 shows protein expression from circular RNA in vivo for prolonged periods of time after intramuscular injection of circular RNA preparations (Trans-IT formulated, protamine formulated, unformulated), protamine vehicle only, and uninjected control mice.

FIG. 8 shows protein expression from circular RNA in vivo for prolonged periods of time after simultaneous intramuscular delivery of Addavax™ adjuvant with (i) unformulated circular RNA preparations (left graph), (ii) circular RNA formulated with TransIT (middle graph), and (iii) circular RNA formulated with protamine (right graph). In each case, Addavax™ adjuvant was delivered as an individual injection at 0 and 24 h.

FIG. 9 shows protein expression from circular RNA in vivo for prolonged periods of time after intradermal delivery of (i) circular RNA formulated with protamine, (ii) circular RNA formulated with protamine, with an injection of Addavax™ adjuvant at 24 hours, (iii) protamine vehicle only, and (iv) uninjected control mice.

FIG. 10 is a schematic of an exemplary circular RNA that includes two expression sequence, where each expression sequence encodes an antigen and where one or both expression sequences encode a coronavirus antigen. The circular RNA includes two open reading frames (ORFs), each ORF encoding an expression sequence, where each ORF is operably linked to an IRES.

FIG. 11 is a schematic of an exemplary circular RNA that includes two expression sequences, wherein each expression sequence is an antigen, and where one or both expression sequences encode a coronavirus antigen. The circular RNA includes two expression sequences separated by a 2A sequence, all operably linked to an IRES

FIG. 12 shows a schematic of a plurality of polyribonucleotides, where each polynucleotide includes an ORF that encodes an antigen, and where one or both ORFs encode a coronavirus antigen.

FIG. 13A shows multi-antigen expression from a circular polyribonucleotide. RBD antigen expression was detected from circular RNAs encoding a SARSs-CoV-2 RBD antigen and a GLuc polypeptide.

FIG. 13B shows multi-antigen expression from a circular polyribonucleotide. GLuc activity was detected from circular RNAs encoding a SARSs-CoV-2 RBD antigen and a GLuc polypeptide.

FIG. 14A demonstrates immunogenicity of multiple antigens from circular RNAs in mouse model. Mice were vaccinated with a first circular RNA encoding a SARS-CoV-2 RBD antigen and a second circular RNA encoding a GLuc polypeptide. Anti-RBD antibodies were obtained at 17 days after injection.

FIG. 14B demonstrates immunogenicity of multiple antigens from circular RNAs in mouse model. Mice were vaccinated with a first circular RNA encoding a SARS-CoV-2 RBD antigen and a second circular RNA encoding a GLuc polypeptide. GLuc activity was detected at 2 days after injection.

FIG. 15A demonstrates immunogenicity of multiple antigens from circular RNAs in mouse model. Mice were vaccinated with a first circular RNA encoding a SARS-CoV-2 RBD antigen and a second circular RNA encoding Influenza hemagglutinin (HA) antigen. Anti-RBD antibodies were obtained at 17 days after injection.

FIG. 15B demonstrates immunogenicity of multiple antigens from circular RNAs in mouse model. Mice were vaccinated with a first circular RNA encoding a SARS-CoV-2 RBD antigen and a second circular RNA encoding Influenza hemagglutinin (HA) antigen. Anti-HA antibodies were obtained at 17 days after injection.

FIG. 16A demonstrates immunogenicity of multiple antigens from circular RNAs in a mouse model. Mice were vaccinated with a first circular RNA encoding a SARS-CoV-2 Spike antigen and a second circular RNA encoding Influenza hemagglutinin (HA) antigen. Anti-RBD (domain of Spike) antibodies were obtained at 17 days after injection.

FIG. 16B demonstrates immunogenicity of multiple antigens from circular RNAs in a mouse model. Mice were vaccinated with a first circular RNA encoding a SARS-CoV-2 Spike antigen and a second circular RNA encoding Influenza hemagglutinin (HA) antigen. Anti-HA antibodies were obtained at 17 days after injection.

FIG. 17 demonstrates an anti-HA antibody response in mice administered circular RNA encoding multiple antigens. Mice were administered a circular RNA encoding: a SARS-CoV-2 RBD antigen, a SARS-CoV-2 Spike antigen, an Influenza HA antigen, a SARS-CoV-2 RBD antigen and an Influenza HA antigen, a SARS-CoV-2 RBD antigen and a GLuc polypeptide, or a SARS-CoV-2 RBD antigen and a SARS-CoV-2 Spike antigen. A hemagglutination inhibition assay (HAI) was used to measure anti-Influenza HA antibodies. FIG. 24 shows HAI titer in samples that were administered circular RNA preparations encoding the Influenza HA antigen when it was administered alone or when administered in combination with SARS-CoV-2 antigens e.g. RBD or Spike.

### DETAILED DESCRIPTION

The disclosure relates generally to circular polyribonucleotides comprising a sequence encoding an antigen and/or epitope from a coronavirus, immunogenic compositions comprising circular polyribonucleotides encoding a coronavirus antigen and/or epitope, and methods for producing circular polyribonucleotides encoding a coronavirus antigen and/or epitope and compositions comprising circular polyribonucleotides encoding a coronavirus antigen and/or epitope. In some embodiments, the circular polyribonucleotides and/or immunogenic compositions are used in methods of generating an immune response against the antigen and/or epitope from a coronavirus by administering the circular polyribonucleotide and/or immunogenic composition to the subject or immunizing the subject with a circular polyribonucleotide comprising a sequence encoding a coronavirus antigen and/or epitope and/or immunogenic composition comprising the circular polyribonucleotide. The subject (e.g., the subject for immunization) can be a mammal, such as an ungulate. The subject for immunization can be a human. In some embodiments, the subject for immunization is a non-human animal having a humanized immune system.

The disclosure also relates generally to linear polyribonucleotides comprising a sequence encoding an antigen and/or epitope from a coronavirus, immunogenic compositions comprising linear polyribonucleotides encoding a coronavirus antigen and/or epitope, and methods for producing linear polyribonucleotides encoding a coronavirus antigen and/or epitope and compositions comprising linear polyribonucleotides encoding a coronavirus antigen and/or epitope. In some embodiments, the linear polyribonucleotides and/or immunogenic compositions are used in methods of generating an immune response against the antigen and/or epitope from a coronavirus by administering the linear polyribonucleotide and/or immunogenic composition to the subject or immunizing the subject with a linear polyribonucleotide comprising a sequence encoding a coronavirus antigen and/or epitope and/or immunogenic composition comprising the linear polyribonucleotide. The subject (e.g., the subject for immunization) can be a mammal, such as an ungulate. The subject for immunization can be a human. In some embodiments, the subject for immunization is a non-human animal having a humanized immune system.

The disclosure also relates generally to methods of generating or producing polyclonal antibodies that bind to an antigen and/or epitope from a coronavirus in a subject using the circular polyribonucleotides or immunogenic compositions described herein. In some embodiments, the subject for immunization is a human. In some embodiments, the subject for immunization is a non-human animal (e.g., an ungulate). In some embodiments, the non-human animal has a humanized immune system. In a particular embodiment, a circular polyribonucleotide that encodes antigens and/or epitopes from a coronavirus and/or an immunogenic composition comprising a circular polyribonucleotide encoding a coronavirus antigen and/or epitope is administered to a non-human animal with a humanized immune system, thereby stimulating production of human polyclonal antibodies that bind to the antigens and/or epitopes from the coronavirus.

The disclosure also relates generally to methods of generating or producing polyclonal antibodies that bind to an antigen and/or epitope from a coronavirus in a subject using linear polyribonucleotides or immunogenic compositions described herein. In some embodiments, the subject for immunization is a human. In some embodiments, the subject for immunization is a non-human animal (e.g., an ungulate). In an embodiment, a linear polyribonucleotide that encodes antigens and/or epitopes from a coronavirus and/or an immunogenic composition comprising a linear polyribonucleotide encoding a coronavirus antigen and/or epitope is administered to a non-human animal with a humanized immune system, thereby stimulating production of human polyclonal antibodies that bind to the antigens and/or epitopes from the coronavirus.

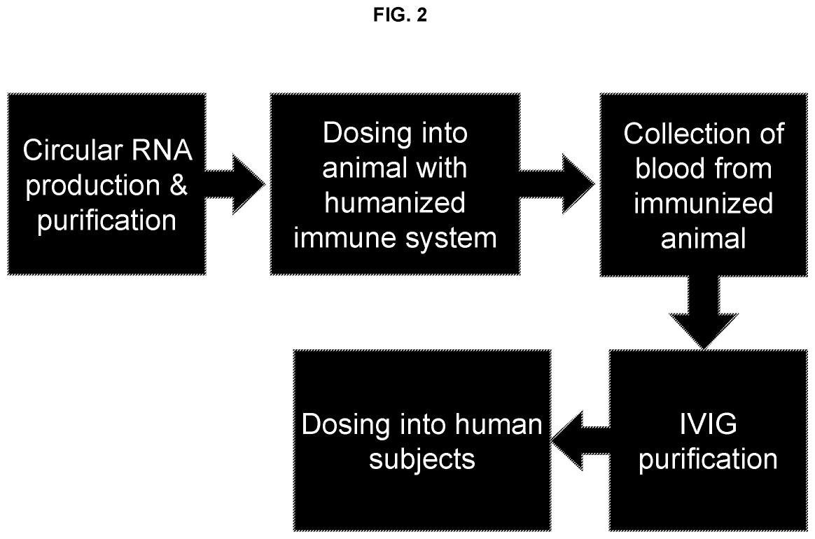

In further embodiments, the produced polyclonal antibodies are purified. The purified polyclonal antibodies are suitable for use as a prophylactic against a coronavirus or treatment of a coronavirus infection. The purified polyclonal antibodies can be administered to a subject for treatment. An schematic example of the methods described herein is provided in FIG. 2.

### Circular Polyribonucleotide

The circular polyribonucleotides as disclosed herein comprise a sequence encoding an antigen and/or epitope from a coronavirus. This circular polyribonucleotide expresses the sequence encoding the antigen and/or epitope from the coronavirus in a subject (e.g., a subject for immunization). In some embodiments, circular polyribonucleotides comprising a coronavirus antigen and/or epitope are used to produce an immune response in a subject (e.g., a subject for immunization). In some embodiments, circular polyribonucleotides comprising a coronavirus antigen and/or epitope are used to produce polyclonal antibodies as described herein.

### Coronavirus Antigens and Epitopes

The circular polyribonucleotide comprises a sequence encoding a coronavirus antigen or epitope. The antigens and/or epitopes disclosed herein are associated with coronaviruses. In some embodiments, the antigens and/or epitopes are expressed by a coronavirus, or derived from an antigen and/or epitope that is expressed by a coronavirus.

An antigen is a molecule containing one or more epitopes (either linear, conformational or both) that will elicit an adaptive immune response in a subject (e.g., a subject for immunization). An epitope can be a part of an antigen that is recognized, targeted, or bound by a given antibody or T cell receptor. An epitope can be a linear epitope, for example, a contiguous sequence of amino acids. An epitope can be a conformational epitope, for example, an epitope that contains amino acids that form an epitope in the folded conformation of the protein. A conformational epitope can contain non-contiguous amino acids from a primary amino acid sequence. Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids.

A coronavirus antigen or epitope can be or can comprise all or a part of a protein, a peptide, a glycoprotein, a lipoprotein, a phosphoprotein, a ribonucleoprotein, a carbohydrate (e.g., a polysaccharide), a lipid (e.g., a phospholipid or triglyceride), or a nucleic acid (e.g., DNA, RNA).

A coronavirus antigen or epitope can comprise a protein antigen or epitope (e.g., a peptide antigen or peptide epitope from a protein, glycoprotein, lipoprotein, phosphoprotein, or ribonucleoprotein). An antigen or epitope can comprise an amino acid, a sugar, a lipid, a phosphoryl, or a sulfonyl group, or a combination thereof.

A coronavirus protein antigen or epitope can comprise a post-translational modification, for example, glycosylation, ubiquitination, phosphorylation, nitrosylation, methylation, acetylation, amidation, hydroxylation, sulfation, or lipidation.

In some embodiments, the coronavirus is a pathogenic coronavirus. In some embodiments, the coronavirus is a respiratory pathogen. In some embodiments, the coronavirus is a blood borne pathogen. In some embodiments, the coronavirus is an enteric pathogen.

Non-limiting examples of coronaviruses of the disclosure include severe acute respiratory syndrome associated coronavirus (SARS-CoV, e.g., SARS-CoV-1, SARS-CoV-2), Middle East respiratory syndrome coronavirus (MERS-CoV), bat coronaviruses, zoonotic coronaviruses that can infect humans or other animals, newly emerged or newly-discovered coronaviruses, and other coronaviruses.

In some embodiments, a circular polyribonucleotide comprises severe acute respiratory syndrome associated coronavirus (SARS-CoV) antigens and/or epitopes. In some embodiments, a circular polyribonucleotide comprises SARS-CoV-1 antigens and/or epitopes. In some embodiments, a circular polyribonucleotide comprises SARS-CoV-2 antigens and/or epitopes. In some embodiments, a circular polyribonucleotide comprises Middle East respiratory syndrome coronavirus (MERS-CoV) antigens and/or epitopes. In some embodiments, a circular polyribonucleotide comprises zoonotic coronavirus antigens and/or epitopes that can infect humans or other animals. In some embodiments, a circular polyribonucleotide comprises antigens and/or epitopes from a newly-emerged coronavirus.

In some embodiments, a circular polyribonucleotide comprises Coronaviridae antigens and/or epitopes.

In some embodiments, a circular polyribonucleotide comprises antigens and/or epitopes from a genus or subgenus that is Alphacoronavirus, Betacoronavirus, Gammacoronavirus, Deltacoronavirus, Merbecovirus, or Sarbecovirus. In some embodiments, a circular polyribonucleotide comprises Betacoronavirus antigens and/or epitopes. In some embodiments, a circular polyribonucleotide comprises Sarbecovirus antigens and/or epitopes. In some embodiments, a circular polyribonucleotide comprises Merbecovirus antigens and/or epitopes.

In some embodiments, a circular polyribonucleotide comprises a sequence for an antigen from a coronavirus that is a biosafety level 2 (BSL-2) pathogen). In some embodiments, a circular polyribonucleotide comprises a sequence from a coronavirus that is a biosafety level 3 (BSL-3) pathogen. In some embodiments, the coronavirus is a biosafety level 4 pathogen (BSL-4). In some embodiments, no approved drugs (e.g., antiviral or antibiotic drugs) are available to treat infection with the coronavirus from which the antigen expressed by the circular polyribonucleotide is derived. In some embodiments, no approved vaccines are available to prevent or reduce the risk of infection with the coronavirus from which the antigen expressed by the circular polyribonucleotide is derived.

An antigen and/or epitope can be from a coronavirus surface protein, a coronavirus membrane protein, a coronavirus envelope protein, a coronavirus capsid protein, a coronavirus nucleocapsid protein, a coronavirus spike protein, a coronavirus receptor binding domain (RBD) of a spike protein, a coronavirus entry protein, a coronavirus membrane fusion protein, a coronavirus structural protein, a coronavirus non-structural protein, a coronavirus regulatory protein, a coronavirus accessory protein, a secreted coronavirus protein, a coronavirus polymerase protein, a coronavirus RNA polymerase, a coronavirus protease, a coronavirus glycoprotein, a coronavirus fusogen, a coronavirus helical capsid protein, a coronavirus icosahedral capsid protein, a coronavirus matrix protein, a coronavirus replicase, a coronavirus transcription factor, or a coronavirus enzyme.

Antigens and/or epitopes from any number of coronaviruses are expressed by the circular polyribonucleotide. In some cases, the antigens and/or epitopes are associated with or expressed by one coronavirus disclosed herein. In some embodiments, the antigens and/or epitopes are associated with or expressed by two or more coronaviruses disclosed herein.

In some cases, two or more coronaviruses are phenotypically related. For example, compositions and methods of the disclosure can utilize antigens and/or epitopes from two or more coronaviruses that are respiratory pathogens, two or more coronaviruses that are associated with severe disease, two or more coronaviruses that are associated with adverse outcomes in immunocompromised subjects (e.g., subjects for immunization), two or more coronaviruses that are associated with acute respiratory distress syndrome (ARDS), two or more coronaviruses that are associated with severe acute respiratory syndrome (SARS), two or more coronaviruses that are associated with middle eastern respiratory syndrome (MERS), or a combination thereof.

A circular polyribonucleotide can comprise or encode, for example, antigens and/or epitopes from at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, or more coronaviruses.

In some embodiments, a circular polyribonucleotide comprises or encodes antigens and/or epitopes from at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, or less coronaviruses.

In some embodiments, a circular polyribonucleotide comprises or encodes antigens and/or epitopes from about 1, 2, 3, 4, 5, 6, 7, 8, 9, 10, 15, 20, 25, 30, 40, 50, 60, 70, 80, 90, or 100, coronaviruses.

In some embodiments, an antigen and/or epitope is from a coronavirus, for example, a severe acute respiratory syndrome associated coronavirus (SARS-CoV, e.g., SARS-CoV-1, SARS-CoV-2), a Middle East respiratory syndrome coronavirus (MERS-CoV), or another coronavirus. In some embodiments, an antigen and/or epitope of the disclosure is from a predicted open reading frame from a coronavirus genome.

New SARS isolates may be identified by a percent homology of 99%, 98%, 97%, 95%, 92%, 90%, 85%, or 80% homology of the polynucleotide sequence for specific genomic regions for the new virus with the polynucleotide sequence for specific genomic regions of the known SARS viruses. Additionally, new SARS isolates may be identified by a percent homology of 99%, 98%, 97%, 95%, 92%, 90%, 85%, or 80% homology of the polypeptide sequence encoded by the polynucleotide of specific genomic regions of the new SARS virus to the polypeptide sequence encoded by the polynucleotides of specific regions of the known SARS virus. These genomic regions may include regions (e.g., gene products or ORFs) which are typically in common among numerous coronaviruses, as well as group specific regions (e.g., antigenic groups), such as, for example, any one of the following genomic regions which could be readily identified by a virologist skilled in the art: 5′ untranslated region (UTR), leader sequence, ORF1a, ORF1b, nonstructural protein 2 (NS2), hemagglutinin-esterase glycoprotein (HE) (also referred to as E3), spike glycoprotein (S) (also referred to as E2), ORF3a, ORF3b, nonstructural protein 4 (NS4), envelope (small membrane) protein (E) (also referred to as sM), membrane glycoprotein (M) (also referred to as E1), ORF5a, ORF5b, nucleocapsid phosphoprotein (N), ORF6, ORF7a, ORF7b, ORF8, ORF8a, ORF8b, ORF9a, ORF9b, ORF10, intergenic sequences, receptor binding domain (RBD) of a spike protein, 3′UTR, or RNA dependent RNA polymerase (pol). The SARS virus may have identifiable genomic regions with one or more the above-identified genomic regions. A SARS viral antigen includes a protein encoded by any one of these genomic regions. A SARS viral antigen may be a protein or a fragment thereof, which is highly conserved with coronaviruses. A SARS viral antigen may be a protein or fragment thereof, which is specific to the SARS virus (as compared to known coronaviruses).

In some embodiments, an antigen and/or epitope of the disclosure is from a predicted transcript from a SARS-CoV genome. In some embodiments, an antigen and/or epitope of the disclosure is from a protein encoded by an open reading frames from a SARS-CoV genome. Non-limiting examples of open reading frames in SARS-CoV genomes can include ORF1a, ORF1b, spike (S), ORF3a, ORF3b, envelope (E), membrane (M), ORF6, ORF7a, ORF7b, ORF8, ORF8a, ORF8b, ORF9a, ORF9b, nucleocapsid (N), and ORF10.

ORF1a and ORF1b encodes 16 non-structural proteins (nsp), for example, nsp1, nsp2, nsp3, nsp4, nsp5, nsp6, nsp7, nsp8, nsp9, nsp10, nsp 11, nsp12, nsp13, nsp14, nsp15, and nsp16. Nonstructural proteins, for example, contribute to viral replication, viral assembly, immune response modulation, or a combination thereof. In some embodiments, the antigen is a non-structural protein or is an antigenic sequence encoding a non-structural protein. In some embodiments, epitopes are from a coronavirus non-structural protein.

Spike (S) encodes a spike protein, which in some embodiments contributes to binding to a host cell receptor, fusion of the virus with the host cell membrane, entry of the virus into a host cell, or a combination thereof. Spike protein can be an antigen. In some embodiments, epitopes of the disclosure are from a spike protein. In some embodiments, epitopes of the disclosure comprise a receptor binding domain of a Spike protein. In some embodiments, epitopes of the disclosure comprise an ACE2 binding domain of a Spike protein.

Envelope (E) encodes envelope protein, which in some embodiments contributes to virus assembly and morphogenesis. Envelope protein can be an antigen. In some embodiments, epitopes of the disclosure are from a coronavirus envelope protein.

Membrane (M) encodes membrane protein, which in some embodiments contributes to viral assembly. Membrane protein can be an antigen. In some embodiments, epitopes of the disclosure are from a coronavirus membrane protein.

Nucleocapsid (N) encodes nucleocapsid protein, which in some embodiments can form complexes with genomic RNA and contribute to viral assembly, and/or interact with M protein. Nucleocapsid protein can be an antigen. In some embodiments, epitopes of the disclosure are from a coronavirus nucleocapsid protein.

ORF3a, ORF3b, ORF6, ORF7a, ORF7b, ORF8, ORF8a, ORF8b, ORF9a, ORF9b, and ORF10 encodes accessory proteins. In some embodiments, accessory proteins can modulate host cell signaling, modulate host cell immune responses, be incorporated into mature virions as minor structural proteins, or a combination thereof. An accessory protein can be an antigen. In some embodiments, epitopes of the disclosure are from a coronavirus accessory protein.

Compositions and methods of the disclosure can utilize antigens and/or epitopes that are encoded by or derived from one or more open reading frames of a SARS-CoV genome. For example, antigens and/or epitopes can be encoded by or derived from ORF1a, ORF1b, spike (S), ORF3a, ORF3b, envelope (E), membrane (M), ORF6, ORF7a, ORF7b, ORF8, ORF8a, ORF8b, ORF9a, ORF9b, nucleocapsid (N), ORF10, or any combination thereof.

In some embodiments, epitopes of the disclosure are from a spike protein. In some embodiments, epitopes of the disclosure comprise a receptor binding domain (RBD) of a Spike protein. In some embodiments, epitopes of the disclosure comprise an ACE2 binding domain of a Spike protein. In some embodiments, epitopes of the disclosure comprise an S1 subunit Spike protein, an S2 subunit of spike protein, or a combination thereof. In some embodiments, epitopes of the disclosure comprise an ectodomain of a spike protein. In some embodiments, an epitope of the disclosure comprises Gln498, Thr500, Asn501, or a combination thereof from a coronavirus spike protein. In some embodiments, an epitope of the disclosure comprises Lys417, Tyr453, or a combination thereof from a coronavirus spike protein. In some embodiments, an epitope of the disclosure comprises Gln474, Phe486, or a combination thereof from a coronavirus spike protein. In some embodiments, an epitope of the disclosure comprises Gln498, Thr500, Asn501, Lys417, Tyr453, Gln474, Phe486, one or more equivalent amino acids from a spike protein variant or derivative, or a combination thereof from a coronavirus spike protein. In some embodiments, the spike protein of the disclosure comprises a D614G mutation, namely having amino acid glycine (G) at the 614 location instead of aspartic acid (D). In some embodiments, an epitope of the disclosure comprises Gly614 from a spike protein variant or derivative, or combination thereof from a coronavirus spike protein. In some cases, the D614G mutation can lead to reduction of S1 shedding and increase in the infectivity of the coronavirus.

In some embodiments, antigens and/or epitopes are encoded by or derived from ORF1a. In some embodiments, antigens and/or epitopes are encoded by or derived from a SARS-CoV ORF1b. In some embodiments, antigens and/or epitopes are encoded by or derived from a SARS-CoV spike. In some embodiments, antigens and/or epitopes are encoded by or derived from a SARS-CoV ORF3a. In some embodiments, antigens and/or epitopes are encoded by or derived from a SARS-CoV ORF3b. In some embodiments, antigens and/or epitopes are encoded by or derived from a SARS-CoV envelope (E). In some embodiments, antigens and/or epitopes are encoded by or derived from a SARS-CoV membrane (M). In some embodiments, antigens and/or epitopes are encoded by or derived from a SARS-CoV ORF6. In some embodiments, antigens and/or epitopes are encoded by or derived from a SARS-CoV ORF7a. In some embodiments, antigens and/or epitopes are encoded by or derived from a SARS-CoV ORF7b. In some embodiments, antigens and/or epitopes are encoded by or derived from a SARS-CoV ORF8. In some embodiments, antigens and/or epitopes are encoded by or derived from a SARS-CoV ORF8a. In some embodiments, antigens and/or epitopes are encoded by or derived from a SARS-CoV ORF9a. In some embodiments, antigens and/or epitopes are encoded by or derived from a SARS-CoV ORF9b. In some embodiments, antigens and/or epitopes are encoded by or derived from a SARS-CoV nucleocapsid (N). In some embodiments, antigens and/or epitopes are encoded by or derived from a SARS-CoV ORF10. In some embodiments, antigens and/or epitopes are encoded by or derived from a SARS-CoV spike (S), envelope (E), membrane (M), and nucleocapsid (N).

In some embodiments, antigens and/or epitopes are not encoded by or derived from a SARS-CoV ORF1a. In some embodiments, antigens and/or epitopes are not encoded by or derived from a SARS-CoV ORF1b. In some embodiments, antigens and/or epitopes are not encoded by or derived from a SARS-CoV spike. In some embodiments, antigens and/or epitopes are not encoded by or derived from a SARS-CoV ORF3a. In some embodiments, antigens and/or epitopes are not encoded by or derived from a SARS-CoV ORF3b. In some embodiments, antigens and/or epitopes are not encoded by or derived from a SARS-CoV envelope (E). In some embodiments, antigens and/or epitopes are not encoded by or derived from a SARS-CoV membrane (M). In some embodiments, antigens and/or epitopes are not encoded by or derived from a SARS-CoV ORF6. In some embodiments, antigens and/or epitopes are not encoded by or derived from a SARS-CoV ORF7a. In some embodiments, antigens and/or epitopes are not encoded by or derived from a SARS-CoV ORF7b. In some embodiments, antigens and/or epitopes are not encoded by or derived from a SARS-CoV ORF8. In some embodiments, antigens and/or epitopes are not encoded by or derived from a SARS-CoV ORF8a. In some embodiments, antigens and/or epitopes are not encoded by or derived from a SARS-CoV ORF9a. In some embodiments, antigens and/or epitopes are not encoded by or derived from a SARS-CoV ORF9b. In some embodiments, antigens and/or epitopes are not encoded by or derived from a SARS-CoV nucleocapsid (N). In some embodiments, antigens and/or epitopes are not encoded by or derived from a SARS-CoV ORF10. In some embodiments, antigens and/or epitopes are not encoded by or derived from a SARS-CoV spike (S), envelope (E), membrane (M), and nucleocapsid (N).

An antigen and/or epitope can be encoded by or derived from SARS-CoV2.

A non-limiting example of a SARS-CoV-2 genome is provided in DB Source accession MN908947.3, the complete genome sequence of a Severe acute respiratory syndrome coronavirus 2 isolate, the content of which is incorporated herein by reference in its entirety. DB Source accession MN908947.3: 21563-25384 corresponds to the S protein, the content of which is incorporated herein by reference in its entirety. A non-limiting example of a SARS-CoV-2 spike protein is provided in GenBank Sequence: QHD43416.1, the sequence of a spike protein of a Severe acute respiratory syndrome coronavirus 2 isolate, the content of which is incorporated herein by reference in its entirety

A non-limiting example of a SARS-CoV-2 genome is provided in sequence NCBI Reference Sequence accession number NC_045512, version NC_045512.2, the complete genome sequence of Severe acute respiratory syndrome coronavirus 2 isolate Wuhan-Hu-1, the content of which is incorporated herein by reference in its entirety.

A non-limiting example of a SARS-CoV-2 genome is provided in sequence NCBI Reference Sequence accession number MW450666, the complete genome sequence of Severe acute respiratory syndrome coronavirus 2 isolate, the content of which is incorporated herein by reference in its entirety.

A non-limiting example of a SARS-CoV-2 genome is provided in sequence NCBI Reference Sequence accession number MW487270, the complete genome sequence of Severe acute respiratory syndrome coronavirus 2 lineage B.1.1.7 virus, the content of which is incorporated herein by reference in its entirety.

A non-limiting example of a SARS-CoV-2 genome is provided in sequence GISAID Reference Sequence accession number EPI_ISL_792683, the complete genome sequence of Severe acute respiratory syndrome coronavirus 2 lineage P.1 virus, the content of which is incorporated herein by reference in its entirety.

A non-limiting example of a SARS-CoV-2 genome is provided in sequence GISAID Reference Sequence accession number EPI_ISL_678615, the complete genome sequence of Severe acute respiratory syndrome coronavirus 2 lineage B.1.351 virus, the content of which is incorporated herein by reference in its entirety.

Non-limiting examples of a SARS-CoV-2 genome are provided in sequence NCBI Reference Sequence accession numbers MW972466-MW974550, the complete genome sequence of Severe acute respiratory syndrome coronavirus 2 lineage B.1.427 and B.1.429 virus, the contents of which are incorporated herein by reference in their entirety.

Non-limiting examples of a SARS-CoV-2 genome are provided in sequence NCBI Reference Sequence accession numbers MZ156756-MZ226428, the complete genome sequence of Severe acute respiratory syndrome coronavirus 2 virus, the contents of which are incorporated herein by reference in their entirety.

In some embodiments, the SAR-CoV-2 genome is provided in the GISAID Database at www.gisaid.org. In some embodiments, the SARS-CoV-2 genome is provided in the International Nucleotide Sequence Database Collaboration (INSDC) at www.insdc.org.

In some embodiments, an antigen and/or epitope of the disclosure is from a predicted transcript from a SARS-CoV-2 genome. In some embodiments, an antigen and/or epitope of the disclosure is from a protein encoded by an open reading frames from a SARS-CoV-2 genome, or a derivative thereof. Non-limiting examples of open reading frames in the SARS-CoV-2 genome include ORF1a, ORF1b, spike (S), ORF3a, envelope (E), membrane (M), ORF6, ORF7a, ORF7b, ORF8, nucleocapsid (N), and ORF10. In some embodiments, a SARS-CoV-2 genome encodes an ORF3b, ORF9a, ORF9b, or a combination thereof. In some embodiments, a SARS-CoV-2 genome does not encode an ORF3b, ORF9a, ORF9b, or any combination thereof.

Nonlimiting examples of amino acid sequences are provided in TABLE 1. In some embodiments, the antigen comprises a sequence having at least about 80%, 85%, 90%, 95%, 97%, 98%, 99%, or 100% sequence identity to a sequence from TABLE 1.

TABLE 1
Examples of amino acid sequence of proteins encoded
by a SARS-CoV-2 genome.

| SEQ ID | | |
|---|---|---|
| NO: | Description | Sequence |
| 1 | Spike (S) | MFVFLVLLPLVSSQCVNLTTRTQLPPAYTNSFTRGVYYPDKVF |
| | protein | RSSVLHSTQDLFLPFFSNVTWFHAIHVSGTNGTKRFDNPVLPF |
| | | NDGVYFASTEKSNIIRGWIFGTTLDSKTQSLLIVNNATNVVIK |
| | | VCEFQFCNDPFLGVYYHKNNKSWMESEFRVYSSANNCTFEYVS |
| | | QPFLMDLEGKQGNFKNLREFVFKNIDGYFKIYSKHTPINLVRD |
| | | LPQGFSALEPLVDLPIGINITRFQTLLALHRSYLTPGDSSSGW |
| | | TAGAAAYYVGYLQPRTFLLKYNENGTITDAVDCALDPLSETKC |
| | | TLKSFTVEKGIYQTSNFRVQPTESIVRFPNITNLCPFGEVFNA |
| | | TRFASVYAWNRKRISNCVADYSVLYNSASFSTFKCYGVSPTKL |
| | | NDLCFTNVYADSFVIRGDEVRQIAPGQTGKIADYNYKLPDDFT |
| | | GCVIAWNSNNLDSKVGGNYNYLYRLFRKSNLKPFERDISTEIY |
| | | QAGSTPCNGVEGFNCYFPLQSYGFQPTNGVGYQPYRVVVLSFE |
| | | LLHAPATVCGPKKSTNLVKNKCVNFNFNGLTGTGVLTESNKKF |
| | | LPFQQFGRDIADTTDAVRDPQTLEILDITPCSFGGVSVITPGT |
| | | NTSNQVAVLYQDVNCTEVPVAIHADQLTPTWRVYSTGSNVFQT |
| | | RAGCLIGAEHVNNSYECDIPIGAGICASYQTQTNSPRRARSVA |
| | | SQSIIAYTMSLGAENSVAYSNNSIAIPTNFTISVTTEILPVSM |
| | | TKTSVDCTMYICGDSTECSNLLLQYGSFCTQLNRALTGIAVEQ |
| | | DKNTQEVFAQVKQIYKTPPIKDFGGFNFSQILPDPSKPSKRSF |
| | | IEDLLFNKVTLADAGFIKQYGDCLGDIAARDLICAQKFNGLTV |
| | | LPPLLTDEMIAQYTSALLAGTITSGWTFGAGAALQIPFAMQMA |
| | | YRFNGIGVTQNVLYENQKLIANQFNSAIGKIQDSLSSTASALG |
| | | KLQDVVNQNAQALNTLVKQLSSNFGAISSVLNDILSRLDKVEA |
| | | EVQIDRLITGRLQSLQTYVTQQLIRAAEIRASANLAATKMSEC |
| | | VLGQSKRVDFCGKGYHLMSFPQSAPHGVVFLHVTYVPAQEKNF |
| | | TTAPAICHDGKAHFPREGVFVSNGTHWFVTQRNFYEPQIITTD |
| | | NTFVSGNCDVVIGIVNNTVYDPLQPELDSFKEELDKYFKNHTS |
| | | PDVDLGDISGINASVVNIQKEIDRLNEVAKNLNESLIDLQELG |
| | | KYEQYIKWPWYIWLGFIAGLIAIVMVTIMLCCMTSCCSCLKGC |
| | | CSCGSCCKFDEDDSEPVLKGVKLHYT |
| 2 | Envelope (E) | MYSFVSEETGTLIVNSVLLFLAFVVFLLVTLAILTALRLCAYC |
| | protein | CNIVNVSLVKPSFYVYSRVKNLNSSRVPDLLV |
| 3 | Membrane (M) | MADSNGTITVEELKKLLEQWNLVIGFLFLTWICLLQFAYANRN |
| | protein | RFLYIIKLIFLWLLWPVTLACFVLAAVYRINWITGGIAIAMAC |
| | | LVGLMWLSYFIASFRLFARTRSMWSFNPETNILLNVPLHGTIL |
| | | TRPLLESELVIGAVILRGHLRIAGHHLGRCDIKDLPKEITVAT |
| | | SRTLSYYKLGASQRVAGDSGFAAYSRYRIGNYKLNTDHSSSSD |
| | | NIALLVQ |
| 4 | Nucleocapsid | MSDNGPQNQRNAPRITFGGPSDSTGSNQNGERSGARSKQRRPQ |
| | (N) | GLPNNTASWFTALTQHGKEDLKFPRGQGVPINTNSSPDDQIGY |
| | protein | YRRATRRIRGGDGKMKDLSPRWYFYYLGTGPEAGLPYGANKDG |
| | | IIWVATEGALNTPKDHIGTRNPANNAAIVLQLPQGTTLPKGFY |
| | | AEGSRGGSQASSRSSSRSRNSSRNSTPGSSRGTSPARMAGNGG |
| | | DAALALLLLDRLNQLESKMSGKGQQQQGQTVTKKSAAEASKKP |
| | | RQKRTATKAYNVTQAFGRRGPEQTQGNFGDQELIRQGTDYKHW |
| | | PQIAQFAPSASAFFGMSRIGMEVTPSGTWLTYTGAIKLDDKDP |
| | | NFKDQVILLNKHIDAYKTFPPTEPKKDKKKKADETQALPQRQK |
| | | KQQTVTLLPAADLDDFSKQLQQSMSSADSTQA |
| 5 | ORF3a accessory | MDLFMRIFTIGTVTLKQGEIKDATPSDFVRATATIPIQASLPF |
| | protein | GWLIVGVALLAVFQSASKIITLKKRWQLALSKGVHFVCNLLLL |
| | | FVTVYSHLLLVAAGLEAPFLYLYALVYFLQSINFVRIIMRLWL |
| | | CWKCRSKNPLLYDANYFLCWHTNCYDYCIPYNSVTSSIVITSG |
| | | DGTTSPISEHDYQIGGYTEKWESGVKDCVVLHSYFTSDYYQLY |
| | | STQLSTDTGVEHVTFFIYNKIVDEPEEHVQIHTIDGSSGVVNP |
| | | VMEPIYDEPTTTTSVPL |
| 6 | ORF6 accessory | MFHLVDFQVTIAEILLIIMRTFKVSIWNLDYIINLIIKNLSKS |
| | protein | LTENKYSQLDEEQPMEID |
| 7 | ORF7a accessory | MKIILFLALITLATCELYHYQECVRGTTVLLKEPCSSGTYEGN |
| | protein | SPFHPLADNKFALTCFSTQFAFACPDGVKHVYQLRARSVSPKL |
| | | FIRQEEVQELYSPIFLIVAAIVFITLCFTLKRKTE |
| 8 | ORF7b accessory | MIELSLIDFYLCFLAFLLFLVLIMLIIFWFSLELQDHNETCHA |
| | protein | |
| 9 | ORF8 accessory | MKFLVFLGIITTVAAFHQECSLQSCTQHQPYVVDDPCPIHFYS |
| | protein | KWYIRVGARKSAPLIELCVDEAGSKSPIQYIDIGNYTVSCLPF |
| | | TINCQEPKLGSLVVRCSFYEDFLEYHDVRVVLDFI |
| 10 | ORF10 accessory | MGYINVFAFPFTIYSLLLCRMNSRNYIAQVDVVNFNLT |
| | protein | |

Additional non-limiting examples of proteins encoded by a SARS-CoV-2 genome include those with the contents of NCBI accession numbers MT334522, MT334523, MT334524, MT334525, MT334526, MT334527, MT334528, MT334529, MT334530, MT334531, MT334532, MT334533, MT334534, MT334535, MT334536, MT334537, MT334538, MT334539, MT334540, MT334541, MT334542, MT334543, MT334544, MT334545, MT334546, MT334555, MT334547, MT334548, MT334549, MT334550, MT334551, MT334552, MT334553, MT334554, MT334556, MT334557, MT334558, MT334559, MT334560, MT334561, MT334562, MT334563, MT334564, MT334565, MT334566, MT334567, MT334568, MT334569, MT334570, MT334571, MT334572, MT334573, MT326097, MT326106, MT326107, MT326116, MT326117, MT326124, MT326125, MT326126, MT326127, MT326134, MT326135, MT326136, MT326137, MT326138, MT326139, MT326140, MT326141, MT326142, MT326143, MT326144, MT326145, MT326146, MT326148, MT326149, MT326150, MT326151, MT326152, MT326158, MT326159, MT326160, MT326161, MT326162, MT326168, MT326169, MT326170, MT326171, MT326172, MT326178, MT326179, MT326180, MT326181, MT326182, MT326183, MT326188, MT326189, MT326190, MT326191, MT326129, MT326121, MT326120, MT326119, MT326118, MT326111, MT326023, MT326025, MT326033, MT326035, MT326036, MT326040, MT326043, MT326045, MT326053, MT326055, MT326056, MT326063, MT326066, MT326070, MT326071, MT326072, MT326075, MT326076, MT326078, MT326079, MT326089, MT325563, MT325565, MT325566, MT326155, MT326163, MT326177, MT326130, MT326128, MT326110, MT326109, MT326108, MT326101, MT326100, MT326099, MT326098, MT326094, MT326093, MT326092, MT325568, MT325569, MT325590, MT325640, MT325606, MT325607, MT325608, MT325609, MT325610, MT325611, MT325616, MT325618, MT325619, MT325620, MT325622, MT325623, MT325624, MT325599, MT325600, MT325601, MT325602, MT325612, MT325613, MT325615, MT325617, MT325625, MT324062, MT324684, MT325573, MT325574, MT325577, MT325579, MT325586, MT325592, MT325593, MT325594, MT325598, MT325605, MT325626, MT325627, MT325633, MT325634, MT326028, MT326031, MT326091, MT326090, MT326085, MT326084, MT326083, MT326082, MT326081, MT326080, MT326077, MT326067, MT326057, MT326024, MT326026, MT326027, MT326032, MT326034, MT326037, MT326039, MT326041, MT326042, MT326044, MT326046, MT326047, MT326049, MT326050, MT326051, MT326052, MT326054, MT326059, MT326060, MT326061, MT326062, MT326064, MT326065, MT326068, MT326069, MT326073, MT326074, MT326088, MT327745, MT324679, MT325561, MT325571, MT325572, MT325575, MT325583, MT325587, MT325588, MT325589, MT325596, MT325597, MT325603, MT325604, MT325614, MT325621, MT325629, MT325630, MT325631, MT325632, MT325635, MT325636, MT325637, MT325638, MT325639, MT326086, MT326096, MT326102, MT326104, MT326105, MT326112, MT326113, MT326114, MT326115, MT326122, MT328034, MT325564, MT325567, MT326164, MT326165, MT326173, MT326174, MT326184, MT326185, MT326186, MT326187, MT325584, MT325585, MT326087, MT326095, MT326103, MT326123, MT326131, MT326132, MT326133, MT328033, MT325562, MT326147, MT326153, MT326154, MT326156, MT326157, MT326166, MT326167, MT326175, MT326176, MT324680, MT325570, MT325576, MT325578, MT325580, MT325581, MT325582, MT325591, MT325595, MT325628, MT326029, MT326030, MT326038, MT326048, MT326058, MT324681, MT324682, MT324683, MT328032, MT328035, MT322404, MT039874, MT322398, MT322409, MT322421, MT322423, MT322408, MT322413, MT322417, MT322394, MT322407, MT322418, MT322424, MT322411, MT077125, MT322395, MT322396, MT322397, MT322399, MT322400, MT322401, MT322402, MT322403, MT322405, MT322406, MT322414, MT322416, MT322419, MT322420, MT322410, MT322412, MT322415, MT322422, MT320538, MT320891, MT308692, MT308693, MT308695, MT308696, MT308698, MT308699, MT308701, MT308703, MT308704, MT308694, MT308697, MT308700, MT308702, MT293547, MT304476, MT304474, MT304475, MT304477, MT304478, MT304479, MT304481, MT304482, MT304484, MT304485, MT304486, MT304487, MT304488, MT304491, MT304480, MT304483, MT304489, MT304490, MT300186, MT292571, MT292576, MT292578, MT293186, MT292570, MT292573, MT293173, MT292575, MT293179, MT293180, MT293184, MT293189, MT293192, MT293193, MT293194, MT293201, MT293202, MT292572, MT292577, MT293185, MT293187, MT293188, MT291826, MT291832, MT291833, MT291835, MT291836, MT291831, MT293170, MT292574, MT293178, MT293181, MT293183, MT293195, MT293196, MT293197, MT293203, MT293204, MT293223, MT293212, MT293214, MT293215, MT293216, MT293219, MT293224, MT293225, MT293206, MT293208, MT293209, MT293221, MT295464, MT293160, MT293166, MT293171, MT293190, MT293161, MT293167, MT293168, MT293174, MT293175, MT293182, MT293191, MT293158, MT293162, MT293163, MT293164, MT293156, MT293157, MT293159, MT291834, MT291829, MT291827, MT291830, MT291828, MT293169, MT293200, MT293210, MT293211, MT293217, MT293218, MT295465, MT293198, MT293205, MT293207, MT293213, MT293220, MT293222, MT292581, MT292569, MT293172, MT293177, MT293176, MT293199, MT292580, MT292582, MT293165, MT292579, MT273658, MT281577, MT281530, MT276597, MT276598, MT276323, MT276328, MT276331, MT276329, MT276330, MT276324, MT276325, MT276327, MT276326, MT263388, MT263392, MT262900, MT262902, MT262906, MT262908, MT262912, MT262913, MT262914, MT262993, MT263074, MT263381, MT263391, MT262901, MT262903, MT262907, MT262909, MT262911, MT262899, MT262904, MT262915, MT262916, MT262897, MT262898, MT262905, MT262910, MT263400, MT263382, MT263383, MT263384, MT263385, MT262896, MT263407, MT263415, MT263406, MT263408, MT263422, MT263469, MT263439, MT263457, MT263459, MT263432, MT263450, MT263458, MT263467, MT263401, MT263411, MT263413, MT263426, MT263421, MT263443, MT263412, MT263416, MT263417, MT263423, MT263431, MT263461, MT263410, MT263424, MT263425, MT263427, MT263442, MT263402, MT263405, MT263409, MT263418, MT263419, MT263398, MT263399, MT263403, MT263404, MT263414, MT263430, MT263390, MT263434, MT263436, MT263446, MT263448, MT263452, MT263453, MT263456, MT263462, MT263463, MT263386, MT263387, MT263389, MT263428, MT263429, MT263433, MT263435, MT263437, MT263438, MT263440, MT263447, MT263449, MT263455, MT263444, MT263445, MT263451, MT263466, MT263420, MT263441, MT263454, MT263464, MT263465, MT263468, MT263460, MT263393, MT263394, MT263395, MT263396, MT263397, MT259226, MT259275, MT259276, MT259279, MT259247, MT258377, MT258378, MT258379, MT259231, MT259228, MT259238, MT259248, MT256917, MT259227, MT259236, MT256918, MT258380, MT259235, MT259237, MT259239, MT259281, MT259282, MT259283, MT259240, MT259243, MT259249, MT259250, MT259251, MT259256, MT259258, MT259266, MT259267, MT259274, MT259286, MT259287, MT259241, MT259242, MT258381, MT259257, MT259261, MT259262, MT259263, MT259264, MT259268, MT259269, MT259270, MT259271, MT259272, MT259273, MT259277, MT259278, MT259280, MT258383, MT258382, MT259246, MT256924, MT259244, MT259245, MT259252, MT259253, MT259254, MT259255, MT259259, MT259284, MT259229, MT259230, MT259265, MT259260, MT259285, LC534419, LC534418, MT253710, MT253709, MT253705, MT253708, MT253701, MT253702, MT253703, MT253704, MT253706, MT253707, MT251972, MT251974, MT251975, MT251973, MT251976, MT251979, MT253697, MT253699, MT253696, MT253698, MT253700, MT251977, MT251978, MT251980, MT246451, MT246461, MT246471, MT246472, MT246474, MT246483, MT246450, MT246453, MT246454, MT246462, MT246463, MT246464, MT246470, MT246473, MT246480, MT246484, MT246449, MT246455, MT246456, MT246478, MT246485, MT246488, MT246452, MT246460, MT246465, MT246481, MT246482, MT246490, MT246459, MT246468, MT246475, MT246477, MT246479, MT246457, MT246458, MT246466, MT246467, MT246469, MT246476, MT246486, MT246487, MT246489, MT233526, MT246667, MT240479, MT232870, MT232871, MT233523, MT232869, MT232872, MT233519, MT233521, MT233522, MT233520, MT226610, MT198653, MT198651, MT198652, MT192773, MT192758, MT192772, MT192765, MT192759, MT188341, MT188340, MT188339, MT186676, MT186681, MT186677, MT186678, MT187977, MT186680, MT186682, MT186679, MT184909, MT184911, MT184912, MT184913, MT184910, MT184907, MT184908, CADDYA000000000, MT163718, MT163719, MT163720, MT163714, MT163715, MT163721, MT163717, MT163737, MT163738, MT163712, MT163716, MT159706, MT159716, MT159719, MT159707, MT159717, MT159709, MT159715, MT159718, MT159722, MT159708, MT161607, MT159705, MT159710, MT159711, MT159712, MT159713, MT159714, MT159720, MT159721, MT121215, MT159778, MT066156, LC529905, MT050493, MT012098, MT152900, MT152824, MT135044, MT135042, MT135041, MT135043, MT126808, MT127113, MT127114, MT127116, MT127115, LC528232, LC528233, MT123293, MT123291, MT123290, MT123292, MT118835, MT111896, MT111895, MT106052, MT106053, MT106054, MT093571, MT093631, MT081061, MT081063, MT081066, MT081062, MT081064, MT081065, MT081067, MT081059, MT081060, MT081068, MT072667, MT072668, MT072688, MT066157, MT066176, MT066159, MT066175, MT066158, LC523809, LC523807, LC523808, MT044258, MT044257, MT050416, MT050417, MT042773, MT042774, MT042775, MT042776, MT049951, MT050414, MT050415, MT042777, MT042778, MT039887, MT039888, MT039890, MT039873, LC522350, MT027062, MT027063, MT027064, MT020881, MT019530, MT019531, MT019533, MT020880, MT019532, MT019529, MT020781, LR757995, LR757998, LR757996, LR757997, MT007544, MT008022, MT008023, MN996531, MN996530, MN996527, MN996528, MN996529, MN997409, MN988668, MN988669, MN994467, MN994468, MN988713, MN938384, MN975262, MN985325, MN938386, MN938388, MN938385, MN938387, MN938390, MN938389, MN975263, MN975267, MN975268, MN975265, MN975264, MN975266, MN970004, MN970003, MN908947 each of which is incorporated herein by reference in its entirety.

In particular embodiments, a circular polyribonucleotide comprises a SARS-CoV-2 antigen described in TABLE 2. In some embodiments, the antigen comprises a sequence having at least about 80%, 85%, 90%, 95%, 97%, 98%, 99%, or 10000 sequence identity to a sequence from TABLE 2.

TABLE 2
Descriptions of constructs and SARS-CoV-2 ORFs

| | ORF | ORF | Proline | Cloning | Circularization | |
|---|---|---|---|---|---|---|
| Construct | (SEQ ID NO.) | Description | substitutions | optimization | optimization | IRES |
| p1 | 1 (13) | S protein | Yes | Yes | No | CVB3 |
| SEQ ID | | transmembrane | | | | |
| NO. 12 | | (TM) domain | | | | |
| | | completely | | | | |
| | | removed, and a | | | | |
| | | trimerization | | | | |
| | | domain added | | | | |
| p3 | 3 (15) | S protein | Yes | Yes | No | CVB3 |
| SEQ ID | | transmembrane | | | | |
| NO. 14 | | (TM) domain | | | | |
| | | fully intact | | | | |
| p5 | 5 (17) | S protein | Yes | Yes | No | CVB3 |
| SEQ ID | | transmembrane | | | | |
| NO. 16 | | (TM) domain | | | | |
| | | partially | | | | |
| | | removed (111 of | | | | |
| | | 195 nucleotides | | | | |
| | | were removed) | | | | |
| | | (nucleotides | | | | |
| | | 3709-3819 were | | | | |
| | | removed) | | | | |
| p7 | 7 (19) | S protein | Yes | Yes | Yes | CVB3 |
| SEQ ID | | transmembrane | | | | |
| NO. 18 | | (TM) domain | | | | |
| | | completely | | | | |
| | | removed, and a | | | | |
| | | trimerization | | | | |
| | | domain added | | | | |
| p9 | 9 (21) | S protein | Yes | Yes | Yes | CVB3 |
| SEQ ID | | transmembrane | | | | |
| NO. 20 | | (TM) domain | | | | |
| | | fully intact | | | | |
| p11 | 11 (23) | S protein | Yes | Yes | Yes | CVB3 |
| SEQ ID | | transmembrane | | | | |
| NO. 22 | | (TM) domain | | | | |
| | | partially | | | | |
| | | removed (111 of | | | | |
| | | 195 nucleotides | | | | |
| | | were removed) | | | | |
| | | (nucleotides | | | | |
| | | 3709-3819 were | | | | |
| | | removed) | | | | |
| p13 | 13 (25) | S protein | N/A | N/A | N/A | CVB3 |
| SEQ ID | | receptor binding | | | | |
| NO. 24 | | domain (RBD) | | | | |
| | | only with | | | | |
| | | secretion signal | | | | |
| | | translationally | | | | |
| | | fused to the 5’ | | | | |
| | | end | | | | |
| p15 | 3 (15) | S protein | Yes | Yes | N/A | EMCV |
| | | transmembrane | | | | |
| | | (TM) domain | | | | |
| | | completely | | | | |
| | | removed, and a | | | | |
| | | trimerization | | | | |
| | | domain added | | | | |
| p17 | 5 (17) | S protein | Yes | Yes | N/A | EMCV |
| | | transmembrane | | | | |
| | | (TM) domain | | | | |
| | | fully intact | | | | |
| p19 | 1 (13) | S protein | Yes | Yes | N/A | EMCV |
| | | transmembrane | | | | |
| | | (TM) domain | | | | |
| | | partially | | | | |
| | | removed (111 of | | | | |
| | | 195 nucleotides | | | | |
| | | were removed) | | | | |
| | | (nucleotides | | | | |
| | | 3709-3819 were | | | | |
| | | removed) | | | | |
| p21 | 7 (19) | S protein | Yes | Yes | Yes | EMCV |
| | | transmembrane | | | | |
| | | (TM) domain | | | | |
| | | completely | | | | |
| | | removed, and a | | | | |
| | | trimerization | | | | |
| | | domain added | | | | |
| p23 | 9 (21) | S protein | Yes | Yes | Yes | EMCV |
| | | transmembrane | | | | |
| | | (TM) domain | | | | |
| | | fully intact | | | | |
| p25 | 11 (23) | S protein | Yes | Yes | Yes | EMCV |
| | | transmembrane | | | | |
| | | (TM) domain | | | | |
| | | partially | | | | |
| | | removed (111 of | | | | |
| | | 195 nucleotides | | | | |
| | | were removed) | | | | |
| | | (nucleotides | | | | |
| | | 3709-3819 were | | | | |
| | | removed) | | | | |
| p27 | 13 (25) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5’ | | | | |
| | | end | | | | |
| p33 | 33 (26) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5’ | | | | |
| | | end and RBD | | | | |
| | | type II | | | | |
| | | terminator | | | | |
| | | removed | | | | |
| p35 | 35 (27) | S protein | Yes | Yes | No | EMCV |
| | | transmembrane | | | | |
| | | (TM) domain | | | | |
| | | fully intact and | | | | |
| | | RBD type II | | | | |
| | | terminator | | | | |
| | | removed | | | | |
| p36 | 36 (28) | S protein | Yes | Yes | Yes | EMCV |
| | | transmembrane | | | | |
| | | (TM) domain | | | | |
| | | fully intact and | | | | |
| | | RBD type II | | | | |
| | | terminator | | | | |
| | | removed | | | | |
| p39 | 39 (29) | S protein | N/A | N/A | N/A | EMCV |
| | | transmembrane | | | | |
| | | (TM) domain | | | | |
| | | fully intact . GC | | | | |
| | | optimized | | | | |
| p41 | 41 (30) | S protein | N/A | N/A | N/A | EMCV |
| | | receptor binding | | | | |
| | | domain (RBD) | | | | |
| | | only with | | | | |
| | | secretion signal | | | | |
| | | translationally | | | | |
| | | fused to the 5’ | | | | |
| | | end and RBD | | | | |
| | | type II | | | | |
| | | terminator | | | | |
| | | removed. GC | | | | |
| | | optimized | | | | |
| p44 | 44 (48) | S protein | N/A | N/A | N/A | EMCV |
| | | receptor binding | | | | |
| | | domain (RBD) | | | | |
| | | only with IL-2 | | | | |
| | | secretion signal | | | | |
| | | translationally | | | | |
| | | fused to the 5’ | | | | |
| | | end and RBD | | | | |
| | | type II | | | | |
| | | terminator | | | | |
| | | removed | | | | |
| p45 | 45 (49) | S protein | N/A | N/A | N/A | EMCV |
| | | receptor binding | | | | |
| | | domain (RBD) | | | | |
| | | only with Gluc | | | | |
| | | secretion signal | | | | |
| | | translationally | | | | |
| | | fused to the 5’ | | | | |
| | | end and RBD | | | | |
| | | type II | | | | |
| | | terminator | | | | |
| | | removed | | | | |

In TABLE 2, “proline substitutions” denote proline substitutions at residues 986 and 987, as well as a “GSAS” substitution at the furin cleavage site (residues 682-685). For “cloning optimization,” single base substitution was made at coordinate 2541 to destroy a BsaI site to assist in Golden Gate Cloning construction of the plasmid DNA template. For “circularization optimizations”: four single nucleotides—at positions 2307, 2790, 159 and 315—were substituted to destroy sites that could potentially bind circularization elements of splint nucleic acid sequences, thereby potentially inhibiting efficient ligation. For constructs that have type II terminator removed (e.g., p33, p35, p36, p39, p41, p44, and p45): two single nucleotides—at positions 1047, 1049 were substituted to destroy type II terminator site. For constructs that have GC optimization (e.g., p39 and p41), GC optimization was performed such that GC content was approximately 50%. All single base pair substitutions were designed to be translationally silent. Further, in TABLE 2, IRES is EMCV (SEQ ID NO: 31) or is CVB3 (SEQ ID NO: 45).

In some embodiments, an antigen or epitope is from a host subject (e.g., a subject for immunization) cell. For example, antibodies that block entry of a coronavirus can be produced by using an antigen or epitope from a component of a host cell that the virus uses as an entry factor.

In some embodiments, a coronavirus epitope comprises or contains at least 4, at least 5, at least 6, at least 7, at least 8, at least 9, at least 10, at least 11, at least 12, at least 13, at least 14, at least 15, at least 16, at least 17, at least 18, at least. 19, at least 20, at least 21, at least 22, at least 23, at least 24, at least 25, at least 26, at least 27, at least 28, at least 29, or at least 30 amino acids, or more. In some embodiments, a coronavirus epitope comprises or contains at most 4, at most 5, at most 6, at most 7, at most 8, at most 9, at most 10, at most 11, at most 12, at most 13, at most 14, at most 15, at most 16, at most 17, at most 18, at most. 19, at most 20, at most 21, at most 22, at most 23, at most 24, at most 25, at most 26, at most 27, at most 28, at most 29, or at most 30 amino acids, or less. In some embodiments, a coronavirus epitope comprises or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, a coronavirus epitope contains 5 amino acids. In some embodiments, a coronavirus epitope contains 6 amino acids. In some embodiments, an epitope contains 7 amino acids. In some embodiments, a coronavirus epitope contains 8 amino acids. In some embodiments, an epitope can be about 8 to about 11 amino acids. In some embodiments, an epitope can be about 9 to about 22 amino acids.

The coronavirus antigens may comprise antigens recognized by B cells, antigens recognized by T cells, or a combination thereof. In some embodiments, the antigens comprise antigens recognized by B cells. In some embodiments, the coronavirus antigens are antigens recognized by B cells. In some embodiments, the coronavirus antigens comprise antigens recognized by T cells. In some embodiments, the antigens are antigens recognized by T cells.

The coronavirus epitopes comprise recognized by B cells, antigens recognized by T cells, or a combination thereof. In some embodiments, the coronavirus epitopes comprise epitopes recognized by B cells. In some embodiments, the epitopes are epitopes recognized by B cells. In some embodiments, the coronavirus epitopes comprise epitopes recognized by T cells. In some embodiments, the coronavirus epitopes are epitopes recognized by T cells.

Techniques for identifying antigens and epitopes in silico have been disclosed, for example, in Sanchez-Trincado, et al. (2017), Fundamentals and methods for T- and B-cell epitope prediction. Journal of immunology research; Grifoni, Alba, et al. A Sequence Homology and Bioinformatic Approach Can Predict Candidate Targets for Immune Responses to SARS-CoV-2. Cell host & microbe (2020); Russi et al. In silico prediction of T- and B-cell epitopes in PmpD: First step towards to the design of a Chlamydia trachomatis vaccine. biomedical journal 41.2 (2018): 109-117; Baruah, et al. Immunoinformatics-aided identification of T cell and B cell epitopes in the surface glycoprotein of 2019-nCoV. Journal of Medical Virology (2020); each of which is incorporated herein by reference in its entirety.

A circular polyribonucleotide of the disclosure may comprise sequences of any number of coronavirus antigens and/or epitopes. A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more coronavirus antigens or epitopes.

In some embodiments, a circular polyribonucleotide comprises a sequence for example, of at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less coronavirus antigens or epitopes.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus antigens or epitopes.

A circular polyribonucleotide may comprise a sequence for one or more coronavirus epitopes from a coronavirus antigen. For example, a coronavirus antigen can comprise an amino acid sequence, which can contain multiple coronavirus epitopes (e.g., epitopes recognized by B cells and/or T cells) therein, and a circular polyribonucleotide can comprise or encode one or more of those coronavirus epitopes.

A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one coronavirus antigen.

In some embodiments, a circular polyribonucleotide comprises, for example, a sequence of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less coronavirus epitopes from one coronavirus antigen.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus epitopes from one coronavirus antigen.

A circular polyribonucleotide may encode variants of a coronavirus antigen or epitope. Variants may be naturally-occurring variants (for example, variants identified in sequence data from different coronavirus genera, species, isolates, or quasispecies), or may be derivative sequences as disclosed herein that have been generated in silico (for example, antigen or epitopes with one or more amino acid insertions, deletions, substitutions, or a combination thereof compared to a wild type antigen or epitope).

A circular polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of a coronavirus antigen or epitope.

In some embodiments, a circular polyribonucleotide comprises a sequence, for example, of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of a coronavirus antigen or epitope.

In some embodiments, a circular polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of a coronavirus antigen or epitope.

A coronavirus antigen and/or epitope sequence of a circular polyribonucleotide can also be referred to as a coronavirus expression sequence. In some embodiments, the circular polyribonucleotide comprises one or more coronavirus expression sequences, each of which may encode a coronavirus polypeptide. The coronavirus polypeptide may be produced in substantial amounts. A coronavirus polypeptide can be a coronavirus polypeptide that is secreted from a cell, or localized to the cytoplasm, nucleus or membrane compartment of a cell. Some coronavirus polypeptides include, but are not limited to, an antigen as disclosed herein, an epitope as disclosed herein, at least a portion of a coronavirus protein (for example, a viral envelope protein, viral matrix protein, viral spike protein, viral receptor binding domain (RBD) of a viral spike protein, viral membrane protein, viral nucleocapsid protein, viral accessory protein, a fragment thereof, or a combination thereof). In some embodiments, a coronavirus polypeptide encoded by a circular polyribonucleotide of the disclosure comprises a fragment of a coronavirus antigen disclosed herein. In some embodiments, a coronavirus polypeptide encoded by a circular polyribonucleotide of the disclosure comprises a fusion protein comprising two or more coronavirus antigens disclosed herein, or fragments thereof. In some embodiments, a coronavirus polypeptide encoded by a circular polyribonucleotide of the disclosure comprises a coronavirus epitope.

In some embodiments, a polypeptide encoded by a circular polyribonucleotide of the disclosure comprises a fusion protein comprising two or more coronavirus epitopes disclosed herein, for example, an artificial peptide sequence comprising a plurality of predicted epitopes from one or more coronavirus s of the disclosure.

In some embodiments, exemplary coronavirus proteins that are expressed from the circular polyribonucleotide disclosed herein include a secreted protein, for example, a protein (e.g., antigen and/or epitope) that naturally includes a signal peptide, or one that does not usually encode a signal peptide, but is modified to contain one.

In some cases, the circular polyribonucleotide expresses a secretary coronavirus protein that has a short half-life in the blood, or is a protein with a subcellular localization signal, or protein with secretory signal peptide. In some cases, the circular polyribonucleotide expresses a transmembrane domain that has a short half-life in the blood, or is a protein with a subcellular localization signal, or protein with secretory peptide.

In some embodiments, the circular polyribonucleotide comprises one or more coronavirus expression sequences and is configured for persistent expression in a cell of a subject (e.g., a subject for immunization) in vivo. In some embodiments, the circular polyribonucleotide is configured such that expression of the one or more coronavirus expression sequences in the cell at a later time point is equal to or higher than an earlier time point. In such embodiments, the expression of the one or more coronavirus expression sequences is either maintained at a relatively stable level or can increase over time. In some embodiments, the expression of the coronavirus expression sequences is relatively stable for an extended period of time.

In some embodiments, the circular polyribonucleotide expresses one or more coronavirus antigens and/or epitopes in a subject (e.g., a subject for immunization), e.g., transiently or long term. In certain embodiments, expression of the coronavirus expression sequences persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, expression of the coronavirus antigens and/or epitopes persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 45 days, 60 days, 75 days, 90 days, or any time therebetween.

In some embodiments, the coronavirus expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the coronavirus expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb or greater).

### Derivatives and Fragments

An antigen or epitope of the disclosure can comprise a wild type sequence. When describing an antigen or epitope, the term “wild type” refers to a sequence (e.g., an amino acid sequence) that is naturally occurring and encoded by a genome (e.g., a coronavirus genome). A coronavirus can have one wild type sequence, or two or more wild type sequences (for example, with one canonical wild type sequence present in a reference coronavirus genome, and additional variant wild type sequences present that have arisen from mutations).

When describing an antigen or epitope, the terms “derivative” and “derived from” refer to a sequence (e.g., amino acid sequence) that differs from a wild type sequence by one or more amino acids, for example, containing one or more amino acid insertions, deletions, and/or substitutions relative to a wild type sequence.

An antigen or epitope derivative sequence is a sequence that has at least 60%, 70%, 75%, 80%, 85%, 90%, 91%, 92%, 93%, 94%, 95%, 96%, 97%, 98%, 99%, or more sequence identity to a wild type sequence, for example, a wild type protein, antigen, or epitope sequence.

“Sequence identity” and “sequence similarity” is determined by alignment of two peptide or two nucleotide sequences using global or local alignment algorithms. Sequences are referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or EmbossWin version 2.10.0 (using the program “needle”). Alternatively or additionally, percent similarity or identity is determined by searching against databases, using algorithms such as FASTA, BLAST, etc. Sequence identity refers to the sequence identity over the entire length of the sequence.

In some embodiments, an antigen or epitope contains one or more amino acid insertions, deletions, substitutions, or a combination thereof that affect the structure of an encoded protein. In some embodiments, an antigen or epitope contains one or more amino acid insertions, deletions, substitutions, or a combination thereof that affect the function of an encoded protein. In some embodiments, an antigen or epitope contains one or more amino acid insertions, deletions, substitutions, or a combination thereof that affect the expression or processing of an encoded protein by a cell.

Amino acid insertions, deletions, substitutions, or a combination thereof can introduce a site for a post-translational modification (for example, introduce a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof remove a site for a post-translational modification (for example, remove a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof modify a site for a post-translational modification (for example, modify a site to alter the efficiency or characteristics of glycosylation, ubiquitination, phosphorylation, nitrosylation, methylation, acetylation, amidation, hydroxylation, sulfation, or lipidation site, or cleavage).

An amino acid substitution can be a conservative or a non-conservative substitution. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val (V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one alihatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group I: ala, pro, gly, gln, asn, ser, thr; Group II: cys, ser, tyr, thr; Group III: val, ile, leu, met, ala, phe; Group IV: lys, arg, his; Group V: phe, tyr, trp, his; and Group VI: asp, glu.

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 amino acid deletions relative to a sequence disclosed herein (e.g., a wild type sequence).

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a sequence disclosed herein (e.g., a wild type sequence).

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid substitutions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid substitutions relative to a sequence disclosed herein (e.g., a wild type sequence).

The one or more amino acid substitutions can be at the N-terminus, the C-terminus, within the amino acid sequence, or a combination thereof. The amino acid substitutions can be contiguous, non-contiguous, or a combination thereof.

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 120, at most 140, at most 160, at most 180, or at most 200 amino acid deletions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, 20-25, 20-30, 30-50, 50-100, or 100-200 amino acid deletions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid deletions relative to a sequence disclosed herein (e.g., a wild type sequence).

The one or more amino acid deletions can be at the N-terminus, the C-terminus, within the amino acid sequence, or a combination thereof. The amino acid deletions can be contiguous, non-contiguous, or a combination thereof.

In some embodiments, an antigen derivative or epitope derivative of the disclosure comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a sequence disclosed herein (e.g., a wild type sequence).

In some embodiments, an antigen derivative or epitope derivative of the disclosure comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a sequence disclosed herein (e.g., a wild type sequence).

In some embodiments, an antigen derivative or epitope derivative of the disclosure comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40,10-15, 15-20, or 20-25 amino acid insertions relative to a sequence disclosed herein (e.g., a wild type sequence).

In some embodiments, an antigen derivative or epitope derivative of the disclosure comprises 1, 2, 3, 4, 5, 6, 7, 8, 9, 10, 11, 12, 13, 14, 15, 16, 17, 18, 19, or 20 amino acid insertions relative to a sequence disclosed herein (e.g., a wild type sequence).

The one or more amino acid insertions can be at the N-terminus, the C-terminus, within the amino acid sequence, or a combination thereof. The amino acid insertions can be contiguous, non-contiguous, or a combination thereof.

### Circular Polyribonucleotide

The circular polyribonucleotide comprises the elements as described below as well as the coronavirus antigen or epitope as described herein.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide may be of a sufficient size to accommodate a binding site for a ribosome. In some embodiments, the maximum size of a circular polyribonucleotide can be as large as is within the technical constraints of producing a circular polyribonucleotide, and/or using the circular polyribonucleotide. Without wishing to be bound by any particular theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, such as antigens and/or epitopes of the disclosure, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful.

### Circular Polyribonucleotide Elements

In some embodiments, the circular polyribonucleotide comprises one or more of the elements as described herein in addition to comprising a sequence encoding a coronavirus antigen and/or epitope. In some embodiments, the circular polyribonucleotide lacks a poly-A sequence, lacks a free 3′ end, lacks an RNA polymerase recognition motif, or any combination thereof. In some embodiments, the circular polyribonucleotide comprises any feature or any combination of features as disclosed in WO2019/118919, which is hereby incorporated by reference in its entirety. For example, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. In addition, one regulatory element can increase an amount of products expressed for multiple expression sequences attached in tandem. Hence, one regulatory element can enhance the expression of one or more expression sequences. Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of WO2019/118919, which is hereby incorporated by reference in its entirety.

In some embodiments, the circular polyribonucleotide encodes an antigen that produces the human polyclonal antibodies of interest and comprises a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of WO2019/118919, which is hereby incorporated by reference in its entirety.

In some embodiments, a circular polyribonucleotide described herein comprises an internal ribosome entry site (IRES) element. A suitable IRES element to include in a circular polyribonucleotide can be an RNA sequence capable of engaging an eukaryotic ribosome. Further examples of an IRES are described in paragraphs [0166]-[0168] of WO2019/118919, which is hereby incorporated by reference in its entirety.

A circular polyribonucleotide can include one or more expression sequences (e.g., encoding an antigen), and each expression sequence may or may not have a termination element. Further examples of termination elements are described in paragraphs [0169]-[0170] of WO2019/118919, which is hereby incorporated by reference in its entirety.

A circular polyribonucleotide of the disclosure can comprise a stagger element. The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence -D(V/I)ExNPGP, where x=any amino acid (SEQ ID NO: 52). In some embodiments, the stagger element may include a chemical moiety, such as glycerol, a non-nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

Examples of stagger elements are described in paragraphs [0172]-[0175] of WO2019/118919, which is hereby incorporated by reference in its entirety.

In some embodiments, the circular polyribonucleotide comprises one or more regulatory nucleic acid sequences or comprises one or more expression sequences that encode regulatory nucleic acid, e.g., a nucleic acid that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

Exemplary regulatory nucleic acids are described in paragraphs [0177]-[0194] of WO2019/118919, which is hereby incorporated by reference in its entirety.

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of WO2019/118919, which is hereby incorporated by reference in its entirety.

In some embodiments, the circular polyribonucleotide includes a poly-A sequence. Exemplary poly-A sequences are described in paragraphs [0202]-[0205] of WO2019/118919, which is hereby incorporated by reference in its entirety. In some embodiments, a circular polyribonucleotide lacks a poly-A sequence.

In some embodiments, the circular polyribonucleotide comprises one or more riboswitches. Exemplary riboswitches are described in paragraphs [0232]-[0252] of WO2019/118919, which is hereby incorporated by reference in its entirety.

In some embodiments, the circular polyribonucleotide comprises an aptazyme. Exemplary aptazymes are described in paragraphs [0253]-[0259] of WO2019/118919, which is hereby incorporated by reference in its entirety.

In some embodiments, the circular polyribonucleotide comprises one or more RNA binding sites. microRNAs (or miRNA) can be short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in US Publication US2005/0261218 and US Publication US2005/0059005, the contents of which are incorporated herein by reference in their entirety. Further examples of RNA binding sites are described in paragraphs [0206]-[0215] of WO2019/118919, which is hereby incorporated by reference in its entirety.

In some embodiments, the circular polyribonucleotide includes one or more protein binding sites that enable a protein, e.g., a ribosome, to bind to an internal site in the RNA sequence. Further examples of protein binding sites are described in paragraphs [0218]-[0221] of WO2019/118919, which is hereby incorporated by reference in its entirety.

In some embodiments, the circular polyribonucleotide comprises a spacer sequence. In some embodiments, elements of a polyribonucleotide may be separated from one another by a spacer sequence or linker. Exemplary of spacer sequences are described in paragraphs [0293]-[0302] of WO2019/118919, which is hereby incorporated by reference in its entirety.

The circular polyribonucleotide described herein may also comprise a non-nucleic acid linker. Exemplary non-nucleic acid linkers are described in paragraphs [0303]-[0307] of WO2019/118919, which is hereby incorporated by reference in its entirety.

In some embodiments, the circular polyribonucleotide further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In some embodiments, the circular polyribonucleotide includes an siRNA to target a different locus of the same gene expression product as the circular polyribonucleotide. In some embodiments, the circular polyribonucleotide includes an siRNA to target a different gene expression product than a gene expression product that is present in the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein In some embodiments, the circular polyribonucleotide lacks a 5′ cap.

In some embodiments, the circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide can be more stable than a linear RNA, especially when incubated in the presence of an exonuclease. The increased stability of the circular polyribonucleotide compared with linear RNA can make the circular polyribonucleotide more useful as a cell transforming reagent to produce polypeptides (e.g., antigens and/or epitopes to elicit antibody responses). The increased stability of the circular polyribonucleotide compared with linear RNA can make the circular polyribonucleotide easier to store for long than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide can be less susceptible to dephosphorylation when the circular polyribonucleotide is incubated with phosphatase, such as calf intestine phosphatase.

In some embodiments, the circular polyribonucleotide comprises particular sequence characteristics. For example, the circular polyribonucleotide may comprise a particular nucleotide composition. In some such embodiments, the circular polyribonucleotide may include one or more purine (adenine and/or guanosine) rich regions. In some such embodiments, the circular polyribonucleotide may include one or more purine poor regions. In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein. In some embodiments, the circular polyribonucleotide comprises one or more modifications described elsewhere herein.

A circular polyribonucleotide may include one or more substitutions, insertions and/or additions, deletions, and covalent modifications with respect to reference sequences. For example, circular polyribonucleotides with one or more insertions, additions, deletions, and/or covalent modifications relative to a parent polyribonucleotide are included within the scope of this disclosure. Exemplary modifications are described in paragraphs [0310]-[0325] of WO2019/118919, which is hereby incorporated by reference in its entirety.

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide comprises a tertiary structure with one or more motifs, e.g., a pseudoknot, a g-quadruplex, a helix, and coaxial stacking.

Further examples of structure of circular polyribonucleotides as disclosed herein are described in paragraphs [0326]-[0333] of WO2019/118919, which is hereby incorporated by reference in its entirety.

### Stability and Half Life

In some embodiments, a circular polyribonucleotide provided herein has increased half-life over a reference, e.g., a linear polyribonucleotide having the same nucleotide sequence that is not circularized (linear counterpart). In some embodiments, the circular polyribonucleotide is substantially resistant to degradation, e.g., exonuclease degradation. In some embodiments, the circular polyribonucleotide is resistant to self-degradation. In some embodiments, the circular polyribonucleotide lacks an enzymatic cleavage site, e.g., a dicer cleavage site. Further examples of stability and half-life of circular polyribonucleotides as disclosed herein are described in paragraphs [0308]-[0309] of WO2019/118919, which is hereby incorporated by reference in its entirety.

### Production Methods

In some embodiments, the circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In some embodiments, the circular polyribonucleotides is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Circularization

In some embodiments, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

a. Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase is used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. As a non-limiting example, the ligase may be a circ ligase or circular ligase.

In some embodiments, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear circular polyribonucleotide is cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an apatamer or a non-nucleic acid linker as described herein.

In some embodiments, a linear circular polyribonucleotide is cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%. In some embodiments, the circularization method provided has a circularization efficiency of between about 10% and about 100%; for example, the circularization efficiency may be about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, the circularization efficiency is between about 20% and about 80%. In some embodiments, the circularization efficiency is between about 30% and about 60%. In some embodiments the circularization efficiency is about 40%.

b. Splicing Element

In some embodiments, the circular polyribonucleotide includes at least one splicing element. Exemplary splicing elements are described in paragraphs [0270]-[0275] of WO2019/118919, which is hereby incorporated by reference in its entirety.

In some embodiments, a circular polyribonucleotide includes at least one splicing element. In a circular polyribonucleotide as provided herein, a splicing element can be a complete splicing element that can mediate splicing of the circular polyribonucleotide. Alternatively, the splicing element can also be a residual splicing element from a completed splicing event. For instance, in some cases, a splicing element of a linear polyribonucleotide can mediate a splicing event that results in circularization of the linear polyribonucleotide, thereby the resultant circular polyribonucleotide includes a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, a circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, a circular polyribonucleotide may include canonical splice sites that flank head-to-tail junctions of the circular polyribonucleotide.

In some embodiments, a circular polyribonucleotide may include a bulge-helix-bulge motif, including a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′-OH group onto the 2′, 3′-cyclic phosphate of the same molecule forming a 3′, 5′-phosphodiester bridge.

In some embodiments, a circular polyribonucleotide may include a multimeric repeating RNA sequence that harbors a HPR element. The HPR includes a 2′,3′-cyclic phosphate and a 5′-OH termini. The HPR element self-processes the 5′- and 3′-ends of the linear polyribonucleotide for circularization, thereby ligating the ends together.

In some embodiments, a circular polyribonucleotide may include a self-splicing element. For example, the circular polyribonucleotide may include an intron from the cyanobacteria Anabaena.

In some embodiments, a circular polyribonucleotide may include a sequence that mediates self-ligation. In one embodiment, the circular polyribonucleotide may include a HDV sequence (e.g., HDV replication domain conserved sequence, GGCUCAUCUCGACAAGAGGCGGCAGUCCUCAGUACUCUUACUCUUUUCUGUAAAGAGGAG ACUGCUGGACUCGCCGCCCAAGUUCGAGCAUGAGCC (SEQ ID NO: 61) or GGCUAGAGGCGGCAGUCCUCAGUACUCUUACUCUUUUCUGUAAAGAGGAGACUGCUGGA CUCGCCGCCCGAGCC (SEQ ID NO: 62)) to self-ligate. In one embodiment, the circular polyribonucleotide may include loop E sequence (e.g., in PSTVd) to self-ligate. In another embodiment, the circular polyribonucleotide may include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motifs useful for replication. Exemplary replication elements are described in paragraphs [0280]-[0286] of WO2019/118919, which is hereby incorporated by reference in its entirety.

### Linear Polyribonucleotide

The linear polyribonucleotides as disclosed herein comprise a sequence encoding an antigen and/or epitope from a coronavirus. This linear polyribonucleotide expresses the sequence encoding the antigen and/or epitope from the coronavirus in a subject (e.g., a subject for immunization). In some embodiments, linear polyribonucleotides comprising a coronavirus antigen and/or epitope are used to produce an immune response in a subject (e.g., a subject for immunization). In some embodiments, the linear polyribonucleotides is an mRNA and comprises a coronavirus antigen and/or epitope are used to produce an immune response in a subject (e.g., a subject for immunization). In some embodiments, linear polyribonucleotides comprising a coronavirus antigen and/or epitope are used to produce polyclonal antibodies as described herein.

### Coronavirus Antigens and Epitopes

The linear polyribonucleotide comprises a sequence encoding a coronavirus antigen or epitope. The antigens and/or epitopes disclosed herein are associated with coronaviruses. In some embodiments, the antigens and/or epitopes are expressed by a coronavirus, or derived from an antigen and/or epitope that is expressed by a coronavirus.

An antigen is a molecule containing one or more epitopes (either linear, conformational or both) that elicit an adaptive immune response in a subject (e.g., a subject for immunization). An epitope can be a part of an antigen that is recognized, targeted, or bound by a given antibody or T cell receptor. An epitope can be a linear epitope, for example, a contiguous sequence of amino acids. An epitope can be a conformational epitope, for example, an epitope that contains amino acids that form an epitope in the folded conformation of the protein. A conformational epitope can contain non-contiguous amino acids from a primary amino acid sequence. Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids.

A coronavirus antigen or epitope can be or can comprise all or a part of a protein, a peptide, a glycoprotein, a lipoprotein, a phosphoprotein, a ribonucleoprotein, a carbohydrate (e.g., a polysaccharide), a lipid (e.g., a phospholipid or triglyceride), or a nucleic acid (e.g., DNA, RNA).

A coronavirus antigen or epitope can comprise a protein antigen or epitope (e.g., a peptide antigen or peptide epitope from a protein, glycoprotein, lipoprotein, phosphoprotein, or ribonucleoprotein). An antigen or epitope can comprise an amino acid, a sugar, a lipid, a phosphoryl, or a sulfonyl group, or a combination thereof.

A coronavirus protein antigen or epitope can comprise a post-translational modification, for example, glycosylation, ubiquitination, phosphorylation, nitrosylation, methylation, acetylation, amidation, hydroxylation, sulfation, or lipidation.

An antigen and/or epitope can be from a coronavirus surface protein, a coronavirus membrane protein, a coronavirus envelope protein, a coronavirus capsid protein, a coronavirus nucleocapsid protein, a coronavirus spike protein, a coronavirus receptor binding domain of a spike protein, a coronavirus entry protein, a coronavirus membrane fusion protein, a coronavirus structural protein, a coronavirus non-structural protein, a coronavirus regulatory protein, a coronavirus accessory protein, a secreted coronavirus protein, a coronavirus polymerase protein, a coronavirus RNA polymerase, a coronavirus protease, a coronavirus glycoprotein, a coronavirus fusogen, a coronavirus helical capsid protein, a coronavirus icosahedral capsid protein, a coronavirus matrix protein, a coronavirus replicase, a coronavirus transcription factor, or a coronavirus enzyme.

In some embodiments, an antigen and/or epitope of the disclosure is from a predicted transcript from a SARS-CoV genome. In some embodiments, an antigen and/or epitope of the disclosure is from a protein encoded by an open reading frames from a SARS-CoV genome. Non-limiting examples of open reading frames in SARS-CoV genomes can include ORF1a, ORF1b, spike (S), ORF3a, ORF3b, envelope (E), membrane (M), ORF6, ORF7a, ORF7b, ORF8, ORF8a, ORF8b, ORF9a, ORF9b, nucleocapsid (N), and ORF10. In some embodiments, the open reading frame from the SARS-CoV genome includes SEQ ID NO: 11.

In particular embodiments, a linear polyribonucleotide comprises a SARS-CoV-2 antigen described in TABLE 3.

TABLE 3
Descriptions of designed linear constructs.

| | ORF | ORF | Proline | Cloning | Circularization | 5’ | 3’ |
|---|---|---|---|---|---|---|---|
| Construct | (SEQ ID NO.) | Description | substitutions | optimization | optimization | element | element |
| p29 | 1 (13) | S protein | Yes | Yes | No | globin | globin |
| | | transmembrane | | | | | |
| | | (TM) domain | | | | | |
| | | completely | | | | | |
| | | removed, and | | | | | |
| | | a trimerization | | | | | |
| | | domain added | | | | | |
| p30 | 3 (15) | S protein | Yes | Yes | No | globin | globin |
| | | transmembrane | | | | | |
| | | (TM) domain | | | | | |
| | | fully intact | | | | | |
| p31 | 13 (12) | S protein | N/A | N/A | N/A | globin | globin |
| | | receptor binding | | | | | |
| | | domain (RBD) | | | | | |
| | | only with | | | | | |
| | | secretion signal | | | | | |
| | | translationally | | | | | |
| | | fused to the 5’ | | | | | |
| | | end | | | | | |
| p32 | 1 (13) | S protein | Yes | Yes | Yes | globin | globin |
| | | transmembrane | | | | | |
| | | (TM) domain | | | | | |
| | | completely | | | | | |
| | | removed, and | | | | | |
| | | a trimerization | | | | | |
| | | domain added | | | | | |

In TABLE 3, “proline substitutions” denotes proline substitutions that are at residues 986 and 987, as well as a “GSAS” substitution at the furin cleavage site (residues 682-685). For cloning optimization, single base substitution was made at coordinate 2541 to destroy a BsaI site to assist in Golden Gate Cloning construction of the plasmid DNA template. For circularization optimizations, four single nucleotides—at positions 2307, 2709, 159 and 315—were substituted to destroy sites that could potentially bind circularization elements of splint nucleic acid sequences, thereby potentially inhibiting efficient ligation. All single bp substitutions were designed to be translationally silent. Further, in TABLE 3, the 5′ Element is Globin (SEQ ID NO: 32); and the 3′ Element: Globin (SEQ ID NO: 33).

In some embodiments, a coronavirus epitope comprises or contains at least 4, at least 5, at least 6, at least 7, at least 8, at least 9, at least 10, at least 11, at least 12, at least 13, at least 14, at least 15, at least 16, at least 17, at least 18, at least. 19, at least 20, at least 21, at least 22, at least 23, at least 24, at least 25, at least 26, at least 27, at least 28, at least 29, or at least 30 amino acids, or more. In some embodiments, a coronavirus epitope comprises or contains at most 4, at most 5, at most 6, at most 7, at most 8, at most 9, at most 10, at most 11, at most 12, at most 13, at most 14, at most 15, at most 16, at most 17, at most 18, at most. 19, at most 20, at most 21, at most 22, at most 23, at most 24, at most 25, at most 26, at most 27, at most 28, at most 29, or at most 30 amino acids, or less. In some embodiments, a coronavirus epitope comprises or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, a coronavirus epitope contains 5 amino acids. In some embodiments, a coronavirus epitope contains 6 amino acids. In some embodiments, an epitope contains 7 amino acids. In some embodiments, a coronavirus epitope contains 8 amino acids. In some embodiments, an epitope can be about 8 to about 11 amino acids. In some embodiments, an epitope can be about 9 to about 22 amino acids.

The coronavirus antigens may comprise antigens recognized by B cells, antigens recognized by T cells, or a combination thereof. In some embodiments, the antigens comprise antigens recognized by B cells. In some embodiments, the coronavirus antigens are antigens recognized by B cells. In some embodiments, the coronavirus antigens comprise antigens recognized by T cells. In some embodiments, the antigens are antigens recognized by T cells.

The coronavirus epitopes comprise epitopes recognized by B cells, epitopes recognized by T cells, or a combination thereof. In some embodiments, the coronavirus epitopes comprise epitopes recognized by B cells. In some embodiments, the epitopes are epitopes recognized by B cells. In some embodiments, the coronavirus epitopes comprise epitopes recognized by T cells. In some embodiments, the coronavirus epitopes are epitopes recognized by T cells.

Techniques for identifying antigens and epitopes in silico have been disclosed, for example, in Sanchez-Trincado, et al. (2017), Fundamentals and methods for T- and B-cell epitope prediction, Journal of immunology research; Grifoni, Alba, et al. A Sequence Homology and Bioinformatic Approach Can Predict Candidate Targets for Immune Responses to SARS-CoV-2. Cell host & microbe (2020); Russi et al. In silico prediction of T- and B-cell epitopes in PmpD: First step towards to the design of a Chlamydia trachomatis vaccine. biomedical journal 41.2 (2018): 109-117; Baruah, et al. Immunoinformatics-aided identification of T cell and B cell epitopes in the surface glycoprotein of 2019-nCoV. Journal of Medical Virology (2020); each of which is incorporated herein by reference in its entirety.

A linear polyribonucleotide of the disclosure may comprise sequences of any number of coronavirus antigens and/or epitopes. A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more coronavirus antigens or epitopes.

In some embodiments, a linear polyribonucleotide comprises a sequence for example, of at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less coronavirus antigens or epitopes.

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus antigens or epitopes.

A linear polyribonucleotide may comprise a sequence for one or more coronavirus epitopes from a coronavirus antigen. For example, a coronavirus antigen can comprise an amino acid sequence, which can contain multiple coronavirus epitopes (e.g., epitopes recognized by a B cell and/or a T cell) therein, and a linear polyribonucleotide can comprise or encode one or more of those coronavirus epitopes.

A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one coronavirus antigen.

In some embodiments, a linear polyribonucleotide comprises, for example, a sequence of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less coronavirus epitopes from one coronavirus antigen.

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus epitopes from one coronavirus antigen.

A linear polyribonucleotide may encode variants of a coronavirus antigen or epitope. Variants may be naturally-occurring variants (for example, variants identified in sequence data from different coronavirus genera, species, isolates, or quasispecies), or may be derivative sequences as disclosed herein that have been generated in silico (for example, antigen or epitopes with one or more amino acid insertions, deletions, substitutions, or a combination thereof compared to a wild type antigen or epitope).

A linear polyribonucleotide comprises a sequence, for example, of at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of a coronavirus antigen or epitope.

In some embodiments, a linear polyribonucleotide comprises a sequence, for example, of at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of a coronavirus antigen or epitope.

In some embodiments, a linear polyribonucleotide comprises a sequence, for example, of about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of a coronavirus antigen or epitope.

A coronavirus antigen and/or epitope sequence of a linear polyribonucleotide can also be referred to as a coronavirus expression sequence. In some embodiments, the linear polyribonucleotide comprises one or more coronavirus expression sequences, each of which may encode a coronavirus polypeptide. The coronavirus polypeptide may be produced in substantial amounts. A coronavirus polypeptide can be a coronavirus polypeptide that is secreted from a cell, or localized to the cytoplasm, nucleus or membrane compartment of a cell. Some coronavirus polypeptides include, but are not limited to, an antigen as disclosed herein, an epitope as disclosed herein, at least a portion of a coronavirus protein (for example, a viral envelope protein, viral matrix protein, viral spike protein, viral membrane protein, viral nucleocapsid protein, viral accessory protein, a fragment thereof, or a combination thereof). In some embodiments, a coronavirus polypeptide encoded by a linear polyribonucleotide of the disclosure comprises a fragment of a coronavirus antigen disclosed herein. In some embodiments, a coronavirus polypeptide encoded by a linear polyribonucleotide of the disclosure comprises a fusion protein comprising two or more coronavirus antigens disclosed herein, or fragments thereof. In some embodiments, a coronavirus polypeptide encoded by a linear polyribonucleotide of the disclosure comprises a coronavirus epitope. In some embodiments, a polypeptide encoded by a linear polyribonucleotide of the disclosure comprises a fusion protein comprising two or more coronavirus epitopes disclosed herein, for example, an artificial peptide sequence comprising a plurality of predicted epitopes from one or more coronavirus of the disclosure.

In some embodiments, exemplary coronavirus proteins that are expressed from the linear polyribonucleotide disclosed herein include a secreted protein, for example, a protein (e.g., antigen and/or epitope) that naturally includes a signal peptide, or one that does not usually encode a signal peptide, but is modified to contain one.

### Linear Polyribonucleotide

The linear polyribonucleotide comprises the elements as described below as well as the coronavirus antigen or epitope as described herein.

Linear polyribonucleotides described herein are a polyribonucleotide molecule having a 5′ and 3′ end. In some embodiments, the linear RNA has a free 5′ end or 3′ end. In some embodiments, the linear RNA has a 5′ end or 3′ end that is modified or protected from degradation. In some embodiments, the linear RNA has non-covalently linked 5′ or 3′ ends. In some embodiments, the linear RNA is an mRNA.

Linear RNA can be modified at its ends to improve stability and/or reduce degradation. For example, the 5′ free end and/or 3′ free comprises a cap, a poly-A tail, a G-quadruplex, a pseudoknot, a stable terminal stem loop, U-rich expression, a nuclear retention element (ENE), or a conjugation moiety. For example, the 5′ free end and/or 3′ free comprises an end protectant, such as a cap, a poly-A tail, a g-quadruplex, a pseudoknot, a stable terminal stem loop, U-rich expression, a nuclear retention element (ENE), or a conjugation moiety.

In some embodiments, the linear polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the linear polyribonucleotide may be of a sufficient size to accommodate a binding site for a ribosome. In some embodiments, the maximum size of a linear polyribonucleotide can be as large as is within the technical constraints of producing a linear polyribonucleotide, and/or using the linear polyribonucleotide. Without wishing to be bound by any particular theory, it is possible that multiple segments of RNA may be produced from DNA and their 5′ and 3′ free ends annealed to produce a “string” of RNA. In some embodiments, the maximum size of a linear polyribonucleotide may be limited by the ability of packaging and delivering the RNA to a target. In some embodiments, the size of a linear polyribonucleotide is a length sufficient to encode useful polypeptides, such as antigens and/or epitopes of the disclosure, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful.

### Linear Polyribonucleotide Elements

In some embodiments, the linear polyribonucleotide comprises one or more of the elements as described herein in addition to comprising a sequence encoding a coronavirus antigen and/or epitope. For example, the linear polyribonucleotide comprises a regulatory element, e.g., a sequence that modifies expression of an expression sequence within the linear polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. In addition, one regulatory element can increase an amount of products expressed for multiple expression sequences attached in tandem. Hence, one regulatory element can enhance the expression of one or more expression sequences. Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the linear polyribonucleotide, for instance, certain riboswitch aptazymes. A regulatory element can also include a selective degradation sequence. As used herein, the term “selective degradation sequence” refers to a nucleic acid sequence that initiates degradation of the linear polyribonucleotide, or an expression product of the linear polyribonucleotide. In some embodiments, the regulatory element is a translation modulator. A translation modulator can modulate translation of the expression sequence in the linear polyribonucleotide. A translation modulator can be a translation enhancer or suppressor. In some embodiments, a translation initiation sequence can function as a regulatory element.

In some embodiments, the linear polyribonucleotide encodes an antigen that produces the polyclonal antibodies of interest and comprises a translation initiation sequence, e.g., a start codon. In some embodiments, the translation initiation sequence includes a Kozak or Shine-Dalgarno sequence. In some embodiments, the linear polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the linear polyribonucleotide includes at least one translation initiation sequence adjacent to an expression sequence. In some embodiments, the translation initiation sequence provides conformational flexibility to the linear polyribonucleotide. In some embodiments, the translation initiation sequence is within a substantially single stranded region of the linear polyribonucleotide.

In some embodiments, a linear polyribonucleotide described herein comprises an internal ribosome entry site (IRES) element. A suitable IRES element to include in a linear polyribonucleotide can be an RNA sequence capable of engaging an eukaryotic ribosome.

A linear polyribonucleotide can include one or more expression sequences (e.g., encoding an antigen), and each expression sequence may or may not have a termination element.

In some embodiments, a linear polynucleotide comprises a 5′ cap, wherein the 5′ cap structure of the mRNA increases mRNA stability. The 5′ cap binds to the mRNA cap Binding Protein (MBP), which contributes to mRNA stability in the cell and translation competency through the association of CBP with the poly-A binding protein to form mature RNA species.

In some embodiments, the linear polynucleotide is 5′ end capped and comprises a 5′-ppp-5′triphosphate linkage between a terminal guanosine cap residue and the 5′ terminal transcribed sense nucleotide of the linear polynucleotide. This 5′ guanosine cap, also known as a 5′ guanylated cap, can be methylated to generate a N7-methyl-guanylate cap.

In some embodiments, the linear polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

In some embodiments, the linear polyribonucleotide includes a poly-A sequence. In some embodiments, the length of a poly-A sequence is greater than 10 nucleotides in length. In some embodiments, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is from about 10 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A sequence is designed relative to the length of the overall linear polyribonucleotide. The design can be based on the length of the coding region, the length of a particular feature or region (such as the first or flanking regions), or based on the length of the ultimate product expressed from the linear polyribonucleotide. In this context, the poly-A sequence can be 10, 20, 30, 40, 50, 60, 70, 80, 90, or 100% greater in length than the linear polyribonucleotide or a feature thereof. The poly-A sequence can also be designed as a fraction of the linear polyribonucleotide. In this context, the poly-A sequence can be 10%, 20%, 30%, 40%, 50%, 60%, 70%, 80%, 90%, or more of the total length of the construct or the total length of the construct minus the poly-A sequence. Further, engineered binding sites and conjugation of linear polyribonucleotide for Poly-A binding protein can enhance expression.

In some embodiments, the linear polyribonucleotide is designed to include a polyA-G quartet. The G-quartet is a cyclic hydrogen bonded array of four guanine nucleotides that can be formed by G-rich sequences in both DNA and RNA. In some embodiments, the G-quartet can be incorporated at the end of the poly-A sequence. The resultant linear polyribonucleotide construct can be assayed for stability, protein production, and/or other parameters including half-life at various time points. In some embodiments, the polyA-G quartet can result in protein production equivalent to at least 75% of that seen using a poly-A sequence of 120 nucleotides alone.

In some embodiments, the linear polyribonucleotide comprises a UTR with one or more stretches of adenosines and uridines embedded within. AU-rich signatures can increase turnover rates of the expression product.

Introduction, removal, or modification of UTR AU-rich elements (AREs) can be useful to modulate the stability or immunogenicity of the linear polyribonucleotide. When engineering specific linear polyribonucleotides, one or more copies of an ARE can be introduced to destabilize the linear polyribonucleotide and the copies of an ARE can decrease translation and/or decrease production of an expression product. Likewise, AREs can be identified and removed or mutated to increase the intracellular stability and thus increase translation and production of the resultant protein.

A UTR from any gene can be incorporated into the respective flanking regions of the linear polyribonucleotide (e.g., at the 5′ end or the 3′ end). Furthermore, multiple wild-type UTRs of any known gene can be utilized. In some embodiments, artificial UTRs that are not variants of wild type genes can be used. These UTRs or portions thereof can be placed in the same orientation as in the transcript from which they were selected or can be altered in orientation or location. Hence a 5′- or 3′-UTR can be inverted, shortened, lengthened, or made chimeric with one or more other 5′- or 3′-UTRs. As used herein, the term “altered” as it relates to a UTR sequence, means that the UTR has been changed in some way in relation to a reference sequence. For example, a 3′- or 5′-UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double UTR, triple UTR, or quadruple UTR, such as a 5′- or 3′-UTR, can be used. As used herein, a “double” UTR is one in which two copies of the same UTR are encoded either in series or substantially in series. For example, a double beta-globin 3′-UTR can be used in some embodiments of the invention.

In some embodiments, the linear polyribonucleotide comprises one or more regulatory nucleic acid sequences or comprises one or more expression sequences that encode regulatory nucleic acid, e.g., a nucleic acid that modifies expression of an endogenous gene and/or an exogenous gene. In some embodiments, the expression sequence of a linear polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

In some embodiments, the linear polyribonucleotide produces stoichiometric ratios of expression products. In some embodiments, the linear polyribonucleotide has a stoichiometric translation efficiency, such that expression products are produced at substantially equivalent ratios. In some embodiments, the linear polyribonucleotide has a stoichiometric translation efficiency of multiple expression products, e.g., products from 2, 3, 4, 5, 6, 7, 8, 9, 10, 11, 12, or more expression sequences.

In some embodiments, the linear polyribonucleotide comprises one or more riboswitches.

In some embodiments, the linear polyribonucleotide comprises an aptazyme.

In some embodiments, the linear polyribonucleotide lacks a 5′-UTR. In some embodiments, the linear polyribonucleotide lacks a 3′-UTR. In some embodiments, the linear polyribonucleotide lacks a poly-A sequence. In some embodiments, the linear polyribonucleotide lacks a termination element. In some embodiments, the linear polyribonucleotide lacks an internal ribosomal entry site. In some embodiments, the linear polyribonucleotide lacks binding to a cap-binding protein. In some embodiments, the linear polyribonucleotide lacks a 5′ cap.

### Production Methods

In some embodiments, the linear polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The linear polyribonucleotide may be prepared according to any available technique including, but not limited to chemical synthesis and enzymatic synthesis. In some embodiments, a linear primary construct or linear mRNA may be or concatemerized to create a linear polyribonucleotide described herein. The mechanism of concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the linear polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing linear polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

### Methods of Producing an Immune Response

The disclosure provides immunogenic compositions comprising a circular polyribonucleotide described above. The disclosure provides immunogenic compositions comprising a linear polyribonucleotide described above. Immunogenic compositions of the invention may comprise a diluent or a carrier, adjuvant, or any combination thereof. Immunogenic compositions of the invention may also comprise one or more immunoregulatory agents, e.g., one or more adjuvants. The adjuvants may include a TH1 adjuvant and/or a TH2 adjuvant, further discussed below. In some embodiments, the immunogenic composition comprises a diluent free of any carrier and is used for naked delivery of the circular polyribonucleotide to a subject (e.g., a subject for immunization). In some embodiments, the immunogenic composition comprises a diluent free of any carrier and is used for naked delivery of the linear polyribonucleotide to a subject.

Immunogenic compositions of the invention are used to raise an immune response in a subject (e.g., a subject for immunization). The immune response may comprise an antibody response (usually including IgG) and/or a cell-mediated immune response. In some embodiments, the immunogenic compositions are used to produce polyclonal antibodies as described herein. For example, a subject is immunized with an immunogenic composition comprising a circular polyribonucleotide comprising a coronavirus antigen and/or epitope to stimulate production of polyclonal antibodies that bind to the coronavirus antigen and/or epitope. In another example, a subject is immunized with an immunogenic composition comprising a linear polyribonucleotide comprising a coronavirus antigen and/or epitope to stimulate production of polyclonal antibodies that bind to the coronavirus antigen and/or epitope. In some embodiments, the subject is a human. In some embodiments, the subject is non-human animal. In some embodiments, the non-human animal has a humanized immune system. In some embodiments, the subject is further immunized with an adjuvant. In some embodiments the subject is further immunized with a vaccine. Optionally, after immunization with the immunogenic composition comprising the circular polyribonucleotide, the produced polyclonal antibodies are collected and purified from the subject. Optionally, after immunization with the immunogenic composition comprising the linear polyribonucleotide, the produced polyclonal antibodies are collected and purified from the subject. In some embodiments, a composition comprises plasma collected after administration of the immunogenic composition described herein.

### Immunization

In some embodiments, methods of the disclosure comprise immunizing a subject (e.g., a subject for immunization) with an immunogenic composition comprising a circular polyribonucleotide as disclosed herein. In some embodiments, a coronavirus antigen and/or epitope is expressed from the circular polyribonucleotide. In some embodiments, immunization induces an immune response in a subject against the coronavirus antigen and/or epitope expressed from the circular polyribonucleotide. In some embodiments, immunization induces the production of polyclonal antibodies that bind to the coronavirus antigen and/or epitope expressed from the circular polyribonucleotide. In some embodiments, an immunogenic composition comprises the circular polyribonucleotide and a diluent, carrier, first adjuvant or a combination thereof in a single composition. In some embodiments, the subject is further immunized with a second adjuvant. In some embodiments, the subject is further immunized with a vaccine.

In some embodiments, methods of the disclosure comprise immunizing a subject (e.g., a subject for immunization) with an immunogenic composition comprising a linear polyribonucleotide as disclosed herein. In some embodiments, a coronavirus antigen and/or epitope is expressed from the linear polyribonucleotide. In some embodiments, immunization induces an immune response in a subject against the coronavirus antigen and/or epitope expressed from the linear polyribonucleotide. In some embodiments, immunization induces the production of polyclonal antibodies that bind to the coronavirus antigen and/or epitope expressed from the linear polyribonucleotide. In some embodiments, an immunogenic composition comprises the linear polyribonucleotide and a diluent, carrier, first adjuvant or a combination thereof in a single composition. In some embodiments, the subject is further immunized with a second adjuvant. In some embodiments, the subject is further immunized with a vaccine.

The circular polyribonucleotide as disclosed herein stimulate the production of human polyclonal antibodies by stimulating the adaptive immune response after immunization of a subject (e.g., a subject for immunization). In some embodiments, the adaptive immune response of the subject comprises a stimulation of B lymphocytes to release polyclonal antibodies that specifically bind to the coronavirus antigen expressed by the circular polyribonucleotide. The linear polyribonucleotide as disclosed herein stimulate the production of human polyclonal antibodies by stimulating the adaptive immune response after immunization of a subject. In some embodiments, the adaptive immune response of the subject comprises a stimulation of B lymphocytes to release polyclonal antibodies that specifically bind to the coronavirus antigen expressed by the linear polyribonucleotide. In some embodiments, the adaptive immune response of the subject comprises stimulating cell-mediated immune responses.

The subject (e.g., a subject for immunization) is immunized with one or more immunogenic composition(s) comprising any number of circular polyribonucleotides. The subject is immunized with, for example, one or more immunogenic composition(s) comprising at least 1 circular polyribonucleotide. A non-human animal having a non-humanized immune system is immunized with, for example, one or more immunogenic composition(s) comprising at least 2, at least 3, at least 4, at least 5, at least 6, at least 7, at least 8, at least 9, at least 10, at least 11, at least 12, at least 13, at least 14, at least 15, at least 20 different circular polyribonucleotides, or more different circular polyribonucleotides. In some embodiments, a subject is immunized with one or more immunogenic composition(s) comprising at most 1 circular polyribonucleotide. In some embodiments, a non-human animal having a humanized immune system is immunized with one or more immunogenic composition(s) comprising at most 2, at most 3, at most 4, at most 5, at most 6, at most 7, at most 8, at most 9, at most 10, at most 11, at most 12, at most 13, at most 14, at most 15, at most 20 different circular polyribonucleotides, or less than 21 different circular polyribonucleotides. In some embodiments, a subject is immunized with one or more immunogenic composition(s) comprising about 1 circular polyribonucleotide. In some embodiments, a non-human animal having a humanized immune system is immunized with one or more immunogenic composition(s) comprising about 2, about 3, about 4, about 5, about 6, about 7, about 8, about 9, about 10, about 11, about 12, about 13, about 14, about 15, or about 20 different circular polyribonucleotides. In some embodiments, a subject is immunized with one or more immunogenic composition(s) comprising about 1-20, 1-15, 1-10, 1-9, 1-8, 1-7, I-6, 1-5, I-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different circular polyribonucleotides. Different circular polyribonucleotides have different sequences from each other. For example, they can comprise or encode different antigens and/or epitopes, overlapping antigens and/or epitopes, similar antigens and/or epitopes, or the same antigens and/or epitopes (for example, with the same or different regulatory elements, initiation sequences, promoters, termination elements, or other elements of the disclosure). In cases where a subject is immunized with one or more immunogenic composition(s) comprising two or more different circular polyribonucleotides, the two or more different circular polyribonucleotides can be in the same or different immunogenic compositions and immunized at the same time or at different times. The immunogenic compositions comprising two or more different circular polyribonucleotides can be administered to the same anatomical location or different anatomical locations.

The two or more different circular polyribonucleotides can comprise or encode antigens and/or epitopes from the same coronavirus, different coronavirus, or different combinations of coronaviruses disclosed herein. The two or more different circular polyribonucleotides can comprise or encode antigens and/or epitopes from the same coronavirus or from different coronaviruses, for example, different isolates.

The subject (e.g., a subject for immunization) is immunized with one or more immunogenic composition(s) comprising any number of linear polyribonucleotides. The subject is immunized with, for example, one or more immunogenic composition(s) comprising at least 1 linear polyribonucleotide. A non-human animal having a non-humanized immune system is immunized with, for example, one or more immunogenic composition(s) comprising at least 2, at least 3, at least 4, at least 5, at least 6, at least 7, at least 8, at least 9, at least 10, at least 11, at least 12, at least 13, at least 14, at least 15, at least 20 different linear polyribonucleotides, or more different linear polyribonucleotides. In some embodiments, a subject is immunized with one or more immunogenic composition(s) comprising at most 1 linear polyribonucleotide. In some embodiments, a non-human animal having a humanized immune system is immunized with one or more immunogenic composition(s) comprising at most 2, at most 3, at most 4, at most 5, at most 6, at most 7, at most 8, at most 9, at most 10, at most 11, at most 12, at most 13, at most 14, at most 15, at most 20 different linear polyribonucleotides, or less than 21 different linear polyribonucleotides. In some embodiments, a subject is immunized with one or more immunogenic composition(s) comprising about 1 linear polyribonucleotide. In some embodiments, a non-human animal having a humanized immune system is immunized with one or more immunogenic composition(s) comprising about 2, about 3, about 4, about 5, about 6, about 7, about 8, about 9, about 10, about 11, about 12, about 13, about 14, about 15, or about 20 different linear polyribonucleotides. In some embodiments, a subject is immunized with one or more immunogenic composition(s) comprising about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different linear polyribonucleotides. Different linear polyribonucleotides have different sequences from each other. For example, they can comprise or encode different antigens and/or epitopes, overlapping antigens and/or epitopes, similar antigens and/or epitopes, or the same antigens and/or epitopes (for example, with the same or different regulatory elements, initiation sequences, promoters, termination elements, or other elements of the disclosure). In cases where a subject is immunized with one or more immunogenic composition(s) comprising two or more different linear polyribonucleotides, the two or more different linear polyribonucleotides can be in the same or different immunogenic compositions and immunized at the same time or at different times. The immunogenic compositions comprising two or more different linear polyribonucleotides can be administered to the same anatomical location or different anatomical locations.

The two or more different linear polyribonucleotides can comprise or encode antigens and/or epitopes from the same coronavirus, different coronavirus, or different combinations of coronaviruses disclosed herein. The two or more different linear polyribonucleotides can comprise or encode antigens and/or epitopes from the same coronavirus or from different coronaviruses, for example, different isolates.

In some embodiments, the subject (e.g., a subject for immunization) is immunized with one or more immunogenic composition(s) comprising any number of circular polyribonucleotides and one or more immunogenic composition(s) comprising any number of linear polyribonucleotides as disclosed herein. In some embodiments, an immunogenic composition disclosed herein comprises one or more circular polyribonucleotides and one or more linear polyribonucleotides as disclosed herein.

In some embodiments, an immunogenic composition comprises a circular polyribonucleotide and a diluent, a carrier, a first adjuvant, or a combination thereof. In a particular embodiment, an immunogenic composition comprises a circular polyribonucleotide described herein and a carrier or a diluent free of any carrier. In some embodiments, an immunogenic composition comprising a circular polyribonucleotide with a diluent free of any carrier is used for naked delivery of the circular polyribonucleotide to a subject. In another particular embodiment, an immunogenic composition comprises a circular polyribonucleotide described herein and a first adjuvant.

In certain embodiments, a subject (e.g., a subject for immunization) is further administered a second adjuvant. An adjuvant enhances the innate immune response, which in turn, enhances the adaptive immune response for the production of polyclonal antibodies in a subject. An adjuvant can be any adjuvant as discussed below. In certain embodiments, an adjuvant is formulated with the circular polyribonucleotide as a part of an immunogenic composition. In certain embodiments, an adjuvant is not part of an immunogenic composition comprising the circular polyribonucleotide. In certain embodiments, an adjuvant is administered separately from an immunogenic composition comprising the circular polyribonucleotide. In this aspect, the adjuvant is co-administered (e.g., administered simultaneously) or administered at a different time than an immunogenic composition comprising the circular polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the circular polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the circular polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition comprising the circular polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition comprising the circular polyribonucleotide. The adjuvant is administered to the same anatomical location or different anatomical location as the immunogenic composition comprising the circular polyribonucleotide.

In some embodiments, an immunogenic composition comprises a linear polyribonucleotide and a diluent, a carrier, a first adjuvant, or a combination thereof. In a particular embodiment, an immunogenic composition comprises a linear polyribonucleotide described herein and a carrier or a diluent free of any carrier. In some embodiments, an immunogenic composition comprising a linear polyribonucleotide with a diluent free of any carrier is used for naked delivery of the linear polyribonucleotide to a subject (e.g., a subject for immunization). In another particular embodiment, an immunogenic composition comprises a linear polyribonucleotide described herein and a first adjuvant.

In certain embodiments, a subject (e.g., a subject for immunization) is further administered a second adjuvant. An adjuvant enhances the innate immune response, which in turn, enhances the adaptive immune response for the production of polyclonal antibodies in a subject. An adjuvant can be any adjuvant as discussed below. In certain embodiments, an adjuvant is formulated with the linear polyribonucleotide as a part of an immunogenic composition. In certain embodiments, an adjuvant is not part of an immunogenic composition comprising the linear polyribonucleotide. In certain embodiments, an adjuvant is administered separately from an immunogenic composition comprising the linear polyribonucleotide. In this aspect, the adjuvant is co-administered (e.g., administered simultaneously) or administered at a different time than an immunogenic composition comprising the linear polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the linear polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the linear polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition comprising the linear polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition comprising the linear polyribonucleotide. The adjuvant is administered to the same anatomical location or different anatomical location as the immunogenic composition comprising the linear polyribonucleotide.

In some embodiments, a subject (e.g., a subject for immunization) is further immunized with a second agent, e.g., a vaccine (as described below) that is not a circular polyribonucleotide. The vaccine is co-administered (e.g., administered simultaneously) or administered at a different time than an immunogenic composition comprising the circular polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the circular polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the circular polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition comprising the circular polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition comprising the circular polyribonucleotide.

In some embodiments, a subject (e.g., a subject for immunization) is further immunized with a second agent, e.g., a vaccine (as described below) that is not a linear polyribonucleotide. The vaccine is co-administered (e.g., administered simultaneously) or administered at a different time than an immunogenic composition comprising the linear polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition comprising the linear polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition comprising the linear polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition comprising the linear polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition comprising the linear polyribonucleotide.

A subject (e.g., a subject for immunization) can be immunized with an immunogenic composition, adjuvant, vaccine (e.g., protein subunit vaccine), or a combination thereof any suitable number of times to achieve a desired response. For example, a prime-boost immunization strategy can be utilized to generate hyperimmune plasma containing a high concentration of antibodies that bind to antigens and/or epitopes of the disclosure. A subject can be immunized with an immunogenic composition, adjuvant, vaccine (e.g., protein subunit vaccine), or a combination thereof, of the disclosure, for example, at least 1, at least 2, at least 3, at least 4, at least 5, at least 6, at least 7, at least 8, at least 9, at least 10, or at least 15 times, or more.

In some embodiments, a subject (e.g., a subject for immunization) can be immunized with an immunogenic composition, adjuvant, vaccine (e.g., protein subunit vaccine), or a combination thereof, of the disclosure at most 2, at most 3, at most 4, at most 5, at most 6, at most 7, at most 8, at most 9, at most 10, at most 15, or at most 20 times, or less.

In some embodiments, a subject (e.g., a subject for immunization) can be immunized with an immunogenic composition, adjuvant, vaccine (e.g., protein subunit vaccine), or a combination thereof, of the disclosure about 1, 2, 3, 4, 5, 6, 7, 8, 9, 10, 15, or 20 times.

In some embodiments, a subject (e.g., a subject for immunization) can be immunized with an immunogenic composition, adjuvant, vaccine (e.g., protein subunit vaccine), or a combination thereof, of the disclosure once. In some embodiments, a subject can be immunized with an immunogenic composition, adjuvant, vaccine (e.g., protein subunit vaccine), or a combination thereof, of the disclosure twice. In some embodiments, a subject can be immunized with an immunogenic composition, adjuvant, vaccine (e.g., protein subunit vaccine), or a combination thereof, of the disclosure three times. In some embodiments, a subject can be immunized with an immunogenic composition, adjuvant, vaccine (e.g., protein subunit vaccine), or a combination thereof, of the disclosure four times. In some embodiments, a subject can be immunized with an immunogenic composition, adjuvant, vaccine (e.g., protein subunit vaccine), or a combination thereof, of the disclosure five times. In some embodiments, a subject can be immunized with an immunogenic composition, adjuvant, vaccine (e.g., protein subunit vaccine), or a combination thereof, of the disclosure seven times.

Suitable time intervals can be selected for spacing two or more immunizations. The time intervals can apply to multiple immunizations with the same immunogenic composition, adjuvant, or vaccine (e.g., protein subunit vaccine), or combination thereof, for example, the same the same immunogenic composition, adjuvant, or vaccine (e.g., protein subunit vaccine), or combination thereof, can be administered in the same amount or a different amount, via the same immunization route or a different immunization route. The time intervals can apply to immunizations with different agents, for example, a first immunogenic composition comprising a first circular polyribonucleotide and a second immunogenic composition comprising s second circular polyribonucleotide. The time intervals can apply to a first immunogenic composition comprising a first linear polyribonucleotide and a second immunogenic composition comprising s second linear polyribonucleotide. For regimens comprising three or more immunizations, the time intervals between immunizations can be the same or different. In some examples, about 1, 2, 3, 4, 5, 6, 7, 8, 9, 10, 11, 12, 14, 16, 17, 18, 20, 22, 24, 26, 28, 30, 32, 34, 36, 40, 48, or 72 hours elapse between two immunizations. In some embodiments, about 1, 2, 3, 4, 5, 6, 7, 8, 9, 10, 11, 12, 14, 16, 17, 18, 20, 21, 24, 28, or 30 days elapse between two immunizations. In some embodiments, about 1, 2, 3, 4, 5, 6, 7, or 8 weeks elapse between two immunizations. In some embodiments, about 1, 2, 3, 4, 5, 6, 7, or 8 months elapse between two immunizations.

In some embodiments, at least 1, at least 2, at least 3, at least 4, at least 5, at least 6, at least 7, at least 8, at least 9, at least 10, at least 15, at least 20, at least 24, at least 36, or at least 72 hours, or more elapse between two immunizations. In some embodiments, at most 1, at most 2, at most 3, at most 4, at most 5, at most 6, at most 7, at most 8, at most 9, at most 10, at most 15, at most 20, at most 24, at most 36, or at most 72 hours, or less elapse between two immunizations.

In some embodiments, at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, elapse between two immunizations. In some embodiments, at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 32, at most 34, or at most 36 days, or less elapse between two immunizations.

In some embodiments, at least 1, at least 2, at least 3, at least 4, at least 5, at least 6, at least 7, or at least 8 weeks, or more elapse between two immunizations. In some embodiments, at most 2, at most 3, at most 4, at most 5, at most 6, at most 7, at most 8 weeks, or less elapse between two immunizations.

In some embodiments, at least 1, at least 2, at least 3, at least 4, at least 5, at least 6, at least 7, or at least 8 months, or more elapse between two immunizations. In some embodiments, at most 2, at most 3, at most 4, at most 5, at most 6, at most 7, at most 8 months, or less elapse between two immunizations.

In some embodiments, a non-human animal having a humanized immune system is immunized 3 times at 3-4 week intervals.

In some embodiments, the method further comprises pre-administering an agent to improve immunogenic responses to the non-human animal (e.g., the non-human animal having a humanized immune system) or human subject (e.g., a non-human animal or human subject for immunization). In some embodiments, the agent is the antigen as disclosed herein (e.g., a protein antigen). For example, the method comprises administering the protein antigen from 1 to 7 days prior to administration of the circular polyribonucleotide comprising the sequence encoding the protein antigen. In some embodiments, the protein antigen is administered 1, 2, 3, 4, 5, 6, or 7 days prior to administration of the circular polyribonucleotide comprising the sequence encoding the protein antigen. For example, the method comprises administering the protein antigen from 1 to 7 days prior to administration of the linear polyribonucleotide comprising the sequence encoding the protein antigen. In some embodiments, the protein antigen is administered 1, 2, 3, 4, 5, 6, or 7 days prior to administration of the linear polyribonucleotide comprising the sequence encoding the protein antigen. The protein antigen may be administered as a protein preparation, encoded in a plasmid (pDNA), presented in a virus-like particle (VLP), formulated in a lipid nanoparticle, or the like.

A subject (e.g., a subject for immunization) can be immunized with an immunogenic composition, an adjuvant, or a vaccine (e.g., protein subunit vaccine), or a combination thereof, at any suitable number anatomical sites. The same immunogenic composition, an adjuvant, a vaccine (e.g., protein subunit vaccine), or a combination thereof can be administered to multiple anatomical sites, different immunogenic compositions comprising the same or different circular polyribonucleotides, adjuvants, vaccines (e.g., protein subunit vaccine) or a combination thereof can be administered to different anatomical sites, different immunogenic compositions comprising the same or different circular polyribonucleotides, adjuvants, vaccines (e.g., protein subunit vaccines) or a combination thereof can be administered to the same anatomical site, or any combination thereof. For example, an immunogenic composition comprising a circular polyribonucleotide can be administered in to two different anatomical sites, and/or an immunogenic composition comprising a circular polyribonucleotide can be administered to one anatomical site, and an adjuvant can be administered to a different anatomical site. The same immunogenic composition, an adjuvant, a vaccine (e.g., protein subunit vaccine), or a combination thereof can be administered to multiple anatomical sites, different immunogenic compositions comprising the same or different linear polyribonucleotides, adjuvants, vaccines (e.g., protein subunit vaccine) or a combination thereof can be administered to different anatomical sites, different immunogenic compositions comprising the same or different linear polyribonucleotides, adjuvants, vaccines (e.g., protein subunit vaccines) or a combination thereof can be administered to the same anatomical site, or any combination thereof. For example, an immunogenic composition comprising a linear polyribonucleotide can be administered in to two different anatomical sites, and/or an immunogenic composition comprising a linear polyribonucleotide can be administered to one anatomical site, and an adjuvant can be administered to a different anatomical site.

Immunization at any two or more anatomical routes can be via the same route of immunization (e.g., intramuscular) or by two or more routes of immunization. In some embodiments, an immunogenic composition comprising a circular polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject (e.g., a subject for immunization). In some embodiments, an immunogenic composition comprising a circular polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition comprising a circular polyribonucleotide or an adjuvant of the disclosure is immunized to 1, 2, 3, 4, 5, 6, 7, 8, 9, 10 anatomical sites of a subject. In some embodiments, an immunogenic composition comprising a linear polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject. In some embodiments, an immunogenic composition comprising a linear polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition comprising a linear polyribonucleotide or an adjuvant of the disclosure is immunized to 1, 2, 3, 4, 5, 6, 7, 8, 9, 10 anatomical sites of a subject.

Immunization can be by any suitable route. Non-limiting examples of immunization routes include intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal, e.g., injection and infusion. In some cases, immunization can be via inhalation. Two or more immunizations can be done by the same route or by different routes.

Any suitable amount of a circular polyribonucleotide can be administered to a subject (e.g., a subject for immunization) of the disclosure. For example, a subject can be immunized with at least about 1 ng, at least about 10 ng, at least about 100 ng, at least about 1 μg, at least about 10 μg, at least about, at least about 100 μg, at least about 1 mg, at least about 10 mg, at least about 100 mg, or at least about 1 g of a circular polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a circular polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a circular polyribonucleotide.

Any suitable amount of a linear polyribonucleotide can be administered to a subject (e.g., a subject for immunization) of the disclosure. For example, a subject can be immunized with at least about 1 ng, at least about 10 ng, at least about 100 ng, at least about 1 μg, at least about 10 μg, at least about 100 g, at least about 1 mg, at least about 10 mg, at least about 100 mg, or at least about 1 g of a linear polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a linear polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a linear polyribonucleotide.

In some embodiments, the method further comprises evaluating the non-human animal or human subject (e.g., a subject for immunization) for antibody response to the antigen. In some embodiments, the evaluating is before and/or after administration of the circular polyribonucleotide comprising a sequence encoding a coronavirus antigen. In some embodiments, the evaluating is before and/or after administration of the linear polyribonucleotide comprising a sequence encoding a coronavirus antigen.

### Diluent

In some embodiments, an immunogenic composition of the invention comprises a circular polyribonucleotide and a diluent. In some embodiments, an immunogenic composition of the invention comprises a linear polyribonucleotide and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the circular polyribonucleotide may be delivered as a naked delivery formulation, such as comprising a diluent. A naked delivery formulation delivers a circular polyribonucleotide, to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without a covalent modification that binds a moiety that aids in delivery to a cell is a polyribonucleotide that is not covalently bound to a protein, small molecule, a particle, a polymer, or a biopolymer. A circular polyribonucleotide without covalent modification that binds a moiety that aids in delivery to a cell does not contain a modified phosphate group. For example, a circular polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, the linear polyribonucleotide may be delivered as a naked delivery formulation, such as comprising a diluent. A naked delivery formulation delivers a linear polyribonucleotide, to a cell without the aid of a carrier and without modification or partial or complete encapsulation of the linear polyribonucleotide, capped polyribonucleotide, or complex thereof.

A naked delivery formulation is a formulation that is free from a carrier and wherein the linear polyribonucleotide is without a covalent modification that binds a moiety that aids in delivery to a cell or without partial or complete encapsulation of the linear polyribonucleotide. In some embodiments, a linear polyribonucleotide without a covalent modification that binds a moiety that aids in delivery to a cell is a polyribonucleotide that is not covalently bound to a protein, small molecule, a particle, a polymer, or a biopolymer. A linear polyribonucleotide without covalent modification that binds a moiety that aids in delivery to a cell does not contain a modified phosphate group. For example, a linear polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In some embodiments, a naked delivery formulation is free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation comprises a non-carrier excipient. In some embodiments, a non-carrier excipient comprises an inactive ingredient that does not exhibit a cell-penetrating effect. In some embodiments, a non-carrier excipient comprises a buffer, for example PBS. In some embodiments, a non-carrier excipient is a solvent, a non-aqueous solvent, a diluent, a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation comprises a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carrier

In some embodiments, an immunogenic composition of the invention comprises a circular polyribonucleotide and a carrier. In some embodiments, an immunogenic composition of the invention comprises a linear polyribonucleotide and a carrier.

In certain embodiments, an immunogenic composition comprises a circular polyribonucleotide as described herein in a vesicle or other membrane-based carrier. In certain embodiments, an immunogenic composition comprises a linear polyribonucleotide as described herein in a vesicle or other membrane-based carrier.

In other embodiments, an immunogenic composition comprises the circular polyribonucleotide in or via a cell, vesicle or other membrane-based carrier. In other embodiments, an immunogenic composition comprises the linear polyribonucleotide in or via a cell, vesicle or other membrane-based carrier. In one embodiment, an immunogenic composition comprises the circular polyribonucleotide in liposomes or other similar vesicles. In one embodiment, an immunogenic composition comprises the linear polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In certain embodiments, an immunogenic composition of the invention comprises a circular polyribonucleotide and lipid nanoparticles, e.g., a lipid nanoparticle formulation described herein. In certain embodiments, an immunogenic composition of the invention comprises a linear polyribonucleotide and lipid nanoparticles. Lipid nanoparticles are another example of a carrier that provides a biocompatible and biodegradable delivery system for a circular polyribonucleotide molecule as described herein. Lipid nanoparticles are another example of a carrier that provides a biocompatible and biodegradable delivery system for a linear polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide or a protein covalently linked to the linear polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as a carrier or drug delivery vehicles for a circular polyribonucleotide molecule described herein. Exosomes can also be used as a carrier or drug delivery vehicles for a linear polyribonucleotide molecule described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular polyribonucleotide molecule described herein. Ex vivo differentiated red blood cells can also be used as a carrier for a linear polyribonucleotide molecule described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a circular polyribonucleotide molecule described herein. Fusosome compositions, e.g., as described in WO2018208728, can also be used as carriers to deliver a linear polyribonucleotide molecule described herein.

Virosomes and virus-like particles (VLPs) can also be used as carriers to deliver a circular polyribonucleotide molecule described herein to targeted cells. Virosomes and virus-like particles (VLPs) can also be used as carriers to deliver a linear polyribonucleotide molecule described herein to targeted cells.

Plant nanovesicles and plant messenger packs (PMPs), e.g., as described in International Patent Publication Nos. WO2011097480, WO2013070324, WO2017004526, or WO2020041784, can also be used as carriers to deliver a circular polyribonucleotide described herein. Plant nanovesicles and plant messenger packs (PMPs) can also be used as carriers to deliver a linear polyribonucleotide molecule described herein

Microbubbles can also be used as carriers to deliver a circular polyribonucleotide molecule described herein. Microbubbles can also be used as carriers to deliver a linear polyribonucleotide described herein. See, e.g., U.S. Pat. No. 7,115,583; Beeri, R. et al., Circulation. 2002 Oct. 1; 106(14):1756-1759; Bez, M. et al., Nat Protoc. 2019 April; 14(4): 1015-1026; Hernot, S. et al., Adv Drug Deliv Rev. 2008 Jun. 30; 60(10): 1153-1166; Rychak, J. J. et al., Adv Drug Deliv Rev. 2014 June; 72: 82-93. In some embodiments, microbubbles are albumin-coated perfluorocarbon microbubbles.

### Lipid Nanoparticles

The compositions, methods, and delivery systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

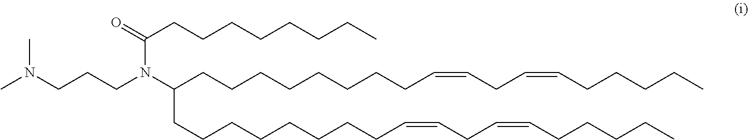 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a polyribonucleotide (e.g., a circular polyribonucleotide, a linear polyribonucleotide) composition described herein to cells.

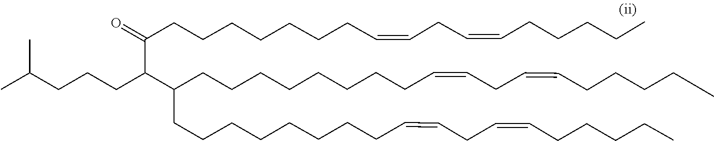 [SMILES: CCCCC/C=C\C/C=C\CCCCCCCCC(CCCCCCCC/C=C\C/C=C\CCCCCC)C(CCCC(C)C)C(=O)CCCCCCC/C=C\C/C=C\CCCCC]

In some embodiments an LNP comprising Formula (ii) is used to deliver a polyribonucleotide (e.g., a circular polyribonucleotide, a linear polyribonucleotide) composition described herein to cells.

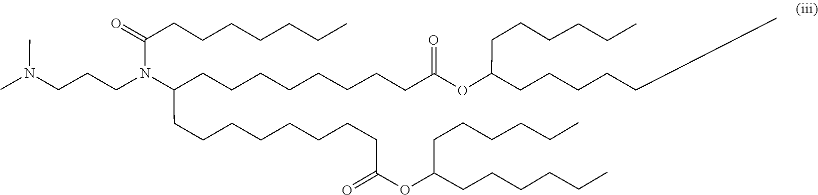 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a polyribonucleotide (e.g., a circular polyribonucleotide, a linear polyribonucleotide) composition described herein to cells.

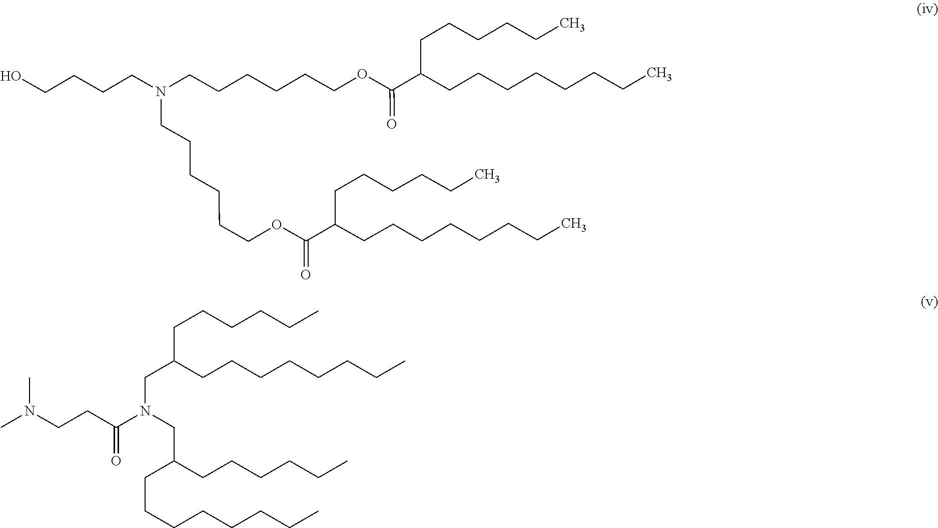

In some embodiments an LNP comprising Formula (v) is used to deliver a polyribonucleotide (e.g., a circular polyribonucleotide, a linear polyribonucleotide) composition described herein to cells.

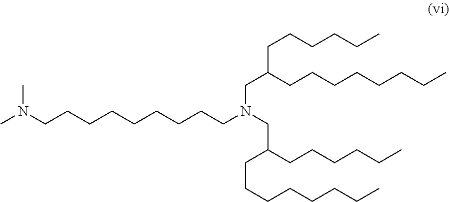 [SMILES: CCCCCCCCC(CCCCCC)CN(CCCCCCCCCN(C)C)CC(CCCCCC)CCCCCCCC]

In some embodiments an LNP comprising Formula (vi) is used to deliver a polyribonucleotide (e.g., a circular polyribonucleotide, a linear polyribonucleotide) composition described herein to cells.

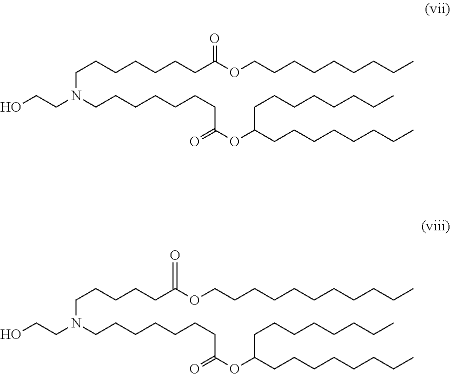 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a polyribonucleotide (e.g., a circular polyribonucleotide, a linear polyribonucleotide) composition described herein to cells.

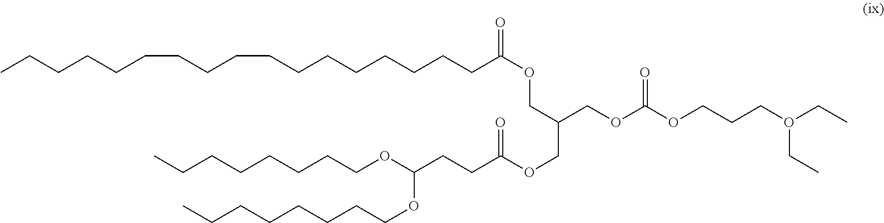

In some embodiments an LNP comprising Formula (ix) is used to deliver a polyribonucleotide (e.g., a circular polyribonucleotide, a linear polyribonucleotide) composition described herein to cells.

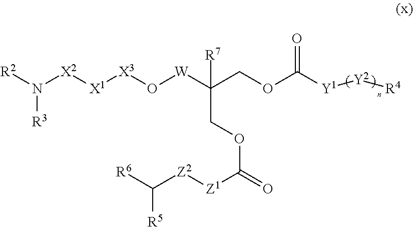

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

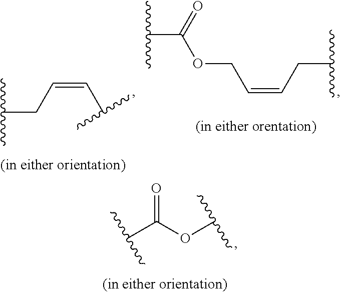 [SMILES: */C=C\C* ; *C/C=C\COC(*)=O ; *OC(*)=O]

n is 0 to 3, R⁴ is C1-15 alkyl, Z¹ is C1-6 alkylene or a direct bond,

Z² is

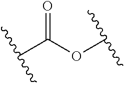 [SMILES: *OC(*)=O]

(in either orientation) or absent provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are (C2 alkyls, X¹ is O, X² is linear C3 alkylene, X³ is C(═O), Y¹ is linear Ce alkylene, (Y²)n-R⁴ is

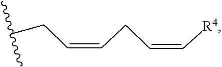

R⁴ is linear C5 alkyl, Z¹ is C2 alkylene Z² is absent, W is methylene, and R⁷ is H, then R⁵ and R⁶ are not Cx alkoxy.

In some embodiments an LNP comprising Formula (xii) is used to deliver a polyribonucleotide (e.g., a circular polyribonucleotide, a linear polyribonucleotide) composition described herein to cells.

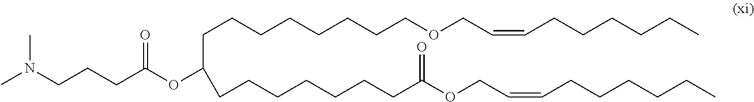 [SMILES: CCCCCC/C=C\COCCCCCCCCC(CCCCCCCC(=O)OC/C=C\CCCCCC)OC(=O)CCCN(C)C]

In some embodiments an LNP comprising Formula (xi) is used to deliver a polyribonucleotide (e.g., a circular polyribonucleotide, a linear polyribonucleotide) composition described herein to cells.

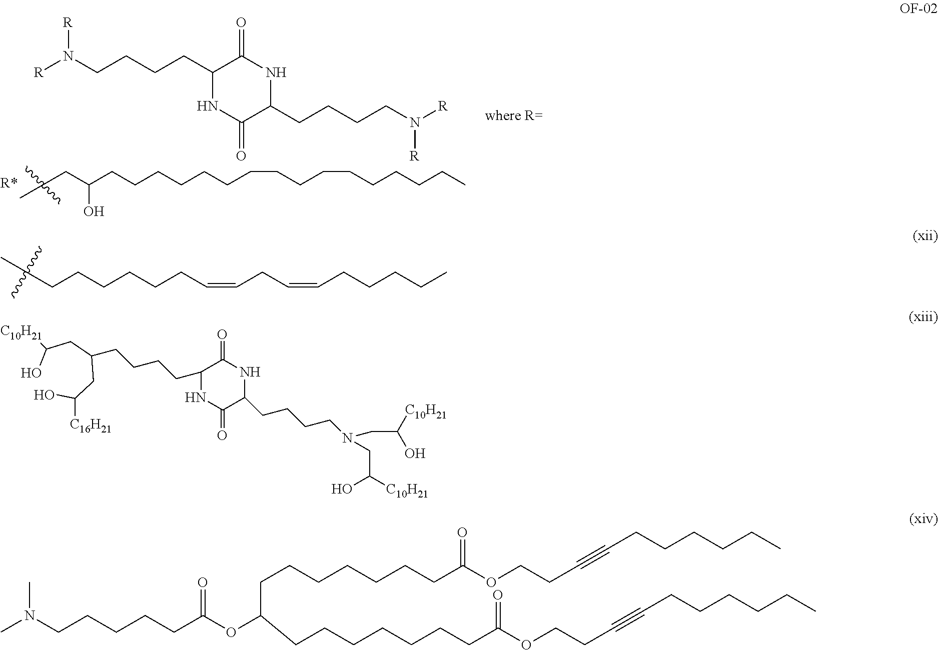

In some embodiments an LNP comprises a compound of Formula (xiii) and a compound of Formula (xiv).

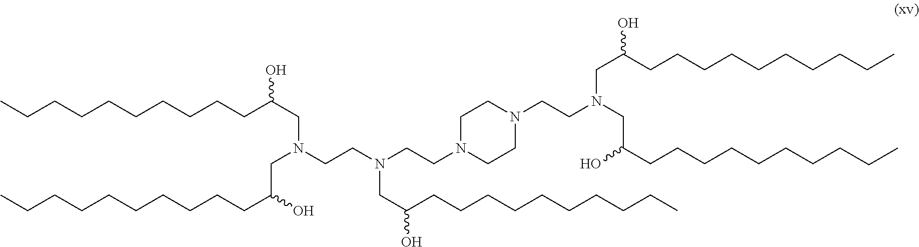 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a polyribonucleotide (e.g., a circular polyribonucleotide, a linear polyribonucleotide) composition described herein to cells.

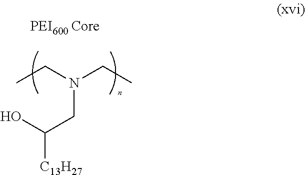

In some embodiments an LNP comprising a formulation of Formula (xvi) is used to deliver a polyribonucleotide (e.g., a circular polyribonucleotide, a linear polyribonucleotide) composition described herein to cells.

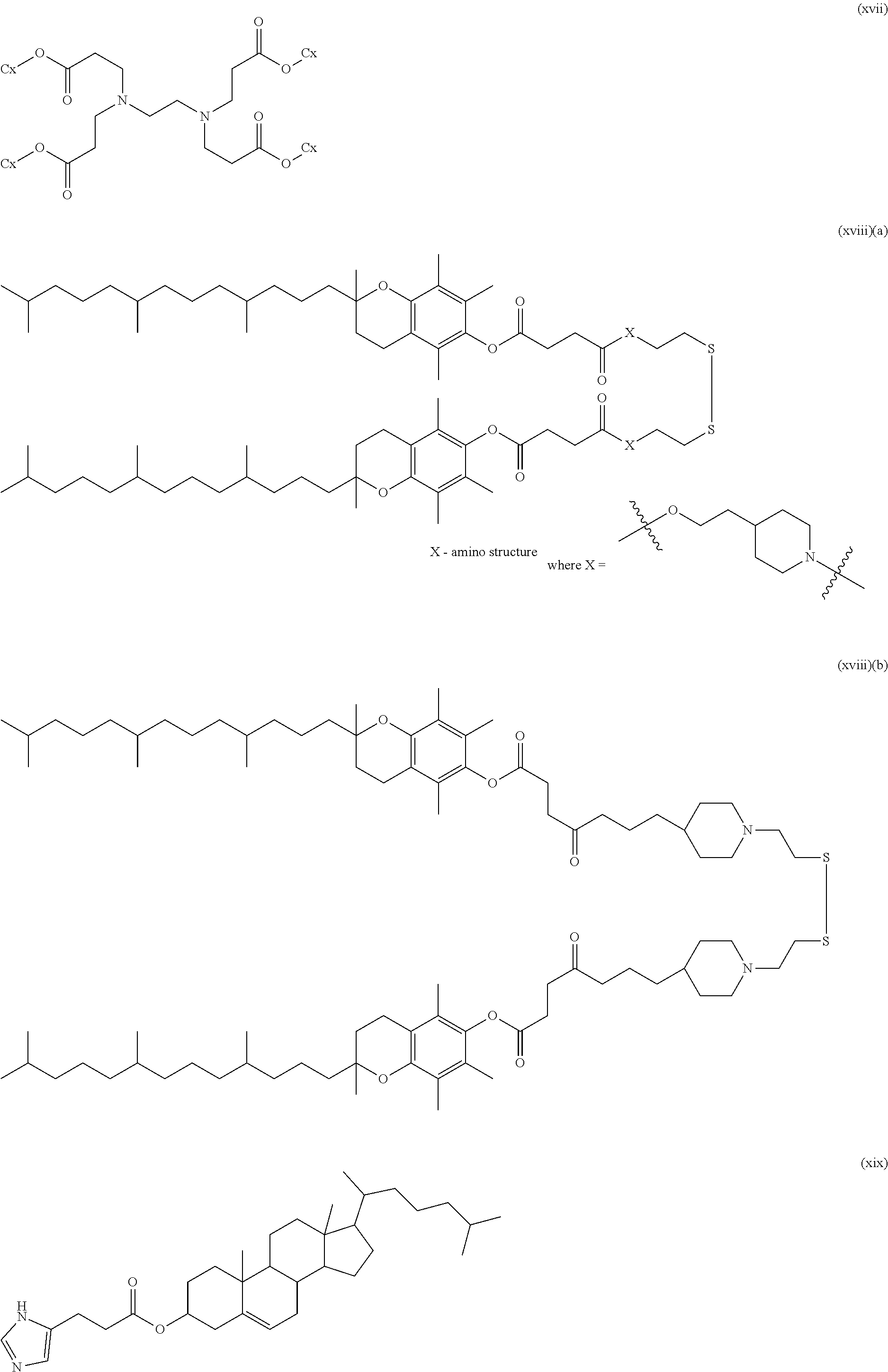

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

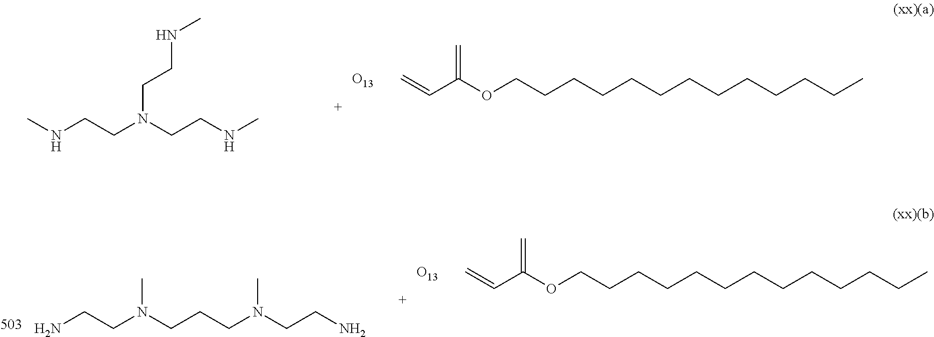

In some embodiments, a composition described herein (e.g., a nucleic acid or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP1), e.g., as synthesized in Example 13 of WO2015/095340 (incorporated by reference herein in its entirety). In some embodiments, the ionizable lipid is DW(Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA (e.g., circular polyribonucleotide, linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 lZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

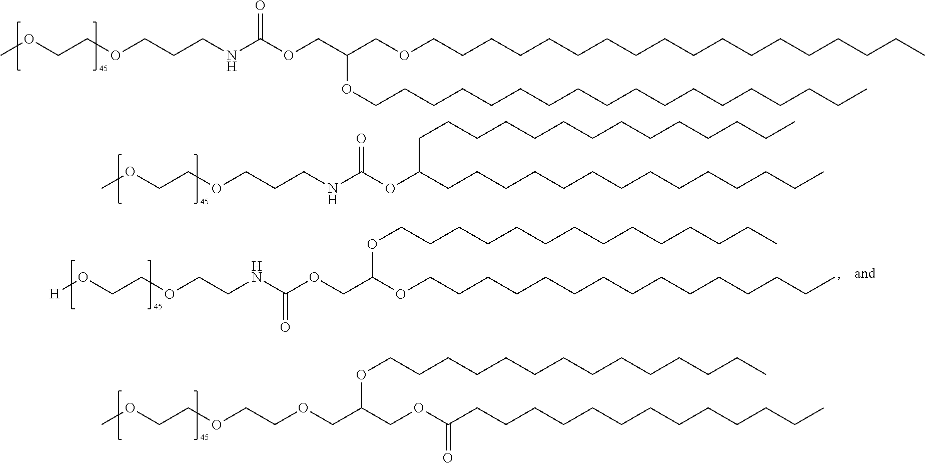

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). Exemplary dosing of AAV comprising a polyribonucleotide described herein may include an MOI of about 10¹¹, 10¹², 10¹³, and 10¹⁴ vg/kg.

### Adjuvant

An adjuvant enhances the immune responses (humoral and/or cellular) elicited in a subject (e.g., a subject for immunization) who receives the adjuvant and/or an immunogenic composition comprising the adjuvant. In some embodiments, an adjuvant is administered to a subject (e.g., a subject for immunization) for the production of polyclonal antibodies from a circular polyribonucleotide as disclosed herein. In some embodiments, an adjuvant is administered to a subject for the production of polyclonal antibodies from a linear polyribonucleotide as disclosed herein. In some embodiments, an adjuvant is used in the methods described herein to produce polyclonal antibodies as described herein. In a particular embodiment, an adjuvant is used to promote production of the polyclonal antibodies in a subject against a coronavirus antigen and/or epitope expressed from a circular polyribonucleotide. In some embodiments, an adjuvant and circular polyribonucleotide are co-administered in separate compositions. In some embodiments, an adjuvant is mixed or formulated with a circular polyribonucleotide in a single composition to obtain an immunogenic composition that is administered to a subject. In a particular embodiment, an adjuvant is used to promote production of the polyclonal antibodies in a subject against a coronavirus antigen and/or epitope expressed from a linear polyribonucleotide. In some embodiments, an adjuvant and linear polyribonucleotide are co-administered in separate compositions. In some embodiments, an adjuvant is mixed or formulated with a linear polyribonucleotide in a single composition to obtain an immunogenic composition that is administered to a subject.

Adjuvants may be a TH1 adjuvant and/or a TH2 adjuvant. Preferred adjuvants include, but are not limited to, one or more of the following:

Mineral-containing compositions. Mineral-containing compositions suitable for use as adjuvants in the invention include mineral salts, such as aluminum salts, and calcium salts. The invention includes mineral salts such as hydroxides (e.g. oxyhydroxides), phosphates (e.g. hydroxyphosphates, orthophosphates), sulphates, etc., or mixtures of different mineral compounds, with the compounds taking any suitable form (e.g. gel, crystalline, amorphous, etc.). Calcium salts include calcium phosphate (e.g., the “CAP”). Aluminum salts include hydroxides, phosphates, sulfates, and the like.

Oil emulsion compositions. Oil-emulsion compositions suitable for use as adjuvants in the invention include squalene-water emulsions, such as MF59 (5% Squalene, 0.5% Tween 80 and 0.5% Span, formulated into submicron particles using a microfluidizer), AS03 (α-tocopherol, squalene and polysorbate 80 in an oil-in-water emulsion), Montanide formulations (e.g. Montanide ISA 51, Montanide ISA 720), incomplete Freunds adjuvant (IFA), complete Freund's adjuvant (CFA), and incomplete Freund's adjuvant (IFA).

Small molecules. Small molecules suitable for use as adjuvants in the invention include imiquimod or 847, resiquimod or R848, or gardiquimod.

Polymeric nanoparticles. Polymeric nanoparticles suitable for use as an adjuvant in the invention include poly(a-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

Saponin (i.e., a glycoside, polycyclic aglycones attached to one or more sugar side chains). Saponin formulations suitable for use as an adjuvant in the invention include purified formulations, such as QS21, as well as lipid formulations, such as ISCOMs and ISCOMs matrix. QS21 is marketed as STIMULON™. Saponin formulations may also comprise a sterol, such as cholesterol. Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent.

Lipopolysaccharides. Adjuvants suitable for use in the invention include non-toxic derivatives of enterobacterial lipopolysaccharide (LPS). Such derivatives include monophosphoryl lipid A (MPLA), glucopyranosyl lipid A (GLA) and 3-O-deacylated MPL (3dMPL). 3dMPL is a mixture of 3 De-O-acylated monophosphoryl lipid A with 4, 5 or 6 acylated chains. Other non-toxic LPS derivatives include monophosphoryl lipid A mimics, such as aminoalkyl glucosaminide phosphate derivatives e.g. RC-529.

Liposomes. Liposomes suitable for use as an adjuvant in the invention include virosomes and CAF01.

Lipid nanoparticles. Adjuvants suitable for use in the invention include lipid nanoparticles (LNPs) and their components.

Lipopeptides (i.e., compounds comprising one or more fatty acid residues and two or more amino acid residues). Lipopeptide suitable for use as an adjuvant in the invention include Pam2 (Pam2CSK4) and Pam3 (Pam3CSK4).

Glycolipids. Glycolipids suitable for use as an adjuvant in the invention include cord factor (trehalose dimycolate).

Peptides and peptidoglycans derived from (synthetic or purified) gram negative or gram positive bacteria, such as MDP (N-acetyl-muramyl-L-alanyl-D-isoglutamine) are suitable for use as an adjuvant in the invention Carbohydrates (carbohydrate containing) or polysaccharides suitable for use as an adjuvant include dextran (e.g., branched microbial polysaccharide), dextran-sulfate, lentinan, zymosan, beta-glucan, deltin, mannan, and chitin.

RNA based adjuvants. RNA based adjuvants suitable for use in the invention are poly IC, poly IC:LC, hairpin RNAs with or without a 5′triphosphate, viral sequences, polyU containing sequence, dsRNA natural or synthetic RNA sequences, and nucleic acid analogs (e.g., cyclic GMP-AMP or other cyclic dinucleotides e.g., cyclic di-GMP, immunostimulatory base analogs e.g., C8-substituted and N7,C8-disubstituted guanine ribonucleotides).

DNA based adjuvants. DNA based adjuvants suitable for use in the invention include CpGs, dsDNA, and natural or synthetic immunostimulatory DNA sequences.

Proteins or peptides. Proteins and peptides suitable for use as an adjuvant in the invention include flagellin-fusion proteins, MBL (mannose-binding lectin), cytokines, and chemokines.

Viral particles. Viral particles suitable for use as an adjuvant include virosomes (phospholipid cell membrane bilayer).

An adjuvant for use in the invention may be bacterial derived, such as a flagellin, LPS, or a bacterial toxin (e.g., enterotoxins (protein), e.g., heat-labile toxin or cholera toxin). An adjuvant for use in the invention may be a hybrid molecule such as CpG conjugated to imiquimod. An adjuvant for use in the invention may be a fungal or oomycete MAMPs, such as chitin or beta-glucan. In some embodiments, an adjuvant is an inorganic nanoparticle, such as gold nanorods or silica-based nanoparticles (e.g., mesoporous silica nanoparticles (MSN)). In some embodiments, an adjuvant is a multi-component adjuvant or adjuvant system, such as AS01, AS03, AS04 (MLP5+alum), CFA (complete Freund's adjuvant: IFA+peptiglycan+trehalose dimycolate), CAF01 (two component system of cationic liposome vehicle (dimethyl dioctadecyl-ammonium (DDA)) stabilized with a glycolipid immunomodulator (trehalose 6,6-dibehenate (TDB), which can be a synthetic variant of cord factor located in the mycobacterial cell wall).

Cytokines. An adjuvant may be a partial or full-length DNA encoding a cytokine such as, a pro-inflammatory cytokine (e.g., GM-CSF, IL-1 alpha, IL-1 beta, TGF-beta, TNF-alpha, TNF-beta), Th-1 inducing cytokines (e.g., IFN-gamma, IL-2, IL-12, IL-15, IL-18), or Th-2 inducing cytokines (e.g., IL-4, IL-5, IL-6, IL-10, IL-13).

Chemokines. An adjuvant may be a partial or full-length DNA encoding a chemokine such as, MCP-1, MIP-1 alpha, MIP-1 beta, Rantes, or TCA-3.

An adjuvant may be a partial or full-length DNA encoding a costimulatory molecule, such as CD80, CD86, CD40-L, CD70, or CD27.

An adjuvant may be a partial or full length DNA encoding for an innate immune sentinel (partial, full-length, or mutated) or a constitutively active (ca) innate immune sentinel, such as caTLR4, casting, caTLR3, caTLR3, caTLR9, caTLR7, caTLR8, caTLR7, caRIG-I/DDX58, or caMDA-5/IFIH1.

An adjuvant may be a partial or full length DNA encoding for an adaptor or signaling molecule, such as STING, TRIF, TRAM, MyD88, IPS1, ASC, MAVS, MAPKs, IKK-alpha, IKK complex, TBK1, beta-catenin, and caspase 1.

An adjuvant may be a partial or full length DNA encoding for a transcriptional activator, such as a transcription activator that can upregulate an immune response (e.g., AP1, NF-kappa B, IRF3, IRF7, IRF1, or IRF5). An adjuvant may be a partial or full length DNA encoding for a cytokine receptor, such as IL-2beta, IFN-gamma, or IL-6.

An adjuvant may be a partial or full length DNA encoding for a bacterial component, such as flagellin or MBL.

An adjuvant may be a partial or full length DNA encoding for any component of the innate immune system.

In a particular embodiment, an adjuvant used in the invention is a SAB's proprietary adjuvant formulation, SAB-adj-1 or SAB-adj-2.

### Vaccine

In some embodiments of methods described herein, a second agent is also administered to the subject (e.g., a subject for immunization), e.g., a second vaccine is also administered to a subject (e.g., a subject for immunization). In some embodiments, a composition that is administered to a subject comprises a circular polyribonucleotide described herein and a second vaccine. In some embodiments, a vaccine and circular polyribonucleotide are co-administered in separate compositions. The vaccine is simultaneously administered with the circular polyribonucleotide immunization, administered before the circular polyribonucleotide immunization, or after the circular polyribonucleotide immunization.

For example, in some embodiments, a subject (e.g., a subject for immunization) is immunized with a non-circular polyribonucleotide coronavirus vaccine (e.g., protein subunit vaccine) and an immunogenic composition comprising a circular polyribonucleotide. In some embodiments, a subject is immunized with a non-polyribonucleotide vaccine for a first microorganism (e.g., pneumococcus) and an immunogenic composition comprising a circular polyribonucleotide as disclosed herein. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

In some embodiments, a composition that is administered to a subject comprises a linear polyribonucleotide and a vaccine. In some embodiments, a vaccine and linear polyribonucleotide are co-administered in separate compositions. The vaccine is simultaneously administered with the linear polyribonucleotide immunization, administered before the linear polyribonucleotide immunization, or after the linear polyribonucleotide immunization.

For example, in some embodiments, a subject (e.g., a subject for immunization) is immunized with a polyribonucleotide (e.g., non-linear polyribonucleotide) coronavirus vaccine (e.g., protein subunit vaccine) and an immunogenic composition comprising a linear polyribonucleotide as disclosed herein comprising a sequence encoding a coronavirus antigen. In some embodiments, a subject is immunized with a non-polyribonucleotide vaccine for a first microorganism (e.g., pneumococcus) and an immunogenic composition comprising a linear polyribonucleotide as disclosed herein comprising a sequence encoding a coronavirus antigen. A vaccine can be any bacterial infection vaccine or viral infection vaccine. In a particular embodiment, a vaccine is a pneumococcal polysaccharide vaccine, such as PCV13 or PPSV23. In some embodiments, the vaccine is an influenza vaccine. In some embodiments, the vaccine is an RSV vaccine (e.g., palivizumap).

### Subject for Immunization

The disclosure provides administering or immunizing a subject (e.g., a subject for immunization) with an immunogenic composition comprising a circular polyribonucleotide comprising sequence encoding a coronavirus antigen and/or epitope. The disclosure provides administering or immunizing a subject (e.g., a subject for immunization) with an immunogenic composition comprising a linear polyribonucleotide comprising sequence encoding a coronavirus antigen and/or epitope. In some embodiments, a subject (e.g., a subject for immunization) is an animal. In a particular embodiment, a subject (e.g., a subject for immunization) is a mammal. In certain embodiments, a subject (e.g., a subject for immunization) is a human. In some embodiments, a subject (e.g., a subject for immunization) is a non-human animal. In some embodiments, a non-human animal has a humanized immune system. The plasma or blood of the subject is used for generating hyperimmune plasma, e.g., plasma with a high concentration of antibodies that bind to the coronavirus antigens and/or epitopes of interest.

Non-Human Animal for Immunization

The disclosure provides administering or immunizing a non-human animal (e.g., a non-human animal for immunization) with an immunogenic composition comprising a circular polyribonucleotide comprising sequence encoding a coronavirus antigen and/or epitope. The disclosure provides administering or immunizing a non-human animal (e.g., a non-human animal for immunization) with an immunogenic composition comprising a linear polyribonucleotide comprising sequence encoding a coronavirus antigen and/or epitope.

In some embodiments, a non-human animal (e.g., a non-human animal for immunization) is a pet. In some embodiments, a non-human animal (e.g., a non-human animal for immunization) is a livestock animal. In some embodiments, a non-human animal (e.g., a non-human animal for immunization) is a farm animal. In some embodiments, a non-human animal (e.g., a non-human animal for immunization) is a zoo animal (e.g., a tiger, a lion, a wolf, etc.).

In some embodiments, a non-human animal (e.g., a non-human animal for immunization) is a mammal. A non-human animal (e.g., a non-human animal for immunization) includes an ungulate, for example, a donkey, a goat, a horse, a cow, or a pig. A non-human animal (e.g., a non-human animal for immunization) also includes a rabbit, rat, or mouse. In some embodiments, a non-human animal (e.g., a non-human animal for immunization) is a cow (bovine). In other embodiments, a non-human animal is a goat.

In some embodiments, a non-human animal (e.g., a non-human animal for immunization) is a chicken.

In some embodiments, a non-human animal (e.g., a non-human animal for immunization) has a humanized immune system and is used for producing human polyclonal antibodies.

Humanized Immune System

A non-human animal having a humanized immune system (e.g., a non-human animal for immunization having a humanized immune system) includes an ungulate, for example, a donkey, a goat, a horse, a cow, or a pig. A non-human animal having a humanized immune system also includes a rabbit, rat, or a mouse. In some embodiments, a non-human animal having a humanized immune system is a cow (bovine). In some embodiments, a non-human animal having a humanized immune system is a goat. In some embodiments, a non-human animal having a humanized immune system is a chicken.

A non-human animal having a humanized immune system (e.g., a non-human animal for immunization having a humanized immune system) is an animal that produces human antibodies, or antibody variants, fragments, and derivatives thereof. A humanized immune system comprises a humanized immunoglobulin gene locus, or multiple humanized immunoglobulin gene loci.

In some embodiments, humanized immunoglobulin gene locus comprises a germ line sequence of human immunoglobulin, allowing the non-human animal to produce humanized antibodies (e.g., fully human antibodies).

In some embodiments, a non-human animal with a humanized immune system of the disclosure comprises non-human B cells with a humanized immunoglobulin gene locus. The humanized immunoglobulin gene locus undergoes VDJ recombination during B cell development, thereby allowing for generation of B cells with great diversity of antigen binging specificity.

The binding specificity of antibodies is generated by the process of VDJ recombination. The exons encoding the antigen binding portions (the variable regions) are assembled by chromosomal breakage and rejoining in developing B cells. The exons encoding the antigen binding domains are assembled from so-called V (variable), D (diversity), and J (joining) gene segments by “cut and paste” DNA rearrangements. This process, termed V(D)J recombination, chooses a pair of segments, introduces double-strand breaks adjacent to each segment, deletes (or, in selected cases, inverts) the intervening DNA, and ligates the segments together. Rearrangements can occur in an ordered fashion, with D to J joining proceeding before a V segment is joined to the rearranged DJ segments. This process of combinatorial assembly—choosing one segment of each type from several (sometimes many) possibilities is the fundamental engine driving antigen receptor diversity in mammals. Diversity is tremendously amplified by the characteristic variability at the junctions (loss or gain of small numbers of nucleotides) between the various segments. This process leverages a relatively small investment in germline coding capacity into an almost limitless repertoire of potential antigen binding specificities.

In some embodiments, a non-human animal with a humanized immune system comprises a plurality of B cells of diverse specificities generated by VDJ recombination, for example, of the humanized immunoglobulin gene locus. A B cell that encodes a B cell receptor (and an antibody) that specifically binds to an antigen and/or epitope of the disclosure is activated upon countering cognate antigen, for example, after encountering the antigen and/or epitope that is expressed from a circular polyribonucleotide of the disclosure. A B cell that encodes a B cell receptor (and an antibody) that specifically binds to an antigen and/or epitope of the disclosure is activated upon countering cognate antigen, for example, after encountering the antigen and/or epitope that is expressed from a linear polyribonucleotide of the disclosure. The activated B cell produces antibodies that specifically bind the antigen and/or epitope of the disclosure. The activated B cell proliferates. In some embodiments, the activated non-human B cell differentiates into memory B cells and/or plasma cells. In some embodiments, the activated non-human B cell undergoes class switching to generate antibodies of different isotypes as disclosed herein. In some embodiments, the non-human B cell undergoes somatic hypermutation to generate antibodies that bind to an antigen and/or epitope with higher affinity.

Upon immunization with one or more immunogenic compositions comprising one or more circular polyribonucleotides of the disclosure that express multiple antigens and/or epitopes, a plurality of B cell clones respond to their respective cognate antigens, leading to the generation of polyclonal antibodies with a plurality of binding specificities. In some embodiments, immunizing a non-human animal of the disclosure with one or more immunogenic compositions comprising one or more circular polyribonucleotides of the disclosure activates at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones. In some embodiments, immunizing a non-human animal of the disclosure with one or more immunogenic compositions comprising one or more circular polyribonucleotides of the disclosure leads to production of polyclonal antiserum that comprises antibodies that specifically bind at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens and/or epitopes of the disclosure.

Upon immunization with one or more immunogenic compositions comprising one or more linear polyribonucleotides of the disclosure that express multiple antigens and/or epitopes, a plurality of B cell clones respond to their respective cognate antigens, leading to the generation of polyclonal antibodies with a plurality of binding specificities. In some embodiments, immunizing a non-human animal of the disclosure with one or more immunogenic compositions comprising one or more linear polyribonucleotides of the disclosure activates at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones. In some embodiments, immunizing a non-human animal of the disclosure with one or more immunogenic compositions comprising one or more linear polyribonucleotides of the disclosure leads to production of polyclonal antiserum that comprises antibodies that specifically bind at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens and/or epitopes of the disclosure.

Various techniques for modifying the genome of non-human animals (e.g., non-human animals for immunization) can be employed to develop an animal capable of producing humanized antibodies. A non-human animal can be a transgenic animal, for example, a transgenic animal comprising all or a substantial portion of the humanized immunoglobulin gene locus or loci. A non-human animal can be a transchromosomal animal, for example, a non-human animal that comprises a human artificial chromosome or a yeast artificial chromosome.

A humanized immunoglobulin gene locus can be present on a vector, for example, a human artificial chromosome or a yeast artificial chromosome (YAC). A human artificial chromosome (HAC) comprising the humanized immunoglobulin gene locus can be introduced into an animal. A vector (e.g., HAC) can contain the germline repertoire of the human antibody heavy chain genes (from human chromosome 14) and the human antibody light chain genes, for example, one or both of kappa (from human chromosome 2) and lambda (from human chromosome 22). The HAC can be transferred into cells of the non-human animal species and the transgenic animals can be produced by somatic cell nuclear transfer. The transgenic animals can also be bred to produce non-human animals comprising the humanized immunoglobulin gene locus.

In some embodiments, a humanized immunoglobulin gene locus is integrated into the non-human animal's genome. For example, techniques comprising homologous recombination or homology-directed repair can be employed to modify the animal's genome to introduce the human nucleotide sequences. Tools such as CRISPR/Cas, TALEN, and zinc finger nucleases can be used to target integration.

Methods of generating non-human animals having humanized immune systems (e.g., non-human animals for immunization having humanized immune systems) have been disclosed. For example, a human artificial chromosome can be generated and transferred into a cell that comprises additional genomic modifications of interest (e.g., deletions of endogenous non-human immune system genes), and the cell can be used as a nuclear donor to generate a transgenic non-human animal.

In some embodiments, the humanized immune system comprises one or more human antibody heavy chains, wherein each gene encoding an antibody heavy chain is operably linked to a class switch regulatory element. Operably linked can mean that a first DNA molecule (e.g., heavy chain gene) is joined to a second DNA molecule (e.g., class switch regulatory element), wherein the first and second DNA molecules are arranged so that the first DNA molecule affects the function of the second DNA molecule. The two DNA molecules may or may not be part of a single contiguous DNA molecule and may or may not be adjacent. For example, a promoter is operably linked to a transcribable DNA molecule if the promoter is capable of affecting the transcription or translation of the transcribable DNA molecule.

In some embodiments, the humanized immune system comprises one or more human antibody light chains. In some embodiments, the humanized immune system comprises one or more human antibody surrogate light chains.

In some embodiments, the humanized immune system comprises an amino acid sequence that is derived from the non-human animal, for example, a constant region, such as a heavy chain constant region or a part thereof. In some embodiments, a humanized immune system comprise an IgM heavy chain constant region from the non-human animal (for example, an ungulate-derived IgM heavy chain constant region). In some embodiments, at least one class switch regulatory element of the genes encoding the one or more human antibody heavy chains is replaced with a non-human (e.g., ungulate-derived) class switch regulatory element, for example, to allow antibody class switching when antibodies are raised against antigens and/or epitopes of the disclosure within the non-human animal.

A humanized immunoglobulin gene locus can comprise non-human elements that are incorporated for compatibility with the non-human animal. In some embodiments, a non-human element can be present in a humanized immunoglobulin gene locus to reduce recognition by any remaining elements of the non-human animal's immune system). In some embodiments, an immunoglobulin gene (e.g., IgM) can be partly replaced with an amino acid sequence from the non-human animal. In some embodiments, a non-human regulatory element present in a humanized immunoglobulin gene locus to facilitate expression and regulation of the locus within the non-human animal.

A humanized immunoglobulin gene locus can comprise a human DNA sequence. A humanized immunoglobulin gene locus can be codon optimized to facilitate expression of the encompassed genes (e.g., antibody genes) in the non-human animal.

A non-human animal having a humanized immune system (e.g., a non-human animal for immunization having a humanized immune system) can comprise or can lack endogenous non-human immune system components. In some embodiments, a non-human animal with a humanized immune system can lack non-human antibodies (e.g., lack the ability to produce non-human antibodies). A non-human animal with a humanized immune system can lack, for example, one or more non-human immunoglobulin heavy chain genes, one or more non-human immunoglobulin light chain genes, or a combination thereof.

A non-human animal with a humanized immune system (e.g., a non-human animal for immunization having a humanized immune system) can retain, for example, non-human immune cells. A non-human animal with a humanized immune system can retain non-human innate immune system components (e.g., cells, complement, antimicrobial peptides, etc.). In some embodiments, a non-human animal with a humanized immune system can retain non-human T cells. In some embodiments, a non-human animal with a humanized immune system can retain non-human B cells. In some embodiments, a non-human animal with a humanized immune system can retain non-human antigen-presenting cells. In some embodiments, a non-human animal with a humanized immune system can retain non-human antibodies.

In some embodiments, a humanized immune system comprises human innate immune proteins, for example, complement proteins.

In some embodiments, a humanized immune system comprises humanized T cells and/or antigen-presenting cells.

In some embodiments, compositions and methods of the disclosure comprise T cells. For example, a circular polyribonucleotide of the disclosure can comprise antigens recognized by B cells and T cells, and upon immunization of a non-human animal with a humanized immune system, the T cells can provide T cell help, thereby increasing antibody production in the non-human animal. In another example, a linear polyribonucleotide of the disclosure can comprise antigens recognized by B cells and T cells, and upon immunization of a non-human animal with a humanized immune system, the T cells can provide T cell help, thereby increasing antibody production in the non-human animal.

In some embodiments, the non-human animal having a humanized immune system (e.g., a non-human animal for immunization having a humanized immune system) comprises any feature or any combination of features or any methods of making as disclosed in US20170233459, which is hereby incorporated by reference in its entirety. In some embodiments, the non-human animal having a humanized immune system (e.g., a non-human animal for immunization having a humanized immune system) comprises any feature or any combination of features or any methods of making as disclosed in Kuroiwa, Y et al. Nat Biotechnol, 2009 February; 27(2):173-81; Matsushita, H. et al. PLos ONE, 2014 Mar. 6; 9(3): e90383; Hooper, J. W. et al. Sci Transl Med, 2014 Nov. 26; 6(264): 264ra162; Matsushit, H. et al., PLoS ONE 2015 Jun. 24; 10(6): e0130699; Luke, T. et al. Sci Transl Med, 2016 Feb. 17; 8(326): 326ra21; Dye, J. et al., Sci Rep. 2016 Apr. 25; 6:24897; Gardner, C. et al. J Virol. 2017 Jun. 26; 91(14); Stein, D. et al., Antiviral Res 2017 October; 146:164-173; Silver, J. N., Clin Infect Dis. 2018 Mar. 19; 66(7):1116-1119; Beigel, J. H. et al., Lancet Infect Dis, 2018 April; 18; (4):410-418; Luke, T. et al., J Inf Dis. 2018 November 33; 218(suppl_5):S636-S648, each of which is hereby incorporated by reference in its entirety.

### Plasma Collection

Plasma comprising polyclonal antibodies produced from immunogenic compositions comprising circular polyribonucleotides encoding a coronavirus antigen and/or epitope expressed from a circular polyribonucleotide as disclosed herein can be collected from a subject (e.g., after immunization of the subject for immunization) that was immunized with the circular polyribonucleotide. These polyclonal antibodies can be used in a prophylactic or treatment of a coronavirus associated with an antigen and/or epitope expressed from the circular polyribonucleotide. Plasma comprising polyclonal antibodies produced from immunogenic compositions comprising linear polyribonucleotides encoding a coronavirus antigen and/or epitope expressed from a linear polyribonucleotide as disclosed herein can be collected from a subject (e.g., after immunization of the subject for immunization) that was immunized with the linear polyribonucleotide. These polyclonal antibodies can be used in a prophylactic or treatment of a coronavirus associated with an antigen and/or epitope expressed from the linear polyribonucleotide. Plasma can be collected via plasmapheresis. Plasma can be collected from the same subject (e.g., after immunization of the subject for immunization) once or multiple times, for example, multiple times each a given period of time after an immunization, multiple times after an immunization, multiple times in between immunizations, or any combination thereof.

Plasma can be collected from a subject (e.g., after immunization of the subject for immunization) any suitable amount of time following an immunization, for example the first immunization, the most recent immunization, or an intermediate immunization. Plasma can be collected from the subject at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some embodiments, plasma is collected from the subject at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some embodiments, plasma is collected from the subject about 1, 2, 3, 4, 5, 6, 7, 8,9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization. In some embodiments, a composition comprises plasma collected after administration of the immunogenic composition described herein.

Plasma can be frozen (e.g., stored or transported frozen). In some embodiments, plasma is maintained fresh, or antibodies are purified from fresh plasma.

In some embodiments, a composition comprises the collected plasma. For example, a composition comprises plasma from a subject and a circular polyribonucleotide comprising a sequence encoding an antigen. In some embodiments, a composition comprises plasma from a subject and a circular polyribonucleotide comprising a sequence encoding an antigen, and the antigen. In an example, a composition comprises plasma from a subject and a linear polyribonucleotide comprising a sequence encoding an antigen. In some embodiments, a composition comprises plasma from a subject and a linear polyribonucleotide comprising a sequence encoding an antigen, and the antigen.

### Polyclonal Antibody Purification

The disclosure provides polyclonal antibodies specific to coronavirus antigens and/or epitopes of the invention, and methods of treatment or prevention of coronavirus-related disease or infection by administering an effective amount of the polyclonal antibodies to a subject (e.g., a subject treatment). Polyclonal antibodies are produced as disclosed herein and purified after plasma collection from the subject (e.g., a subject for immunization) that was immunized with the immunogenic compositions comprising a circular polyribonucleotide. Polyclonal antibodies are produced as disclosed herein and purified after plasma collection from the subject (e.g., the subject for immunization) that was immunized with the immunogenic compositions comprising a linear polyribonucleotide.

Polyclonal antibodies are purified from plasma using techniques well known to those of skill in the art. For example, plasma is pH-adjusted to 4.8 (e.g., with dropwise addition of 20% acetic acid), fractionated by caprylic acid at a caprylic acid/total protein ratio of 1.0, and then clarified by centrifugation (e.g., at 10,000 g for 20 min at room temperature). The supernatant containing polyclonal antibodies (e.g., IgG polyclonal antibodies) is neutralized to pH 7.5 with 1 M tris, 0.22 μM filtered, and affinity-purified with an anti-human immunoglobulin-specific column (e.g., anti-human IgG light chain-specific column). The polyclonal antibodies are further purified by passage over an affinity column that specifically binds impurities, for example, non-human antibodies from the non-human animal. The polyclonal antibodies are stored in a suitable buffer, for example, a sterile-filtered buffer consisting of 10 mM glutamic acid monosodium salt, 262 mM D-sorbitol, and Tween (0.05 mg/ml) (pH 5.5). The quantity and concentration of the purified polyclonal antibodies are determined. HPLC size exclusion chromatography is conducted to determine whether aggregates or multimers are present.

In some embodiments, the human polyclonal antibodies are purified from a non-human animal having a humanized immune system according to Beigel, J H et al. (Lancet Infect. Dis., 18:410-418 (2018), including Supplementary appendix), which is herein incorporated by reference in its entirety. Briefly, humanized IgG polyclonal antibodies from a non-human animal having a humanized immune system are purified using chromatography. Fully human IgG is separated from the non-human animal IgG using a human IgG kappa chain specific affinity column (e.g., KappaSelect from GE healthcare) as a capture step. The human IgG kappa chain specific affinity column specifically binds the fully human IgG with minimum cross-reactivity to non-human animal IgG Fc and IgG. Further non-human animal IgG is removed using an IgG Fc specific affinity column that binds to the specifically binds to the non-human animal IgG (e.g., for bovine, Capto HC15 from GE healthcare), which is used as a negative affinity step to specifically clear the non-human animal IgG. An anion exchange chromatography step is also be used to further reduce contaminants, such as host DNA, endotoxin, IgG aggregates and leached affinity ligands.

### Polyclonal Antibodies

The polyclonal antibodies produced as disclosed bind to coronavirus antigens and/or epitopes (e.g., SARS-CoV-2 antigens and/or epitopes). These polyclonal antibodies are used in methods of treatment or prevention of coronavirus-related disease or infection (e.g., COVID-19 or SARS-CoV-2 infection), for example, the antibodies can provide protection against a coronavirus that expresses the antigens and/or epitopes or similar antigens and/or epitopes.

Polyclonal antibodies of the disclosure bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more coronavirus antigens or epitopes.

In some embodiments, polyclonal antibodies of the disclosure bind to, for example, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less coronavirus antigens or epitopes.

In some embodiments, polyclonal antibodies of the disclosure bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 coronavirus antigens or epitopes.

Polyclonal antibodies of the disclosure bind to one or more epitopes from a coronavirus antigen. For example, a coronavirus antigen comprises an amino acid sequence, which contains multiple epitopes (e.g., epitopes recognized by B cells and/or T cells) therein, and antibody clones bind to one or more of those epitopes.

In some embodiments, polyclonal antibodies of the disclosure bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more epitopes from one coronavirus antigen.

In some embodiments, polyclonal antibodies of the disclosure bind to, for example, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, or at most 500, or less epitopes from one coronavirus antigen.

In some embodiments, polyclonal antibodies of the disclosure bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 epitopes from one coronavirus antigen.

Polyclonal antibodies of the disclosure bind to variants of a coronavirus antigen or epitope. Variants can be naturally-occurring variants (for example, variants identified in sequence data from different coronavirus species, isolates, or quasispecies), or can be derivative sequences as disclosed herein that have been generated in silico (for example, antigen or epitopes with one or more amino acid insertions, deletions, substitutions, or a combination thereof compared to a wild type antigen or epitope).

In some embodiments, polyclonal antibodies of the disclosure bind to, for example, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more variants of a coronavirus antigen or epitope.

In some embodiments, polyclonal antibodies of the disclosure bind to, for example, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less variants of a coronavirus antigen or epitope.

In some embodiments, polyclonal antibodies of the disclosure bind to, for example, about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 variants of a coronavirus antigen or epitope.

In a particular embodiment, antibodies of the disclosure are neutralizing antibodies, non-neutralizing antibodies, or a combination thereof.

Humanized antibodies, or variants, fragments, and derivatives thereof can be antibodies that can be formulated for administration to a human. Humanized antibodies can be chimeric humanized antibodies or fully human antibodies.

Humanized antibodies can be chimeric humanized antibodies, for example, comprising an amino acid sequence from or with similarity to a human antibody amino acid sequence, and a non-human amino acid sequence. For example, a portion of the heavy and/or light chain of a chimeric humanized antibody can be identical to or similar to a corresponding sequence in a human antibody, while the remainder of the chain(s) can be non-human, for example, identical or similar to a corresponding sequence in an antibody derived from another species, or belonging to another antibody class or subclass. The non-human sequence can be humanized to reduce the likelihood of immunogenicity while preserving target specificity, for example, by incorporation of human DNA to the genetic sequence of the genes that produce the antibodies in the non-human animal.

Humanized antibodies can be fully human antibodies, for example, containing an amino acid sequence that is a human antibody amino acid sequence.

In some embodiments, a non-human animal with a humanized immune system produces only fully human antibodies.

Antibodies of the disclosure can be antibodies that comprise the basic four chain antibody unit. The basic four chain antibody unit can comprise two heavy chain (H) polypeptide sequences and two light chain (L) polypeptide sequences. Each of the heavy chains can comprise one N-terminal variable (V_{H}) region and three or four C-terminal constant (C_{H}1, C_{H}2, C_{H}3, and C_{H}4) regions. Each of the light chains can comprise one N-terminal variable (V_{L}) region and one C-terminal constant (C_{L}) region. The light chain variable region is aligned with the heavy chain variable region and the light chain constant region is aligned with first heavy chain constant region C_{H1}. The pairing of a heavy chain variable region and light chain variable region together forms a single antigen-binding site. Each light chain is linked to a heavy chain by one covalent disulfide bond. The two heavy chains are linked to each other by one or more disulfide bonds depending on the heavy chain isotype. Each heavy and light chain can also comprise regularly-spaced intrachain disulfide bridges. The C-terminal constant regions of the heavy chains comprise the Fc region of the antibody, which can mediate effector functions, for example, through interactions with Fc receptors or complement proteins.

The light chain can be designated kappa or lambda based on the amino acid sequence of the constant region. The heavy chain can be designated alpha, delta, epsilon, gamma, or mu based on the amino acid sequence of the constant region. Antibodies are categorized into five immunoglobulin classes, or isotypes, based on the heavy chain. IgA comprises alpha heavy chains, IgD comprises delta heavy chains, IgE comprises epsilon heavy chains, IgG comprises gamma heavy chains, and IgM comprises mu heavy chains. Antibodies of the IgG, IgD, and IgE classes comprise monomers of the four chain unit described above (two heavy and two light chains), while the IgM and IgA classes can comprise multimers of the four chain unit. The alpha and gamma classes are further divided into subclasses on the basis of differences in the sequence and function of the heavy chain constant region. Subclasses of IgA and IgG expressed by humans include IgG1, IgG2, IgG3, IgG4, IgA1 and IgA2.

Illustrative amino acid sequences of human constant domain sequences are provided in TABLE 4. In some embodiments, an antibody, non-human animal, or non-human B cell of the disclosure comprises a human IgG1 constant domain sequence, for example, comprises SEQ ID NO: 34, or a variant, derivative, or fragment thereof. In some embodiments, an antibody, non-human animal, or non-human B cell of the disclosure comprises a human IgG2 constant domain sequence, for example, comprises SEQ ID NO: 35 or a variant, derivative, or fragment thereof. In some embodiments, an antibody, non-human animal, or non-human B cell of the disclosure comprises a human IgG3 constant domain sequence, for example, comprises SEQ ID NO: 36 or a variant, derivative, or fragment thereof. In some embodiments, an antibody, non-human animal, or non-human B cell of the disclosure comprises a human IgG4 constant domain sequence, for example, comprises SEQ ID NO: 37 or a variant, derivative, or fragment thereof. In some embodiments, an antibody, non-human animal, or non-human B cell of the disclosure comprises a human IgE constant domain sequence, for example, comprises SEQ ID NO: 38 or a variant, derivative, or fragment thereof. In some embodiments, an antibody, non-human animal, or non-human B cell of the disclosure comprises a human IgA1 constant domain sequence, for example, comprises SEQ ID NO: 39 or a variant, derivative, or fragment thereof. In some embodiments, an antibody, non-human animal, or non-human B cell of the disclosure comprises a human IgA2 constant domain sequence, for example, comprises SEQ ID NO: 40 or a variant, derivative, or fragment thereof. In some embodiments, an antibody, non-human animal, or non-human B cell of the disclosure comprises a human IgM constant domain sequence, for example, comprises SEQ ID NO: 41 or a variant, derivative, or fragment thereof. In some embodiments, an antibody, non-human animal, or non-human B cell of the disclosure comprises a human IgD constant domain sequence, for example, comprises SEQ ID NO: 42 or a variant, derivative, or fragment thereof.

TABLE 4
Illustrative amino acid sequences of
human constant domain sequences.

| SEQ ID | | |
|---|---|---|
| NO: | Name | Amino acid sequence |
| 34 | IgG1 | ASTKGPSVFPLAPSSKSTSGGTAALGCLVKDYFPEPVTVSWNSGALT |
| | constant | SGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSPG |
| | | K |
| 35 | IgG2 | ASTKGPSVFPLAPCSRSTSESTAALGCLVKDYFPEPVTVSWNSGALT |
| | constant | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCV |
| | | VVDVSHEDPEVQFNWYVDGVEVHNAKTKPREEQFNSTFRVVSVLTVV |
| | | HQDWLNGKEYKCKVSNKGLPAPIEKTISKTKGQPREPQVYTLPPSRE |
| | | EMTKNQVSLTCLVKGFYPSDISVEWESNGQPENNYKTTPPMLDSDGS |
| | | FFLYSKLTVDKSRWQQGNVFSCSVMHEALHNHYTQKSLSLSPGK |
| 36 | IgG3 | ASTKGPSVFPLAPCSRSTSGGTAALGCLVKDYFPEPVTVSWNSGALT |
| | constant | SGVHTFPAVLQSSGLYSLSSVVTVPSSSLGTQTYTCNVNHKPSNTKV |
| | | DKRVELKTPLGDTTHTCPRCPEPKSCDTPPPCPRCPEPKSCDTPPPC |
| | | PRCPEPKSCDTPPPCPRCPAPELLGGPSVFLFPPKPKDTLMISRTPE |
| | | VTCVVVDVSHEDPEVQFKWYVDGVEVHNAKTKPREEQYNSTFRVVSV |
| | | LTVLHQDWLNGKEYKCKVSNKALPAPIEKTISKTKGQPREPQVYTLP |
| | | PSREEMTKNQVSLTCLVKGFYPSDIAVEWESSGQPENNYNTTPPMLD |
| | | SDGSFFLYSKLTVDKSRWQQGNIFSCSVMHEALHNRFTQKSLSLSPG |
| | | K |
| 37 | IgG4 | ASTKGPSVFPLAPCSRSTSESTAALGCLVKDYFPEPVTVSWNSGALT |
| | constant | SGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTV |
| | | LHQDWLNGKEYKCKVSNKGLPSSIEKTISKAKGQPREPQVYTLPPSQ |
| | | EEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 38 | IgE | ASTQSPSVFPLTRCCKNIPSNATSVTLGCLATGYFPEPVMVTWDTGS |
| | constant | LNGTTMTLPATTLTLSGHYATISLLTVSGAWAKQMFTCRVAHTPSST |
| | | DWVDNKTFSVCSRDFTPPTVKILQSSCDGGGHFPPTIQLLCLVSGYT |
| | | PGTINITWLEDGQVMDVDLSTASTTQEGELASTQSELTLSQKHWLSD |
| | | RTYTCQVTYQGHTFEDSTKKCADSNPRGVSAYLSRPSPFDLFIRKSP |
| | | TITCLVVDLAPSKGTVNLTWSRASGKPVNHSTRKEEKQRNGTLTVTS |
| | | TLPVGTRDWIEGETYQCRVTHPHLPRALMRSTTKTSGPRAAPEVYAF |
| | | ATPEWPGSRDKRTLACLIQNFMPEDISVQWLHNEVQLPDARHSTTQP |
| | | RKTKGSGFFVFSRLEVTRAEWEQKDEFICRAVHEAASPSQTVQRAVS |
| | | VNPGK |
| 39 | IgA1 | ASPTSPKVFPLSLCSTQPDGNVVIACLVQGFFPQEPLSVTWSESGQG |
| | constant | VTARNFPPSQDASGDLYTTSSQLTLPATQCLAGKSVTCHVKHYTNPS |
| | | QDVTVPCPVPSTPPTPSPSTPPTPSPSCCHPRLSLHRPALEDLLLGS |
| | | EANLTCTLTGLRDASGVTFTWTPSSGKSAVQGPPERDLCGCYSVSSV |
| | | LPGCAEPWNHGKTFTCTAAYPESKTPLTATLSKSGNTFRPEVHLLPP |
| | | PSEELALNELVTLTCLARGFSPKDVLVRWLQGSQELPREKYLTWASR |
| | | QEPSQGTTTFAVTSILRVAAEDWKKGDTFSCMVGHEALPLAFTQKTI |
| | | DRLAGKPTHVNVSVVMAEVDGTCY |
| 40 | IgGA2 | ASPTSPKVFPLSLDSTPQDGNVVVACLVQGFFPQEPLSVTWSESGQN |
| | constant | VTARNFPPSQDASGDLYTTSSQLTLPATQCPDGKSVTCHVKHYTNSS |
| | | QDVTVPCRVPPPPPCCHPRLSLHRPALEDLLLGSEANLTCTLTGLRD |
| | | ASGATFTWTPSSGKSAVQGPPERDLCGCYSVSSVLPGCAQPWNHGET |
| | | FTCTAAHPELKTPLTANITKSGNTFRPEVHLLPPPSEELALNELVTL |
| | | TCLARGFSPKDVLVRWLQGSQELPREKYLTWASRQEPSQGTTTYAVT |
| | | SILRVAAEDWKKGETFSCMVGHEALPLAFTQKTIDRMAGKPTHINVS |
| | | VVMAEADGTCY |
| 41 | IgM | GSASAPTLFPLVSCENSPSDTSSVAVGCLAQDFLPDSITFSWKYKNN |
| | constant | SDISSTRGFPSVLRGGKYAATSQVLLPSKDVMQGTDEHVVCKVQHPN |
| | | GNKEKNVPLPVIAELPPKVSVFVPPRDGFFGNPRKSKLICQATGFSP |
| | | RQIQVSWLREGKQVGSGVTTDQVQAEAKESGPTTYKVTSTLTIKESD |
| | | WLGQSMFTCRVDHRGLTFQQNASSMCVPDQDTAIRVFAIPPSFASIF |
| | | LTKSTKLTCLVTDLnYDSVTISWTRQNGEAVKTHTNISESHPNATFS |
| | | AVGEASICEDDWNSGERFTCTVTHTDLPSPLKQTISRPKGVALHRPD |
| | | VYLLPPAREQLNLRESATITCLVTGFSPADVFVQWMQRGQPLSPEKY |
| | | VTSAPMPEPQAPGRYFAHSILTVSEEEWNTGETYTCVVAHEALPNRV |
| | | TERTVDKSTGKPTLYNVSLVMSDTAGTCY |
| 42 | IgD | APTKAPDVFPIISGCRHPKDNSPVVLACLITGYHPTSVTVTWYMGTQ |
| | constant | SQPQRTFPEIQRRDSYYMTSSQLSTPLQQWRQGEYKCVVQHTASKSK |
| | | KEIFRWPESPKAQASSVPTAQPQAEGSLAKATTAPATTRNTGRGGEE |
| | | KKKEKEKEEQEERETKTPECPSHTQPLGVYLLTPAVQDLWLRDKATF |
| | | TCFVVGSDLKDAHLTWEVAGKVPTGGVEEGLLERHSNGSQSQHSRLT |
| | | LPRSLWNAGTSVTCTLNHPSLPPQRLMALREPAAQAPVKLSLNLLAS |
| | | SDPPEAASWLLCEVSGFSPPNILLMWLEDQREVNTSGFAPARPPPQP |
| | | RSTTFWAWSVLRVPAPPSPQPATYTCVVSHEDSRTLLNASRSLEVSY |
| | | VTDHGPMK |

An antibody of the disclosure can comprise a human light chain constant domain sequence, e.g. a kappa (IgK) or lambda (IgL) chain. In some embodiments, an antibody, non-human animal, or non-human B cell of the disclosure comprises a human IgK constant domain sequence, for example, comprises SEQ ID NO: 43 or a variant, derivative, or fragment thereof. In some embodiments, an antibody, non-human animal, or non-human B cell of the disclosure comprises a human IgL constant domain sequence, for example, comprises SEQ ID NO: 44 or a variant, derivative, or fragment thereof.

TABLE 5 provides example light chain constant domain sequences.

TABLE 5

| SEQ ID | | |
|---|---|---|
| NO: | Name | Amino acid sequence |
| 43 | IgK | TVAAPSVFIFPPSDEQLKSGTASVVCLLNNFYP |
| | constant | REAKVQWKVDNALQSGNSQESVTEQDSKDSTYS |
| | | LSSTLTLSKADYEKHKVYACEVTHQGLSSPVTK |
| | | SFNRGEC |
| 44 | IgL | GQPKANPTVTLFPPSSEELQANKATLVCLISDF |
| | constant | YPGAVTVAWKADGSPVKAGVETTKPSKQSNNKY |
| | | AASSYLSLTPEQWKSHRSYSCQVTHEGSTVEKT |
| | | VAPTECS |

Signal peptides can result in higher protein expression and/or secretion by a cell. In some embodiments, an antibody of the disclosure comprises a signal peptide. Signal peptidases can cleave a signal peptide off a protein, for example, during a secretion process, generating a mature protein that does not comprise the signal peptide sequence. In some embodiments, a signal peptide is cleaved off a compound or antibody of the disclosure. In some embodiments, a mature compound or antibody of the disclosure does not comprise a signal peptide.

The constant regions can mediate various effector functions, and can be minimally involved in antigen binding. Different IgG isotypes or subclasses can be associated with different effector functions or therapeutic characteristics, for example, because of interactions with different Fc receptors and/or complement proteins. Antibodies comprising Fc regions that engage activating Fc receptors can, for example, participate in antibody-dependent cell-mediated cytotoxicity (ADCC), antibody-dependent cellular phagocytosis (ADCP), complement-dependent cytotoxicity (CDC), induction of signaling through immunoreceptor tyrosine-based activation motifs (ITAMs), and induction of cytokine secretion. Antibodies comprising Fc regions that engage inhibitory Fc receptors can, for example, induce signaling through immunoreceptor tyrosine-based inhibitory motifs (ITIMs).

Different antibody subclasses comprise different abilities to elicit immune effector functions. For example, IgG1 and IgG3 can effectively recruit complement to activate CDC, IgG2 elicits minimal ADCC. IgG4 has a lesser ability to trigger immune effector functions. Modifications to the constant regions can also affect antibody characteristics, for example, enhancement or reduction of Fc receptor ligation, enhancement or reduction of ADCC, enhancement or reduction of ADCP, enhancement or reduction of CDC, enhancement or reduction of signaling through ITAMs, enhancement or reduction of cytokine induction, enhancement or reduction of signaling through ITIMs, enhancement or reduction of half-life, or enhancement or reduction of co-engagement of antigen with Fc receptors. Modifications can include, for example, amino acid mutations, altering post-translational modifications (e.g., glycosylation), combining domains from different isotypes or subclasses, or a combination thereof.

Antibodies of the disclosure can comprise constant regions or Fc regions that are selected or modified to provide suitable antibody characteristics, for example, suitable characteristics for treating a disease or condition as disclosed herein. In some embodiments, IgG1 can be used, for example, to promote inflammation, immune activation, and immune effector functions for the treatment of an infection. In some embodiments, IgG4 can be used, for example, in cases where antagonistic properties of the antibody with reduced immune effector functions are desired (e.g., to neutralize coronavirus antigens and inhibit viral entry into cells without promoting inflammation and immune activation).

Non-limiting examples of antibody modifications and their effects are provided in TABLE 6.

TABLE 6

| Effect | Isotype | Mutation(s)/modification(s) |
|---|---|---|
| Enhanced ADCC | IgG1 | F243L/R292P/Y300L/V305I/P396L |
| Enhanced ADCC | IgG1 | S239D/I332E |
| Enhanced ADCC | IgG1 | S239D/I332E/A330L |
| Enhanced ADCC | IgG1 | S298A/E333A/K334A |
| Enhanced ADCC | IgG1 | In one heavy chain: L234Y/L235Q/G236W/S239M/ |
| | | H268D/D270E/S298A |
| | | In the opposing heavy chain: D270E/K326D/A330M/ |
| | | K334E |
| Enhanced ADCP | IgG1 | G236A/S239D/I332E |
| Enhanced CDC | IgG1 | K326W/E333S |
| Enhanced CDC | IgG1 | S267E/H268F/S324T |
| Enhanced CDC | IgG1, | Combination of domains from IgG1/IgG3 |
| | IgG3 | |
| Enhanced CDC | IgG1 | E345R/E430G/S440Y |
| Loss of glycosylation, reduced | IgG1 | N297A or N297Q or N297G |
| effector functions | | |
| Reduced effector functions | IgG1, | L235E |
| | IgG4 | |
| Reduced effector functions | IgG1 | L234A/L235A |
| Reduced effector functions | IgG4 | F234A/L235A |
| Reduced effector functions | IgG4 | F234A/L235A/G237A/P238S |
| Reduced effector functions | IgG4 | F234A/L235A/ΔG236/G237A/P238S |
| Reduced effector functions | IgG2, | Combination of domains from IgG2/IgG4 |
| | IgG4 | |
| Reduced effector functions | IgG2 | H268Q/V309L/A330S/P331S |
| Reduced effector functions | IgG2 | V234A/G237A/P238S/H268A/V309L/A330S/ |
| | | P331S |
| Reduced effector functions | IgG1 | L234A/L235A/G237A/P238S/H268A/A330S/ |
| | | P331S |
| Increased half-life | IgG1 | M252Y/S254T/T256E |
| Increased half-life | IgG1 | M428L/N434S |
| Increased antigen/Fc receptor | IgG1 | S267E/L328F |
| co-engagement | | |
| Altered antigen/Fc receptor | IgG1 | N325S/L328F |
| co-engagement | | |
| Reduced Fab arm exchange | IgG4 | S228P |

The variable (V) regions can mediate antigen binding and define the specificity of a particular antibody for an antigen. The variable region comprises relatively invariant sequences called framework regions, and hypervariable regions, which differ considerably in sequence among antibodies of different binding specificities. The variable region of each antibody heavy or light chain comprises four framework regions separated by three hypervariable regions. The variable regions of heavy and light chains fold in a manner that brings the hypervariable regions together in close proximity to create an antigen binding site. The four framework regions largely adopt an f3-sheet configuration, while the three hypervariable regions form loops connecting, and in some cases forming part of, the f3-sheet structure.

Within hypervariable regions are amino acid residues that primarily determine the binding specificity of the antibody. Sequences comprising these residues are known as complementarity determining regions (CDRs). One antigen binding site of an antibody can comprise six CDRs, three in the hypervariable regions of the light chain, and three in the hypervariable regions of the heavy chain. The CDRs in the light chain can be designated LCDR1, LCDR2, LCDR3, while the CDRs in the heavy chain can be designated HCDR1, HCDR2, and HCDR3.

In some embodiments, antibodies of the disclosure include variants, derivatives, and antigen-binding fragments thereof. For example, a non-human animal can be genetically modified to produce antibody variants, derivatives, and antigen-binding fragments thereof. In some embodiments, an antibody can be a single domain antibody (sdAb), for example, a heavy chain only antibody (HCAb) VHH, or nanobody. Non-limiting examples of antigen-binding fragments include Fab, Fab′, F(ab′)₂, dimers and trimers of Fab conjugates, Fv, scFv, minibodies, dia-, tria-, and tetrabodies, and linear antibodies. Fab and Fab′ are antigen-binding fragments that can comprise the V_{H} and C_{H}1 domains of the heavy chain linked to the V_{L} and C_{L} domains of the light chain via a disulfide bond. A F(ab′)₂ can comprise two Fab or Fab′ that are joined by disulfide bonds. A Fv can comprise the V_{H} and V_{L} domains held together by non-covalent interactions. A scFv (single-chain variable fragment) is a fusion protein that can comprise the V_{H} and V_{L} domains connected by a peptide linker. Manipulation of the orientation of the V_{H} and V_{L} domains and the linker length can be used to create different forms of molecules that can be monomeric, dimeric (diabody), trimeric (triabody), or tetrameric (tetrabody). Minibodies are scFv-C_{H}3fusion proteins that assemble into bivalent dimers.

In some embodiments, an antibody of this disclosure is an anti-coronavirus antibody produced by administering the immunogenic composition as disclosed herein to a non-human animal or human subject (e.g., a non-human animal or human subject for immunization). In some embodiments, a plurality of antibodies of this disclosure is a plurality of anti-coronavirus polyclonal antibodies produced by immunizing a non-human animal or human subject (e.g., a non-human animal or human subject for immunization) with the immunogenic composition as disclosed herein. In some embodiments, the anti-coronavirus antibody or the plurality of anti-coronavirus polyclonal antibodies further comprises a pharmaceutically acceptable carrier or excipient. In some embodiments, the non-human animal (e.g., the non-human animal subject for immunization) is a non-human animal having a humanized immune system.

### Pharmaceutical Compositions

In some embodiments, the immunogenic compositions administered to a subject (e.g., a subject for immunization) is a pharmaceutical composition. The pharmaceutical compositions as contemplated by the current invention may also include a pharmaceutically acceptable excipient.

The disclosure also provides pharmaceutical compositions comprising a plurality of polyclonal antibodies or a polyclonal antibody preparation against coronavirus disclosed herein and a pharmaceutically acceptable excipient.

A pharmaceutically acceptable excipient can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

A pharmaceutical composition of the disclosure can comprise polyclonal antibodies of the disclosure, a circular polyribonucleotide of the disclosure, or a combination thereof. A pharmaceutical composition of the disclosure can comprise polyclonal antibodies of the disclosure, a linear polyribonucleotide of the disclosure, or a combination thereof. A pharmaceutical composition of the disclosure can comprise polyclonal antibodies of the disclosure, a circular polyribonucleotide of the disclosure, a linear polyribonucleotide of the disclosure, or a combination thereof.

In some embodiments, pharmaceutical compositions provided herein are suitable for administration to humans. In some embodiments, pharmaceutical compositions (e.g., comprising a circular polyribonucleotide, a linear polyribonucleotide, or an immunogenic composition as described herein) provided herein are suitable for administration to a subject (e.g., a subject for immunization), wherein the subject is a human. In some embodiments, pharmaceutical compositions (e.g., comprising a plurality of polyclonal antibodies or a polyclonal antibody preparation as described herein) provided herein are suitable for administration to a subject (e.g., a subject for treatment), wherein the subject is a human.

In some embodiments, pharmaceutical compositions (e.g., comprising a circular polyribonucleotide, a linear polyribonucleotide, or an immunogenic composition as described herein) provided herein are suitable for administration to a subject (e.g., a subject for immunization), wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, cats, dogs, mice, and/or rats; and/or birds, including commercially relevant birds such as parrots, poultry, chickens, ducks, geese, hens or roosters, and/or turkeys; zoo animals, e.g., a feline; non-mammal animals, e.g., reptiles, fish, amphibians, etc.

In some embodiments, pharmaceutical compositions (e.g., comprising a plurality of polyclonal antibodies or a polyclonal antibody preparation as described herein) provided herein are suitable for administration to a subject (e.g., a subject for treatment), wherein the subject is non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, cats, dogs, mice, and/or rats; and/or birds, including commercially relevant birds such as parrots, poultry, chickens, ducks, geese, hens or roosters, and/or turkeys; zoo animals, e.g., a feline; non-mammal animals, e.g., reptiles, fish, amphibians, etc.

Subjects (e.g., subjects for immunization or subjects for treatment) to which administration of the pharmaceutical compositions is contemplated include any ungulates.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

Pharmaceutical compositions can be sterile and stable under the conditions of manufacture and storage. The composition can be formulated as a solution, microemulsion, liposome, or other ordered structure suitable to high drug concentration. Examples of suitable aqueous and non-aqueous compositions which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Sterile injectable solutions can be prepared by incorporating the active compound (e.g., an agent, such as a circular polyribonucleotide, linear polyribonucleotide, or antibody) in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Agents (e.g., circular polyribonucleotides, linear polyribonucleotides, or antibodies) of the disclosure can be prepared in a composition that will protect them against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978. A composition of the disclosure can be, for example, an immediate release form or a controlled release formulation. An immediate release formulation can be formulated to allow the compounds (e.g., agents, such as a circular polyribonucleotide, linear polyribonucleotide or antibody) to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

Pharmaceutical formulations for administration can include aqueous solutions of the active compounds (e.g., agents, such as a circular polyribonucleotide, linear polyribonucleotide, or antibody) in water soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the agents to allow for the preparation of highly concentrated solutions. The active ingredient can be in powder form for constitution with a suitable vehicle, for example, sterile pyrogen-free water, before use.

Methods for the preparation of compositions comprising the agents described herein include formulating the agents with one or more inert, pharmaceutically-acceptable excipients or carriers to form a solid, semi-solid, or liquid composition. Solid compositions include, for example, powders, dispersible granules, and cachets. Liquid compositions include, for example, solutions in which an agent is dissolved, emulsions comprising an agent, or a solution containing liposomes, micelles, or nanoparticles comprising an agent as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

In some embodiments, a formulation of the disclosure contains a thermal stabilizer, such as a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol or trehalose.

Pharmaceutical compositions as described herein can be formulated for example to include a pharmaceutical excipient or carrier. A pharmaceutical carrier may be a membrane, lipid bilayer, and/or a polymeric carrier, e.g., a liposome or particle such as a nanoparticle, e.g., a lipid nanoparticle, and delivered by known methods, such as via partial or full encapsulation of the circular polyribonucleotide, to a subject (e.g., a subject for immunization or a subject for treatment) in need thereof (e.g., a human or non-human agricultural or domestic animal, e.g., cattle, dog, cat, horse, poultry).

### Methods of Delivery

The circular polyribonucleotide as described herein or a pharmaceutical composition thereof as described herein can be administered to a cell in a vesicle or other membrane-based carrier as described herein. The linear polyribonucleotide as described herein or a pharmaceutical composition thereof as described herein can be administered to a cell in a vesicle or other membrane-based carrier as described herein.

In some embodiments, the cell is a eukaryotic cell. In some embodiments, the cell is a mammalian cell. In some embodiments, the cell is an ungulate cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.). In some embodiments, the subject (e.g., a subject for immunization) is a mammal. In some embodiments, the subject (e.g., a subject for immunization) is an ungulate.

In some embodiments, a pharmaceutical formulation disclosed herein can comprise: (i) a compound (e.g., circular polyribonucleotide or antibody) disclosed herein; (ii) a buffer; (iii) a non-ionic detergent; (iv) a tonicity agent; and (v) a stabilizer. In some embodiments, a pharmaceutical formulation disclosed herein can comprise: (i) a compound (e.g., linear polyribonucleotide or antibody) disclosed herein; (ii) a buffer; (iii) a non-ionic detergent; (iv) a tonicity agent; and (v) a stabilizer. In some embodiments, the pharmaceutical formulation disclosed herein is a stable liquid pharmaceutical formulation.

### Therapeutic Methods

The disclosure provides compositions and methods that are useful as treatments or prophylactics, for example, compositions and methods that comprise antibodies that can be used to protect a subject (e.g., the subject for immunization or the subject for treatment) against the effects of a coronavirus infection. For example, a circular polyribonucleotide of the disclosure can be administered to a subject (e.g., a subject for immunization) to stimulate production of antibodies (e.g., human polyclonal antibodies) that bind to desired coronavirus antigens/and or epitopes. A linear polyribonucleotide of the disclosure can be administered to a subject (e.g., a subject for immunization) to stimulate production of antibodies (e.g., human polyclonal antibodies) that bind to desired coronavirus antigens/and or epitopes. The antibodies can be obtained from the subject (e.g., after immunization of the subject for immunization) and formulated for administration to a subject (e.g., a subject for treatment, such as a human subject for treatment), for example, as a treatment or prophylactic. The antibodies can provide protection against, for example, a coronavirus that expresses the antigens and/or epitopes. In another example, a circular polyribonucleotide can be administered to a human subject (e.g., a human subject for immunization) to stimulate production of antibodies in the human subject that bind to desired antigens/and or epitopes. In another example, a linear polyribonucleotide can be administered to a human subject (e.g., a human subject for immunization) to stimulate production of antibodies in the human subject that bind to desired antigens/and or epitopes. In some embodiments, the disclosure provides compositions for use in treating or prophylaxis of a coronavirus infection.

Non-limiting examples of conditions and diseases that can be treated by compositions and methods of the disclosure include those caused by or associated with a coronavirus disclosed herein, for example coronavirus infections. In some embodiments, a condition is caused by or associated with a SARS-CoV. In some embodiments, a condition is caused by or associated with SARS-CoV-2. In some embodiments, a condition is coronavirus disease of 2019 (COVID-19). In some embodiments, a condition is caused by or associated with MERS-CoV.

In some embodiments, the polyclonal antibodies are produced by immunizing a non-human animal or human subject (e.g., a non-human animal or human subject for immunization) with a circular polyribonucleotide of the disclosure, plasma are collected from the non-human animal or human subject (e.g., after immunization of the non-human animal or human subject for immunization), and polyclonal antibodies are purified from the plasma. In some embodiments, the polyclonal antibodies are produced by immunizing a non-human animal or human subject (e.g., a non-human animal or human subject for immunization) with a linear polyribonucleotide of the disclosure, plasma are collected from the non-human animal or human subject (e.g., after immunization of the non-human animal or human subject for immunization), and polyclonal antibodies are purified from the plasma. Optionally, purified polyclonal antibodies from more than one non-human animal or human subject (e.g., after immunization of the more than one non-human animal or human subject for immunization), multiple purified polyclonal antibody samples from the same non-human animal or human subject (e.g., after immunization of the non-human animal or human subject for immunization), or a combination thereof, are pooled together and administered to a subject (e.g., a subject for treatment) in need thereof, e.g., a human subject (e.g., a human subject for treatment) in need thereof. In some embodiments, the polyclonal antibodies are formulated in a polyclonal antibody preparation, e.g., a polyclonal antibody preparation against a coronavirus. A method of producing a human polyclonal antibody preparation against a coronavirus comprising (a) administering to an animal (e.g., an animal for immunization) capable of producing antibodies an immunogenic composition comprising a polyribonucleotide (e.g., a circular polyribonucleotide or a linear polyribonucleotide) that comprises a sequence encoding a coronavirus antigen, (b) collecting blood or plasma from the mammal, (c) purifying polyclonal antibodies against the coronavirus from the blood or plasma, and (d) formulating polyclonal antibodies as a therapeutic or pharmaceutical preparation for human use (e.g., administration to a human subject for treatment) or a veterinarian preparation for non-human animal use (e.g., administration to a non-human animal subject for treatment).

In some embodiments, the method further comprises monitoring the subject (e.g., the subject for treatment) having a coronavirus infection, the subject (e.g., the subject for treatment) at risk for exposure to a coronavirus infection, or the subject (e.g., the subject for treatment) in need thereof for the presence of the polyclonal antibodies for the coronavirus antigen. In some embodiments, the monitoring is prior to administration of the polyclonal antibodies and/or after the administration of the polyclonal antibodies.

In practicing the methods of treatment or use provided herein, therapeutically-effective amounts of the compounds (e.g., agents, such as a circular polyribonucleotide or antibody) described herein are administered in pharmaceutical compositions to a subject (e.g., the subject for immunization or the subject for treatment) having a disease or condition to be treated, or requiring prophylaxis. In practicing the methods of treatment or use provided herein, therapeutically-effective amounts of the compounds (e.g., agents, such as a linear polyribonucleotide or antibody) described herein are administered in pharmaceutical compositions to a subject (e.g., the subject for immunization or the subject for treatment) having a disease or condition to be treated, or requiring prophylaxis. In some embodiments, the subject (e.g., the subject for immunization or the subject for treatment) is a mammal such as a human. A therapeutically-effective amount can vary widely depending on the severity of the disease, the age and relative health of the subject (e.g., the subject for immunization or the subject for treatment), the potency of the compounds used, characteristics of a given coronavirus, and other factors.

### Methods and Routes of Administering

A composition (e.g., a pharmaceutical composition) disclosed herein can be administered in a therapeutically-effective amount by various forms and routes including, for example, oral, or topical administration. In some embodiments, a composition can be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition can be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period, such as more than 24 hours. In some embodiments, the composition is administered as an intravenous injection or a short infusion.

A pharmaceutical composition can be administered in a local manner, for example, via injection of the agent directly into an organ, optionally in a depot or sustained release formulation or implant. A pharmaceutical composition can be provided in the form of a rapid release formulation, in the form of an extended release formulation, or in the form of an intermediate release formulation. A rapid release form can provide an immediate release. An extended release formulation can provide a controlled release or a sustained delayed release. In some embodiments, a pump can be used for delivery of the pharmaceutical composition. In some embodiments, a pen delivery device can be used, for example, for subcutaneous delivery of a composition of the disclosure.

A pharmaceutical composition provided herein can be administered in conjunction with other therapies, for example, an antiviral therapy, an antibiotic, a cell therapy, a cytokine therapy, or an anti-inflammatory agent. In some embodiments, a circular polyribonucleotide or antibody described herein can be used singly or in combination with one or more therapeutic agents as a component of mixtures. In some embodiments, a linear polyribonucleotide or antibody described herein can be used singly or in combination with one or more therapeutic agents as a component of mixtures.

### Doses and Frequency

Therapeutic agents described herein can be administered before, during, or after the occurrence of a disease or condition, and the timing of administering the composition containing a therapeutic agent can vary. In some cases, the compositions can be used as a prophylactic and can be administered continuously to subjects (e.g., the subject for immunization or the subject for treatment) with a susceptibility to a coronavirus or a propensity to a condition or disease associated with a coronavirus. Prophylactic administration can lessen a likelihood of the occurrence of the infection, disease or condition, or can reduce the severity of the infection, disease or condition.

The compositions can be administered to a subject (e.g., the subject for immunization or the subject for treatment) after (e.g., as soon as possible after) the onset of the symptoms. The compositions can be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent can be administered after (e.g., as soon as is practicable after) the onset of a disease or condition is detected or suspected. A therapeutic agent can be administered after (e.g., as soon as is practicable after) a potential exposure to a coronavirus, for example, after a subject (e.g., the subject for immunization or the subject for treatment) has contact with an infected subject, or learns they had contact with an infected subject that may be contagious.

A circular polyribonucleotide, antibody, or therapeutic agent described herein are administered at any interval desired. A linear polyribonucleotide, antibody, or therapeutic agent described herein are administered at any interval desired.

Actual dosage levels of an agent of the disclosure (e.g., circular polyribonucleotide, linear polyribonucleotide, antibody, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level can depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure can be determined by and directly dependent on (a) the unique characteristics of the active agent and the particular therapeutic effect to be achieved, and (b) the limitations inherent in the art of compounding such an active agent for the treatment of sensitivity in individuals. A dose can be determined by reference to a plasma concentration or a local concentration of the circular polyribonucleotide or antibody. A dose can be determined by reference to a plasma concentration or a local concentration of the linear polyribonucleotide or antibody.

A pharmaceutical composition described herein can be in a unit dosage form suitable for a single administration of a precise dosage. In unit dosage form, the formulation can be divided into unit doses containing appropriate quantities of one or more circular polyribonucleotides, antibodies, and/or therapeutic agents. In unit dosage form, the formulation can be divided into unit doses containing appropriate quantities of one or more linear polyribonucleotides, antibodies, and/or therapeutic agents. The unit dosage can be in the form of a package containing discrete quantities of the formulation. Non-limiting examples are packaged injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

A dose can be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days.

### Subjects

A composition is provided for use in treatment or prophylaxis of a condition disclosed herein, such as an infection with a coronavirus. The composition can be administered to a subject (e.g., the subject for immunization or the subject for treatment) that has a coronavirus infection or an associated disease or condition. The composition can be administered as a prophylactic to subjects (e.g., subjects for immunization or subjects for treatment) with a propensity for coronavirus infection or a susceptibility to an associated condition or disease in order to lessen a likelihood of the infection, disease or condition, or to reduce the severity of the infection, disease or condition.

A subject (e.g., the subject for immunization or the subject for treatment) can be a subject that is infected with a coronavirus. A subject (e.g., the subject for immunization or the subject for treatment) can be a subject that tested positive for the coronavirus. A subject (e.g., the subject for immunization or the subject for treatment) can be a subject that has been exposed to a coronavirus. A subject (e.g., the subject for immunization or the subject for treatment) can be a subject that has potentially been exposed to a coronavirus. A subject (e.g., the subject for immunization or the subject for treatment) can be a subject that is exhibiting one or more signs and/or symptoms consistent with infection with a coronavirus.

In some embodiments, a subject (e.g., the subject for immunization or the subject for treatment) is a subject that is at high risk of coming into contact with a coronavirus of the disclosure. For example, a subject (e.g., the subject for immunization or the subject for treatment) may be a health care worker, a laboratory worker, or a first responder that is more likely to come into contact with a coronavirus (e.g., SARS-CoV2) of the disclosure. A subject (e.g., the subject for immunization or the subject for treatment) may work at a health care facility, e.g., a hospital, doctor's surgery, inpatient facility, outpatient facility, urgent care facility, retirement home, aged care facility, or nursing home.

In some embodiments, a subject (e.g., the subject for immunization or the subject for treatment) is a subject that is at high risk of complications if infected with a coronavirus of the disclosure. For example, a subject (e.g., the subject for immunization or the subject for treatment) can have a co-morbidity, an age over 50, type 1 diabetes mellitus, type 2 diabetes mellitus, insulin resistance, or a combination thereof. In some embodiments, a subject is an immunocompromised subject. In some embodiments, a subject (e.g., the subject for immunization or the subject for treatment) is on immunosuppressive drugs. In some embodiments, a subject (e.g., the subject for immunization or the subject for treatment) is a transplant recipient that is on immunosuppressive drugs. In some embodiments, a subject (e.g., the subject for immunization or the subject for treatment) is undergoing therapy for cancer, e.g., chemotherapy, that may decrease the function of the immune system.

A subject (e.g., the subject for immunization or the subject for treatment) can be a mammal. A subject (e.g., the subject for immunization or the subject for treatment) can be a human. A subject (e.g., the subject for immunization or the subject for treatment) can be a non-human animal. The non-human animal can be an agricultural animal, e.g., a cow, pig, sheep, horse, or goat; a pet, e.g., a cat or dog; or a zoo animal, e.g., a feline.

### EXAMPLES

The following examples are included for illustrative purposes only and are not intended to limit the scope of the invention.

### Example 1: Circular RNA Constructs

This example describes design of novel SARS-CoV-2 open reading frames (ORFs) and circRNA constructs.

In this Example, SARS-CoV-2 ORFs and circular RNA constructs encoding SARS-CoV-2 ORFs were designed as described in TABLE 2.

### Example 2: In Vitro Production of Circular RNAs Encoding SARS-CoV-2 Antigens

This example demonstrates in vitro production of circular RNAs.

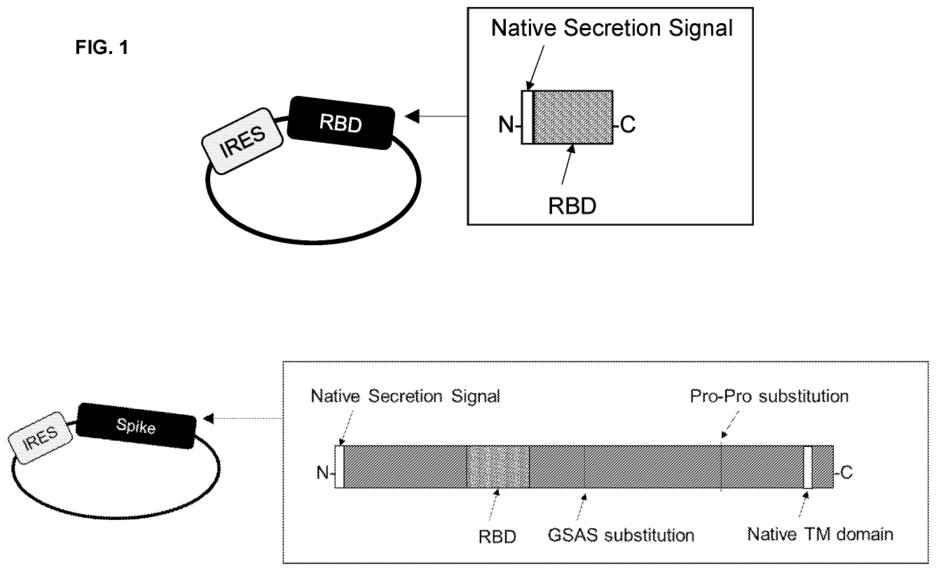

Circular RNAs were designed to include an IRES, an ORF encoding a modified SARS-CoV-2 spike antigen or RBD antigen (as described in Example 1), and two spacer elements flanking the IRES-ORF. Circularization enables rolling circle translation, multiple ORFs with alternating stagger elements for discrete ORF expression and controlled protein stoichiometry, and an IRES that targets RNA for ribosomal entry. An exemplary drawing of circular polyribonucleotide comprising a sequence encoding a coronavirus antigen is shown in FIG. 1.

In this Example, circular RNAs were generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′phosphohydrolase (RppH) (New England Biolabs, M0356) following the manufacturer's instructions, and purified again with the RNA purification system. RppH-treated linear RNA was circularized using a splint DNA. Alternately or in addition to treatment with 5′RppH, the RNA was transcribed under conditions with excess GMP over GTP.

Splint-ligation was performed as follows: circular RNA was generated by treatment of the transcribed linear RNA and a DNA splint (5′-GTTTTTCGGCTATTCCCAATAGCCGTTTTG-3′) (SEQ ID NO: 47) using T4 DNA ligase 1 (New England Bio, Inc., M0437M). To purify the circular RNAs, ligation mixtures were resolved on 4% denaturing PAGE and RNA corresponding to each of the circular RNAs were excised. Excised RNA gel fragments were crushed, and RNA was eluted with gel elution buffer (0.5 M Sodium Acetate, 0.1% SDS, 1 mM EDTA) for one hour at 37° C. The eluted buffer was harvested, and RNA was eluted again by adding gel elution buffer to the crushed gel and incubated for one hour. Gel debris was removed by centrifuge filters and RNA was precipitated with ethanol Agarose gel electrophoresis was used as a quality control measurement for validating purity and circularization. Additionally or alternatively, the circular RNAs were purified using column chromatography.

### Example 3: mRNA Constructs

This example describes design of novel mRNA constructs encoding SARS-CoV-2 ORFs.

In this Example, linear RNA constructs encoding SARS-CoV-2 ORFs were designed as described in Table 3.

### Example 4: In Vitro Production of mRNAs Encoding SARS-CoV-2 Antigens

This example demonstrates in vitro production of mRNAs.

In this Example, mRNA was designed with an ORF encoding a modified SARS-CoV-2 spike antigen or RBD as described in Example 3.

In this Example, modified mRNA was made by in vitro transcription. RNA was fully substituted with Pseudo-Uridine and 5-Methyl-C, capped with CleanCap™ AG, included 5′ and 3′ human alpha-globin UTRs, and was polyadenylated. mRNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000). Agarose gel electrophoresis was used as a quality control measurement for validating purity and circularization.

### Example 5: Expression of Secreted SARS-CoV-2 Antigen from Circular RNA in Mammalian Cells

This example demonstrates the ability to express viral antigens from circular RNA in mammalian cells.

In this Example, circular RNAs encoding SARS-CoV-2 RBD antigens were designed, and produced and purified by the methods described herein.

The expression of RBD-encoding circular RNA was tested by immunoprecipitation coupled with Western blot (IP-Western). Briefly, circular RNA encoding RBD (0.1 picomoles) was transfected into BJ Fibroblasts and HeLa cells (10,000 cells per well in a 96 well plate) using Lipofectamine MessengerMax (ThermoFisher, LMRNA015). MessengerMax alone was used as a control. Supernatant was collected at 24 hours and immunoprecipitation was performed with a rabbit antibody specific to the SARS-CoV-2 RBD-Spike Glycoprotein (Sino Biologicals, Cat: 40592-T62) coupled to Protein G-Dynabeads (Invitrogen, 10003D) and the same antibody was used to detect the immunoprecipitated products resolved by PAGE. A recombinant RBD (42ng) Immunoprecipitation was used as control and to quantify cell protein expression. Membrane chemiluminescence was quantified using the Image Studio™ Lite western blot quantification software (Li-COR Biosciences).

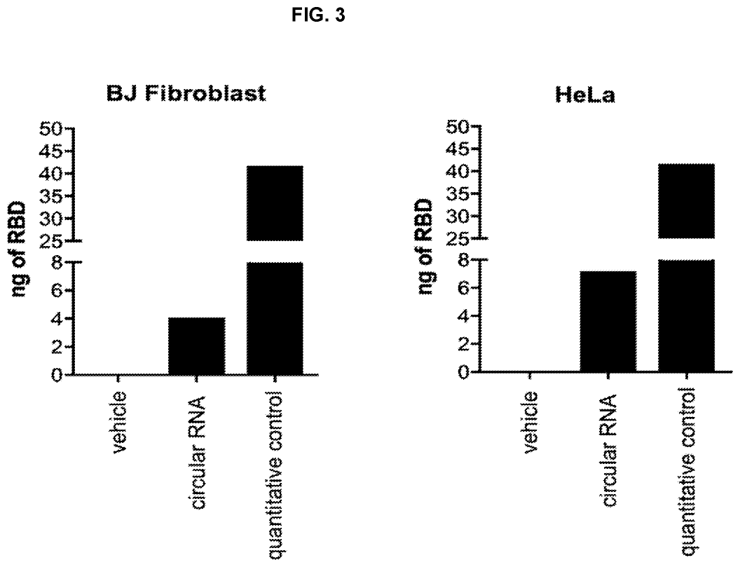

RBD antigen encoded by circular RNA was detected in BJ Fibroblast and HeLa cell supernatants and not in the controls (FIG. 3).

This Example shows that SAR-CoV-2 RBD antigens (which are secreted proteins) were expressed from circular RNA in mammalian cells.

### Example 6: Expression of Non-Secreted SARS-CoV-2 Antigen from RNA in Mammalian Cells

In this Example, circular RNA or mRNA encoding SARS-CoV-2 spike antigens were designed, and produced and purified by the methods described herein. Circular RNAs and mRNAs are formulated in MessengerMax and 0.1 picomoles of circular RNA is transfected into HEK293 cells (10 000 cells per well) according to the manufacturer's instructions.

Spike antigen expression is measured using a SARS-CoV-2 spike antigen-specific ELISA at 24, 48, and 72 hours. To measure expression, cells are lysed in each well at the appropriate timepoint, using a lysis buffer and a protease inhibitor. The cell lysate is retrieved and centrifuged at 12,000 rpm for 10 minutes. Supernatant is collected. In this example, a SARS-CoV-2 2019 spike antigen detection sandwich ELISA kit is used (SARS-CoV-2 (2019-nCoV) Spike Detection ELISA Kit, Sino Biological, KIT40591) according to the manufacturer's instructions.

### Example 7: Formulation of RNA for Administration to Non-Human Animal

In this Example, circular RNA or mRNA encoding SARS-CoV-2 RBD antigens were designed, and produced and purified by the methods descried herein.

After purification, the circular RNA or mRNA was formulated as follows:

A. circular RNA or mRNA was diluted in PBS to a final concentration of 2.5 or 25 picomoles in 50 uL, to obtain a circular RNA preparation or a linear RNA preparation (unformulated).

B. circular RNA or mRNA was formulated with a lipid carrier (e.g., TransIT (Mirus Bio)) and mRNA Boost Reagent (Mirus Bio) according to the manufacturer's instructions (15% TransIT, 5% Boost) to obtain a final RNA concentration of 2.5 or 25 picomoles in 50 uL, to obtain a circular RNA preparation or a linear RNA preparation.

C. circular RNA or mRNA was formulated with a cationic polymer (e.g., protamine). Briefly, circular RNA or mRNA was diluted in pure water. Protamine sulfate was dissolved in Ringer lactate solution (4000 ng/uL). While stirring, the protamine-Ringer lactate solution was added to half of the circular RNA or mRNA solution until a weight ratio of RNA:protamine is 2:1. The solution was stirred for another 10 minutes to ensure the formation of stable complexes. The remaining circular RNA or mRNA was then added (i.e., remaining circular RNA to circular RNA solution, remaining mRNA to mRNA solution) and the solution stirred briefly. The final concentration of the mixture (i.e., circular RNA mixture or mRNA mixture) was adjusted using Ringer lactate solution to obtain a circular RNA preparation or a linear RNA preparation with a final RNA concentration of 2.5 or 25 picomoles in per 50 uL

D. circular RNA or mRNA was formulated with a lipid nanoparticle. Briefly, circular RNA or mRNA was diluted in 25 mM acetate buffer pH=4 (filtered through 0.2 um filter) to a concentration of 0.2 ug/uL. Lipid nanoparticles (LNPs) were formulated by first dissolving the ionizable lipid (e.g. ALC0315), cholesterol, DSPC, and DMG-PEG2000 in ethanol (filtered through 0.2 um sterile filter) in a molar ratio of 50/38.5/10/1.5 mol %. The final ionizable lipid/RNA weight ratio was 8/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 h to remove ethanol. The RNA concentration inside the LNPs and the encapsulation efficiency were measured using Ribogreen assay. If necessary, the LNPs were concentrated down to the desired RNA concentration using Amicon centrifugation filters, 100 kDa cut off. The size, concentration, and charge of the particles were measured using Zetasizer Ultra (Malvern Pananaytical). The RNA concentration was adjusted with PBS to a final concentration of 0.1 or 0.2 ug/ul. For formulations containing two RNA sequences the RNAs were either mixed before formulating in LNPs or after each RNA was formulated separately. For in vivo experiments, the final RNA formulated in LNPs were filtered through sterile 0.2 um regenerated cellulose filters.

### Example 8: Administration of RNA to Non-Human Animal

In this example, mice received 50 uL injections of each circular RNA preparation or linear RNA preparation via either a single intramuscular injection in a hind leg or a single intradermal injection to the back.

### Example 9: Detection of Secreted Antigen in Blood

Blood samples (˜25 μL) are collected from each mouse for analysis by submalar drawing. Blood is collected into EDTA tubes, at 0, 6 hours, 24, 48 hours and 7 days post-dosing of the circular RNA. Plasma is isolated by centrifugation for 30 minutes at 1300 g at 4° C. Expression of secreted antigen is assessed using an ELISA or Western blot, e.g. for RBD antigen, using methods as described in Example 5.

### Example 10: Detection of Antibodies to Antigen

This example describes how to determine the presence of antibodies to antigen.

An ELISA is used as described by Chen X et al. (medRxiv, doi: doi.org/10.1101/2020.04.06.20055475 (2020)). Briefly, SARS-CoV-2 protein in 100 uL PBS per well is coated on ELISA plates overnight at 4° C. ELISA plates are then blocked for 1 hour with blocking buffer (5% FBS plus 0.05% Tween 20). 10-fold diluted plasma is then added to each well in 100 uL blocking buffer over 1 hour. After washing with 1× phosphate-buffered saline with Tween® detergent (PBST), bound antibodies are incubated with anti-mouse IgG HRP detection antibody (Invitrogen) for 30 mins, followed by wash with PBST, then PBS, and addition of tetramethylbenzene. The ELISA plate is allowed to react for 5 min and then quenched using 1 M HCl Stop buffer. The optical density (OD) value is determined at 450 nm.

A. For antibodies to SARS-CoV-2 RBD antigen, the SARS-CoV-2 protein used is SARS-CoV-2 RBD (Sino Biological, 40592-V08B).

B. For antibodies to SARS-CoV-2 spike antigen, the SARS-CoV-2 protein used is SARS-CoV-2 spike protein (Sino Biological, 40591-V08H)

### Example 11: Evaluation of Neutralizing Antibodies to SARS-CoV-2

A SARS-CoV-2 viral neutralization assay is used to test neutralization ability of antibodies against SARS-CoV-2 infection. An example of such an assay is described by Okba N M A et al. (Emerg Infect Dis., doi: 10.3201/eid2607.200841 (2020)). This assay detects the production of antibodies that functionally inhibit viral infection demonstrated by a reduction in the number of viral plaques. Slight variations of this assay are described in Gauger P C & Vincent A L (in Animal Influenza Virus: Methods and Protocols, 3rd edition, ed. E. Spackman, pp. 311-320 (2014)) and Wilson H L et al. (J. Clin. Microbiol., 55(10):3104-3112 (2017)). Briefly, a SARS-CoV-2 viral neutralization assay determines the neutralization ability of plasma containing anti-SARS-CoV-2 antibodies produced by mice in response to immunization with circular RNA encoding SARS-CoV-2 antigens. Plasma from naïve mice injected with vehicle only (no circular RNA) is used as a control.

### Example 12: Immunogenicity of SARS-CoV-2 RBD Antigens in Mouse Model

The immunogenicity of a circular RNA encoding a SARS-CoV-2 RBD antigen, formulated with a cationic polymer (e.g., protamine), was evaluated in a mouse model. Production of antibodies to a SARS-CoV-2 RBD antigen, formulated with the cationic polymer, was also evaluated in the mouse model.

In this example, circular RNA was designed with an IRES and ORF encoding a SARS-CoV-2 RBD antigen by the methods described herein. Unmodified linear RNA was synthesized by in vitro transcription with an excess of guanosine 5′ monophosphate using T7 RNA polymerase from a DNA segment. Transcribed RNA was purified with an RNA purification system (New England Biolabs, Inc.) following the manufacturer's instructions. Purified linear RNA was circularized using a splint DNA.

Circular RNA was generated by splint-ligation as follows: Transcribed linear RNA and a DNA splint (5′-GTTTTTCGGCTATTCCCAATAGCCGTTTTG-3′) (SEQ ID NO: 47) were mixed and annealed and treated with an RNA ligase. To purify the circular RNAs, ligation mixtures were resolved by reverse-phase chromatography. Circular RNA was selectively eluted from linear RNA by increasing the organic content of the mobile phase. Eluted RNA was fractionally collected and assayed for circular RNA purity. Selected fractions were combined and buffer exchanged to remove mobile phase salts and solvents. Acrylamide gel electrophoresis was used as a quality control measurement for validating purity and circularization.

The purified circular RNA was diluted in pure water to a concentration of 1100 ng/uL. Protamine sulfate was dissolved in Ringer's lactate solution (4000 ng/uL). While stirring, the protamine-Ringer lactate solution was added to half of the circular RNA solution until a weight ratio of RNA:protamine is 2:1. The solution was stirred for another 10 minutes to ensure the formation of stable complexes. The remaining circular RNA was then added (i.e., remaining circular RNA to circular RNA:protamine solution) and the solution stirred briefly. The final concentration of the mixture (i.e., circular RNA mixture) was adjusted using Ringer's lactate solution to obtain a circular RNA preparation with a final RNA concentration of 2 ug or 10 ug of RNA in 50 uL.

Three mice per group were vaccinated intramuscularly or intradermally with a 2 ug or 10 ug dose of the circular RNA preparation, or a protamine vehicle control at day 0 and day 21. Addavax™ adjuvant (Invivogen) was administered once to each mouse, intramuscularly or intradermally, 24 hours after administration of the circular RNA preparation at day 0 and day 21. Addavax™ adjuvant was dosed at 50% in 1×PBS in 50 uL following to the manufacturer's instructions.

Blood collection from each mouse was by submalar drawing. Blood was collected into dry-anticoagulant free-tubes, at day 7, 14, 21, 23, 28, 35, 41, 49, 56, 63, 69, 77, 84, 108 and 115 days post-dosing of the circular RNA. Serum was separated from whole blood by centrifugation at 1200 g for 30 minutes at 4 C. The serum was heat-inactivated by heating at 56° C. for 1 hour. Individual heat-inactivated serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 uL PBS. The plates were then blocked for 1 hour with blocking buffer (TBS with 2% FBS and 0.05% Tween 20). Serum dilutions were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

The optical density of each serum sample was divided by that of the background (plates coated with RBD, incubated only with secondary antibody). The fold over background of each sample was plotted.

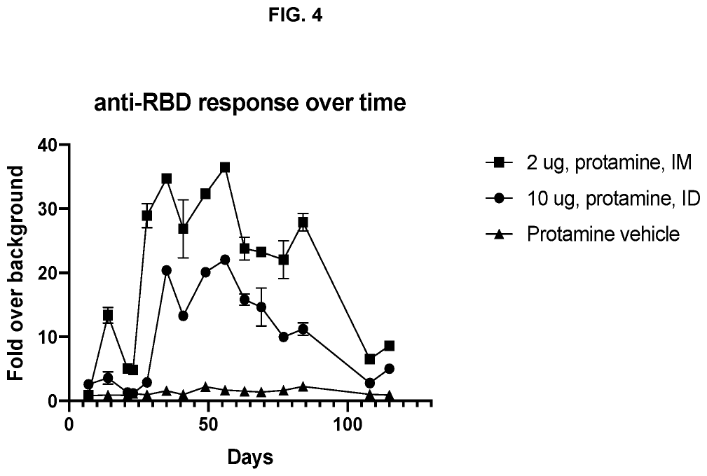

The results showed that anti-RBD antibodies were obtained at days 14, 21, 23, 28, 35, 41, 49, 56, 63, 69, 77, 84, 108 and 115 after injection with the circular RNA preparations (FIG. 4). Anti-RBD antibodies were not obtained after injection with the protamine vehicle. These results also showed that circular RNA encoding the RBD antigen induced an antigen-specific immune response in mice.

A similar ELISA was used to assay serum samples for the presence of Spike-specific IgG. ELISA plates (MaxiSorp 442404 96-well, Nunc) were coated overnight at 4° C. with SARS-CoV-2 Spike (Sino Biological, 40589-V08B1; 100 ng) in 100 uL PBS. The plates were then blocked for 1 hour with blocking buffer (TBS with 2% FBS and 0.05% Tween 20). Serum dilutions were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Jackson 115-035-071) for 1 hour followed by three washes with TBS-T, then addition of tetramethylbenzene (Pierce 34021). The ELISA plate was allowed to react for 5 min and then quenched using 2N sulfuric acid. The optical density (OD) value was determined at 450 nm.

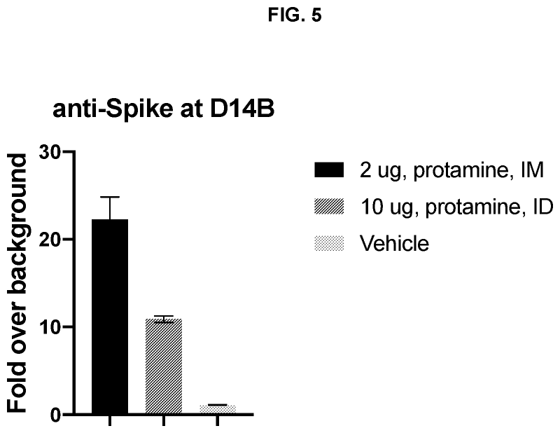

The results showed that anti-Spike antibodies were obtained at 35 days after injection with the circular RNA preparations (FIG. 5). Anti-Spike antibodies were not obtained after injection with vehicle.

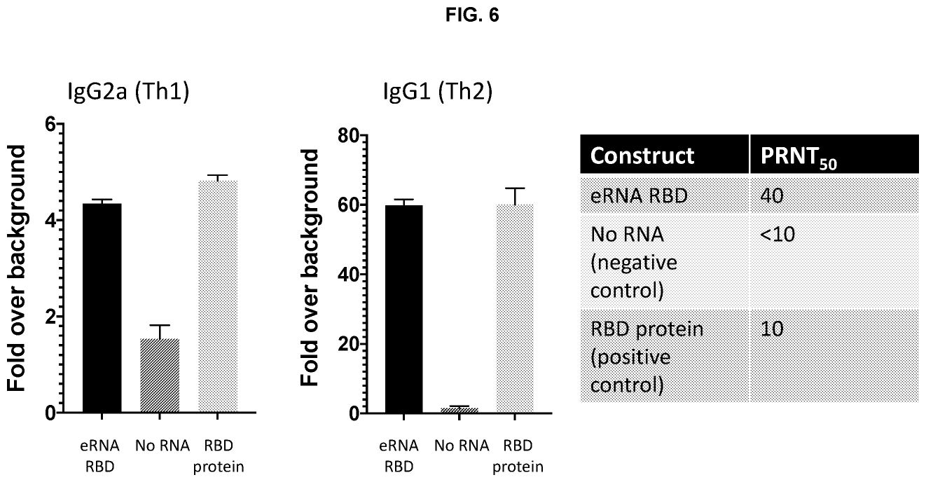

Serum antibodies at day 14 post-dosing were characterized using an assay to measure relative IgG1 vs IgG2a isotypes (FIG. 6), and the ability of serum antibodies to neutralize the virus was characterized using a PRNT neutralization assay. The results showed that 2 ug RBD eRNA dosed intramuscularly with adjuvant had neutralizing ability.

### Example 13: Modulation of In Vivo Production of Gaussia Luciferase from Circular RNA in Mice Using Timed Adjuvant Delivery

This example demonstrates the expression of proteins from circular RNA in vivo whilst also delivering an adjuvant to stimulate an immune response.

In this example, circular RNA was designed with an IRES and ORF encoding a Gaussia Luciferase (GLuc) polypeptide. In this Example, circular RNAs were produced and purified by the methods described herein. Circular RNAs were formulated as described in Example 7 to obtain circular RNA preparations (e.g., TransIT formulated, protamine formulated, PBS/unformulated). Mice are administered each circular RNA preparation intramuscularly as described in Example 8.

To stimulate the immune response, Addavax™ adjuvant (Invivogen), which is a squalene-based oil-in-water nano-emulsion with a formulation similar to MF59® adjuvant, was injected into the mouse hind leg at 0 hours (simultaneous delivery with a circular RNA preparation) or at 24 hours. Addavax™ adjuvant was dosed at 50 uL according to the manufacturer's instructions.

Blood samples (˜25 μL) were collected from each mouse by submalar drawing. Blood was collected into EDTA tubes, at 0, 6, 24 and 48 hours post-dosing of the circular RNA. Plasma was isolated by centrifugation for 30 minutes at 1300 g at 4° C. and the activity of Gaussia Luciferase, a secreted enzyme, was tested using a Gaussia Luciferase activity assay (Thermo Scientific Pierce). 50 μL of 1×GLuc substrate was added to 5 μL of plasma to carry out the GLuc luciferase activity assay. Plates were read immediately after mixing in a luminometer instrument (Promega).

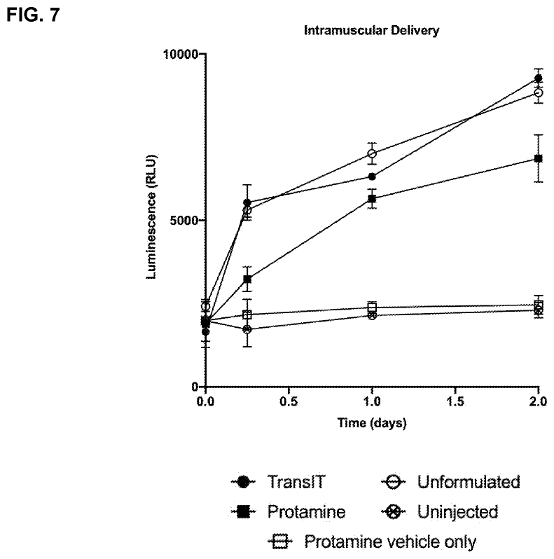

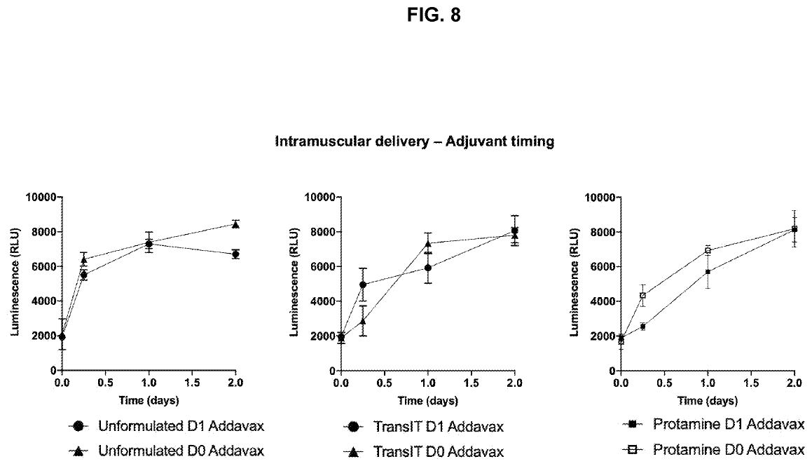

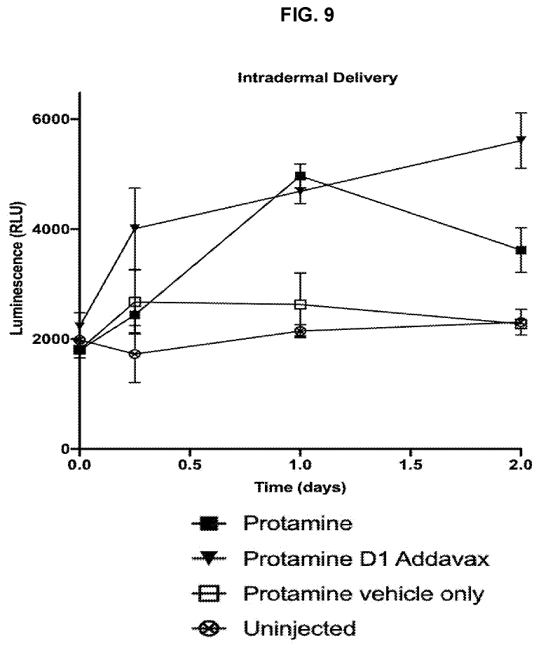

This example demonstrated successful protein expression from circular RNA in vivo for prolonged periods of time using: (a) intramuscular injection of TransIT formulated, protamine formulated and unformulated circular RNA preparations without adjuvant (FIG. 7), and with adjuvant delivered at 0 and 24 h (FIG. 8); and (b) intradermal injection of protamine formulated circular RNA preparation without adjuvant, and with adjuvant delivered at 24 h (FIG. 9).

### Example 14: Administration of RNA Encoding SARS-CoV-2 Antigens to Transchromosomal (Tc) Bovine

This example describes production of fully human neutralizing polyclonal antibodies to a coronavirus antigen in non-human mammals with humanized immune system from circular RNA encoding the coronavirus antigen.

In this Example, circular RNA or mRNA encoding SARS-CoV-2 antigens were designed, and produced and purified by the methods described herein.

In this example, in one approach, RNA is formulated as described in Example 7 (e.g., formulated with a lipid carrier (e.g., TransIT), formulated with a cationic polymer (e.g., protamine) or unformulated), to obtain a first set of circular RNA preparations or a first set of linear RNA preparations. In a second approach, Addavax™ adjuvant (Invivogen), MF59® adjuvant, complete Freund's adjuvant, AS03 or SAB's proprietary adjuvant formulation (SAB-adj-1) is formulated with the RNA-lipid carrier mixture or the unformulated RNA preparation (e.g., circular RNA preparation or linear RNA preparation), as described in Beigel J H et al. (Lancet Infect. Dis., 18: 410-418 (2018)), to obtain a second set of circular RNA preparations or a second set of linear RNA preparations with a final RNA concentration of 25 picomoles in 100 uL. For each approach, a total volume of 8 mL is generated, corresponding to 2 nanomoles of circular RNA or linear RNA. Circular RNA or linear RNA is formulated to obtain the circular RNA preparations or linear RNA preparations shortly before injection into animals.

In this example, Tc bovine are immunized with the circular RNA preparations (i.e., a first circular RNA preparation or a second circular RNA preparation), linear RNA preparations (i.e., a first circular RNA preparation or a second circular RNA preparation) or a vehicle only control (i.e. no RNA control) via intramuscular or intradermal injection.

A. Intramuscular injection: A total of 4 injections are administered at each time point at the following sites: one injections of 2 mL (each) behind each ear; and one injection of 2 mL (each) to either side of the neck.

B. Intradermal injection: A total of 4 injections are administered at each time point at the following sites: four injections of 2 mL to individual sites at the neck-shoulder border.

A total of 8 timepoints are used: 0, 3, 6, 9, 12, 15, 18 and 21 weeks.

Where the first set of RNA preparations (e.g., circular RNA preparations or linear RNA preparations) is administered, Addavax™ adjuvant (Invivogen), MF59® adjuvant, complete Freund's adjuvant, AS03 or SAB's SAB-adj-1 is separately administered adjacent (1-2 cm) to each injection site (2 mL total) for the first 3 timepoints. Prior to the first injection (V1), a volume of pre-injection plasma is collected from each study Tc bovine to be used as negative control. Blood samples, up to 2.1% of the bovine's body weight, are collected via jugular venipuncture at days 8, 9, 10, 11, 12 and 14 days post-injection at each timepoint and at an additional timepoint, 60 days, post-final injection. Plasma is collected using an automated plasmapheresis system (Baxter Healthcare, Autopheresis C Model 200). Plasma is then verified for antigen-specific antibodies using an antigen-based ELISA. Human polyclonal antibodies are purified from the plasma using Cohn-Oncley purification and Caprylate fractionation, for antigen-specific polyclonal antibodies as described below in Example 21).

### Example 15: Detection of a Secreted Antigen Expressed from Circular RNA Administered to Tc Bovine

To detect expression of SARS-CoV-2 RBD antigen, a secreted protein, from circular RNA, blood samples, up to 2.10% of the bovine's body weight, are collected via jugular venipuncture at days 1, 3, 5, 7, 14 and 21 post-injection. Plasma is collected using an automated plasmapheresis system (Baxter Healthcare, Autopheresis C Model 200). Plasma is then verified for expression of SARS-CoV-2 RBD antigens. Expression of RBD antigen is assessed as described in Example 5. For these assays, an anti-human IgG HRP detection antibody (Invitrogen) is used.

### Example 16: Detection of a Non-Secreted Antigen Expressed from Circular RNA Administered to Tc Bovine

To detect expression of SARS-CoV-2 spike antigen, a non-secreted protein, from circular RNA, tissues are harvested for analysis of protein expression. At 0, 2, 5, 7, and 21 days post-dosing, Tc bovine is sacrificed and liver, spleen and muscle (from the site of injection) are harvested. Expression of spike antigen is assessed as described in Example 6 on protein extracted from each tissue. In these ELISAs, an anti-human IgG HRP detection antibody (Invitrogen) is used in place of the anti-mouse IgG HRP detection antibody.

### Example 17: Production of Human Polyclonal Antibodies Specific to SARS-CoV-2 Antigens from Circular RNA Administered to Tc Bovine

To determine the presence of antibodies to SARS-CoV-2 antigens, blood samples, up to 2.1% of the bovine subject's body weight, are collected via jugular venipuncture at days 8, 9, 10, 11, 12, 14, 20, 40, and 60 days post-injection. Plasma is collected using an automated plasmapheresis system (Baxter Healthcare, Autopheresis C Model 200). Plasma is then verified for antigen-specific antibodies. Presence of antibodies to SARS-CoV-2 antigens is determined as described in Example 10. In these assays, an anti-human IgG HRP detection antibody (Invitrogen) is used.

### Example 18: Production of Human Neutralizing Polyclonal Antibodies Against SARS-CoV-2 from Circular RNA Administered to Tc Bovine

Blood samples, up to 2.1% of the bovine subject's body weight, are collected via jugular venipuncture at days 8, 9, 10, 11, 12 and 14 days post-injection at each timepoint and at an additional timepoint, 60 days, post-final injection. Plasma is collected using an automated plasmapheresis system (Baxter Healthcare, Autopheresis C Model 200). Plasma is then verified for antigen-specific antibodies. A SARS-CoV-2 viral neutralization assay is performed to determine the neutralization ability of the antibodies in the plasma as described in Example 11.

### Example 19: Administration of RNA Encoding SARS-CoV-2 Antigens to Transchromosomal (Tc) Bovine with Adjuvant Administration

In this Example, circular RNA or mRNA encoding SARS-CoV-2 antigens were designed, produced, and purified by the methods described herein.

Circular RNA and mRNA are formulated with or without adjuvant as follows:

A. RNA (e.g., circular RNA or mRNA) and adjuvant are administered independently. RNA is formulated as described in Example 7 (e.g., formulated with a lipid carrier (e.g., TransIT), formulated with a cationic polymer (e.g., protamine) or unformulated), to obtain circular RNA preparations or linear RNA preparations. The final RNA concentration is 25 picomoles in 100 uL. Total volume of 8 mL is generated, corresponding to 2 nanomoles of circular RNA or mRNA. Circular RNA or mRNA is formulated shortly before injection into animals. For a total of 8 injections, a total of 64 mL of circular RNA or mRNA is formulated. In this example, Tc bovines are immunized with circular RNA preparations, linear RNA preparations or a vehicle only control (i.e., a no RNA control) via intramuscular injection or intradermal injection.
(i) Intramuscular injection: A total of 4 injections are administered at each time point at the following sites: one injection of 2 mL (each) behind each ear; and one injection of 2 mL (each) to each hind leg.
(ii) Intradermal injection: A total of 4 injections are administered at each time point at the following sited: 4 injections of 2 mL to individual sites at the neck-shoulder border.

A total of 8 timepoints are used: 0, 3, 6, 9, 12, 15, 18 and 21 weeks. Addavax™ adjuvant (Invivogen), MF59® adjuvant, complete Freund's adjuvant, AS03 or SAB's proprietary adjuvant formulation (SAB-adj-1) is administered adjacent (1-2 cm) to each vaccination site (2 mL total) for the first 3 timepoints.

B. RNA (e.g., circular RNA or mRNA) and adjuvant are co-administered. RNA is formulated as described in Example 7 (e.g., formulated with a lipid carrier (e.g., TransIT), formulated with a cationic polymer (e.g., protamine) or unformulated). Addavax™ adjuvant (Invivogen), MF59® adjuvant, complete Freund's adjuvant, AS03 or SAB's proprietary adjuvant formulation (SAB-adj-1) is then formulated with the RNA-carrier formulation, RNA-polymer formulation or unformulated RNA, as described in Beigel J H et al. (Lancet Infect. Dis., 18: 410-418 (2018)), to obtain circular preparations or linear RNA preparations, with a final concentration of RNA of 25 picomoles in 100 uL. Total volume of 8 mL is generated, corresponding to 2 nanomoles of RNA. Circular RNA and mRNA are formulated shortly before injection into animals. For a total of 8 injections, a total of 64 mL of circular RNA and a total of 64 mL of mRNA is formulated.

In this example, Tc bovines are immunized with the circular RNA preparations, linear RNA preparations or a vehicle only control (i.e., a no RNA control) via intramuscular injection or intradermal injection.

A. Intramuscular injection. A total of 4 injections are administered at each time point at the following sites: one injection of 2 mL (each) behind each ear; and one injection of 2 mL (each) to each hind leg.

B. Intradermal injection. A total of 4 injections are administered at each time point at the following sites: 4 injections of 2 mL to individual sites at the neck-shoulder border.

### Example 20: Production of Neutralizing Polyclonal Antibodies Specific to SARS-CoV-2 from Circular RNA in Tc Caprine

In this Example, circular RNA or mRNA encoding SARS-CoV-2 antigens were designed, produced, and purified by the methods described herein.

Circular RNA and mRNA are formulated as described in Example 7 (e.g., formulated with a lipid carrier (e.g., TransIT), formulated with a cationic polymer (e.g., protamine) or unformulated), to obtain circular RNA preparations or linear RNA preparations. The final RNA concentration is 25 picomoles in 100 uL. Total volume of 1 mL is generated, corresponding to 0.25 nanomoles of circular RNA or 0.25 nanomoles of mRNA. Circular RNA and mRNA are formulated to obtain a circular RNA preparations and linear RNA preparations shortly before injection into animals. For a total of 4 injections, a total of 4 mL of circular RNA and a total of 4 mL of linear RNA are formulated.

In this example, a transchromosomal goats (Tc caprine), in which a human artificial chromosome (HAC) comprising the entire human immunoglobulin (Ig) gene repertoire in the germline configuration was introduced into the genetic makeup of the domestic goat, are used. Tc caprine produces human polyclonal antibodies in their sera (see Wu H et al. (Sci Rep, 9(1): 366, doi: doi.org/10.1038/s41598-018-36961-5 (2019)).

In this example, Tc caprine are immunized with circular RNA preparations, linear RNA preparations or a vehicle only control (i.e., a no RNA control) via intramuscular or intradermal injection.

A. Intramuscular injection. A total of 2 injections are administered at each time point at the following sites: one injection of 0.5 mL (each) to either side of the neck.

B. Intradermal injection. A total of 2 injections are administered at each time point at the following sites: one injection of 0.5 mL (each) to opposing sides of the lower neck-shoulder.

A total of 4 timepoints are used: 0, 3, 6 and 9 weeks.

Addavax™ adjuvant (Invivogen), MF59® adjuvant, complete Freund's adjuvant, AS03 or SAB's proprietary adjuvant formulation (SAB-adj-1) is administered adjacent (1-2 cm) to each injection site (0.5 mL total) for the first 3 timepoints.

Blood samples (40 mL) are collected via jugular venipuncture at days 8 and 14 post-injection at each timepoint and at an additional timepoint, 60 days, post-final injection. Plasma is collected using an automated plasmapheresis system (Baxter Healthcare, Autopheresis C Model 200). Plasma is then verified for antigen-specific antibodies.

### Example 21: Purification of Polyclonal Antibodies Fractionation

This example describes purification of human polyclonal antibodies from plasma of non-human mammal with a humanized immune system.

For purification of human anti-SARS-CoV-2 polyclonal antibodies from collected plasma and subsequent use in human subjects, protein antigen-inactivation and removal is required. In this example, human polyclonal anti-SARS-CoV-2 antibodies are purified from plasma using the Cohn-Oncley method as described in (Ofosu et al. FA (Thromb. Haemost., 99(5):851-862 (2008)); Buchacher A and Iberer G (Biotechnol. J., 1(2): 148-163 (2006)); Buchacher A and Curling J M (in Biopharm. Process., Chap 42, pp. 857-876, doi: https://doi.org/10.1016/B978-0-08-100623-8.00043-8 (2018)). Fraction (I+) II+III obtained by the Cohn-Oncley method is collected, and human polyclonal anti-SARS-CoV-2 antibodies are purified from this fraction using methods described by Lebing et al. (Vox Sanguinis, 84(3):193-201 (2003)). Briefly, Fraction II+III is suspended in 12 volumes of water for injection (WFI) at pH 4.2. Sodium caprylate (20 mM) is added and pH is adjusted to pH 5.1 with sodium hydroxide. During this step, lipoproteins, albumin and a portion of caprylate is precipitated. The precipitate is removed by cloth filtration in the presence of filter aid. After filtration, the caprylate concentration is readjusted to 20 mM and the solution is incubated at pH 5.1 for 1 hour at 25° C., to inactivate enveloped virus. The solution is clarified by depth filtration with filter aid. The filtrate is then passed through two successive anion-exchange chromatography columns (Q Sepharose FF followed by ANX Sepharose FF) at pH 5.2. The eluate is concentrated by ultrafiltration (BioMax 50 KDa cassettes, Millipore) and diafiltered against WFI using the same system. The purified IgG solution is adjusted to pH 4.25, 0.2 M glycine and 100 mg/mL protein. Bulk IVIG is sterile filtered and used to fill 10, 50, 100, or 200 mL vials. The final product is incubated for 21 days at 23-27° C. for virus inactivation before storage at 2-8° C.

To verify enrichment of the IVIG, cellulose acetate electrophoresis is used. For clinical use, 95% purity is typical and is expected as a result of this purification procedure.

### Example 22: Formulation of Fully Human Polyclonal Antibodies for Treatment of Human Subjects

In this example, purified antibodies are formulated at neutral pH (pH 7.2) and diluted in an ionic solution containing sodium chloride. A United States Pharmacopoeia (USP) grade infusion solution, 0.9% sodium chloride, is used. The clinical formulation can be based on a few solution compositions which include:
1. Trehalose, sodium citrate, citric acid, polysorbate 80.
2. Sodium succinate, sucrose, polysorbate 20.
3. Sodium chloride, tromethamine, polysorbate 80.
4. Sucrose, sodium chloride, sodium phosphate, dextran 40.

### Example 23: Treatment of Human Subjects Infected with SARS-CoV-2

This example describes administration of fully human anti-SARS-CoV-2 polyclonal antibodies to human subjects with a SARS-CoV-2.

In this example, adult human subjects with COVID-19 are administered a single dose (400 mg/kg) of formulated polyclonal antibodies intravenously by infusion. Infusion is started at a rate of 1.0 mg/kg/min, increasing to 1.5-2.5 mg/kg/min after 20 minutes. Other suitable rates of infusion known in the art can also be used.

The effect of the polyclonal antibodies on COVID-19 is assessed by evaluating markers of COVID-19, such as viral load, serum antibody titer, changes in body temperature, Sequential Organ Failure Assessment (SOFA) score (range 0-24, with higher scores indicating more severe illness), Pao2/Fio2, routine blood biochemical index, ARDS, and ventilatory and extracorporeal membrane oxygenation (ECMO) supports in the human subjects pre- and post-infusion.

### Example 24: Passive Immunization of Healthy Human Subjects Against SARS-CoV-2 Infection

This example describes the passive immunization a human subject from SARS-CoV-2 infection with fully human polyclonal antibodies against SARS-CoV-2 produced in non-human mammals with a humanized immune system.

In this example, healthy human subjects are administered a single dose (400 mg/kg) of formulated polyclonal antibodies or a placebo (normal saline control) intravenously by infusion. Infusion is started at a rate of 1.0 mg/kg/min, increasing to 1.5-2.5 mg/kg/min after 20 minutes. Other suitable rates of infusions known in the art can be used. After 3 days, blood is drawn from treated subjects and plasma is tested for neutralization ability of the antibodies using a plaque-reduction neutralization assay as described in Example 11.

In this example, serological tests are performed on human subjects 14 days after administration of formulated polyclonal antibodies. Serological tests for SARS-CoV-2 are known in the art, including for example, Gonzalez J M et al. medRxiv, (doi: doi.org/10.1101/2020.04.10.20061150 (2020)).

### Example 25: Prophylactic Treatment of Healthy Human Subjects

This example describes the prophylactic treatment of a human subject against SARS-CoV-2 infection with fully human polyclonal antibodies against SARS-CoV-2 produced in non-human mammals with a humanized immune system.

For this Example, purified human polyclonal antibodies against SARS-CoV-2 are obtained as described in Example 21. Purified polyclonal antibodies are formulated as described in Example 22, and subsequently administered to healthy human subjects as described in Example 24.

After 3 days, blood is drawn from healthy human subjects administered the formulated polyclonal antibodies or a placebo (normal saline control) and plasma is tested for neutralization ability of the antibodies using a plaque-reduction neutralization assay as described in Example 11.

### Example 26: Prophylactic Treatment of Non-Human Primates

This example describes the prophylactic treatment of a non-human primate against SARS-CoV-2 infection with fully human polyclonal antibodies against SARS-CoV-2 produced in non-human mammals with a humanized immune system.

In this example, purified human polyclonal antibodies against SARS-CoV-2 are obtained as described in Example 21. Purified polyclonal antibodies are formulated as described in Example 22, and subsequently administered to adult rhesus macaques. Briefly, the polyclonal antibody formulation is administered intravenously at a dose of 10 mg/kg to the rhesus macaques. As a control, polyclonal antibodies from a transchromosomal bovine injected with vehicle only (no circular RNA) are used.

The rhesus macaques are then intratracheally challenged with SARS-CoV-2 at 1×10⁶ 50% tissue-culture infectious doses (TCID₅₀), and the body weight, body temperature, X-ray, sampling of sera, nasal/throat swabs and all primary tissues are carried out on schedule, as described in Bao L et al. (bioRxiv, doi: doi.org/10.1101/2020.03.13.990226 (2020)). Sampling is taken up to 30 days post-challenge and assessed for viral load.

### Example 27: Administration of RNA Encoding SARS-CoV-2 Antigens to a Human Subject

This example describes the administration of a circular RNA encoding a SARS-CoV-2 antigen to a human subject.

In this Example, circular RNA or mRNA encoding SARS-CoV-2 antigens were designed, produced, and purified by the methods described herein.

In this example, in one approach, RNA is formulated as described in Example 7 (e.g., formulated with a lipid carrier (e.g., TransIT), formulated with a cationic polymer (e.g., protamine), formulated with a lipid nanoparticle, or unformulated), to obtain a first set of circular RNA preparations or a first set of linear RNA preparations. In a second approach, Addavax™ adjuvant (Invivogen), MF59® adjuvant, or complete Freund's adjuvant, is formulated with the RNA-lipid carrier mixture or the unformulated RNA preparation (e.g., circular RNA preparation or linear RNA preparation), as described in Beigel J H et al. (Lancet Infect. Dis., 18: 410-418 (2018)), to obtain a second set of circular RNA preparations or a second set of linear RNA preparations. Circular RNA or linear RNA is formulated to obtain the circular RNA preparations or linear RNA preparations shortly before injection into the human subject.

In this example, a human subject is immunized with the circular RNA preparations (i.e., a first circular RNA preparation or a second circular RNA preparation), linear RNA preparations (i.e., a first circular RNA preparation or a second circular RNA preparation) via intramuscular or intradermal injection. The circular RNA preparations or linear RNA preparations are administered to the human subject at least one time, at least two times, at least 3 times, or more to elicit an immunogenic response in the human subject.

### Example 28: Expression of Multiple Antigens from Circular RNAs in Mammalian Cells

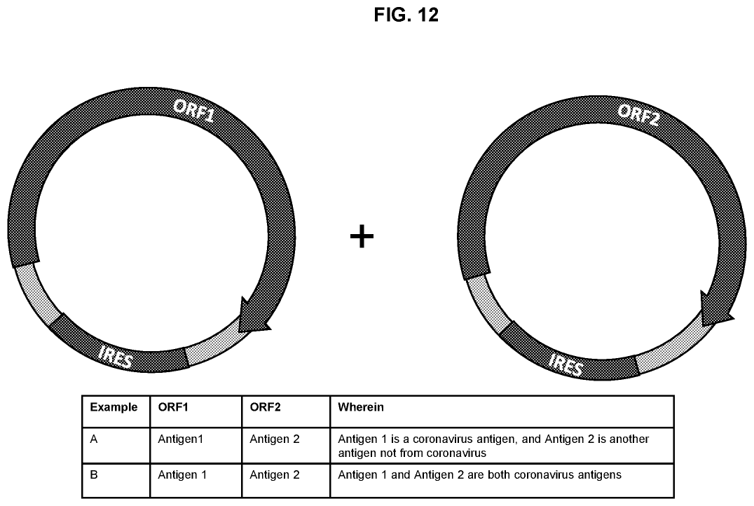

This example demonstrates expression of multiple antigens from circular RNAs in mammalian cells. An exemplary schematic of these constructs is shown in FIG. 12.

### Experiment 1

A first circular RNA encoding a SARS-CoV-2 RBD antigen (Nucleic acid SEQ ID NO: 56; Amino acid SEQ ID NO: 55) was designed, produced, and purified by the methods described herein. A second circular RNA encoding a SARS-CoV-2 Spike antigen (Nucleic acid SEQ ID NO: 54; Amino acid SEQ ID NO. 53) was designed, produced, and purified by the methods described herein. The first circular RNA and the second circular RNA were mixed together to obtain a mixture. The mixture (1 picomoles of each of the circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD antigen expression was measured at 24 hours using a SARS-CoV-2 RBD antigen-specific ELISA. Spike antigen expression was measured at 24 hours by flow cytometry.

From the transfection with the mixture, SARS-Co-V-2 RBD antigen was detected in the HeLa cell supernatant and SARS-CoV-2 Spike antigen was detected on the cell surface of the HeLa cells. From the transfection with the first circular RNA, SARS-CoV-2 RBD antigen was detected, but SARS-CoV-2 Spike antigen was not detected. From the transfection with the second circular RNA, SARS-CoV-2 Spike antigen was detected, but SARS-CoV-2 RBD antigen was not detected. This demonstrates that both SAR-CoV-2 RBD and SARS-CoV-2 Spike antigens were expressed in mammalian cells from a combination mixture of circular RNAs.

### Experiment 2

A first circular RNA encoding a SARS-CoV-2 RBD antigen (Nucleic acid SEQ ID NO: 56; Amino acid SEQ ID NO. 55) was designed, and produced and purified by the methods described herein. A second circular RNA was designed with an IRES and ORF encoding a Gaussia Luciferase (GLuc) polypeptide (Nucleic acid SEQ ID NO: 58; Amino acid SEQ ID NO. 57) as a model antigen, and produced and purified as described by the methods described herein. The first circular RNA and the second circular RNA were separately complexed with Lipofectamine MessengerMax (ThermoFisher, LMRNA015), and then mixed together to obtain a mixture. The mixture (0.1 picomoles of each circular RNAs) was transfected into HeLa cells (20,000 cells per well in a 96 well plate). As controls, the first circular RNA and the second circular RNA were also separately transfected into HeLa cells using MessengerMax.

RBD antigen expression was measured at 24 hours using a SARS-CoV-2 RBD antigen-specific ELISA. GLuc activity was measured at 24 hours using a Gaussia Luciferase activity assay (Thermo Scientific Pierce).

From the transfection with the mixture, SARS-CoV-2 RBD antigen and GLuc activity were detected in the HeLa cell supernatant at 24 hrs. From the transfection with the first circular RNA, SARS-CoV-2 RBD antigen was detected, but GLuc activity was not detected. From the transfection with the second circular RNA, GLuc activity was detected, but SARS-CoV-2 RBD antigen was not detected. This demonstrates that both SAR-CoV-2 RBD and GLuc antigens were expressed in mammalian cells from a combination mixture of circular RNAs.

### Experiment 3

A first circular RNA encoding a SARS-CoV-2 RBD antigen (Nucleic acid SEQ ID NO: 56; Amino acid SEQ ID NO. 55) was designed, produced, and purified by the methods described herein. A second circular RNA was designed to include an IRES followed by an ORF encoding hemagglutinin (HA) antigen from Influenza A H1N1, A/California/07/2009 (Nucleic acid SEQ ID NO: 60; Amino acid SEQ ID NO: 59), and produced and purified by the methods described herein. The first circular RNA and the second circular RNA were mixed together to obtain a mixture. The mixture (1 picomoles of each circular RNA) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

RBD antigen expression was measured at 24 hours using a SARS-CoV-2 RBD antigen-specific ELISA. HA antigen expression was measured at 24 hours using immunoblot. Briefly, for immunoblot, 24 hours after transfection, cells were lysed and Western blot was performed to detect the HA antigen using Influenza A H1N1 HA (A/California/07/2009) monoclonal antibody (MA5-29920 (Thermo Fisher)) as the primary antibody and goat anti-mouse IgG H&L (HRP) as the secondary antibody (Abcam, ab 97023). For loading control alpha tubulin was used with alpha tubulin (DM1A) mouse antibody as the primary antibody (Cell Signaling Technology, CST #3873) and goat anti-mouse IgG H&L (HRP) as the secondary antibody (Abcam, ab 97023).

From the transfection with the mixture, both SARS-CoV-2 RBD and Influenza HA antigens were detected. From the transfection with the first circular RNA, SARS-CoV-2 RBD was detected, but Influenza HA antigen was not detected. From the transfection with the second circular RNA, Influenza HA antigen was detected, but SARS-CoV-2 RBD antigen was not detected. This demonstrates that both SAR-CoV-2 RBD and Influenza HA antigens were expressed in mammalian cells from a combination mixture of circular RNAs.

### Experiment 4

A first circular RNA encoding a SARS-CoV-2 Spike antigen (Nucleic acid SEQ ID NO: 45; Amino acid SEQ ID NO: 53) was designed, produced, and purified by the methods described herein. A second circular RNA was designed to include an IRES followed by an ORF encoding HA from Influenza A H1N1, A/California/07/2009 (Nucleic acid SEQ ID NO: 60; Amino acid SEQ ID NO: 59), and produced and purified by the methods described herein. The first circular RNA and the second circular RNA were mixed together to obtain a mixture. The mixture (1 picomoles of each circular RNAs) was transfected into HeLa cells (100,000 cells per well in a 24 well plate) using Lipofectamine MessengerMax (ThermoFisher, LMRNA015). As controls, the first circular RNA and the second circular RNA were also separately transfected into HeLa cells using MessengerMax.

Spike antigen expression was measured at 24 hours by flow cytometry. HA antigen expression was measured at 24 hours by immunoblot as described above in Experiment 3.

From the transfection with the mixture, both SARS-CoV-2 Spike antigen and Influenza HA antigen were detected. From the transfection with the first circular RNA, SARS-CoV-2 Spike antigen was detected, but Influenza HA antigen was not detected. From the transfection with the second circular RNA, Influenza HA antigen was detected, but SARS-CoV-2 Spike antigen was not detected. This demonstrates that both SAR-CoV-2 Spike and Influenza HA antigens were expressed in mammalian cells from a combination mixture of circular RNAs.

This Example shows that multiple antigens were expressed in mammalian cells from circular RNA preparations comprising different combinations of circular RNAs.

### Example 29: Multi-Antigen Expression from Circular RNA

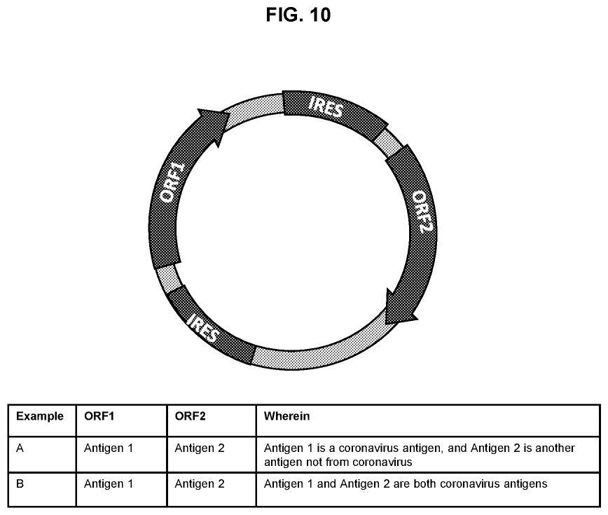

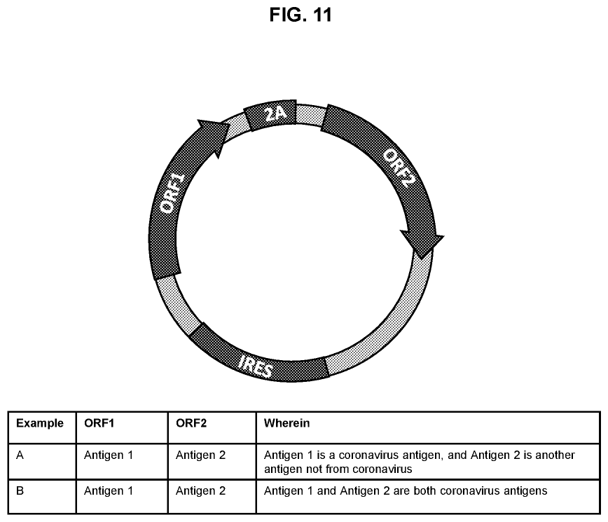

This example demonstrates expression of multiple antigens from a circular RNA in mammalian cells. Exemplary schematics of these constructs are shown in FIGS. 10 and 11.

### Experiment 1

In this Example, a circular RNA was designed to include an IRES followed by an ORF encoding a GLuc polypeptide, a stop codon, a spacer, an IRES, an ORF encoding a SARS-CoV-2 RBD antigen, and a stop codon. The circular RNA was produced and purified by the methods described herein. As controls, the following circular RNAs were produced as described above: (i) a circular RNA with an IRES and ORF encoding a SARS-CoV-2 RBD antigen; (ii) a circular RNA with an IRES and ORF encoding a GLuc polypeptide.

The circular RNAs (0.1 picomoles) were transfected into HeLa cells (10,000 cells per well in a 96 well plate) using Lipofectamine MessengerMax (ThermoFisher, LMRNA015).

RBD antigen expression was measured at 24 hours using a SARS-CoV-2 RBD antigen-specific ELISA. GLuc activity was measured at 24 hours using a Gaussia Luciferase activity assay (Thermo Scientific Pierce).

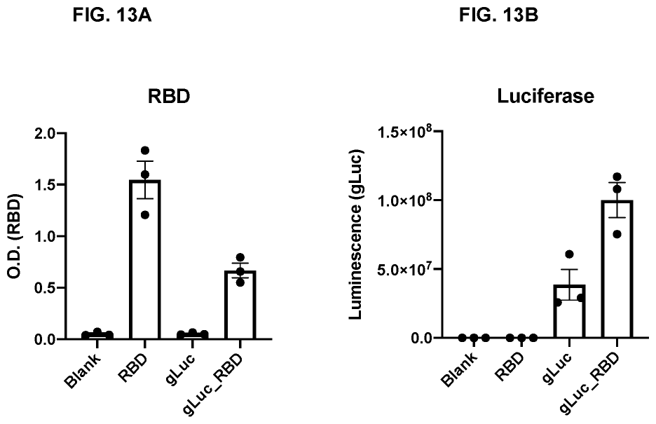

RBD antigen expression was detected from circular RNAs encoding a SARS-CoV-2 RBD antigen and GLuc polypeptide (FIG. 13A). GLuc activity was detected from circular RNAs encoding GLuc polypeptide (FIG. 13B). This demonstrates that both SAR-CoV-2 RBD and GLuc antigens were expressed in mammalian cells from a circular RNA encoding both SARS-CoV-2 RBD and GLuc antigens.

### Experiment 2

In this Example, a circular RNA designed to include an IRES followed by an ORF encoding a SARS-CoV-2 RBD antigen, a stop codon, a spacer, an IRES, an ORF encoding a Middle Eastern Respiratory Syndrome (MERS) RBD antigen, and a stop codon. The circular RNA is produced and purified by the methods described herein.

The circular RNAs are transfected at various concentrations into HeLa cells (10,000 cells per well in a 96 well plate) using Lipofectamine MessengerMax (ThermoFisher, LMRNA015).

SARS-CoV-2 RBD antigen expression is measured at 24 hours using a SARS-CoV-2 RBD antigen-specific ELISA. MERS RBD antigen expression is measured at 24 hours using a MERS RBD antigen specific antibody capable of detection.

### Example 30: Immunogenicity of Multiple Antigens from Circular RNAs in Mouse Model

This example describes expression of multiple antigens in a subject by administrating multiple circular RNA molecules.

### Experiment 1

The immunogenicity of a circular RNA preparation comprising (a) a circular RNA encoding a SARS-CoV-2 RBD antigen and (b) a circular RNA encoding a GLuc polypeptide as a model antigen, formulated in lipid nanoparticles, was evaluated in a mouse model. Production of antibodies to the SARS-CoV-2 RBD antigen and GLuc activity were also evaluated in the mouse model.

A first circular RNA encoding a SARS-CoV-2 RBD antigen (Nucleic acid SEQ ID NO: 56; Amino acid SEQ ID NO: 55) was designed, and produced and purified by the methods described herein. A second circular RNA was designed with an IRES and ORF encoding a GLuc polypeptide (Nucleic acid SEQ ID NO: 58; Amino acid SEQ ID NO. 57), and produced and purified by the methods described herein. The first circular RNA and the second circular RNA were mixed together to obtain a mixture. This mixture was then formulated with lipid nanoparticles as described in Example 7 to obtain a first circular RNA preparation. The first circular RNA and the second circular RNA were also separately formulated with lipid nanoparticles as described in Example 7, and then mixed together to obtain a second circular RNA preparation.

Three mice were vaccinated intramuscularly with the first circular RNA preparation (for a total dose of 10 ug RBD+10 ug GLuc) at day 0 and with the second circular RNA preparation (for a total dose of 10 ug RBD+10 ug GLuc) at day 12. Additional mice (3 or 4 per group) were also vaccinated intramuscularly at day 0 and day 12 with: (i) a 10 ug dose of the first circular RNA formulated with lipid nanoparticles; (ii) a 10 ug dose of the second circular RNA formulated with lipid nanoparticles; or (iii) PBS.

Blood collection from each mouse was by submandibular drawing. Blood was collected into dry-anticoagulant free-tubes, at 2 and 17, days post-priming with the first circular RNA preparation. Serum was separated from whole blood by centrifugation at 1200 g for 30 minutes at 4° C. Individual serum samples were assayed for the presence of RBD-specific IgG by enzyme-linked immunosorbent assay (ELISA). ELISA plates (MaxiSorp 442404 96-well, Nunc) were coated overnight at 4° C. with SARS-CoV-2 RBD (Sino Biological, 40592-V08B; 100 ng) in 100 uL of 1× coating buffer (Biolegend, 421701). The plates were then blocked for 1 hour with blocking buffer (TBS with 2% BSA and 0.05% Tween 20). Serum dilutions (1:500, 1:1500, 1:4500, and 1:13,500) were then added to each well in 100 uL blocking buffer and incubated at room temperature for 1 hour. After washing three times with 1× Tris-buffered saline with Tween® detergent (TBS-T), plates were incubated with anti-mouse IgG HRP detection antibody (Abcam, ab97023) for 1 hour followed by three washes with TBS-T, then addition of tetramethylbenzene (Biolegend, 421101). The ELISA plate was allowed to react for 10-20 minutes and then quenched using 0.2N sulfuric acid. The optical density (O.D.) value was determined at 450 nm.

The optical density of each serum sample was divided by that of the background (plates coated with RBD, incubated only with secondary antibody). The fold over background of each sample was plotted.

The activity of GLuc was tested using a Gaussia Luciferase activity assay (Thermo Scientific Pierce). 50 uL of 1×GLuc substrate was added to 10 uL of serum to carry out the GLuc luciferase activity assay. Plates were read immediately after mixing in a luminometer instrument (Promega).

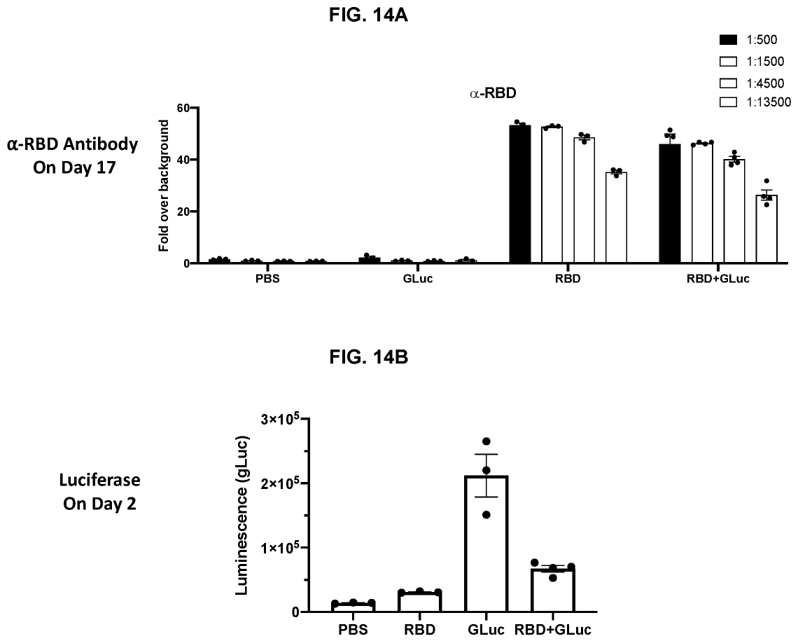

The results showed that anti-RBD antibodies were obtained at 17 days post prime (i.e., 17 days after injection with the first circular RNA preparation) (FIG. 14A) and GLuc activity was detected at 2 days post prime (i.e. 2 days after injection with the first circular RNA preparation) (FIG. 14B).

These results showed that circular RNA preparations comprising two circular RNAs encoding different antigens induced antigen-specific responses in mice.

### Experiment 2

The immunogenicity of a circular RNA preparation comprising (a) a circular RNA encoding a SARS-CoV-2 RBD antigen and (b) a circular RNA encoding an Influenza hemagglutinin (HA) antigen, formulated in lipid nanoparticles, was evaluated in a mouse model. Production of antibodies to the SARS-CoV-2 RBD and Influenza HA antigens were also evaluated in the mouse model.

A first circular RNA encoding a SARS-CoV-2 RBD antigen (Nucleic acid SEQ ID NO: 56; Amino acid SEQ ID NO: 55) was designed, and produced and purified by the methods described herein. A second circular RNA was designed to include an IRES followed by an ORF encoding hemagglutinin (HA) from Influenza A H1N1, A/California/07/2009 (Nucleic acid SEQ ID NO: 60; Amino acid SEQ ID NO: 59), and produced and purified by the methods described herein. The first circular RNA and the second circular RNA were mixed together to obtain a mixture. This mixture was then formulated with lipid nanoparticles as described in Example 7 to obtain a first circular RNA preparation. The first circular RNA and the second circular RNA were also separately formulated with lipid nanoparticles as described in Example 7, and then mixed together to obtain a second circular RNA preparation.

Three mice were vaccinated intramuscularly with the first circular RNA preparation (for a total dose of 10 ug RBD+10 ug HA) at day 0 with the second circular RNA preparation (for a total dose of 10 ug RBD+10 ug HA) and at day 12. Additional mice (3 or 4 per group) were also vaccinated intramuscularly at day 0 and day 12 with: (i) a 10 ug dose of the first circular RNA formulated with lipid nanoparticles; (ii) a 10 ug dose of the second circular RNA formulated with lipid nanoparticles; or (iii) PBS.

Blood collection was as described in Experiment 1. The presence of RBD-specific IgG by ELISA was determined as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

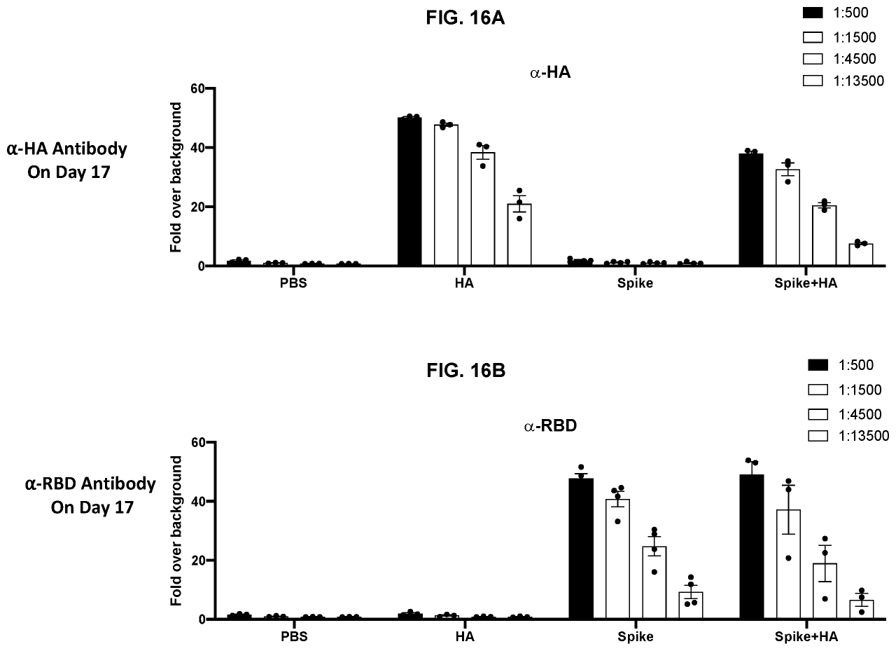

The results showed that anti-RBD and anti-HA antibodies were obtained at 17 days post prime (i.e., 17 days after injection with the first circular RNA preparation (FIGS. 16A and 16B).

The results also showed that circular RNA preparations comprising two circular RNAs encoding different antigens induce an antigen-specific immune response in mice.

Experiment 3

The immunogenicity of a circular RNA preparation comprising (a) a circular RNA encoding a SARS-CoV-2 Spike antigen and (b) a circular RNA encoding an Influenza hemagglutinin (HA) antigen, formulated in lipid nanoparticles, was evaluated in a mouse model. Production of antibodies to the SARS-CoV-2 Spike and Influenza HA antigens were also evaluated in the mouse model.

A first circular RNA encoding a SARS-CoV-2 Spike antigen (Nucleic acid SEQ ID NO: 54; Amino acid SEQ ID NO: 53) was designed, and produced and purified by the methods described herein. A second circular RNA was designed to include an IRES followed by an ORF encoding hemagglutinin (HA) from Influenza A H1N1, A/California/07/2009 (Nucleic acid SEQ ID NO: 60; Amino acid SEQ ID NO: 59), and produced and purified by the methods described herein. The first circular RNA and the second circular RNA were mixed together to obtain a mixture. This mixture was then formulated with lipid nanoparticles as described in Example 7 to obtain a first circular RNA preparation. The first circular RNA and the second circular RNA were also separately formulated with lipid nanoparticles as described in Example 7, and then mixed together to obtain a second circular RNA preparation.

Three mice were vaccinated intramuscularly with the first circular RNA preparation at day 0 (for a total dose of 10 ug Spike+10 ug HA) and with the second circular RNA preparation (for a total dose of 10 ug Spike+10 ug HA) at day 12. Additional mice (3 or 4 per group) were also vaccinated intramuscularly at day 0 and day 12 with: (i) a 10 ug dose of the first circular RNA formulated with lipid nanoparticles; (ii) a 10 ug dose of the second circular RNA formulated with lipid nanoparticles; or (iii) PBS.

Blood collection was as described in Experiment 1 Serum was separated from whole blood by centrifugation at 1200 g for 30 minutes at 40 C. Individual serum samples were assayed for the presence of RBD (i.e., RBD of Spike)-specific IgG by ELISA as described in Experiment 1.

Individual serum samples were assayed for the presence of HA-specific IgG by ELISA. ELISA plates were coated overnight at 4° C. with HA recombinant protein (Sino Biological, 11085-V08B; 100 ng) and plates were processed as described in Experiment 1. The optical density of each serum sample was divided by that of the background (plates coated with HA, incubated only with secondary antibody). The fold over background of each sample was plotted.

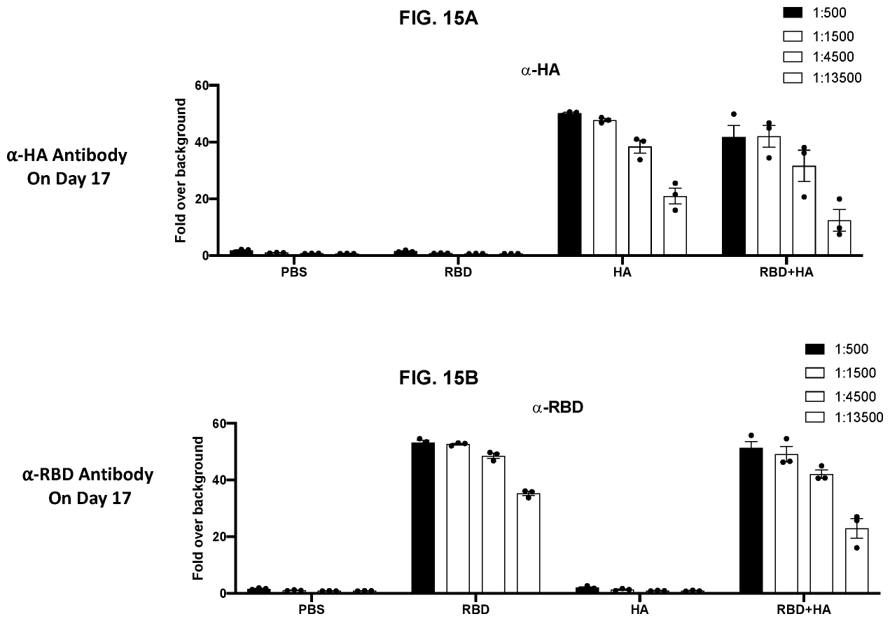

The results showed that anti-RBD antibodies and anti-HA antibodies were obtained at 17 days post prime (i.e., 17 days after injection with the first circular RNA preparation (FIGS. 15A and 15B).

The results also showed that circular RNA preparations comprising two circular RNAs encoding different antigens induced antigen-specific immune responses in mice.

### Example 31: Immunogenicity of a Circular RNA Comprising Multiple Antigens in a Mouse Model

This Example describes the immunogenicity of a circular RNA comprising multiples antigens. This example also describes production of antibodies in a mouse model to multiple antigens encoded by a single circular RNA.

Experiment 1

In this Example, a circular RNA is designed to include an IRES followed by an ORF encoding a GLuc polypeptide (as a model antigen), a stop codon, a spacer, an IRES, an ORF encoding a SARS-CoV-2 RBD antigen, and a stop codon, produced and purified as described in Example 29. As controls, the following circular RNAs are produced as described above: (i) a circular RNA with an IRES and ORF encoding a SARS-CoV-2 RBD antigen; (ii) a circular RNA with an IRES and ORF encoding a GLuc polypeptide.

The circular RNAs are formulated with lipid nanoparticles as described in Example 7 to obtain a circular RNA preparation.

Three mice per group are vaccinated intramuscularly with a 10 ug or 20 ug total dose of circular RNA preparation at day 0 and at day 12.

Blood collection is as described in Example 30. The presence of RBD-specific IgG by ELISA is determined as described in Example 30. GLuc activity is measured as described in Example 30.

Experiment 2

The immunogenicity of a circular RNA preparation comprising a circular RNA designed to include an IRES followed by an ORF encoding a SARS-CoV-2 RBD antigen, a stop codon, a spacer, an IRES, an ORF encoding a MERS RBD antigen, and a stop codon, formulated in lipid nanoparticles, is evaluated in a mouse model. Production of antibodies to the SARS-CoV-2 RBD and MERS RBD antigens are also evaluated in the mouse model.

This circular RNA is then formulated with lipid nanoparticles as described in Example 7 to obtain a circular RNA preparation.

Mice are vaccinated intramuscularly or intradermally with the circular RNA preparation with amounts of 5 μg, 10 μg, 20 μg, or 50 μg at day 0 and again at least one day after the initial administration.

Blood collection is as described in Experiment 1. The presence of SARS-CoV-2 RBD-specific IgG by ELISA is determined as described in Experiment 1. The presence of MERS RBD-specific IgG is also determined by ELISA.

Individual serum samples are assayed for the presence of anti-SARS-CoV-2 RBD binding antibodies, anti-MERS RBD binding antibodies, neutralizing antibodies against the SARS-CoV-2 RBD antigen, neutralizing antibodies against the MERS RBD antigen, a cellular response to the SARS-CoV-2 antigen, and a cellular response to the MERS RBD antigen.

### Example 32: Evaluation of T Cell Responses

An ELISpot assay is used to detect the presence of SARS-CoV-2 Spike or RBD-specific T cells or Influenza HA-specific T cells. This assay is performed on the following groups of mice from Example 30:

1. RBD

2. GLuc

3. HA

4. Spike

5. RBD+HA

6. Spike+HA

7. PBS

Mice spleens are harvested on day 30 post boost (i.e., 30 days after injection with the first circular RNA preparation), and processed into a single cell suspension. Splenocytes are plated at 0.5M cells per well on IFN-g or IL-4 ELISpot plates (ImmunoSpot). Splenocytes are either left unstimulated or stimulated with SARS CoV-2 and HA peptide pools (JPT, PM-WCPV-SRB and PM-IFNA_HACal). ELISPOT plates are processed according to manufacturer's protocol.

### Example 33: Evaluation of Antibody Response in Mice Administered Circular RNA Encoding Multiple Antigens

This example demonstrates an antibody response resulting from administration of a circular RNA encoding the expression of the multiple antigens.

A hemagglutination inhibition assay (HAI) was used to measure anti-Influenza HA antibodies that prevent hemagglutination in serum from mice. Mice were administered a preparation of circular RNA each of which was designed and produced by the methods described herein, and which encode for the expression of: a SARS-CoV-2 RBD antigen, a SARS-CoV-2 Spike antigen, an Influenza HA antigen, a SARS-CoV-2 RBD antigen and an Influenza HA antigen, a SARS-CoV-2 RBD antigen and a GLuc polypeptide, or a SARS-CoV-2 RBD antigen and a SARS-CoV-2 Spike antigen. Blood collection was as described in Example 30, Experiment 1 and was performed on day 2 and day 17 after injection.

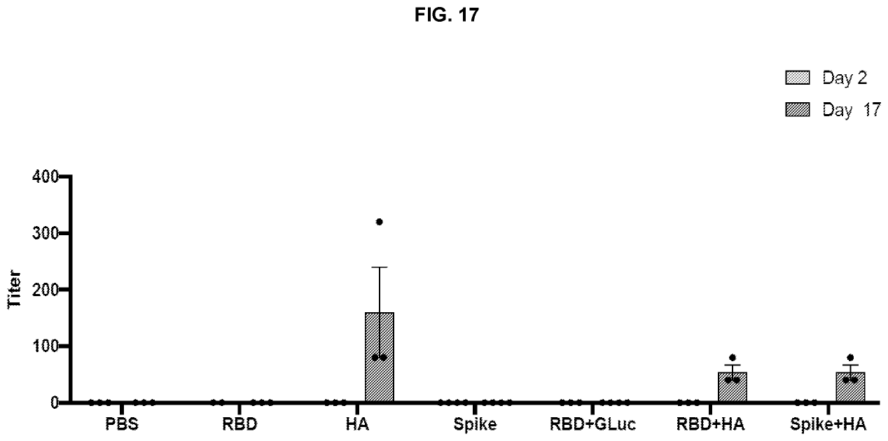

Two-fold serial dilutions of the collected sample from mice on day 2 and day 17 were prepared. A fixed amount of influenza virus with known hemagglutinin (HA) titer was added to every well of a 96-well plate, to a concentration equivalent to 4 hemagglutinin units, with the exception of the serum control wells, where no virus was added. The plate was allowed to stand at room temperature for 60 minutes, after which the red blood cell samples were added and allowed to incubate at 4° C. for 30 minutes. The highest serum dilution that prevented hemagglutination was determined to be the HAI titer of the serum. The sample collected on day 17 showed HAI titer in samples that were administered circular RNA preparations encoding the Influenza HA antigen when it was administered alone or when administered in combination with SARS-CoV-2 antigens e.g. RBD or Spike (FIG. 17). HAI titers on day 17 were not seen from samples where HA antigen had not been administered e.g. the SARS-CoV-2 RBD antigen alone or SARS-CoV-2 Spike antigen alone.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

| SEQ | | |
|---|---|---|
| ID NO: | Comment | Sequence |
| 11 | QHD42416.1 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctcggcgggcacgtagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctaga |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgacaaagttgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacataa |
| 12 | p1 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagGGGTATATCCCTGAAGCCCCCAGGGACGGCCAGGCTTACGTCAGAAAGGATGGAGAGTGGGTGCTCTTGAGC |
| | | ACCTTCCTGTAAAAAAAACAAAAAACAAAACGGCTATTATGCGTTACCGGCGAGACGCTACGGACTTAAATAATTGA |
| | | GCCTTAAAGAAGAAATTCTTTAAGTGGATGCTCTCAAACTCAGGGAAACCTAAATCTAGTTATAGACAAGGCAATCC |
| | | TGAGCCAAGCCGAAGTAGTAATTAGTAAGACCAGTGGACAATCGACGGATAACAGCATATCTAGCTGGGCCTCATGG |
| | | GCCTTCCTTTCACTGCCCGCTTTCCAGTCGGGAAACCTGTCGTGCCAGCTGCATTAACATGGTCATAGCTGTTTCCT |
| | | TGCGTATTGGGCGCTCTCCGCTTCCTCGCTCACTGACTCGCTGCGCTCGGTCGTTCGGGTAAAGCCTGGGGTGCCTA |
| | | ATGAGCAAAAGGCCAGCAAAAGGCCAGGAACCGTAAAAAGGCCGCGTTGCTGGCGTTTTTCCATAGGCTCCGCCCCC |
| | | CTGACGAGCATCACAAAAATCGACGCTCAAGTCAGAGGTGGCGAAACCCGACAGGACTATAAAGATACCAGGCGTTT |
| | | CCCCCTGGAAGCTCCCTCGTGCGCTCTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTGTAGGTCGTTCGCTCCAAGCTGGGCT |
| | | GTGTGCACGAACCCCCCGTTCAGCCCGACCGCTGCGCCTTATCCGGTAACTATCGTCTTGAGTCCAACCCGGTAAGA |
| | | CACGACTTATCGCCACTGGCAGCAGCCACTGGTAACAGGATTAGCAGAGCGAGGTATGTAGGCGGTGCTACAGAGTT |
| | | CTTGAAGTGGTGGCCTAACTACGGCTACACTAGAAGAACAGTATTTGGTATCTGCGCTCTGCTGAAGCCAGTTACCT |
| | | TCGGAAAAAGAGTTGGTAGCTCTTGATCCGGCAAACAAACCACCGCTGGTAGCGGTGGTTTTTTTGTTTGCAAGCAG |
| | | CAGATTACGCGCAGAAAAAAAGGATCTCAAGAAGATCCTTTGATCTTTTCTACGGGGTCTGACGCTCAGTGGAACGA |
| | | AAACTCACGTTAAGGGATTTTGGTCATG |
| 13 | ORF1 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagGGGTATATCCCTGAAGCCCCCAGGGACGGCCAGGCTTACGTCAGAAAGGATGGAGAGTGGGTGCTCTTGAGC |
| | | ACCTTCCTGTAA |
| 14 | p3 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAAAAAAAACAAAAAACAAAACGGCTATTAT |
| | | GCGTTACCGGCGAGACGCTACGGACTTAAATAATTGAGCCTTAAAGAAGAAATTCTTTAAGTGGATGCTCTCAAACT |
| | | CAGGGAAACCTAAATCTAGTTATAGACAAGGCAATCCTGAGCCAAGCCGAAGTAGTAATTAGTAAGACCAGTGGACA |
| | | ATCGACGGATAACAGCATATCTAGCTGGGCCTCATGGGCCTTCCTTTCACTGCCCGCTTTCCAGTCGGGAAACCTGT |
| | | CGTGCCAGCTGCATTAACATGGTCATAGCTGTTTCCTTGCGTATTGGGCGCTCTCCGCTTCCTCGCTCACTGACTCG |
| | | CTGCGCTCGGTCGTTCGGGTAAAGCCTGGGGTGCCTAATGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCAAGTCAGAGGTG |
| | | GCGAAACCCGACAGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCACGCTGTAGGTAT |
| | | CTCAGTTCGGTGTAGGTCGTTCGCTCCAAGCTGGGCTGTGTGCACGAACCCCCCGTTCAGCCCGACCGCTGCGCCTT |
| | | ATCCGGTAACTATCGTCTTGAGTCCAACCCGGTAAGACACGACTTATCGCCACTGGCAGCAGCCACTGGTAACAGGA |
| | | TTAGCAGAGCGAGGTATGTAGGCGGTGCTACAGAGTTCTTGAAGTGGTGGCCTAACTACGGCTACACTAGAAGAACA |
| | | GTATTTGGTATCTGCGCTCTGCTGAAGCCAGTTACCTTCGGAAAAAGAGTTGGTAGCTCTTGATCCGGCAAACAAAC |
| | | CACCGCTGGTAGCGGTGGTTTTTTTGTTTGCAAGCAGCAGATTACGCGCAGAAAAAAAGGATCTCAAGAAGATCCTT |
| | | TGATCTTTTCTACGGGGTCTGACGCTCAGTGGAACGAAAACTCACGTTAAGGGATTTTGGTCATG |
| 15 | ORF3 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAA |
| 16 | p5 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtTAAAAAAAACAAAAAACAAAACGGCTATTATGCGTTACCGGCGAGACGCTACGGACTTAAATAAT |
| | | TGAGCCTTAAAGAAGAAATTCTTTAAGTGGATGCTCTCAAACTCAGGGAAACCTAAATCTAGTTATAGACAAGGCAA |
| | | TCCTGAGCCAAGCCGAAGTAGTAATTAGTAAGACCAGTGGACAATCGACGGATAACAGCATATCTAGCTGGGCCTCA |
| | | TGGGCCTTCCTTTCACTGCCCGCTTTCCAGTCGGGAAACCTGTCGTGCCAGCTGCATTAACATGGTCATAGCTGTTT |
| | | CCTTGCGTATTGGGCGCTCTCCGCTTCCTCGCTCACTGACTCGCTGCGCTCGGTCGTTCGGGTAAAGCCTGGGGTGC |
| | | CTAATGAGCAAAAGGCCAGCAAAAGGCCAGGAACCGTAAAAAGGCCGCGTTGCTGGCGTTTTTCCATAGGCTCCGCC |
| | | CCCCTGACGAGCATCACAAAAATCGACGCTCAAGTCAGAGGTGGCGAAACCCGACAGGACTATAAAGATACCAGGCG |
| | | TTTCCCCCTGGAAGCTCCCTCGTGCGCTCTCCTGTTCCGACCCTGCCGCTTACCGGATACCTGTCCGCCTTTCTCCC |
| | | TTCGGGAAGCGTGGCGCTTTCTCATAGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAACTATCGTCTTGAGTCCAACCCGGTA |
| | | AGACACGACTTATCGCCACTGGCAGCAGCCACTGGTAACAGGATTAGCAGAGCGAGGTATGTAGGCGGTGCTACAGA |
| | | GTTCTTGAAGTGGTGGCCTAACTACGGCTACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGCTGGTAGCGGTGGTTTTTTTGTTTGCAAG |
| | | CAGCAGATTACGCGCAGAAAAAAAGGATCTCAAGAAGATCCTTTGATCTTTTCTACGGGGTCTGACGCTCAGTGGAA |
| | | CGAAAACTCACGTTAAGGGATTTTGGTCATG |
| 17 | ORF5 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtTAA |
| 18 | P7 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagGGGTATATCCCTGAAGCCCCCAGGGACGGCCAGGCTTACGTCAGAAAGGATGGAGAGTGGGTGCTCTTGAGC |
| | | ACCTTCCTGTAAAAAAAACAAAAAACAAAACGGCTATTATGCGTTACCGGCGAGACGCTACGGACTTAAATAATTGA |
| | | GCCTTAAAGAAGAAATTCTTTAAGTGGATGCTCTCAAACTCAGGGAAACCTAAATCTAGTTATAGACAAGGCAATCC |
| | | TGAGCCAAGCCGAAGTAGTAATTAGTAAGACCAGTGGACAATCGACGGATAACAGCATATCTAGCTGGGCCTCATGG |
| | | GCCTTCCTTTCACTGCCCGCTTTCCAGTCGGGAAACCTGTCGTGCCAGCTGCATTAACATGGTCATAGCTGTTTCCT |
| | | TGCGTATTGGGCGCTCTCCGCTTCCTCGCTCACTGACTCGCTGCGCTCGGTCGTTCGGGTAAAGCCTGGGGTGCCTA |
| | | ATGAGCAAAAGGCCAGCAAAAGGCCAGGAACCGTAAAAAGGCCGCGTTGCTGGCGTTTTTCCATAGGCTCCGCCCCC |
| | | CTGACGAGCATCACAAAAATCGACGCTCAAGTCAGAGGTGGCGAAACCCGACAGGACTATAAAGATACCAGGCGTTT |
| | | CCCCCTGGAAGCTCCCTCGTGCGCTCTCCTGTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTTCGGTGTAGGTCGTTCGCTCCAAGCTGGGCT |
| | | GTGTGCACGAACCCCCCGTTCAGCCCGACCGCTGCGCCTTATCCGGTAACTATCGTCTTGAGTCCAACCCGGTAAGA |
| | | CACGACTTATCGCCACTGGCAGCAGCCACTGGTAACAGGATTAGCAGAGCGAGGTATGTAGGCGGTGCTACAGAGTT |
| | | CTTGAAGTGGTGGCCTAACTACGGCTACACTAGAAGAACAGTATTTGGTATCTGCGCTCTGCTGAAGCCAGTTACCT |
| | | TCGGAAAAAGAGTTGGTAGCTCTTGATCCGGCAAACAAACCACCGCTGGTAGCGGTGGTTTTTTTGTTTGCAAGCAG |
| | | CAGATTACGCGCAGAAAAAAAGGATCTCAAGAAGATCCTTTGATCTTTTCTACGGGGTCTGACGCTCAGTGGAACGA |
| | | AAACTCACGTTAAGGGATTTTGGTCATG |
| 19 | ORF7 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagGGGTATATCCCTGAAGCCCCCAGGGACGGCCAGGCTTACGTCAGAAAGGATGGAGAGTGGGTGCTCTTGAGC |
| | | ACCTTCCTGTAA |
| 20 | P9 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAAAAAAAACAAAAAACAAAACGGCTATTAT |
| | | GCGTTACCGGCGAGACGCTACGGACTTAAATAATTGAGCCTTAAAGAAGAAATTCTTTAAGTGGATGCTCTCAAACT |
| | | CAGGGAAACCTAAATCTAGTTATAGACAAGGCAATCCTGAGCCAAGCCGAAGTAGTAATTAGTAAGACCAGTGGACA |
| | | ATCGACGGATAACAGCATATCTAGCTGGGCCTCATGGGCCTTCCTTTCACTGCCCGCTTTCCAGTCGGGAAACCTGT |
| | | CGTGCCAGCTGCATTAACATGGTCATAGCTGTTTCCTTGCGTATTGGGCGCTCTCCGCTTCCTCGCTCACTGACTCG |
| | | CTGCGCTCGGTCGTTCGGGTAAAGCCTGGGGTGCCTAATGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCAAGTCAGAGGTG |
| | | GCGAAACCCGACAGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCACGCTGTAGGTAT |
| | | CTCAGTTCGGTGTAGGTCGTTCGCTCCAAGCTGGGCTGTGTGCACGAACCCCCCGTTCAGCCCGACCGCTGCGCCTT |
| | | ATCCGGTAACTATCGTCTTGAGTCCAACCCGGTAAGACACGACTTATCGCCACTGGCAGCAGCCACTGGTAACAGGA |
| | | TTAGCAGAGCGAGGTATGTAGGCGGTGCTACAGAGTTCTTGAAGTGGTGGCCTAACTACGGCTACACTAGAAGAACA |
| | | GTATTTGGTATCTGCGCTCTGCTGAAGCCAGTTACCTTCGGAAAAAGAGTTGGTAGCTCTTGATCCGGCAAACAAAC |
| | | CACCGCTGGTAGCGGTGGTTTTTTTGTTTGCAAGCAGCAGATTACGCGCAGAAAAAAAGGATCTCAAGAAGATCCTT |
| | | TGATCTTTTCTACGGGGTCTGACGCTCAGTGGAACGAAAACTCACGTTAAGGGATTTTGGTCATG |
| 21 | ORF9 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAA |
| 22 | P11 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtTAAAAAAAACAAAAAACAAAACGGCTATTATGCGTTACCGGCGAGACGCTACGGACTTAAATAAT |
| | | TGAGCCTTAAAGAAGAAATTCTTTAAGTGGATGCTCTCAAACTCAGGGAAACCTAAATCTAGTTATAGACAAGGCAA |
| | | TCCTGAGCCAAGCCGAAGTAGTAATTAGTAAGACCAGTGGACAATCGACGGATAACAGCATATCTAGCTGGGCCTCA |
| | | TGGGCCTTCCTTTCACTGCCCGCTTTCCAGTCGGGAAACCTGTCGTGCCAGCTGCATTAACATGGTCATAGCTGTTT |
| | | CCTTGCGTATTGGGCGCTCTCCGCTTCCTCGCTCACTGACTCGCTGCGCTCGGTCGTTCGGGTAAAGCCTGGGGTGC |
| | | CTAATGAGCAAAAGGCCAGCAAAAGGCCAGGAACCGTAAAAAGGCCGCGTTGCTGGCGTTTTTCCATAGGCTCCGCC |
| | | CCCCTGACGAGCATCACAAAAATCGACGCTCAAGTCAGAGGTGGCGAAACCCGACAGGACTATAAAGATACCAGGCG |
| | | TTTCCCCCTGGAAGCTCCCTCGTGCGCTCTCCTGTTCCGACCCTGCCGCTTACCGGATACCTGTCCGCCTTTCTCCC |
| | | TTCGGGAAGCGTGGCGCTTTCTCATAGCTCACGCTGTAGGTATCTCAGTTCGGTGTAGGTCGTTCGCTCCAAGCTGG |
| | | GCTGTGTGCACGAACCCCCCGTTCAGCCCGACCGCTGCGCCTTATCCGGTAACTATCGTCTTGAGTCCAACCCGGTA |
| | | AGACACGACTTATCGCCACTGGCAGCAGCCACTGGTAACAGGATTAGCAGAGCGAGGTATGTAGGCGGTGCTACAGA |
| | | GTTCTTGAAGTGGTGGCCTAACTACGGCTACACTAGAAGAACAGTATTTGGTATCTGCGCTCTGCTGAAGCCAGTTA |
| | | CCTTCGGAAAAAGAGTTGGTAGCTCTTGATCCGGCAAACAAACCACCGCTGGTAGCGGTGGTTTTTTTGTTTGCAAG |
| | | CAGCAGATTACGCGCAGAAAAAAAGGATCTCAAGAAGATCCTTTGATCTTTTCTACGGGGTCTGACGCTCAGTGGAA |
| | | CGAAAACTCACGTTAAGGGATTTTGGTCATG |
| 23 | ORF11 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatctgtttatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtTAA |
| 24 | P13 | AGATTATCAAAAAGGATCTTCACCTAGATCCTTTTAAATTAAAAATGAAGTTTTAAATCAATCTAAAGTATATATGA |
| | | GTAAACTTGGTCTGACAGTTATTAGAAAAATTCATCCAGCAGACGATAAAACGCAATACGCTGGCTATCCGGTGCCG |
| | | CAATGCCATACAGCACCAGAAAACGATCCGCCCATTCGCCGCCCAGTTCTTCCGCAATATCACGGGTGGCCAGCGCA |
| | | ATATCCTGATAACGATCCGCCACGCCCAGACGGCCGCAATCAATAAAGCCGCTAAAACGGCCATTTTCCACCATAAT |
| | | GTTCGGCAGGCACGCATCACCATGGGTCACCACCAGATCTTCGCCATCCGGCATGCTCGCTTTCAGACGCGCAAACA |
| | | GCTCTGCCGGTGCCAGGCCCTGATGTTCTTCATCCAGATCATCCTGATCCACCAGGCCCGCTTCCATACGGGTACGC |
| | | GCACGTTCAATACGATGTTTCGCCTGATGATCAAACGGACAGGTCGCCGGGTCCAGGGTATGCAGACGACGCATGGC |
| | | ATCCGCCATAATGCTCACTTTTTCTGCCGGCGCCAGATGGCTAGACAGCAGATCCTGACCCGGCACTTCGCCCAGCA |
| | | GCAGCCAATCACGGCCCGCTTCGGTCACCACATCCAGCACCGCCGCACACGGAACACCGGTGGTGGCCAGCCAGCTC |
| | | AGACGCGCCGCTTCATCCTGCAGCTCGTTCAGCGCACCGCTCAGATCGGTTTTCACAAACAGCACCGGACGACCCTG |
| | | CGCGCTCAGACGAAACACCGCCGCATCAGAGCAGCCAATGGTCTGCTGCGCCCAATCATAGCCAAACAGACGTTCCA |
| | | CCCACGCTGCCGGGCTACCCGCATGCAGGCCATCCTGTTCAATCATACTCTTCCTTTTTCAATATTATTGAAGCATT |
| | | TATCAGGGTTATTGTCTCATGAGCGGATACATATTTGAATGTATTTAGAAAAATAAACAAATAGGGGTTCCGCGCAC |
| | | ATTTCCCCGAAAAGTGCCACCTAAATTGTAAGCGTTAATATTTTGTTAAAATTCGCGTTAAATTTTTGTTAAATCAG |
| | | CTCATTTTTTAACCAATAGGCCGAAATCGGCAAAATCCCTTATAAATCAAAAGAATAGACCGAGATAGGGTTGAGTG |
| | | GCCGCTACAGGGCGCTCCCATTCGCCATTCAGGCTGCGCAACTGTTGGGAAGGGCGTTTCGGTGCGGGCCTCTTCGC |
| | | TATTACGCCAGCTGGCGAAAGGGGGATGTGCTGCAAGGCGATTAAGTTGGGTAACGCCAGGGTTTTCCCAGTCACGA |
| | | CGTTGTAAAACGACGGCCAGTGAGCGCGACGTAATACGACTCACTATAGGGCGAATTGAAGGAAGGCCGTCAAGGCC |
| | | GCATGGGAAGCCCTCGACCGTCGATTGTCCACTGGTCAACAATAGATGACTTACAACTAATCGGAAGGTGCAGAGAC |
| | | TCGACGGGAGCTACCCTAACGTCAAGACGAGGGTAAAGAGAGAGTCCAATTCTCAAAGCCAATAGGCAGTAGCGAAA |
| | | GCTGCAAGAGAATGAAAATCCGTTGACCTTAAACGGTCGTGTGGGTTCAAGTCCCTCCACCCCCACGCCGGAAACGC |
| | | AATAGCCGAAAAACAAAAAACAAAAAAAACAAAAAAAAAACCAAAAAAACAAAACACAacgttactggccgaagccg |
| | | cttggaataaggccggtgtgcgtttgtctatatgttattttccaccatattgccgtcttttggcaatgtgagggccc |
| | | ggaaacctggccctgtcttcttgacgagcattcctaggggtctttcccctctcgccaaaggaatgcaaggtctgttg |
| | | aatgtcgtgaaggaagcagttcctctggaagcttcttgaagacaaacaacgtctgtagcgaccctttgcaggcagcg |
| | | gaaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctg |
| | | aaggatgcccagaaggtaccccattgtatgggatctgatctggggcctcggtgcacatgctttacatgtgtttagtc |
| | | gaggttaaaaaacgtctaggccccccgaaccacggggacgtggttttcctttgaaaaacacgatgataataGCCACC |
| | | atgtttgtttttcttgttttattgccactagtctctagtcagtgtagagtccaaccaacagaatctattgttagatt |
| | | tcctaatattacaaacttgtgcccttttggtgaagtttttaacgccaccagatttgcatctgtttatgcttggaaca |
| | | ggaagagaatcagcaactgtgttgctgattattctgtcctatataattccgcatcattttccacttttaagtgttat |
| | | ggagtgtctcctactaaattaaatgatctctgctttactaatgtctatgcagattcatttgtaattagaggtgatga |
| | | agtcagacaaatcgctccagggcaaactggaaagattgctgattataattataaattaccagatgattttacaggct |
| | | gcgttatagcttggaattctaacaatcttgattctaaggttggtggtaattataattacctgtatagattgtttagg |
| | | aagtctaatctcaaaccttttgagagagatatttcaactgaaatctatcaggccggtagcacaccttgtaatggtgt |
| | | tgaaggttttaattgttactttcctttacaatcatatggtttccaacccactaatggtgttggttaccaaccataca |
| | | gagtagtagtactttcttttgaacttctacatgcaccagcaactgtttgtggacctaaaaagtctactaatttggtt |
| | | aaaaacaaatgtgtcaatttcTAAAAAAAACAAAAAACAAAACGGCTATTATGCGTTACCGGCGAGACGCTACGGAC |
| | | TTAAATAATTGAGCCTTAAAGAAGAAATTCTTTAAGTGGATGCTCTCAAACTCAGGGAAACCTAAATCTAGTTATAG |
| | | ACAAGGCAATCCTGAGCCAAGCCGAAGTAGTAATTAGTAAGACCAGTGGACAATCGACGGATAACAGCATATCTAGC |
| | | TGGGCCTCATGGGCCTTCCTTTCACTGCCCGCTTTCCAGTCGGGAAACCTGTCGTGCCAGCTGCATTAACATGGTCA |
| | | TAGCTGTTTCCTTGCGTATTGGGCGCTCTCCGCTTCCTCGCTCACTGACTCGCTGCGCTCGGTCGTTCGGGTAAAGC |
| | | CTGGGGTGCCTAATGAGCAAAAGGCCAGCAAAAGGCCAGGAACCGTAAAAAGGCCGCGTTGCTGGCGTTTTTCCATA |
| | | GGCTCCGCCCCCCTGACGAGCATCACAAAAATCGACGCTCAAGTCAGAGGTGGCGAAACCCGACAGGACTATAAAGA |
| | | TACCAGGCGTTTCCCCCTGGAAGCTCCCTCGTGCGCTCTCCTGTTCCGACCCTGCCGCTTACCGGATACCTGTCCGC |
| | | CTTTCTCCCTTCGGGAAGCGTGGCGCTTTCTCATAGCTCACGCTGTAGGTATCTCAGTTCGGTGTAGGTCGTTCGCT |
| | | CCAAGCTGGGCTGTGTGCACGAACCCCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGATTAGCAGAGCGAGGTATGTAGGCGG |
| | | TGCTACAGAGTTCTTGAAGTGGTGGCCTAACTACGGCTACACTAGAAGAACAGTATTTGGTATCTGCGCTCTGCTGA |
| | | AGCCAGTTACCTTCGGAAAAAGAGTTGGTAGCTCTTGATCCGGCAAACAAACCACCGCTGGTAGCGGTGGTTTTTTT |
| | | GTTTGCAAGCAGCAGATTACGCGCAGAAAAAAAGGATCTCAAGAAGATCCTTTGATCTTTTCTACGGGGTCTGACGC |
| | | TCAGTGGAACGAAAACTCACGTTAAGGGATTTTGGTCATG |
| 25 | ORF13 | atgtttgtttttcttgttttattgccactagtctctagtcagtgtagagtccaaccaacagaatctattgttagatt |
| | | tcctaatattacaaacttgtgcccttttggtgaagtttttaacgccaccagatttgcatctgtttatgcttggaaca |
| | | ggaagagaatcagcaactgtgttgctgattattctgtcctatataattccgcatcattttccacttttaagtgttat |
| | | ggagtgtctcctactaaattaaatgatctctgctttactaatgtctatgcagattcatttgtaattagaggtgatga |
| | | agtcagacaaatcgctccagggcaaactggaaagattgctgattataattataaattaccagatgattttacaggct |
| | | gcgttatagcttggaattctaacaatcttgattctaaggttggtggtaattataattacctgtatagattgtttagg |
| | | aagtctaatctcaaaccttttgagagagatatttcaactgaaatctatcaggccggtagcacaccttgtaatggtgt |
| | | tgaaggttttaattgttactttcctttacaatcatatggtttccaacccactaatggtgttggttaccaaccataca |
| | | gagtagtagtactttcttttgaacttctacatgcaccagcaactgtttgtggacctaaaaagtctactaatttggtt |
| | | aaaaacaaatgtgtcaatttcTAA |
| 26 | ORF33 | atgtttgtttttcttgttttattgccactagtctctagtcagtgtagagtccaaccaacagaatctattgttagatt |
| | | tcctaatattacaaacttgtgcccttttggtgaagtttttaacgccaccagatttgcatccgtgtatgcttggaaca |
| | | ggaagagaatcagcaactgtgttgctgattattctgtcctatataattccgcatcattttccacttttaagtgttat |
| | | ggagtgtctcctactaaattaaatgatctctgctttactaatgtctatgcagattcatttgtaattagaggtgatga |
| | | agtcagacaaatcgctccagggcaaactggaaagattgctgattataattataaattaccagatgattttacaggct |
| | | gcgttatagcttggaattctaacaatcttgattctaaggttggtggtaattataattacctgtatagattgtttagg |
| | | aagtctaatctcaaaccttttgagagagatatttcaactgaaatctatcaggccggtagcacaccttgtaatggtgt |
| | | tgaaggttttaattgttactttcctttacaatcatatggtttccaacccactaatggtgttggttaccaaccataca |
| | | gagtagtagtactttcttttgaacttctacatgcaccagcaactgtttgtggacctaaaaagtctactaatttggtt |
| | | aaaaacaaatgtgtcaatttcTAA |
| 27 | ORF35 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggacttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatttttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatccgtgtatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactggaata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgctattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAA |
| 28 | ORF36 | atgtttgtttttcttgttttattgccactagtctctagtcagtgtgttaatcttacaaccagaactcaattaccccc |
| | | tgcatacactaattctttcacacgtggtgtttattaccctgacaaagttttcagatcctcagttttacattcaactc |
| | | aggatttgttcttacctttcttttccaatgttacttggttccatgctatacatgtctctgggaccaatggtactaag |
| | | aggtttgataaccctgtcctaccatttaatgatggtgtttattttgcttccactgagaagtctaacataataagagg |
| | | ctggatctttggtactactttagattcgaagacccagtccctacttattgttaataacgctactaatgttgttatta |
| | | aagtctgtgaatttcaattttgtaatgatccatttttgggtgtttattaccacaaaaacaacaaaagttggatggaa |
| | | agtgagttcagagtttattctagtgcgaataattgcacttttgaatatgtctctcagccttttcttatggaccttga |
| | | aggaaaacagggtaatttcaaaaatcttagggaatttgtgtttaagaatattgatggttattttaaaatatattcta |
| | | agcacacgcctattaatttagtgcgtgatctccctcagggtttttcggctttagaaccattggtagatttgccaata |
| | | ggtattaacatcactaggtttcaaactttacttgctttacatagaagttatttgactcctggtgattcttcttcagg |
| | | ttggacagctggtgctgcagcttattatgtgggttatcttcaacctaggacttttctattaaaatataatgaaaatg |
| | | gaaccattacagatgctgtagactgtgcacttgaccctctctcagaaacaaagtgtacgttgaaatccttcactgta |
| | | gaaaaaggaatctatcaaacttctaactttagagtccaaccaacagaatctattgttagatttcctaatattacaaa |
| | | cttgtgcccttttggtgaagtttttaacgccaccagatttgcatccgtgtatgcttggaacaggaagagaatcagca |
| | | actgtgttgctgattattctgtcctatataattccgcatcattttccacttttaagtgttatggagtgtctcctact |
| | | aaattaaatgatctctgctttactaatgtctatgcagattcatttgtaattagaggtgatgaagtcagacaaatcgc |
| | | tccagggcaaactggaaagattgctgattataattataaattaccagatgattttacaggctgcgttatagcttgga |
| | | attctaacaatcttgattctaaggttggtggtaattataattacctgtatagattgtttaggaagtctaatctcaaa |
| | | ccttttgagagagatatttcaactgaaatctatcaggccggtagcacaccttgtaatggtgttgaaggttttaattg |
| | | ttactttcctttacaatcatatggtttccaacccactaatggtgttggttaccaaccatacagagtagtagtacttt |
| | | cttttgaacttctacatgcaccagcaactgtttgtggacctaaaaagtctactaatttggttaaaaacaaatgtgtc |
| | | aatttcaacttcaatggtttaacaggcacaggtgttcttactgagtctaacaaaaagtttctgcctttccaacaatt |
| | | tggcagagacattgctgacactactgatgctgtccgtgatccacagacacttgagattcttgacattacaccatgtt |
| | | cttttggtggtgtcagtgttataacaccaggaacaaatacttctaaccaggttgctgttctttatcaggatgttaac |
| | | tgcacagaagtccctgttgctattcatgcagatcaacttactcctacttggcgtgtttattctacaggttctaatgt |
| | | ttttcaaacacgtgcaggctgtttaataggggctgaacatgtcaacaactcatatgagtgtgacatacccattggtg |
| | | caggtatatgcgctagttatcagactcagactaattctcctggcagcgccagcagtgtagctagtcaatccatcatt |
| | | gcctacactatgtcacttggtgcagaaaattcagttgcttactctaataactctattgccatacccacaaattttac |
| | | tattagtgttaccacagaaattctaccagtgtctatgaccaagacatcagtagattgtacaatgtacatttgtggtg |
| | | attcaactgaatgcagcaatcttttgttgcaatatggcagtttttgtacacaattaaaccgtgctttaactgggata |
| | | gctgttgaacaagacaaaaacacccaagaagtttttgcacaagtcaaacaaatttacaaaacaccaccaattaaaga |
| | | ttttggtggttttaatttttcacaaatattaccagatccatcaaaaccaagcaagaggtcatttattgaagatctac |
| | | ttttcaacaaagtgacacttgcagatgctggcttcatcaaacaatatggtgattgccttggtgatattgctgctagg |
| | | gacctcatttgtgcacaaaagtttaacggccttactgttttgccacctttgctcacagatgaaatgattgctcaata |
| | | cacttctgcactgttagcgggtacaatcacttctggttggacctttggtgcaggtgctgcattacaaataccatttg |
| | | ctatgcaaatggcttataggtttaatggtattggagttacacagaatgttctctatgagaaccaaaaattgattgcc |
| | | aaccaatttaatagtgccattggcaaaattcaagactcactttcttccacagcaagtgcacttggaaaacttcaaga |
| | | tgtggtcaaccaaaatgcacaagctttaaacacgcttgttaaacaacttagctccaattttggtgcaatttcaagtg |
| | | ttttaaatgatatcctttcacgtcttgaccctcccgaggctgaagtgcaaattgataggttgatcacaggcagactt |
| | | caaagtttgcagacatatgtgactcaacaattaattagagctgcagaaatcagagcttctgctaatcttgctgctac |
| | | taaaatgtcagagtgtgtacttggacaatcaaaaagagttgatttttgtggaaagggctatcatcttatgtccttcc |
| | | ctcagtcagcacctcatggtgtagtcttcttgcatgtgacttatgtccctgcacaagaaaagaacttcacaactgct |
| | | cctgccatttgtcatgatggaaaagcacactttcctcgtgaaggtgtctttgtttcaaatggcacacactggtttgt |
| | | aacacaaaggaatttttatgaaccacaaatcattactacagacaacacatttgtgtctggtaactgtgatgttgtaa |
| | | taggaattgtcaacaacacagtttatgatcctttgcaacctgaattagactcattcaaggaggagttagataaatat |
| | | tttaagaatcatacatcaccagatgttgatttaggtgacatctctggcattaatgcttcagttgtaaacattcaaaa |
| | | agaaattgaccgcctcaatgaggttgccaagaatttaaatgaatctctcatcgatctccaagaacttggaaagtatg |
| | | agcagtatataaaatggccatggtacatttggctaggttttatagctggcttgattgccatagtaatggtgacaatt |
| | | atgctttgctgtatgaccagttgctgtagttgtctcaagggctgttgttcttgtggatcctgctgcaaatttgatga |
| | | agacgactctgagccagtgctcaaaggagtcaaattacattacacaTAA |
| 29 | ORF39 | ATGTTCGTTTTCCTTGTTTTACTGCCCCTCGTGTCTTCACAGTGTGTGAACCTCACCACTCGCACACAGCTACCTCC |
| | | CGCGTACACTAATTCATTTACCAGGGGCGTCTATTATCCTGATAAGGTGTTCCGGAGTTCAGTGTTGCATAGCACTC |
| | | AAGACCTGTTCCTGCCCTTCTTCTCCAATGTCACTTGGTTTCATGCGATACATGTGTCTGGTACCAACGGAACGAAG |
| | | AGATTTGATAACCCCGTACTGCCATTCAATGATGGCGTATACTTTGCTTCGACTGAAAAATCCAACATCATCAGGGG |
| | | CTGGATTTTTGGTACAACGCTTGATTCCAAGACCCAGTCCCTCCTTATTGTGAACAATGCGACCAACGTCGTGATAA |
| | | AGGTCTGTGAGTTTCAGTTCTGCAATGACCCATTCCTTGGAGTGTATTATCACAAGAACAACAAATCGTGGATGGAG |
| | | TCAGAGTTTAGGGTGTACAGCAGCGCTAACAACTGTACATTTGAGTATGTGAGTCAGCCGTTTCTGATGGATCTTGA |
| | | GGGCAAACAGGGCAATTTTAAGAATCTCAGAGAATTTGTGTTCAAGAACATTGATGGTTACTTTAAGATCTATAGCA |
| | | AACATACGCCAATCAACTTGGTTCGTGATCTGCCACAGGGATTTAGCGCACTGGAACCTCTCGTTGACTTGCCCATA |
| | | GGTATTAACATCACCAGGTTCCAGACGCTCTTGGCATTACACCGTAGTTATCTGACCCCCGGGGACTCCAGTTCCGG |
| | | ATGGACTGCAGGAGCCGCTGCCTACTATGTGGGTTACCTCCAGCCCAGGACCTTTCTTTTGAAATATAACGAGAACG |
| | | GCACAATCACTGATGCTGTGGACTGCGCATTGGATCCTTTGTCAGAGACTAAGTGCACTCTGAAGTCATTCACAGTC |
| | | GAGAAAGGCATTTACCAGACGTCTAACTTCAGGGTTCAGCCTACTGAGTCCATCGTGAGATTCCCAAACATCACAAA |
| | | TCTTTGTCCCTTCGGTGAGGTATTCAATGCGACACGATTTGCCTCAGTGTACGCGTGGAATCGGAAGAGGATCTCCA |
| | | ATTGCGTGGCCGACTACTCCGTCTTATACAACTCAGCTAGCTTTTCAACATTCAAGTGCTATGGCGTGAGCCCTACC |
| | | AAGCTCAATGACCTGTGCTTCACTAATGTGTATGCCGACTCTTTTGTCATTCGCGGCGACGAGGTCCGACAAATCGC |
| | | ACCGGGCCAAACCGGTAAAATTGCCGACTACAACTACAAGCTGCCTGACGACTTCACCGGCTGCGTAATCGCCTGGA |
| | | ACAGCAATAACCTGGATAGCAAAGTGGGCGGAAACTACAACTACCTGTACCGGCTCTTTAGAAAGTCCAACCTGAAA |
| | | CCATTCGAGCGCGATATCTCGACCGAAATCTACCAGGCGGGCAGCACCCCCTGTAATGGTGTAGAAGGGTTCAATTG |
| | | TTACTTTCCACTCCAGAGTTATGGGTTCCAGCCGACCAATGGCGTCGGTTATCAACCATATCGCGTTGTGGTGTTGT |
| | | CCTTTGAGCTGCTACACGCCCCAGCTACAGTGTGCGGGCCAAAGAAAAGCACAAATCTTGTCAAGAACAAATGCGTT |
| | | AACTTTAATTTTAATGGACTCACAGGTACAGGAGTCCTGACCGAATCTAATAAGAAGTTCCTGCCCTTTCAACAGTT |
| | | CGGACGAGACATTGCCGACACCACCGATGCCGTTCGGGACCCACAGACCTTAGAAATTCTGGATATTACTCCATGTA |
| | | GTTTTGGGGGAGTGTCTGTCATCACCCCTGGCACTAATACATCTAACCAGGTTGCAGTCCTCTACCAGGATGTGAAC |
| | | TGTACCGAAGTGCCGGTCGCTATTCACGCAGACCAGCTCACTCCTACCTGGCGGGTGTACTCCACAGGCTCTAACGT |
| | | GTTTCAGACACGTGCAGGGTGCCTAATCGGCGCAGAGCATGTAAATAACTCCTATGAGTGTGATATCCCCATCGGAG |
| | | CCGGGATCTGCGCTTCCTACCAGACACAAACGAATAGTCCCGGATCTGCCTCAAGCGTGGCATCTCAATCCATTATA |
| | | GCATATACGATGTCCCTTGGAGCTGAAAACAGCGTTGCGTATTCAAACAATAGTATCGCTATTCCAACCAATTTTAC |
| | | AATTAGCGTGACCACAGAAATACTCCCTGTGAGCATGACCAAGACCAGTGTAGACTGTACTATGTACATCTGCGGCG |
| | | ACAGTACTGAGTGTAGCAATCTGCTGCTACAGTATGGGTCCTTCTGTACTCAGCTTAATCGGGCTCTCACCGGAATC |
| | | GCTGTAGAGCAAGATAAAAACACACAAGAAGTGTTTGCTCAAGTGAAGCAGATCTATAAGACACCTCCCATCAAGGA |
| | | TTTCGGTGGGTTCAACTTTAGCCAGATTCTGCCCGATCCGTCTAAACCGTCCAAGCGAAGTTTCATCGAAGACCTGC |
| | | TTTTCAATAAGGTCACGCTGGCAGATGCTGGATTTATCAAACAGTACGGCGACTGTCTGGGCGATATCGCCGCAAGA |
| | | GACTTGATATGCGCCCAAAAGTTTAATGGGTTAACCGTCCTTCCACCGCTCCTGACAGACGAGATGATCGCCCAGTA |
| | | TACAAGTGCCTTATTAGCTGGGACCATTACTAGTGGATGGACATTTGGCGCCGGGGCTGCTCTACAGATACCCTTCG |
| | | CCATGCAGATGGCTTACCGCTTCAACGGAATCGGAGTTACCCAGAACGTACTGTACGAAAATCAGAAACTCATAGCT |
| | | AATCAATTTAACTCTGCCATCGGGAAGATTCAGGATTCCCTGTCGTCTACAGCGTCCGCCTTGGGGAAACTGCAAGA |
| | | TGTAGTGAACCAGAACGCCCAGGCCTTAAATACTCTGGTCAAGCAGTTATCTTCAAATTTCGGAGCAATTAGCTCTG |
| | | TGTTGAACGATATTCTTTCCAGGCTGGACCCTCCAGAAGCCGAAGTGCAAATAGACCGGCTCATCACGGGGCGCTTG |
| | | CAAAGCCTGCAAACCTATGTCACCCAGCAACTGATTCGAGCAGCCGAGATCCGGGCCAGTGCTAATCTGGCCGCCAC |
| | | AAAAATGAGCGAGTGCGTCCTCGGGCAGAGCAAACGCGTAGACTTCTGCGGTAAAGGCTATCACCTGATGAGCTTCC |
| | | CTCAGAGCGCACCCCACGGGGTGGTCTTCCTCCACGTTACCTACGTCCCTGCGCAGGAGAAGAACTTCACTACGGCC |
| | | CCTGCAATTTGCCACGATGGCAAGGCCCACTTTCCCAGGGAGGGGGTCTTCGTTTCCAACGGGACTCATTGGTTCGT |
| | | GACTCAGAGAAATTTTTATGAACCTCAGATCATTACCACTGATAATACATTCGTGTCTGGCAACTGTGATGTGGTTA |
| | | TTGGGATAGTTAATAATACGGTATACGACCCACTCCAGCCCGAGCTGGACTCCTTCAAAGAGGAGCTGGACAAGTAC |
| | | TTTAAAAATCACACCTCACCTGATGTGGACCTAGGTGACATATCTGGCATAAATGCTAGCGTGGTTAACATTCAGAA |
| | | GGAAATCGACAGACTCAACGAGGTGGCCAAAAATCTGAACGAGAGTCTGATCGACCTGCAGGAGTTGGGAAAATATG |
| | | AACAGTACATCAAATGGCCATGGTACATCTGGCTGGGCTTCATAGCAGGCCTGATCGCCATCGTCATGGTGACTATT |
| | | ATGCTGTGCTGCATGACATCCTGTTGTAGCTGTTTGAAGGGGTGTTGCTCCTGCGGCTCATGCTGCAAATTCGACGA |
| | | GGACGATTCAGAACCTGTGCTGAAGGGAGTGAAGCTGCATTACACATAA |
| 30 | ORF41 | ATGTTCGTTTTCCTTGTTTTACTGCCCCTCGTGTCTTCACAGTGTAGGGTTCAGCCTACTGAGTCCATCGTGAGATT |
| | | CCCAAACATCACAAATCTTTGTCCCTTCGGTGAGGTATTCAATGCGACACGATTTGCCTCAGTGTACGCGTGGAATC |
| | | GGAAGAGGATCTCCAATTGCGTGGCCGACTACTCCGTCTTATACAACTCAGCTAGCTTTTCAACATTCAAGTGCTAT |
| | | GGCGTGAGCCCTACCAAGCTCAATGACCTGTGCTTCACTAATGTGTATGCCGACTCTTTTGTCATTCGCGGCGACGA |
| | | GGTCCGACAAATCGCACCGGGCCAAACCGGTAAAATTGCCGACTACAACTACAAGCTGCCTGACGACTTCACCGGCT |
| | | GCGTAATCGCCTGGAACAGCAATAACCTGGATAGCAAAGTGGGCGGAAACTACAACTACCTGTACCGGCTCTTTAGA |
| | | AAGTCCAACCTGAAACCATTCGAGCGCGATATCTCGACCGAAATCTACCAGGCGGGCAGCACCCCCTGTAATGGTGT |
| | | AGAAGGGTTCAATTGTTACTTTCCACTCCAGAGTTATGGGTTCCAGCCGACCAATGGCGTCGGTTATCAACCATATC |
| | | GCGTTGTGGTGTTGTCCTTTGAGCTGCTACACGCCCCAGCTACAGTGTGCGGGCCAAAGAAAAGCACAAATCTTGTC |
| | | AAGAACAAATGCGTTAACTTTTAA |
| 31 | EMCV | ACGTTACTGGCCGAAGCCGCTTGGAATAAGGCCGGTGTGCGTTTGTCTATATGTTATTTTCCACCATATTGCCGTCT |
| | IRES | TTTGGCAATGTGAGGGCCCGGAAACCTGGCCCTGTCTTCTTGACGAGCATTCCTAGGGGTCTTTCCCCTCTCGCCAA |
| | | AGGAATGCAAGGTCTGTTGAATGTCGTGAAGGAAGCAGTTCCTCTGGAAGCTTCTTGAAGACAAACAACGTCTGTAG |
| | | CGACCCTTTGCAGGCAGCGGAACCCCCCACCTGGCGACAGGTGCCTCTGCGGCCAAAAGCCACGTGTATAAGATACA |
| | | CCTGCAAAGGCGGCACAACCCCAGTGCCACGTTGTGAGTTGGATAGTTGTGGAAAGAGTCAAATGGCTCTCCTCAAG |
| | | CGTATTCAACAAGGGGCTGAAGGATGCCCAGAAGGTACCCCATTGTATGGGATCTGATCTGGGGCCTCGGTGCACAT |
| | | GCTTTACATGTGTTTAGTCGAGGTTAAAAAACGTCTAGGCCCCCCGAACCACGGGGACGTGGTTTTCCTTTGAAAAA |
| | | CACGATGATAATA |
| 32 | 5′ UTR | ACTCTTCTGGTCCCCACAGACTCAGAGAGAACCCACC |
| | Globin | |
| 33 | 3′ UTR | GCTGGAGCCTCGGTAGCCGTTCCTCCTGCCCGCTGGGCCTCCCAACGGGCCCTCCTCCCCTCCTTGCACCGGCCCTT |
| | Globin | CCTGGTCTTTGAATAAAGTCTGAGTGGGCAGCA |
| 45 | CVB3 | TTAAAACAGCCTGTGGGTTGATCCCACCCACAGGCCCATTGGGCGCTAGCACTCTGGTATCACGGTACCTTTGTGCG |
| | | CCTGTTTTATACCCCCTCCCCCAACTGTAACTTAGAAGTAACACACACCGATCAACAGTCAGCGTGGCACACCAGCC |
| | | ACGTTTTGATCAAGCACTTCTGTTACCCCGGACTGAGTATCAATAGACTGCTCACGCGGTTGAAGGAGAAAGCGTTC |
| | | GTTATCCGGCCAACTACTTCGAAAAACCTAGTAACACCGTGGAAGTTGCAGAGTGTTTCGCTCAGCACTACCCCAGT |
| | | GTAGATCAGGTCGATGAGTCACCGCATTCCCCACGGGCGACCGTGGCGGTGGCTGCGTTGGCGGCCTGCCCATGGGG |
| | | AAACCCATGGGACGCTCTAATACAGACATGGTGCGAAGAGTCTATTGAGCTAGTTGGTAGTCCTCCGGCCCCTGAAT |
| | | GCGGCTAATCCTAACTGCGGAGCACACACCCTCAAGCCAGAGGGCAGTGTGTCGTAACGGGCAACTCTGCAGCGGAA |
| | | CCGACTACTTTGGGTGTCCGTGTTTCATTTTATTCCTATACTGGCTGCTTATGGTGACAATTGAGAGATCGTTACCA |
| | | TATAGCTATTGGATTGGCCATCCGGTGACTAATAGAGCTATTATATATCCCTTTGTTGGGTTTATACCACTTAGCTT |
| | | GAAAGAGGTTAAAACATTACAATTCATTGTTAAGTTGAATACAGCAAA |
| 46 | WT EMCV | CGCGGATCCTAATACGACTCACTATAGGGAATAGCCGAAAAACAAAAAACAAAAAAAACAAAAAAAAAACCAAAAAA |
| | GLuc IS | ACAAAACACAACGTTACTGGCCGAAGCCGCTTGGAATAAGGCCGGTGTGCGTTTGTCTATATGTTATTTTCCACCAT |
| | | ATTGCCGTCTTTTGGCAATGTGAGGGCCCGGAAACCTGGCCCTGTCTTCTTGACGAGCATTCCTAGGGGTCTTTCCC |
| | | CTCTCGCCAAAGGAATGCAAGGTCTGTTGAATGTCGTGAAGGAAGCAGTTCCTCTGGAAGCTTCTTGAAGACAAACA |
| | | ACGTCTGTAGCGACCCTTTGCAGGCAGCGGAACCCCCCACCTGGCGACAGGTGCCTCTGCGGCCAAAAGCCACGTGT |
| | | ATAAGATACACCTGCAAAGGCGGCACAACCCCAGTGCCACGTTGTGAGTTGGATAGTTGTGGAAAGAGTCAAATGGC |
| | | TCTCCTCAAGCGTATTCAACAAGGGGCTGAAGGATGCCCAGAAGGTACCCCATTGTATGGGATCTGATCTGGGGCCT |
| | | CGGTGCACATGCTTTACATGTGTTTAGTCGAGGTTAAAAAACGTCTAGGCCCCCCGAACCACGGGGACGTGGTTTTC |
| | | CTTTGAAAAACACGATGATAATAGCCACCATGGGAGTCAAAGTTCTGTTTGCCCTGATCTGCATCGCTGTGGCCGAG |
| | | GCCAAGCCCACCGAGAACAACGAAGACTTCAACATCGTGGCCGTGGCCAGCAACTTCGCGACCACGGATCTCGATGC |
| | | TGACCGCGGGAAGTTGCCCGGCAAGAAGCTGCCGCTGGAGGTGCTCAAAGAGATGGAAGCCAATGCCCGGAAAGCTG |
| | | GCTGCACCAGGGGCTGTCTGATCTGCCTGTCCCACATCAAGTGCACGCCCAAGATGAAGAAGTTCATCCCAGGACGC |
| | | TGCCACACCTACGAAGGCGACAAAGAGTCCGCACAGGGCGGCATAGGCGAGGCGATCGTCGACATTCCTGAGATTCC |
| | | TGGGTTCAAGGACTTGGAGCCCATGGAGCAGTTCATCGCACAGGTCGATCTGTGTGTGGACTGCACAACTGGCTGCC |
| | | TCAAAGGGCTTGCCAACGTGCAGTGTTCTGACCTGCTCAAGAAGTGGCTGCCGCAACGCTGTGCGACCTTTGCCAGC |
| | | AAGATCCAGGGCCAGGTGGACAAGATCAAGGGGGCCGGTGGTGACTAAAAAAAACAAAAAACAAAACGGCTATT |
| 47 | Splint | GTTTTTCGGCTATTCCCAATAGCCGTTTTG |
| 48 | ORF44 | ATGTACCGGATGCAGTTGTTGTCCTGTATAGCTCTTTCCCTTGCATTGGTCACTAATTCTagagtccaaccaacaga |
| | | atctattgttagatttcctaatattacaaacttgtgcccttttggtgaagtttttaacgccaccagatttgcatccg |
| | | tgtatgcttggaacaggaagagaatcagcaactgtgttgctgattattctgtcctatataattccgcatcattttcc |
| | | acttttaagtgttatggagtgtctcctactaaattaaatgatctctgctttactaatgtctatgcagattcatttgt |
| | | aattagaggtgatgaagtcagacaaatcgctccagggcaaactggaaagattgctgattataattataaattaccag |
| | | atgattttacaggctgcgttatagcttggaattctaacaatcttgattctaaggttggtggtaattataattacctg |
| | | tatagattgtttaggaagtctaatctcaaaccttttgagagagatatttcaactgaaatctatcaggccggtagcac |
| | | accttgtaatggtgttgaaggttttaattgttactttcctttacaatcatatggtttccaacccactaatggtgttg |
| | | gttaccaaccatacagagtagtagtactttcttttgaacttctacatgcaccagcaactgtttgtggacctaaaaag |
| | | tctactaatttggttaaaaacaaatgtgtcaatttcTAA |
| 49 | ORF45 | ATGGGAGTCAAAGTTCTGTTTGCCCTGATCTGCATCGCTGTGGCCGAGGCCAAGagagtccaaccaacagaatctat |
| | | tgttagatttcctaatattacaaacttgtgcccttttggtgaagtttttaacgccaccagatttgcatccgtgtatg |
| | | cttggaacaggaagagaatcagcaactgtgttgctgattattctgtcctatataattccgcatcattttccactttt |
| | | aagtgttatggagtgtctcctactaaattaaatgatctctgctttactaatgtctatgcagattcatttgtaattag |
| | | aggtgatgaagtcagacaaatcgctccagggcaaactggaaagattgctgattataattataaattaccagatgatt |
| | | ttacaggctgcgttatagcttggaattctaacaatcttgattctaaggttggtggtaattataattacctgtataga |
| | | ttgtttaggaagtctaatctcaaaccttttgagagagatatttcaactgaaatctatcaggccggtagcacaccttg |
| | | taatggtgttgaaggttttaattgttactttcctttacaatcatatggtttccaacccactaatggtgttggttacc |
| | | aaccatacagagtagtagtactttcttttgaacttctacatgcaccagcaactgtttgtggacctaaaaagtctact |
| | | aatttggttaaaaacaaatgtgtcaatttcTAA |
| 50 | IL2 | ATGTACCGGATGCAGTTGTTGTCCTGTATAGCTCTTTCCCTTGCATTGGTCACTAATTCT |
| | secretion | |
| | signal | |
| 51 | Glue | ATGGGAGTCAAAGTTCTGTTTGCCCTGATCTGCATCGCTGTGGCCGAGGCCAAG |
| | secretion | |
| | signal | |

1. An immunogenic composition comprising a circular polyribonucleotide comprising a sequence encoding a coronavirus antigen.

2. A immunogenic composition comprising a circular polyribonucleotide comprising a sequence encoding a coronavirus antigen, wherein the coronavirus antigen comprises a sequence having at least about 80%, 85%, 90%, 95%, 97%, 98%, 99%, or 100% sequence identity to a coronavirus antigen selected from any one of SEQ ID NOs: 1-10, 13, 15, 17 19, 21, 23, 25-30, 48, and 49, or the circular polyribonucleotide comprises a sequence having at least about 80%, 85%, 90%, 95%, 97%, 98%, 99%, or 100% sequence identity to a circular polyribonucleotide selected from SEQ ID NOs: 12, 14, 16, 18, 20, 22, and 24.

3. The immunogenic composition of any one of the preceding claims, further comprising the coronavirus antigen.

4. The immunogenic composition of any one of the preceding claims, wherein the coronavirus antigen is from a betacoronavirus or a fragment thereof or a sarbecovirus or a fragment thereof.

5. The immunogenic composition of any one of the preceding claims, wherein the coronavirus antigen is from severe acute respiratory syndrome coronavirus 2 (SARS-CoV-2) or a fragment thereof, severe acute respiratory syndrome coronavirus 1 (SARS-CoV-1) or a fragment thereof, or Middle East respiratory syndrome coronavirus (MERS-CoV) or a fragment thereof.

6. The immunogenic composition of any one of the preceding claims, wherein the coronavirus antigen is a membrane protein or a variant or fragment thereof, an envelope protein of a virus or a variant or fragment thereof, a spike protein of a virus or a variant or fragment thereof, a nucleocapsid protein of a virus or a variant or fragment thereof, an accessory protein of a virus or a variant or fragment thereof.

7. The immunogenic composition of any one of the preceding claims wherein the coronavirus antigen is a receptor binding domain of spike protein or a variant or fragment thereof.

8. The immunogenic composition of claim 7, wherein the spike protein lacks a cleavage site.

9. The immunogenic composition of any one of the preceding claims, wherein an accessory protein of a coronavirus is selected from a group consisting of ORF3a, ORF7a, ORF7b, ORF8, ORF10, or any variant or fragment thereof.

10. The immunogenic composition of any one of the preceding claims, wherein the circular polyribonucleotide comprises a plurality of sequences, each encoding an antigen, and at least one sequence encodes a coronavirus antigen.

11. The immunogenic composition of any one of the preceding claims, wherein the circular polyribonucleotide comprises two or more ORFs.

12. The immunogenic composition of any one of the preceding claims, wherein the circular polyribonucleotide comprises sequences encoding antigens from at least two different microorganisms, and at least one microorganism is a coronavirus.

13. The immunogenic composition of any one of the preceding claims, wherein the coronavirus antigen comprises an epitope.

14. The immunogenic composition of any one of the preceding claims, wherein the coronavirus antigen comprises an epitope recognized by a B cell.

15. The immunogenic composition of any one of the preceding claims, further comprising a second circular polyribonucleotide comprising a sequence encoding a second antigen.

16. The immunogenic composition of any one of the preceding claims, further comprising a second circular polyribonucleotide comprising a second ORF.

17. The immunogenic composition of any one of the preceding claims, further comprising a third, fourth, or fifth circular polyribonucleotide comprising a sequence encoding a third, fourth, or fifth antigen.

18. The immunogenic composition of any one of the preceding claims, wherein the first antigen, second antigen, third antigen, fourth antigen, and fifth antigen are different antigens.

19. The immunogenic composition of any one of the preceding claims, wherein the immunogenic composition further comprises a pharmaceutically acceptable carrier or excipient.

20. The immunogenic composition of any one of the preceding claims, wherein the immunogenic composition further comprises a pharmaceutically acceptable excipient and is free of any carrier.

21. An immunogenic composition comprising a linear polyribonucleotide comprising a sequence selected from any one of SEQ ID NOs: 13, 15, and 12.

22. The immunogenic composition of claim 21, wherein the linear polyribonucleotide comprises sequences encoding two or more antigens and at least one antigen is the coronavirus antigen.

23. The immunogenic composition of claim 21 or claim 22, wherein the linear polyribonucleotide comprises sequences encoding at least 2, 3, 4, or 5 antigens and at least one antigen is a coronavirus antigen encoded by a sequence of SEQ ID NOs: 13, 15, and 12.

24. A method of delivering an immunogenic composition to a human subject comprising: a) administering the immunogenic composition of any one of the preceding claims to the human subject.

25. A method of inducing an immune response against a coronavirus antigen in a non-human animal or human subject comprising: a) administering the immunogenic composition of any one of the preceding claims to the non-human animal or human subject.

26. A method of delivering an immunogenic composition to a human subject comprising: a) administering the immunogenic composition of any one of the preceding claims to the human subject and b) collecting antibodies against the coronavirus antigen from the non-human animal or human subject.

27. A method of inducing an immune response against a coronavirus antigen in a non-human animal or human subject comprising: a) administering the immunogenic composition of any one of the preceding claims to the non-human animal or human subject, and b) collecting antibodies against the coronavirus antigen from the non-human animal or human subject.

28. The method of any one of the preceding claims, further comprising administering an adjuvant to the non-human animal or human subject.

29. The method of claim 28, wherein the adjuvant is co-formulated and co-administered with the immunogenic composition, or is formulated and administered separately from the immunogenic composition.

30. The method of any one of the preceding claims, further comprising formulating the immunogenic composition with a carrier.

31. The method of any one of the preceding claims, further comprising administering or immunizing the circular polyribonucleotide at least two times to the non-human animal or human subject.

32. The method of any one of the preceding claims, further comprising administering or immunizing the non-human animal or human subject with a vaccine.

33. The method of claim 32, wherein the vaccine is pneumococcal polysaccharide vaccine.

34. The method of claim 32, wherein the vaccine is for a bacterial infection.

35. The method of any one of the preceding claims, wherein the non-human animal or human subject is immunized with the circular polyribonucleotide by injection.

36. The method of any one of the preceding claims, wherein an antibody of the polyclonal antibodies specifically binds to the coronavirus antigen.

37. The method of any one of the preceding claims, wherein an antibody of the polyclonal antibodies is a humanized antibody or a fully human antibody.


SEQUENCE LISTING (62 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 3 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 6 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 7 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 8 - protein, 43 aa, Severe acute respiratory syndrome coronavirus 2
MIELSLIDFYLCFLAFLLFLVLIMLIIFWFSLELQDHNETCHA

SEQ ID NO: 9 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 10 - protein, 38 aa, Severe acute respiratory syndrome coronavirus 2
MGYINVFAFPFTIYSLLLCRMNSRNYIAQVDVVNFNLT

SEQ ID NO: 11 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 12 - DNA, 7035 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcaggggtatatccctgaagcccccagggacggccaggcttacgtcagaaaggatggagagtgggtgctcttgagcaccttcctgtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 13 - DNA, 3708 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcaggggtatatccctgaagcccccagggacggccaggcttacgtcagaaaggatggagagtgggtgctcttgagcaccttcctgtaa

SEQ ID NO: 14 - DNA, 7149 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 15 - DNA, 3822 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 16 - DNA, 7038 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgttaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 17 - DNA, 3711 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgttaa

SEQ ID NO: 18 - DNA, 7035 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcaggggtatatccctgaagcccccagggacggccaggcttacgtcagaaaggatggagagtgggtgctcttgagcaccttcctgtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 19 - DNA, 3708 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcaggggtatatccctgaagcccccagggacggccaggcttacgtcagaaaggatggagagtgggtgctcttgagcaccttcctgtaa

SEQ ID NO: 20 - DNA, 7149 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 21 - DNA, 3822 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 22 - DNA, 7038 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgttaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 23 - DNA, 3711 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgttaa

SEQ ID NO: 24 - DNA, 4044 bp, Artificial Sequence
  Synthetic Construct
agattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgggaagccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgtttgtttttcttgttttattgccactagtctctagtcagtgtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatg

SEQ ID NO: 25 - DNA, 717 bp, Artificial Sequence
  Synthetic Construct
atgtttgtttttcttgttttattgccactagtctctagtcagtgtagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctaa

SEQ ID NO: 26 - DNA, 717 bp, Artificial Sequence
  Synthetic Construct
atgtttgtttttcttgttttattgccactagtctctagtcagtgtagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctaa

SEQ ID NO: 27 - DNA, 3822 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 28 - DNA, 3822 bp, Artificial Sequence
  Synthetic Construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggcagcgccagcagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 29 - DNA, 3822 bp, Artificial Sequence
  Synthetic Construct
atgttcgttttccttgttttactgcccctcgtgtcttcacagtgtgtgaacctcaccactcgcacacagctacctcccgcgtacactaattcatttaccaggggcgtctattatcctgataaggtgttccggagttcagtgttgcatagcactcaagacctgttcctgcccttcttctccaatgtcacttggtttcatgcgatacatgtgtctggtaccaacggaacgaagagatttgataaccccgtactgccattcaatgatggcgtatactttgcttcgactgaaaaatccaacatcatcaggggctggatttttggtacaacgcttgattccaagacccagtccctccttattgtgaacaatgcgaccaacgtcgtgataaaggtctgtgagtttcagttctgcaatgacccattccttggagtgtattatcacaagaacaacaaatcgtggatggagtcagagtttagggtgtacagcagcgctaacaactgtacatttgagtatgtgagtcagccgtttctgatggatcttgagggcaaacagggcaattttaagaatctcagagaatttgtgttcaagaacattgatggttactttaagatctatagcaaacatacgccaatcaacttggttcgtgatctgccacagggatttagcgcactggaacctctcgttgacttgcccataggtattaacatcaccaggttccagacgctcttggcattacaccgtagttatctgacccccggggactccagttccggatggactgcaggagccgctgcctactatgtgggttacctccagcccaggacctttcttttgaaatataacgagaacggcacaatcactgatgctgtggactgcgcattggatcctttgtcagagactaagtgcactctgaagtcattcacagtcgagaaaggcatttaccagacgtctaacttcagggttcagcctactgagtccatcgtgagattcccaaacatcacaaatctttgtcccttcggtgaggtattcaatgcgacacgatttgcctcagtgtacgcgtggaatcggaagaggatctccaattgcgtggccgactactccgtcttatacaactcagctagcttttcaacattcaagtgctatggcgtgagccctaccaagctcaatgacctgtgcttcactaatgtgtatgccgactcttttgtcattcgcggcgacgaggtccgacaaatcgcaccgggccaaaccggtaaaattgccgactacaactacaagctgcctgacgacttcaccggctgcgtaatcgcctggaacagcaataacctggatagcaaagtgggcggaaactacaactacctgtaccggctctttagaaagtccaacctgaaaccattcgagcgcgatatctcgaccgaaatctaccaggcgggcagcaccccctgtaatggtgtagaagggttcaattgttactttccactccagagttatgggttccagccgaccaatggcgtcggttatcaaccatatcgcgttgtggtgttgtcctttgagctgctacacgccccagctacagtgtgcgggccaaagaaaagcacaaatcttgtcaagaacaaatgcgttaactttaattttaatggactcacaggtacaggagtcctgaccgaatctaataagaagttcctgccctttcaacagttcggacgagacattgccgacaccaccgatgccgttcgggacccacagaccttagaaattctggatattactccatgtagttttgggggagtgtctgtcatcacccctggcactaatacatctaaccaggttgcagtcctctaccaggatgtgaactgtaccgaagtgccggtcgctattcacgcagaccagctcactcctacctggcgggtgtactccacaggctctaacgtgtttcagacacgtgcagggtgcctaatcggcgcagagcatgtaaataactcctatgagtgtgatatccccatcggagccgggatctgcgcttcctaccagacacaaacgaatagtcccggatctgcctcaagcgtggcatctcaatccattatagcatatacgatgtcccttggagctgaaaacagcgttgcgtattcaaacaatagtatcgctattccaaccaattttacaattagcgtgaccacagaaatactccctgtgagcatgaccaagaccagtgtagactgtactatgtacatctgcggcgacagtactgagtgtagcaatctgctgctacagtatgggtccttctgtactcagcttaatcgggctctcaccggaatcgctgtagagcaagataaaaacacacaagaagtgtttgctcaagtgaagcagatctataagacacctcccatcaaggatttcggtgggttcaactttagccagattctgcccgatccgtctaaaccgtccaagcgaagtttcatcgaagacctgcttttcaataaggtcacgctggcagatgctggatttatcaaacagtacggcgactgtctgggcgatatcgccgcaagagacttgatatgcgcccaaaagtttaatgggttaaccgtccttccaccgctcctgacagacgagatgatcgcccagtatacaagtgccttattagctgggaccattactagtggatggacatttggcgccggggctgctctacagatacccttcgccatgcagatggcttaccgcttcaacggaatcggagttacccagaacgtactgtacgaaaatcagaaactcatagctaatcaatttaactctgccatcgggaagattcaggattccctgtcgtctacagcgtccgccttggggaaactgcaagatgtagtgaaccagaacgcccaggccttaaatactctggtcaagcagttatcttcaaatttcggagcaattagctctgtgttgaacgatattctttccaggctggaccctccagaagccgaagtgcaaatagaccggctcatcacggggcgcttgcaaagcctgcaaacctatgtcacccagcaactgattcgagcagccgagatccgggccagtgctaatctggccgccacaaaaatgagcgagtgcgtcctcgggcagagcaaacgcgtagacttctgcggtaaaggctatcacctgatgagcttccctcagagcgcaccccacggggtggtcttcctccacgttacctacgtccctgcgcaggagaagaacttcactacggcccctgcaatttgccacgatggcaaggcccactttcccagggagggggtcttcgtttccaacgggactcattggttcgtgactcagagaaatttttatgaacctcagatcattaccactgataatacattcgtgtctggcaactgtgatgtggttattgggatagttaataatacggtatacgacccactccagcccgagctggactccttcaaagaggagctggacaagtactttaaaaatcacacctcacctgatgtggacctaggtgacatatctggcataaatgctagcgtggttaacattcagaaggaaatcgacagactcaacgaggtggccaaaaatctgaacgagagtctgatcgacctgcaggagttgggaaaatatgaacagtacatcaaatggccatggtacatctggctgggcttcatagcaggcctgatcgccatcgtcatggtgactattatgctgtgctgcatgacatcctgttgtagctgtttgaaggggtgttgctcctgcggctcatgctgcaaattcgacgaggacgattcagaacctgtgctgaagggagtgaagctgcattacacataa

SEQ ID NO: 30 - DNA, 717 bp, Artificial Sequence
  Synthetic Construct
atgttcgttttccttgttttactgcccctcgtgtcttcacagtgtagggttcagcctactgagtccatcgtgagattcccaaacatcacaaatctttgtcccttcggtgaggtattcaatgcgacacgatttgcctcagtgtacgcgtggaatcggaagaggatctccaattgcgtggccgactactccgtcttatacaactcagctagcttttcaacattcaagtgctatggcgtgagccctaccaagctcaatgacctgtgcttcactaatgtgtatgccgactcttttgtcattcgcggcgacgaggtccgacaaatcgcaccgggccaaaccggtaaaattgccgactacaactacaagctgcctgacgacttcaccggctgcgtaatcgcctggaacagcaataacctggatagcaaagtgggcggaaactacaactacctgtaccggctctttagaaagtccaacctgaaaccattcgagcgcgatatctcgaccgaaatctaccaggcgggcagcaccccctgtaatggtgtagaagggttcaattgttactttccactccagagttatgggttccagccgaccaatggcgtcggttatcaaccatatcgcgttgtggtgttgtcctttgagctgctacacgccccagctacagtgtgcgggccaaagaaaagcacaaatcttgtcaagaacaaatgcgttaacttttaa

SEQ ID NO: 31 - DNA, 552 bp, Artificial Sequence
  Synthetic Construct
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 32 - DNA, 37 bp, Artificial Sequence
  Synthetic Construct
actcttctggtccccacagactcagagagaacccacc

SEQ ID NO: 33 - DNA, 110 bp, Artificial Sequence
  Synthetic Construct
gctggagcctcggtagccgttcctcctgcccgctgggcctcccaacgggccctcctcccctccttgcaccggcccttcctggtctttgaataaagtctgagtgggcagca

SEQ ID NO: 34 - protein, 330 aa, Homo sapiens [sequence omitted]

SEQ ID NO: 35 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 36 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 37 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 39 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 40 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 41 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 42 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 43 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 45 - DNA, 741 bp, Artificial Sequence
  Synthetic Construct
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 46 - DNA, 1229 bp, Artificial Sequence
  Synthetic Construct
cgcggatcctaatacgactcactatagggaatagccgaaaaacaaaaaacaaaaaaaacaaaaaaaaaaccaaaaaaacaaaacacaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctatt

SEQ ID NO: 47 - DNA, 30 bp, Artificial Sequence
  Synthetic Construct
gtttttcggctattcccaatagccgttttg

SEQ ID NO: 48 - DNA, 732 bp, Artificial Sequence
  Synthetic Construct
atgtaccggatgcagttgttgtcctgtatagctctttcccttgcattggtcactaattctagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctaa

SEQ ID NO: 49 - DNA, 726 bp, Artificial Sequence
  Synthetic Construct
atgggagtcaaagttctgtttgccctgatctgcatcgctgtggccgaggccaagagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttctaa

SEQ ID NO: 50 - DNA, 60 bp, Artificial Sequence
  Synthetic Construct
atgtaccggatgcagttgttgtcctgtatagctctttcccttgcattggtcactaattct

SEQ ID NO: 51 - DNA, 54 bp, Artificial Sequence
  Synthetic Construct
atgggagtcaaagttctgtttgccctgatctgcatcgctgtggccgaggccaag

SEQ ID NO: 52 - protein, 8 aa, Artificial Sequence
  Synthetic Construct
  MISC_FEATURE (2)..(2): Xaa is Val or Ile
  MISC_FEATURE (4)..(4): Xaa can be any naturally occurring amino acid
DXEXNPGP

SEQ ID NO: 53 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - DNA, 3819 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggatttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatctttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactgggatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagggacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgccattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcccgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca

SEQ ID NO: 55 - protein, 241 aa, Artificial Sequence
  Synthetic Construct
MGVKVLFALICIAVAEAKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 56 - DNA, 723 bp, Artificial Sequence
  Synthetic Construct
atgggagtcaaagttctgtttgccctgatctgcattgctgtggccgaggccaagagagtccaaccaacagaatctattgttagatttcctaatattacaaacttgtgcccttttggtgaagtttttaacgccaccagatttgcatccgtgtatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 57 - protein, 185 aa, Artificial Sequence
  Synthetic Construct
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 58 - DNA, 558 bp, Artificial Sequence
  Synthetic Construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 59 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 60 - DNA, 1698 bp, Influenza A virus
atgaaagcaatactagtagttcttctatatacatttgcaaccgctaacgctgatacattgtgtataggatatcacgcgaacaactccaccgatacagtagatacagtactagagaagaacgtaacagtaacacattctgttaatcttctagaagacaagcataacggcaaactgtgcaaactaagaggtgtagccccattgcatctaggaaagtgtaatatagctggctggattttgggaaatccagagtgtgaatcattaagtacagcaagctcctggtcctatatagtggaaacacctagtagtgataacggaacgtgttacccaggagattttatagattacgaggagctaagagagcagctgtcgtcagtatcatcatttgaaaggtttgaaattttcccgaaaacatcctcctggcccaatcacgatagtaacaaaggagtaacagcagcctgtcctcacgctggagcaaaaagcttctataaaaatttaatctggctagtgaagaagggaaattcatatccaaagctaagtaaaagttatataaacgataagggcaaggaagtactcgtactgtggggcattcatcatccatctactagtgctgatcaacaaagtttatatcaaaacgcagacgcatacgtttttgtggggtcaagtagatatagcaagaaatttaaaccagaaatagcaataagacctaaagtaagggatcaagaaggcagaatgaactattattggacactagtagaaccgggagataaaataacttttgaagcaacaggaaatctagtggttcccaggtacgcatttgcaatggaaagaaacgctggatcaggcatcattatatctgatacaccagtccacgattgtaatacaacttgtcaaacacctaaaggagctataaacaccagcttaccatttcaaaatattcatcctatcacaattggaaagtgtccaaaatacgtaaaaagtacaaaattgagattggccacaggattacgaaatattccatcaattcaatctagaggactttttggtgcaattgcaggtttcatagaaggaggctggactgggatggtagacggctggtacggttatcatcatcaaaacgaacagggaagtggatacgcagctgatcttaaaagtacacaaaacgcaattgacgagattactaataaagtaaattctgtaattgaaaaaatgaatactcagtttacagcagtagggaaagagtttaaccacctggaaaaaagaatagaaaatttaaataaaaaagtagacgacggatttcttgacatttggacttataacgccgaactattggtattactagaaaacgaaagaactctagattatcacgattcaaacgtaaaaaatttatacgaaaaagtaagaagccaacttaaaaataacgcaaaagaaataggaaacggctgttttgaattttatcacaagtgtgataatacctgcatggaaagtgttaaaaacgggacatacgattatccaaaatactcagaagaagcaaaattaaatagagaagaaatagacggcgtaaaattagaatcaacaaggatatatcaaatattagcaatatattcaactgtcgcttcttcattggtactggtagtttctctaggtgcaatatcattttggatgtgctctaacggctccctacagtgtagaatttgtata

SEQ ID NO: 61 - RNA, 96 nt, Hepatitis D virus
ggcucaucucgacaagaggcggcaguccucaguacucuuacucuuuucuguaaagaggagacugcuggacucgccgcccaaguucgagcaugagcc

SEQ ID NO: 62 - RNA, 74 nt, Hepatitis D virus
ggcuagaggcggcaguccucaguacucuuacucuuuucuguaaagaggagacugcuggacucgccgcccgagcc